(19) United States
(12) Patent Application Publication
(10) Pub. No.: US 2025/0049867 A1
(43) Pub. Date: Feb. 13, 2025

(54) ENGINEERED ONCOLYTIC HERPESVIRUSES

(71) Applicant: VIROGIN BIOTECH CANADA LTD., Richmond (CA)

(72) Inventors: Michael FLOYD, Revere, MA (US); Peter GRZESIK, Seattle, WA (US); Edward M. KENNEDY, Andover, MA (US); Terry FARKALY, Malden, MA (US); Lorena LERNER, Andover, MA (US); Sonia FEAU, Chestnut Hill, MA (US); Craig A. STRATHDEE, Cambridge, MA (US); Christophe QUÉVA, Andover, MA (US)

(21) Appl. No.: 18/686,229

(22) PCT Filed: Aug. 31, 2022
(86) PCT No.: PCT/US2022/075767

§ 371 Date: Feb. 23, 2024

Related U.S. Application Data
(60) Provisional application No. 63/301,419, filed on Jan. 20, 2022.
(60) Provisional application No. 63/278,569, filed on Nov. 12, 2021.
(60) Provisional application No. 63/238,997, filed on Aug. 31, 2021.

(51) Int. Cl. A61K 35/763; C07K 14/52; C07K 14/54; C07K 16/22; C07K 16/28; C12N 7/00; C12N 9/04; C12N 9/26; C12N 9/78; C12N 15/86

(52) U.S. Cl. CPC A61K 35/763; C07K 14/521; C07K 14/5434; C07K 16/22; C07K 16/2818; C12N 7/00; C12N 9/0006; C12N 9/2474; C12N 9/78; C12N 15/86; C07K 2317/565; C07K 2317/76; C12N 2710/16632; C12N 2710/16643; C12N 2710/16645; C12N 2710/16662; C12N 2800/22; C12Y 101/01141; C12Y 302/01035; C12Y 305/04004

(57) ABSTRACT

The present disclosure relates to recombinant oncolytic viral vectors for the treatment and prevention of cancers such as glioblastoma. The oncolytic viral vectors comprise one or more of the following features: miRNA target sequence(s) for viral replication restriction; transgene(s) encoding one or more payload molecules; a retargeting domain; mutation(s) in both UL30 and UL23 genes; and a protein with a syncytial mutation and a counterpart protein without the syncytial mutation.

### CROSS-REFERENCE TO RELATED APPLICATIONS

This application claims the benefit of U.S. Provisional Application No. 63/238,997, filed Aug. 31, 2021, U.S. Provisional Application No. 63/278,569, filed Nov. 12, 2021, and U.S. Provisional Application No. 63/301,419, filed Jan. 20, 2022, all of which are herein incorporated by reference in their entireties.

### REFERENCE TO AN ELECTRONIC SEQUENCE LISTING

The contents of the electronic sequence listing (ONCR_022_02WO_SeqList_ST26.xml; Size: 1,315,454 bytes; and Date of Creation: Aug. 30, 2022) is herein incorporated by reference in its entirety.

### FIELD

The present disclosure relates to recombinant viral vectors for the treatment and prevention of cancer. In some embodiments, the disclosure relates to oncolytic HSV vectors for treating glioblastoma.

### BACKGROUND

Oncolytic viruses, such as oncolytic herpes simplex virus (HSV), preferentially infect cancer cells and have been used in multiple pre-clinical and clinical studies for cancer treatment. But many hurdles remain, including the needs for enhanced safety profile, prolonged antitumor activity, and enhanced ability to overcome the immunosuppressive tumor microenvironment.

Glioblastoma (GBM) is the most common type of primary brain tumor in adults, with a 5-year overall survival of 6.8%. Therapeutic intervention of GBM must overcome three major impediments: drug delivery that is impeded by the blood-brain barrier; a high degree of intra-tumoral heterogeneity with at least three molecular subtypes co-existing within each tumor; and a strong immunosuppressive tumor microenvironment.

There remains a need in the art for improved oncolytic viral vectors. The present disclosure provides such improved oncolytic viral vectors, and more.

### SUMMARY

In one aspect, the disclosure provides recombinant herpesviruses, wherein the viral genome of the recombinant herpesvirus:
(a) comprises one or more transgenes, optionally wherein the open reading frame(s) (ORFs) of the transgene(s) have a G/C content of at least 60%, at least 61%, at least 62%, at least 63%, or at least 64%;
(b) comprises one or more miRNA target sequences;
(c) comprises a polynucleotide encoding a retargeting domain;
(d) comprises a UL30 viral gene encoding a DNA polymerase catalytic subunit (DPCS) and a UL23 viral gene encoding a thymidine kinase (TK), wherein the DPCS comprises a mutation, and wherein the TK comprises a mutation;
(e) encodes a first gB and/or a second gB, wherein the first gB comprises a syncytial mutation, and wherein the second gB comprises no syncytial mutation;
(f) encodes a first gK and/or a second gK, wherein the first gK comprises a syncytial mutation, and wherein the second gK comprises no syncytial mutation;
(g) encodes a first gH and/or a second gH, wherein the first gH comprises a syncytial mutation, and wherein the second gH comprises no syncytial mutation;
(h) encodes a first UL20 and/or a second UL20, wherein the first UL20 comprises a syncytial mutation, and wherein the second UL20 comprises no syncytial mutation; and/or
(i) encodes a first UL24 and/or a second UL24, wherein the first UL24 comprises a syncytial mutation, and wherein the second UL24 comprises no syncytial mutation.

In some embodiments, the recombinant herpesvirus comprises one or more transgenes encoding one or more payload proteins selected from 15-hydroxyprostaglandin dehydrogenase [NAD(+)] (HPGD), adenosine deaminase 2 (ADA2), hyaluronidase-1 (HYAL1), hemotaxis inhibitory protein (CHP), C-C motif chemokine 21 (CCL21), interleukin-12 (IL-12), a CD47 antagonist, a transforming growth factor beta (TGFβ) antagonist, a programmed death-1 (PD1) antagonist, a triggering receptor expressed on myeloid cells-2 (TREM2) antagonist, a biomolecule comprising chlorotoxin (CTX), or any combinations thereof. In some embodiments, the one or more payload proteins comprise or consist of IL-12, a PD1 antagonist, and a TREM2 antagonist. In some embodiments, the one or more payload proteins comprise HPGD. In some embodiments, the one or more payload proteins comprise a biomolecule comprising CTX.

In some embodiments, the one or more payload proteins comprise or consist of one of the combinations of payload proteins listed in Tables 4-7 of the disclosure.

In some embodiments, the one or more payload proteins comprise HPGD. In some embodiments, the one or more payload proteins comprise ADA2. In some embodiments, the one or more payload proteins comprise HYAL1. In some embodiments, the one or more payload proteins comprise CHP. In some embodiments, the one or more payload proteins comprise CCL21. In some embodiments, the one or more payload proteins comprise IL-12. In some embodiments, the one or more payload proteins comprise the CD47 antagonist. In some embodiments, the one or more payload proteins comprise the TGFβ antagonist. In some embodiments, the one or more payload proteins comprise the PD1 antagonist. In some embodiments, the one or more payload proteins comprise the TREM2 antagonist. In some embodiments, the antagonist comprises an antibody or antigen binding fragment thereof. In some embodiments, the one or more payload proteins comprise the biomolecule comprising CTX. In some embodiments, the biomolecule comprising CTX further comprises a T-cell engager moiety specifically binding to a protein expressed on the surface of the T-cell. In some embodiments, the protein expressed on the surface of the T-cell is CD3. In some embodiments, the T-cell engager moiety comprises an amino acid sequence at least 90%, at least 95%, at least 97%, at least 98%, at least 99%, or 100% identical to SEQ ID NO: 914. In some embodiments, the CTX comprises or consists of an amino acid sequence at least 95% identical to SEQ ID NO: 913.

In some embodiments, the HPGD comprises an amino acid sequence at least 90%, at least 95%, at least 97%, at least 98%, at least 99%, or 100% identical to SEQ ID NO: 875. In some embodiments, the ADA2 comprises an amino acid sequence at least 90%, at least 95%, at least 97%, at least 98%, at least 99%, or 100% identical to SEQ ID NO: 877. In some embodiments, the HYAL1 comprises an amino acid sequence at least 90%, at least 95%, at least 97%, at least 98%, at least 99%, or 100% identical to SEQ ID NO: 878. In some embodiments, the CHP comprises an amino acid sequence at least 90%, at least 95%, at least 97%, at least 98%, at least 99%, or 100% identical to SEQ ID NO: 880. In some embodiments, the CCL21 comprises an amino acid sequence at least 90%, at least 95%, at least 97%, at least 98%, at least 99%, or 100% identical to SEQ ID NO: 881. In some embodiments, the IL-12 comprises a subunit alpha comprising an amino acid sequence at least 90%, at least 95%, at least 97%, at least 98%, at least 99%, or 100% identical to SEQ ID NO: 883 and a subunit beta comprising an amino acid sequence at least 90%, at least 95%, at least 97%, at least 98%, at least 99%, or 100% identical to SEQ ID NO: 884. In some embodiments, the CD47 antagonist comprises VHH CDR1 of SEQ ID NO: 895, VHH CDR2 of SEQ ID NO: 896, VHH CDR3 of SEQ ID NO: 897, and/or an amino acid sequence at least 90%, at least 95%, at least 97%, at least 98%, at least 99%, or 100% identical to SEQ ID NO: 887 or 888. In some embodiments, the TGFβ antagonist comprises a heavy chain variable domain (VH) comprising CDR1 of SEQ ID NO: 898, CDR2 of SEQ ID NO: 899, and CDR3 of SEQ ID NO: 900, and/or a light chain variable domain (VL) comprising CDR1 of SEQ ID NO: 901, CDR2 of SEQ ID NO: 902, and CDR3 of SEQ ID NO: 903. In some embodiments, the TGFβ antagonist comprises an amino acid sequence at least 90%, at least 95%, at least 97%, at least 98%, at least 99%, or 100% identical to SEQ ID NO: 889 or 890. In some embodiments, the PD1 antagonist comprises VHH CDR1 of SEQ ID NO: 904, VHH CDR2 of SEQ ID NO: 905, VHH CDR3 of SEQ ID NO: 906, and/or an amino acid sequence at least 90%, at least 95%, at least 97%, at least 98%, at least 99%, or 100% identical to SEQ ID NO: 891 or 892. In some embodiments, the TREM2 antagonist comprises a heavy chain variable domain (VH) comprising CDR1 of SEQ ID NO: 907, CDR2 of SEQ ID NO: 908, CDR3 of SEQ ID NO: 909, and/or an amino acid sequence at least 90%, at least 95%, at least 97%, at least 98%, at least 99%, or 100% identical to SEQ ID NO: 893; and/or a light chain variable domain (VL) comprising CDR1 of SEQ ID NO: 910, CDR2 of SEQ ID NO: 911, CDR3 of SEQ ID NO: 912, and/or an amino acid sequence at least 90%, at least 95%, at least 97%, at least 98%, at least 99%, or 100% identical to SEQ ID NO: 894. In some embodiments, the biomolecule comprising CTX comprises an amino acid sequence at least 90%, at least 95%, at least 97%, at least 98%, at least 99%, or 100% identical to SEQ ID NO: 915 or 916.

In some embodiments, the ORF of at least one of the transgene(s) has G/C content of at least 60%, at least 61%, at least 62%, at least 63%, or at least 64%. In some embodiments, the ORFs of all of the transgene(s) have G/C content of at least 60%, at least 61%, at least 62%, at least 63%, or at least 64%. In some embodiments, the ORFs of the transgene(s) encoding IL-12, the PD1 antagonist, the TREM2 antagonist, HPGD, and/or the biomolecule comprising CTX have a G/C content of at least 60%, at least 61%, at least 62%, at least 63%, or at least 64%. In some embodiments, the expression of a payload protein encoded by the ORF of the transgene is at least 2-fold, at least 3-fold, at least 5-fold, at least 8-fold, or at least 10-fold higher than the expression of the payload protein encoded by a control ORF having a G/C content of about 52% in a control recombinant herpesvirus. In some embodiments, the control ORF is codon optimized based on the codon usage of Homo sapiens. In some embodiments, the ORF(s) of the transgene(s) are codon optimized based on the codon usage of Anaeromyxobacter dehalogenans. In some embodiments, the transgene(s) encode an antibody or antigen binding fragment thereof. In some embodiments, the antibody or antigen binding fragment thereof comprises a heavy chain variable domain (VH) and a light chain variable domain (VL). In some embodiments, the transgene encoding the TREM2 antagonist comprises a polynucleotide sequence at least 90%, at least 95%, at least 97%, at least 98%, at least 99%, or 100% identical to SEQ ID NO: 938. In some embodiments, the antibody or antigen binding fragment thereof comprises a VHH domain derived from a single domain antibody (sdAb). In some embodiments, the antibody or antigen binding fragment thereof comprises an IgG-Fc. In some embodiments, the IgG is IgG1. In some embodiments, the transgene encoding the PD1 antagonist comprises a polynucleotide sequence at least 90%, at least 95%, at least 97%, at least 98%, at least 99%, or 100% identical to SEQ ID NO: 937. In some embodiments, the transgene encoding the biomolecule comprising CTX comprises a polynucleotide sequence at least 90%, at least 95%, at least 97%, at least 98%, at least 99%, or 100% identical to SEQ ID NO: 940 or 941. In some embodiments, the transgene(s) encode cytokine, a chemokine, a receptor, a receptor ligand, an enzyme, and/or a reporter protein. In some embodiments, the transgene encoding IL-12 comprise a polynucleotide sequence at least 90%, at least 95%, at least 97%, at least 98%, at least 99%, or 100% identical to SEQ ID NO: 936. In some embodiments, the transgene encoding HPGD comprises a polynucleotide sequence at least 90%, at least 95%, at least 97%, at least 98%, at least 99%, or 100% identical to SEQ ID NO: 939.

In some embodiments, the recombinant herpesvirus comprises the miRNA target sequences for miR-34b-5p, miR-34b-3p, miR-34c-5p, miR-122-5p, miR-124-3p, miR-128T, miR-129-5p, miR-129-2-3p, miR-132-3p, miR-137-3p, miR-145-5p, or any combination thereof. In some embodiments, the recombinant herpesvirus comprises the miRNA target sequences for miR-34b-5p, miR-34c-5p, miR-122-5p, miR-124-3p, miR-128T, miR-129-2-3p, miR-132-3p, miR-137-3p, miR-145-5p, or any combination thereof. In some embodiments, the recombinant herpesvirus comprises the miRNA target sequences for miR-34b-5p, miR-34c-5p, miR-122-5p, miR-124-3p, miR-128T, miR-129-2-3p, miR-132-3p, miR-137-3p, and miR-145-5p.

In some embodiments, the recombinant herpesvirus comprises:
(a) one or more miRNA target sequences for miR-122-5p;
(b) one or more miRNA target sequences for miR-145-5p;
(c) one or more miRNA target sequences for miR-124-3p, miR-128T, miR-137-3p, or any combination thereof;
(d) one or more miRNA target sequences for miR-34b-5p, miR-34c-5p, or any combination thereof; and
(e) one or more miRNA target sequences for miR-129-2-3p, miR-132-3p, or any combination thereof.

In some embodiments, the recombinant herpesvirus comprises a first miR-TS cassette inserted into a first viral gene, wherein the first miR-TS cassette comprises one or more miRNA target sequences for each of miR-34c-5p, miR-124-3p, miR-129-2-3p, and miR-132-3p. In some embodiments, the miRNA target sequences in the first miR-TS cassette are arranged as (34c-5p)-(124-3p)-(132-3p)-(129-2-3p)-(34c-5p)-(124-3p)-(129-2-3p)-(132-3p)-(124-3p)-(129-2-3p)-(132-3p)-(34c-5p). In some embodiments, the first miR-TS cassette comprises a nucleic acid sequence at least 90%, at least 95%, at least 96%, at least 97%, at least 98%, at least 99%, or 100% identical to SEQ ID NO: 859. In some embodiments, the first viral gene is ICP8.

In some embodiments, the recombinant herpesvirus comprises a second miR-TS cassette inserted into a second viral gene, wherein the second miR-TS cassette comprises one or more miRNA target sequences for each of miR-122-5p, miR-124-3p, miR-128T, and miR-137-3p. In some embodiments, the miRNA target sequences in the second miR-TS cassette are arranged as (137-3p)-(128T)-(122-5p)-(124-3p)-(122-5p)-(128T)-(137-3p)-(124-3p)-(128T)-(137-3p)-(124-3p)-(122-5p). In some embodiments, the second miR-TS cassette comprises a nucleic acid sequence at least 90%, at least 95%, at least 96%, at least 97%, at least 98%, at least 99%, or 100% identical to SEQ ID NO: 858. In some embodiments, the second viral gene is ICP4. In some embodiments, the recombinant herpesvirus comprises the second miR-TS cassette in both ICP4 viral genes of the viral genome.

In one aspect, the disclosure provides recombinant herpesviruses comprising one or more miRNA target sequences in both ICP4 viral genes of the viral genome. In some embodiments, the miRNA target sequences are the same in both said ICP4 viral genes.

In some embodiments, the recombinant herpesvirus comprises a third miR-TS cassette inserted into a third viral gene, wherein the third miR-TS cassette comprises one or more miRNA target sequences for each of miR-34c-5p, miR-124-3p, miR-128T, and miR-137-3p.

In some embodiments, the miRNA target sequences in the third miR-TS cassette are arranged as (124-3p)-(128T)-(34c-5p)-(137-3p)-(128T)-(34c-5p)-(137-3p)-(124-3p)-(128T)-(137-3p)-(124-3p)-(34c-5p). In some embodiments, the third miR-TS cassette comprises a nucleic acid sequence at least 90%, at least 95%, at least 96%, at least 97%, at least 98%, at least 99%, or 100% identical to SEQ ID NO: 873. In some embodiments, the recombinant herpesvirus comprises a third miR-TS cassette inserted into a third viral gene, wherein the third miR-TS cassette comprises one or more miRNA target sequences for each of miR-34b-3p, miR-34c-5p, miR-128T, miR-137-3p. In some embodiments, the third viral gene is ICP27.

In some embodiments, the recombinant herpesvirus comprises a fourth miR-TS cassette inserted into a fourth viral gene, wherein:
(i) the fourth miR-TS cassette comprises one or more miRNA target sequences for each of miR-34b-5p, miR-34c-5p, miR-129-5p, and miR-145-5p;
(ii) the fourth miR-TS cassette comprises one or more miRNA target sequences for each of miR-34b-5p, miR-34c-5p, miR-132-3p, and miR-145-5p; or
(iii) the fourth miR-TS cassette comprises one or more miRNA target sequences for each of miR-34b-3p, miR-34c-5p, miR-132-3p, and miR-145-5p.
   In some embodiments, wherein the fourth miR-TS cassette comprises one or more miRNA target sequences for each of miR-34b-5p, miR-34c-5p, miR-132-3p, and miR-145-5p. In some embodiments, the miRNA target sequences in the fourth miR-TS cassette are arranged as (145-5p)-(34b-5p)-(132-3p)-(34c-5p)-(145-5p)-(34c-5p)-(34b-5p)-(132-3p)-(34b-5p)-(145-5p)-(132-3p)-(34c-5p). In some embodiments, the fourth miR-TS cassette comprises a nucleic acid sequence at least 90%, at least 95%, at least 96%, at least 97%, at least 98%, at least 99%, or 100% identical to SEQ ID NO: 874. In some embodiments, the fourth viral gene is UL8.

In some embodiments, each of the miR-TS cassettes comprises at least 2, at least 3, or at least 4 copies of each of the miRNA target sequences. In some embodiments, each of the miR-TS cassettes comprises 3 copies of each of the miRNA target sequences.

In some embodiments, the replication of the recombinant HSV is reduced in a non-cancerous cell compared to the replication of the recombinant HSV in a cancerous cell. In some embodiments, the cancerous cell is a glioblastoma cell. In some embodiments, the non-cancerous cell is selected from the group consisting of a neuron, an ependymal cell, an oligodendrocyte, an endothelial cell, a hepatocyte, an astrocyte, and any combination thereof. In some embodiments, wherein the non-cancerous cell is an astrocyte.

In some embodiments, the one or more miRNA target sequences for miR-34b-5p comprise or consist of SEQ ID NO: 867. In some embodiments, the one or more miRNA target sequences for miR-34b-3p comprise or consist of SEQ ID NO: 868. In some embodiments, the one or more miRNA target sequences for miR-34c-5p comprise or consist of SEQ ID NO: 869. In some embodiments, the one or more miRNA target sequences for miR-122-5p comprise or consist of SEQ ID NO: 804. In some embodiments, the one or more miRNA target sequences for miR-124-3p comprise or consist of SEQ ID NO: 805. In some embodiments, the one or more miRNA target sequences for miR-128T comprise or consist of SEQ ID NO: 870. In some embodiments, the one or more miRNA target sequences for miR-129-5p comprise or consist of SEQ ID NO: 813. In some embodiments, the one or more miRNA target sequences for miR-129-2-3p comprise or consist of SEQ ID NO: 871. In some embodiments, the one or more miRNA target sequences for miR-132-3p comprise or consist of SEQ ID NO: 872. In some embodiments, the one or more miRNA target sequences for miR-137-3p comprise or consist of SEQ ID NO: 819. In some embodiments, the one or more miRNA target sequences for miR-145-5p comprise or consist of SEQ ID NO: 823.

In some embodiments, the recombinant herpesvirus comprises the polynucleotide encoding the retargeting domain, wherein the retargeting domain specifically binds a target protein expressed by a target cell. In some embodiments, the polynucleotide encoding the retargeting domain is inserted into the open reading frame of a US6 gene encoding a glycoprotein D (gD). In some embodiments, the polynucleotide encoding the retargeting domain replaces the US6 gene region encoding an amino acid sequence corresponding to amino acids 6-24 of SEQ ID NO: 921. In some embodiments, the target protein expressed by the target cell comprises integrin α5β1, integrin αvβ1, integrin αvβ3, integrin αvβ6, or a combination thereof. In some embodiments, the target protein expressed by the target cell comprises epidermal growth factor receptor (EGFR). In some embodiments, the retargeting domain comprises a knottin peptide capable of specifically binding to the target protein expressed by the target cell. In some embodiments, the retargeting domain comprises no more than 50, no more than 45, no more than 40, or no more than 35 amino acids. In some embodiments, the retargeting domain comprises an amino acid sequence having at least 80%, at least 85%, at least 90%, or 100% identity to SEQ ID NO: 922. In some embodiments, the retargeting domain comprises an immunoglobulin domain capable of specifically binding to the target protein expressed by the target cell. In some embodiments, the retargeting domain comprises a binding domain of, or a binding domain derived from, a variable domain of a heavy chain-only antibody (VHH) or a variable domain of new antigen receptor immunoglobulin (V-NAR). In some embodiments, the retargeting domain comprises no more than 150, no more than 140, or no more than 130 amino acids. In some embodiments, the retargeting domain comprises an amino acid sequence having at least 80%, at least 85%, at least 90%, or 100% identity to SEQ ID NO: 923. In some embodiments, the herpesvirus is capable of infecting the target cell expressing the target protein. In some embodiments, the herpesvirus is capable of infecting cells without Nectin-1 expression. In some embodiments, the cells are Vero cells.

In some embodiments, the recombinant herpesvirus comprises the UL30 viral gene encoding the DPCS comprising the mutation and the UL23 viral gene encoding the TK comprising the mutation. In some embodiments, the mutation in the DPCS increases DNA replication fidelity of the herpesvirus by at least 10%, at least 20%, at least 30%, at least 50%, at least 100%, at least 2-fold, at least 3-fold, or at least 5-fold. In some embodiments, the mutation in the DPCS is at an amino acid position corresponding to L774 of SEQ ID NO: 917. In some embodiments, the mutation is an amino acid substitution. In some embodiments, the mutation in the DPCS is the amino acid substitution corresponding to L774F of SEQ ID NO: 917. In some embodiments, the DPCS comprises an amino acid sequence at least 90%, at least 95%, at least 97%, at least 98%, at least 99%, or 100% identical to SEQ ID NO: 917, except for the mutation in the DPCS. In some embodiments, the IC50 of acyclovir is less than 0.5 ug/ml, less than 1.0 ug/ml, less than 1.5 ug/ml, or less than 2.0 ug/ml for the herpesvirus. In some embodiments, the mutation in the TK decreases the IC50 of acyclovir for the herpesvirus by at least 1-fold, at least 2-fold, at least 5-fold, at least 10-fold, at least 20-fold, at least 50-fold, or at least 100-fold. In some embodiments, the mutation in the TK is at one or more amino acid positions corresponding to L159, 1160, F161, A168 and/or L169 of SEQ ID NO: 918. In some embodiments, the mutation is amino acid substitution. In some embodiments, the mutation in the TK comprises one or more amino acid substitutions of:
(a) L159I or L159L;
(b) I160L or I160F;
(c) F161A, F161V, F161P, or F161L;
(d) A168D, A168Y, A168V or A168F; and/or
(e) L169F, L169Y, L169L, L169I, L169M, L169N, or L169K.
   In some embodiments, the mutation in the TK comprises amino acid substitutions corresponding to L159I, 1160F, F161L, A168F and L169M of SEQ ID NO: 918. In some embodiments, the mutation in the TK comprises amino acid substitutions corresponding to I160F, F161A, and A168F of SEQ ID NO: 918. In some embodiments, the mutation in the TK comprises amino acid substitutions corresponding to 1160F, F161L, A168F, and L169N of SEQ ID NO: 918. In some embodiments, the TK comprises an amino acid sequence at least 90%, at least 95%, at least 97%, at least 98%, at least 99%, or 100% identical to SEQ ID NO: 918, except the mutation in the TK.

In some embodiments, the viral genome of the herpesvirus encodes:
(i) the first gB and/or the second gB, wherein the first gB comprises the syncytial mutation, and wherein the second gB comprises no syncytial mutation;
(ii) the first gK and/or the second gK, wherein the first gK comprises the syncytial mutation, and wherein the second gK comprises no syncytial mutation;
(iii) the first gH and/or the second gH, wherein the first gH comprises the syncytial mutation, and wherein the second gH comprises no syncytial mutation;
(iv) the first UL20 and/or the second UL20, wherein the first UL20 comprises the syncytial mutation, and wherein the second UL20 comprises no syncytial mutation; and/or
(v) the first UL24 and/or the second UL24, wherein the first UL24 comprises the syncytial mutation, and wherein the second UL24 comprises no syncytial mutation.
   In some embodiments, the first gB is encoded by an endogenous gB-encoding gene locus and the second gB is encoded by an exogenous expression cassette. In some embodiments, the first gB is encoded by an exogenous expression cassette and the second gB is encoded by an endogenous gB-encoding gene locus. In some embodiments, the first gK is encoded by an endogenous gK-encoding gene locus and the second gK is encoded by an exogenous expression cassette. In some embodiments, the first gK is encoded by an exogenous expression cassette and the second gK is encoded by an endogenous gK-encoding gene locus. In some embodiments, the first gH is encoded by an endogenous gH-encoding gene locus and the second gH is encoded by an exogenous expression cassette. In some embodiments, the first gH is encoded by an exogenous expression cassette and the second gH is encoded by an endogenous gH-encoding gene locus. In some embodiments, the first UL20 is encoded by an endogenous UL20 gene locus and the second UL20 is encoded by an exogenous expression cassette. In some embodiments, the first UL20 is encoded by an exogenous expression cassette and the second UL20 is encoded by an endogenous UL20 gene locus. In some embodiments, the first UL24 is encoded by an endogenous UL24 gene locus and the second UL24 is encoded by an exogenous expression cassette. In some embodiments, the first UL24 is encoded by an exogenous expression cassette and the second UL24 is encoded by an endogenous UL24 gene locus.

In some embodiments, the viral genome of the herpesvirus encodes the first gB but not the second gB. In some embodiments, the viral genome of the herpesvirus encodes the first gK but not the second gK. In some embodiments, the viral genome of the herpesvirus encodes the first gH but not the second gH. In some embodiments, the viral genome of the herpesvirus encodes the first UL20 but not the second UL20. In some embodiments, the viral genome of the herpesvirus encodes the first UL24 but not the second UL24. In some embodiments, the viral genome of the herpesvirus encodes the first gB and the first gK; optionally, the viral genome of the herpesvirus further encodes the first gH and the first UL24.

In some embodiments, the exogenous expression cassette is located at UL3-UL4 intergenic region. In some embodiments, the exogenous expression cassette is located at UL50-UL51 intergenic region.

In some embodiments, the recombinant herpesvirus displays syncytial phenotype in cancer cells.

In one aspect, the disclosure provides cells comprising a recombinant nucleic acid encoding the recombinant herpesvirus of the disclosure.

In one aspect, the disclosure provides cells comprising a first nucleic acid encoding a recombinant herpesvirus and a second nucleic acid, wherein:
(i) the viral genome of the herpesvirus encodes a first gB, wherein the first gB comprises a syncytial mutation, the second nucleic acid encodes a second gB, wherein the second gB comprises no syncytial mutation; or, the viral genome of the herpesvirus encodes a second gB, wherein the second gB comprises no syncytial mutation, the second nucleic acid encodes a first gB, wherein the first gB comprises a syncytial mutation;
(ii) the viral genome of the herpesvirus encodes a first gK, wherein the first gK comprises a syncytial mutation, the second nucleic acid encodes a second gK, wherein the second gK comprises no syncytial mutation; or the viral genome of the herpesvirus encodes a second gK, wherein the second gK comprises no syncytial mutation, the second nucleic acid encodes a first gK, wherein the first gK comprises a syncytial mutation;
(iii) the viral genome of the herpesvirus encodes a first gH, wherein the first gH comprises a syncytial mutation, the second nucleic acid encodes a second gH, wherein the second gH comprises no syncytial mutation; or the viral genome of the herpesvirus encodes a second gH, wherein the second gH comprises no syncytial mutation, the second nucleic acid encodes a first gH, wherein the first gH comprises a syncytial mutation;
(iv) the viral genome of the herpesvirus encodes a first UL20, wherein the first UL20 comprises a syncytial mutation, the second nucleic acid encodes a second UL20, wherein the second UL20 comprises no syncytial mutation; or the viral genome of the herpesvirus encodes a second UL20, wherein the second UL20 comprises no syncytial mutation, the second nucleic acid encodes a first UL20, wherein the first UL20 comprises a syncytial mutation; and/or
(v) the viral genome of the herpesvirus encodes a first UL24, wherein the first UL24 comprises a syncytial mutation, the second nucleic acid encodes a second UL24, wherein the second UL24 comprises no syncytial mutation; or the viral genome of the herpesvirus encodes a second UL24, wherein the second UL24 comprises no syncytial mutation, the second nucleic acid encodes a first UL24, wherein the first UL24 comprises a syncytial mutation.

In some embodiments, the viral genome of the herpesvirus encodes a first gB, wherein the first gB comprises a syncytial mutation, wherein the second nucleic acid encodes a second gB, wherein the second gB comprises no syncytial mutation. In some embodiments, the viral genome of the herpesvirus encodes a second gB, wherein the second gB comprises no syncytial mutation, wherein the second nucleic acid encodes a first gB, wherein the first gB comprises a syncytial mutation. In some embodiments, the viral genome of the herpesvirus encodes a first gK, wherein the first gK comprises a syncytial mutation, wherein the second nucleic acid encodes a second gK, wherein the second gK comprises no syncytial mutation. In some embodiments, the viral genome of the herpesvirus encodes a second gK, wherein the second gK comprises no syncytial mutation, wherein the second nucleic acid encodes a first gK, wherein the first gK comprises a syncytial mutation. In some embodiments, the viral genome of the herpesvirus encodes a first gH, wherein the first gH comprises a syncytial mutation, wherein the second nucleic acid encodes a second gH, wherein the second gH comprises no syncytial mutation. In some embodiments, the viral genome of the herpesvirus encodes a second gH, wherein the second gH comprises no syncytial mutation, wherein the second nucleic acid encodes a first gH, wherein the first gH comprises a syncytial mutation. In some embodiments, the viral genome of the herpesvirus encodes a first UL20, wherein the first UL20 comprises a syncytial mutation, wherein the second nucleic acid encodes a second UL20, wherein the second UL20 comprises no syncytial mutation. In some embodiments, the viral genome of the herpesvirus encodes a second UL20, wherein the second UL20 comprises no syncytial mutation, wherein the second nucleic acid encodes a first UL20, wherein the first UL20 comprises a syncytial mutation. In some embodiments, the viral genome of the herpesvirus encodes a first UL24, wherein the first UL24 comprises a syncytial mutation, wherein the second nucleic acid encodes a second UL24, wherein the second UL24 comprises no syncytial mutation. In some embodiments, the viral genome of the herpesvirus encodes a second UL24, wherein the second UL24 comprises no syncytial mutation, wherein the second nucleic acid encodes a first UL24, wherein the first UL24 comprises a syncytial mutation. In some embodiments, the recombinant herpesvirus comprises a single copy of gB-encoding viral gene, a single copy of gK-encoding viral gene, a single copy of gH-encoding viral gene, a single copy of UL20 viral gene, and/or a single copy of UL24 viral gene. In some embodiments, the first nucleic acid and the second nucleic acid are comprised within a single polynucleotide molecule. In some embodiments, the first nucleic acid and the second nucleic acid are comprised within two different polynucleotide molecules. In some embodiments, the cell is a Vero cell.

In some embodiments, the gB syncytial mutation comprises a mutation at one or more amino acid residues corresponding to R796, R800, T813, L817, S854, A855, R858, or A874, an insertion between E816 and L817, a deletion of S869 to C-terminus, a deletion of T877 to C-terminus, or a combination thereof, of SEQ ID NO: 919. In some embodiments, the gB syncytial mutation comprises one or more mutations corresponding to R796C, R800W, T813I, L817H, L817P, S854F, A855V, R858C, R858H, A874P, an insertion of VN or VNVN between E816 and L817, a deletion of S869 to C-terminus, or a deletion of T877 to C-terminus, of SEQ ID NO: 919. In some embodiments, the gB syncytial mutation comprises a deletion of T877 to C-terminus according to SEQ ID NO: 919. In some embodiments, the first and/or the second gB comprise a mutation corresponding to D285N and/or A549T of SEQ ID NO: 919.

In some embodiments, the gK syncytial mutation comprises a mutation at one or more amino acid residues corresponding to P33, A40, L86, D99, A111, L118, T121, C243, L304, 1307, or R310 of SEQ ID NO: 920. In some embodiments, the gK syncytial mutation comprises one or more mutations corresponding to P33S, A40V, A40T, L86P, D99N, A111V, L118Q, T121I, C243Y, L304P, 1307N, or R310L of SEQ ID NO: 920. In some embodiments, the gK syncytial mutation comprises 1307N according to SEQ ID NO: 920.

In some embodiments, the gH syncytial mutation comprises a mutation at one or more amino acid residues corresponding to N753 or A778 of SEQ ID NO: 943. In some embodiments, the gH syncytial mutation comprises one or more mutations corresponding to N753K or A778V of SEQ ID NO: 943.

In some embodiments, the UL20 syncytial mutation comprises a mutation at one or more amino acid residues corresponding to Y49, S50, R51, R209, T212, R213, or C-terminal deletion after N217, of SEQ ID NO: 944. In some embodiments, the UL20 syncytial mutation comprises one or more mutations corresponding to Y49A, S50A, R51A, R209A, T212A, R213A, or C-terminal deletion after N217, of SEQ ID NO: 944.

In some embodiments, the UL24 syncytial mutation comprises a mutation at one or more amino acid residues corresponding to T64, R63, or V64 of SEQ ID NO: 942. In some embodiments, the UL24 syncytial mutation comprises one or more mutations corresponding to T64G, R63V, or V64S of SEQ ID NO: 942.

In some embodiments, the open reading frame encoding the first gB is operably linked to a CMV promoter and/or a bGH polyA tail. In some embodiments, the open reading frame encoding the second gB is operably linked to a CMV promoter and/or a bGH polyA tail. In some embodiments, the open reading frame encoding the first gK is operably linked to a CMV promoter and/or a bGH poly A tail. In some embodiments, the open reading frame encoding the second gK is operably linked to a CMV promoter and/or a bGH polyA tail.

In some embodiments, the yield of the recombinant herpesvirus is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than the yield of a control herpesvirus or a control cell that does not encode the second gB, the second gK, the second gH, the second UL20, or the second UL24.

In some embodiments, the gene encoding the first gB comprises a miRNA target sequence (miR-TS) cassette, wherein the miR-TS cassette comprises one or more target sequences of one or more miRNAs. In some embodiments, the gene encoding the first gK comprises a miRNA target sequence (miR-TS) cassette, wherein the miR-TS cassette comprises one or more target sequences of one or more miRNAs. In some embodiments, the gene encoding the first gH comprises a miRNA target sequence (miR-TS) cassette, wherein the miR-TS cassette comprises one or more target sequences of one or more miRNAs. In some embodiments, the gene encoding the first UL20 comprises a miRNA target sequence (miR-TS) cassette, wherein the miR-TS cassette comprises one or more target sequences of one or more miRNAs. In some embodiments, the gene encoding the first UL24 comprises a miRNA target sequence (miR-TS) cassette, wherein the miR-TS cassette comprises one or more target sequences of one or more miRNAs.

In some embodiments, the one or more miRNAs comprise at least one of miR-34c-5p, miR-299-5p, and miR-582-5p. In some embodiments, the one or more miRNAs comprise at least two of miR-34c-5p, miR-299-5p, and miR-582-5p. In some embodiments, the one or more miRNAs comprise miR-34c-5p, miR-299-5p, and miR-582-5p. In some embodiments, the miR-TS cassette comprises at least three copies, or at least four copies of the target sequences of each of the miRNA separated by a 4 bp spacer. In some embodiments, the miR-TS cassette is located at the 3′UTR of the gene. In some embodiments, the target sequence of the miRNA comprises or consists of the reverse complement of the miRNA. In some embodiments, the miR-TS cassette comprises the polynucleotide sequence of SEQ ID NO: 930. In some embodiments, the yield of the recombinant herpesvirus is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than the yield of a control herpesvirus or a control cell that does not comprise the miR-TS cassette.

In one aspect, the disclosure provides recombinant herpesviruses produced by culturing the cell of the disclosure and recovering the recombinant herpesvirus from the cell culture.

In one aspect, the disclosure provides recombinant herpesviruses wherein the viral genome of the herpesvirus encodes a gK comprising a syncytial mutation corresponding to 1307N of SEQ ID NO: 920.

In some embodiments, the herpesvirus is an alphaherpesvirus. In some embodiments, the alphaherpesvirus is a herpes simplex virus. In some embodiments, the herpes simplex virus is a herpes simplex virus-1 (HSV-1).

In some embodiments, the recombinant herpesvirus is oncolytic.

In some embodiments, the recombinant herpesvirus is derived from an encephalitic HSV isolate according to SEQ ID NO: 857. In some embodiments, the recombinant herpesvirus comprises a polynucleotide sequence having at least 80%, at least 85%, at least 90%, at least 95%, at least 96%, at least 97%, at least 98%, at least 99%, at least 99.5%, or 100% identity to SEQ ID NO: 857.

In some embodiments, the recombinant herpesvirus is defective for anterograde transport.

In some embodiments, the recombinant herpesvirus comprises a mutation in the UL37 viral gene. In some embodiments, the UL37 viral gene encodes a UL37 protein comprising a mutation at at least 1, at least 2, at least 3, at least 4, or all 5 amino acid positions corresponding to Q403, E452, Q455, Q511, and R515 of SEQ ID NO: 856. In some embodiments, the mutation in the UL37 viral gene comprises Q403A/E452A/Q455A/Q511A/R515A according to SEQ ID NO: 856.

In some embodiments, the recombinant herpesvirus encodes a gB comprising the mutations corresponding to A549T/D285N of SEQ ID NO: 919.

In some embodiments, the recombinant herpesvirus retains the function of ICP6, ICP34.5, and/or ICP47.

In some embodiments, the one or more transgenes are inserted in the UL50-UL51 intergenic region.

In one aspect, the disclosure provides recombinant viruses comprising one or more transgenes encoding one or more payload proteins selected from HPGD, ADA2, HYAL1, CHP, CCL21, IL-12, a CD47 antagonist, a TGFβ antagonist, a PD1 antagonist, a TREM2 antagonist, a biomolecule comprising chlorotoxin (CTX), or any combinations thereof. In some embodiments, the one or more payload proteins comprise or consist of IL-12, a PD1 antagonist, and a TREM2 antagonist. In some embodiments, the one or more payload proteins comprise HPGD. In some embodiments, the one or more payload proteins comprise a biomolecule comprising CTX. In some embodiments, the one or more payload proteins comprise or consist of one of the combinations of payload proteins listed in Tables 4-7.

In some embodiments, the one or more payload proteins comprise HPGD. In some embodiments, the one or more payload proteins comprise ADA2. In some embodiments, the one or more payload proteins comprise HYAL1. In some embodiments, the one or more payload proteins comprise CHP. In some embodiments, the one or more payload proteins comprise CCL21. In some embodiments, the one or more payload proteins comprise IL-12. In some embodiments, the one or more payload proteins comprise the CD47 antagonist. In some embodiments, the one or more payload proteins comprise the TGFβ antagonist. In some embodiments, the one or more payload proteins comprise the PD1 antagonist. In some embodiments, the one or more payload proteins comprise the TREM2 antagonist. In some embodiments, the antagonist comprises an antibody or antigen binding fragment thereof. In some embodiments, the one or more payload proteins comprise the biomolecule comprising CTX. In some embodiments, the biomolecule comprising CTX further comprises a T-cell engager moiety specifically binding to a protein expressed on the surface of the T-cell. In some embodiments, the protein expressed on the surface of the T-cell is CD3.

In one aspect, the disclosure provides recombinant viruses comprising:
(a) one or more miRNA target sequences for miR-122-5p;
(b) one or more miRNA target sequences for miR-145-5p;
(c) one or more miRNA target sequences for miR-124-3p, miR-128T, miR-137-3p, or any combination thereof;
(d) one or more miRNA target sequences for miR-34b-5p, miR-34c-5p, or any combination thereof; and
(e) one or more miRNA target sequences for miR-129-2-3p, miR-132-3p, or any combination thereof.
   In some embodiments, the recombinant virus comprises the miRNA target sequences for miR-34b-5p, miR-34c-5p, miR-122-5p, miR-124-3p, miR-128T, miR-129-2-3p, miR-132-3p, miR-137-3p, and miR-145-5p.

In one aspect, the disclosure provides recombinant viruses wherein the viral genome of the recombinant virus encodes a protein comprising a syncytial mutation and a counterpart protein without the syncytial mutation. In some embodiments, the protein comprising the syncytial mutation and the counterpart protein without the syncytial mutation share at least 90%, at least 95%, at least 96%, at least 97%, at least 98%, at least 99%, or 100% sequence identity, except the syncytial mutation. In some embodiments, the protein comprising the syncytial mutation is encoded by an endogenous viral gene and the counterpart protein without the syncytial mutation is encoded by an exogenous expression cassette. In some embodiments, the protein comprising the syncytial mutation is encoded by an exogenous expression cassette and the counterpart protein without the syncytial mutation is encoded by an endogenous viral gene. In some embodiments, both the protein comprising the syncytial mutation and the counterpart protein without the syncytial mutation are encoded by one exogenous expression cassette or by different exogenous expression cassettes.

In one aspect, the disclosure provides cells comprising a recombinant nucleic acid encoding the recombinant virus of the disclosure.

In one aspect, the disclosure provides cells comprising a first nucleic acid encoding a recombinant virus and a second nucleic acid, wherein the viral genome of the recombinant virus encodes a protein comprising a syncytial mutation, wherein the second nucleic acid encodes a a counterpart protein without the syncytial mutation. In some embodiments, the protein comprising the syncytial mutation and the counterpart protein without the syncytial mutation share at least 90%, at least 95%, at least 96%, at least 97%, at least 98%, at least 99%, or 100% sequence identity, except the syncytial mutation.

In some embodiments, the yield of the recombinant virus is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than the yield of a control virus or a control cell that does not encodes the counterpart protein without the syncytial mutation.

In some embodiments, the gene encoding the protein comprising the syncytial mutation comprises a miRNA target sequence (miR-TS) cassette, wherein the miR-TS cassette comprises one or more target sequences of one or more miRNAs. In some embodiments, the one or more miRNAs comprise at least one, at least two, or all of miRNAs selected from miR-34c-5p, miR-299-5p, and miR-582-5p. In some embodiments, the yield of the recombinant virus is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than the yield of a control virus or a control cell that does not comprise the miR-TS cassette.

In some embodiments, the recombinant virus is derived from a herpes simplex virus, an adenovirus, a polio virus, a vaccinia virus, a measles virus, a vesicular stomatitis virus, an orthomyxovirus, a parvovirus, a maraba virus, or a coxsackievirus. In some embodiments, the recombinant virus is oncolytic.

In one aspect, the disclosure provides recombinant viruses produced by culturing the cell of the disclosure and recovering the recombinant herpesvirus from the cell culture.

In one aspect, the disclosure provides nucleic acid molecules encoding the recombinant herpesvirus of the disclosure, or the recombinant virus of the disclosure. In some embodiments, the nucleic acid molecule is DNA. In some embodiments, the nucleic acid molecule is RNA.

In one aspect, the disclosure provides viral stocks comprising the recombinant herpesvirus of the disclosure, or the recombinant virus of the disclosure.

In one aspect, the disclosure provides particle comprising the nucleic acid molecule of the disclosure. In some embodiments, the particle is selected from the group consisting of a nanoparticle, an exosome, a liposome, and a lipoplex. In some embodiments, the particle is a lipid nanoparticle. In some embodiments, contacting a eukaryotic cell with the particle results in production of infectious virus particles by the eukaryotic cell.

In one aspect, the disclosure provides pharmaceutical compositions comprising:
(i) the recombinant herpesvirus of the disclosure, the recombinant virus of the disclosure, the nucleic acid molecule of the disclosure, or the particle of the disclosure; and
(ii) a pharmaceutically-acceptable carrier.

In one aspect, the disclosure provides methods of killing a cancerous cell, comprising exposing the cancerous cell to the recombinant herpesvirus of the disclosure, the recombinant virus of the disclosure, the nucleic acid molecule of the disclosure, or the particle of the disclosure, or the pharmaceutical composition of the disclosure, under conditions sufficient for the virus or particle to infect and the virus to replicate within said cancerous cell, and wherein replication of the virus within the cancerous cell results in cell death. In some embodiments, the cell is in vitro or in vivo. In some embodiments, the cancerous cell has a reduced expression of a miRNA capable of binding to the one or more miRNA target sequences compared to the expression of the miRNA in a non-cancerous cell. In some embodiments, replication of the virus is increased or maintained in the cancerous cell with a reduced expression of the miR capable of binding to the one or more miRNA target sequences. In some embodiments, the cancerous cell is a cell of lung cancer, breast cancer, ovarian cancer, cervical cancer, prostate cancer, testicular cancer, colorectal cancer, colon cancer, pancreatic cancer, liver cancer, gastric cancer, head and neck cancer, thyroid cancer, malignant glioma, glioblastoma, melanoma, non-melanoma skin cancer, B-cell chronic lymphocytic leukemia, diffuse large B-cell lymphoma (DLBCL), or marginal zone lymphoma (MZL). In some embodiments, the cancerous cell is a glioblastoma cell.

In one aspect, the disclosure provides methods of treating cancer in a subject in need thereof, comprising administering the recombinant herpesvirus of the disclosure, the recombinant virus of the disclosure, the nucleic acid molecule of the disclosure, or the particle of the disclosure, or the pharmaceutical composition of the disclosure to the subject. In some embodiments, the virus, the particle, or the composition is administered intravenously, subcutaneously, intratumorally, intramuscularly, or intranasally. In some embodiments, the virus, the particle, or the composition is administered intratumorally. In some embodiments, the virus, the particle, or the composition is administered intravenously. In some embodiments, the virus, the particle, or the composition is administered only once. In some embodiments, the cancer is selected from the group consisting of lung cancer, breast cancer, ovarian cancer, cervical cancer, prostate cancer, testicular cancer, colorectal cancer, colon cancer, pancreatic cancer, liver cancer, gastric cancer, head and neck cancer, thyroid cancer, malignant glioma, glioblastoma, melanoma, non-melanoma skin cancer, B-cell chronic lymphocytic leukemia, diffuse large B-cell lymphoma (DLBCL), and marginal zone lymphoma (MZL). In some embodiments, the cancer is glioblastoma.

In one aspect, the disclosure provides cell lines comprising the cell of the disclosure.

In one aspect, the disclosure provides methods of producing a recombinant herpesvirus, comprising culturing the cell of the disclosure, or the cell line of the disclosure, and recovering the recombinant herpesvirus from the cell culture.

In one aspect, the disclosure provides uses of the recombinant herpesvirus of the disclosure in combination with a small molecule for imaging the infection site of the herpesvirus.

In one aspect, the disclosure provides methods of imaging the infection site of an herpesvirus in vivo, comprising administering the recombinant herpesvirus of the disclosure and a small molecule.

In some embodiments, the small molecule is radioisotope labeled acyclovir. In some embodiments, the radioisotope label comprises fluorine-18 (¹⁸F) label.

### BRIEF DESCRIPTION OF THE DRAWINGS

FIG. 1 is a schematic showing the organization of a non-limiting example of a HSV backbone vector of the disclosure. CAG: CAG promoter; rBG: rabbit beta-globin polyadenylation signal; BFP: Blue fluorescent protein; DTA: diphtheria toxin subunit A.

FIG. 2A is a plot showing the expression level of each indicated miRNA in normal brain cells and glioblastoma cells. FIG. 2B is a plot showing the expression level of each indicated miRNA in ependymal cells. FIG. 2C are cell images showing (a) upper left: miR-124-3p miRNA expression level in neurons; (b) upper right: miR-124-3p miRNA expression level in oligodentrocytes; (c) lower left: Ccdc153 expression level indicating the positions of ependymal cells; (d) lower right: miR-34c-5p miRNA expression level in ependymal cells.

FIG. 3A shows exemplary designs of miR-TS cassettes in various viral genes; as well as the types of CNS cells that each corresponding miRNA is highly expressed in. FIG. 3B is a plot showing the number of plaque forming units (pfu) per mL of the HSV vector at the presence of the indicated miRNA mimic. FIG. 3C shows another exemplary design of miR-TS cassettes in various viral genes; as well as the types of CNS cells that each corresponding miRNA is highly expressed in. Each miR-T cassette incorporates 4 miR target sequences assembled in triplicate and is designed using an algorithm to minimize RNA secondary structure for maximum activity.

FIGS. 4A and 4B are plots showing the propagation of the indicated HSV vectors in Vero cells over time, as measured by PFU on the y-axis.

FIG. 5A is a plot showing the overall survival (%) of mice after intracranial injection (at day 1) of the indicated HSV vector. FIG. 5B is a plot showing the body weight change (%) of mice after intracranial injection (at day 1) of the indicated HSV vector.

FIG. 6A shows the protected cell types for each miRNA target sequence. FIG. 6B shows the miR-TS cassettes designed for the indicated HSV genes. FIG. 6C shows the configuration of miR target sequences within the indicated cassettes. FIG. 6D shows the relative expression level of luciferase under the regulatory control of the indicated miR-TS cassette at the presence of various miRNA mimics.

FIG. 7A shows virus yield of the ONCR-2169 in A431 cells at the presence of indicated miRNA mimics. FIG. 7B shows non-attenuated growth of indicated HSV virus in Vero cells.

FIG. 7C shows body weight changes of female BALB/c mice injected with the indicated HSV virus.

FIG. 8 is a schematic showing the design of the indicated HSV viral constructs. KPI: a knottin peptide that binds integrins; VE: a VHH nanobody that binds the extracellular domain of EGFR. UL23 SR39: UL23 encoding a thymidine kinase comprising L159I, 1160F, F161L, A168F and L169M combo mutations. gB N/T: UL27 encoding a glycoprotein B (gB) comprising D285N/A549T double mutations. UL30 HiFi: UL30 encoding a DNA polymerase catalytic subunit comprising L774F mutation. UL37 R2: UL37 encoding a tegument protein comprising the “R2” combo mutations (Q403A/E452A/Q455A/Q511A/R515A). The HSV construct further comprises miR-T 3060 cassette (SEQ ID NO: 858) in both ICP4 loci, miR-T 9919 cassette (SEQ ID NO: 859) in ICP8 locus, miR-T 3012 cassette (SEQ ID NO: 873) in ICP27 locus, miR-T 3096 cassette (SEQ ID NO: 874) in UL8 locus, and a mCherry-Fluc expression cassette in the UL50-UL51 intergenic region.

FIG. 9A shows the results of plaque assay of the indicated viruses on Vero cells. FIG. 9B shows the results of proliferation assays with the indicated viruses and cells.

FIG. 10 shows the results of proliferation assays with the indicated viruses in various glioblastoma cell lines.

FIG. 11 shows immunohistochemistry results of representative GBM cores.

FIG. 12 is a schematic of the ONCR-1012 viral construct.

FIG. 13 is a diagram showing the number of sequence variants over multiple passages when the indicated viruses were cultured.

FIG. 14A is an image showing the plagues generated by the infection of the indicated viruses. FIG. 14B is a diagram showing the number of plague-forming units (PFUs) per ml of the indicated viruses.

FIG. 15A is a diagram showing the relative plaque titer at different concentrations of acyclovir for the indicated viruses. FIG. 15B is a diagram showing the percentage of the remaining plaques at different concentrations of acyclovir for the indicated viruses.

FIG. 16 is a diagram showing the percentage of the remaining plaques at different concentrations of acyclovir for the indicated viruses, as well as the calculated IC50 values.

FIG. 17A is an image showing the plagues generated by the infection of the indicated viruses. FIG. 17B is a diagram showing the number of plague-forming units (PFUs) per cell for the indicated viruses.

FIG. 18A is a schematic showing the co-expression of wildtype gK from its endogenous locus and a syncytial gK mutant from a cDNA cassette. FIG. 18B is a schematic showing the co-expression of a syncytial gB mutant from its endogenous locus and a non-syncytial gB from a cDNA cassette. FIG. 18C is a schematic showing the ONCR-142 viral construct. FIG. 18D is a table showing the virus yield and syncytial phenotype of the indicated HSV constructs.

FIG. 19A is a schematic showing the miR-T2310 cassette for attenuating gene expression in Vero cells. FIG. 19B is a schematic showing the design of miR-T2310 mediated expression control of a syncytial gK mutant in a cDNA cassette. FIG. 19C is a schematic showing the design of miR-T2310 mediated expression control of a syncytial gB mutant in its endogenous locus. FIG. 19D contains schematics showing the ONCR-2112 and ONCR-2008 viral constructs, including the relative locations of the endogenous gB and gK loci as well as the cDNA expression cassette in either the UL3-UL4 or UL50-UL51 intergenic locus, respectively. FIG. 19E is a table showing the virus yield and syncytial phenotype of the indicated HSV constructs.

FIG. 20 is a chart showing the protein expression level of transgenes encoding mIL12 or anti-PD1 payload with the indicated G/C content in the ORF. The transgene(s) were inserted into the backbone of an oncolytic HSV (ONCR-GBM).

FIG. 21A is a schematic showing the HSV backbone used to express payload(s) in the screening experiments. The loxP-BAC replicon is located in the UL37-UL38 intergenic region, and the cDNA expression cassette for payload(s) is located in the UL3-UL4 intergenic region. The ICP27 locus comprises miRNA target sequences for miR-128, miR-129a, and miR-122; the ICP4 locus comprises miRNA target sequences for miR-124, miR-1, and miR-143; and the ICP34.5 locus comprises the target sequences for miR-128*, miR-204, and miR-219. The virus backbone further comprises D285N/A549T mutations in gB, eGFR inserted in gC, and a mutant US12. FIG. 21B is a table showing the payload molecule(s) for each indicated HSV. The cDNA payloads are expressed from a cassette inserted in the UL3-UL4 intergenic locus of ONCR-142, which utilizes the hybrid CAG promoter and rabbit b-Globin polyadenylation signal to control transgene expression.

FIG. 22A is a plot showing the overall survival (%) of mice after intratumoral injection of the indicated HSV vector over time in an orthotopic CT2A cell line model. FIG. 22B is a plot showing the overall survival (%) of mice after intratumoral injection of the indicated HSV vector over time in an orthotopic mNectin1-expressing GL261 cell line model. FIG. 22C is a plot showing a re-challenge study using the GL261 model. FIG. 22D is a plot showing the percentage of various immune cells in the CT2A model. FIG. 22E contains plots showing the immune cell counts after the administration of the indicated oncolytic viruses.

FIG. 23 is a plot showing the dose-response effect of the ONCR-GBM vector encoding IL12 and anti-PD1 antibody payloads in the orthotopic mNectin1-expressing GL261 cell line model, as measured by overall survival (%).

FIGS. 24A-24J. show survival plots of GL261 model mice administered with the indicated HSV combination encoding various payload molecules.

FIG. 25A is a schematic showing the HSV construct design of ONCR-2183 comprising an integrin-targeting knottin peptide in the gD. FIG. 25B shows schematics of expression cassettes for expressing payload combinations. rBG pA: Rabbit Beta Globin gene polyadenylation signal; CAG: CAG promoter; CMV: cytomegalovirus (CMV) promoter; UBC pA: Human Ubiquitin C gene polyadenylation signal; COXIV pA: Human Cytochrome Oxidase IV gene polyadenylation signal; GAPDH: human glyceraldehyde-3-phosphate dehydrogenase (GAPDH) promoter; EF1a: EF-1α promoter; EIF4A1 pA: eukaryotic initiation factor 4A-I polyA. FIG. 25C shows overall survival plots of mice in the GL261 or CT2A model administered with the indicated HSV.

FIG. 26A is a schematic showing the HSV construct design of ONCR-2204 comprising an EGFR-targeting VHH in the gD. FIG. 26B shows schematics of expression cassettes for expressing payload combinations. The corresponding cDNA payloads are indicated for each virus construct. The naming of regulatory elements follows those in FIG. 25B.

### DETAILED DESCRIPTION

The present disclosure provides recombinant viral vectors that exhibit superior properties compared to those in the prior art, including improved specificity/efficacy towards cancer cells and lower off-target infection and toxicity. The recombinant engineering of the viral vectors include:
Insertion of microRNA (miR) target sequences (miR-TS) cassettes that restrict viral vector replication to cancer cells;
Insertion of transgene encoding one or more of payload molecules (e.g., those that modulates anti-tumor immune response);
Codon optimization of genes to increase protein expression level;
Insertion of retargeting domain to allow viral infection of cancer cells expressing specific protein(s);
Introduction of syncytial mutations that increases virus fusogenicity; and/or
Introduction of mutations that improves replication fidelity and/or sensitivity to acyclovir.

Compositions of the viral vectors and methods of use in killing of cancerous cells and cancer treatment are further provided herein.

Use of oncolytic viruses carries the risk of non-specific viral infection of healthy cells, leading to the death of non-cancerous cells and tissues. However, genetic manipulation of the viruses to exploit pathways, proteins, genes, and/or miRNAs that are differentially expressed in normal vs. cancerous tissue can improve the specificity of the oncolytic viruses. Non-limiting examples of such genetic manipulation include insertion of miRNA target sequence(s) and retargeting domain.

The safety profile of the viral vectors can also be improved by additional genetic manipulation to improve the improves replication fidelity of the virus and/or the sensitivity to anti-viral drugs such as acyclovir.

The oncolytic viruses described herein can also express proteins that stimulates host immune response against tumor cells, modifying the immunosuppressive microenvironment of the tumor, and/or facilitating viral spread throughout a tumor, thereby increasing their therapeutic efficacy. In addition, the transgene may be codon optimized (e.g., raising G/C content) to increase the expression level of the proteins.

Accordingly, in some embodiments, the present disclosure provides recombinant viral vectors that can stimulate a productive and durable anti-tumor immune response against cancer (e.g., glioblastoma) in vivo following a single injection (e.g., intratumoral injection). In some embodiments, the recombinant viral vectors are engineered for efficient oncolysis of glioblastoma cells.

In some embodiments, the recombinant viral vector is a recombinant HSV vector. In some embodiments, the recombinant HSV vector is derived from an HSV isolate with proven ability to replicate in the CNS. In some embodiments, the recombinant HSV is defective for anterograde and retrograde transport in neurons. In some embodiments, the recombinant HSV retains ICP47, ICP34.5, and/or ICP6 gene functions.

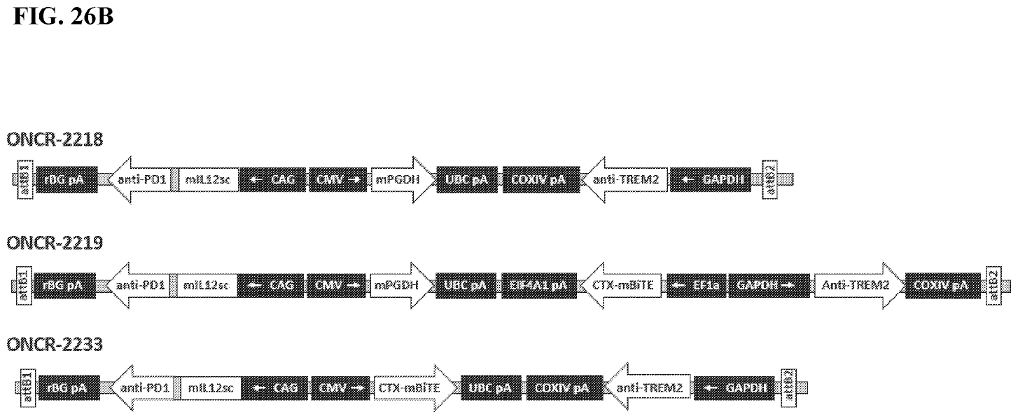

Schematics of exemplary oncolytic HSVs are provided in FIG. 26A and FIG. 26B.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

### Definitions

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. As used herein, “plurality” may refer to one or more components (e.g., one or more miRNA target sequences).

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 10% in either direction (greater than or less than) of the stated reference value unless otherwise stated or otherwise evident from the context (except where such number would exceed 100% of a possible value).

“Decrease” or “reduce” refers to a decrease or a reduction in a particular value of at least 5%, for example, 5, 6, 7, 8, 9, 10, 15, 20, 25, 30, 35, 40, 45, 50, 55, 60, 65, 70, 75, 80, 85, 90, 95, 99 or 100% as compared to a reference value. A decrease or reduction in a particular value may also be represented as a fold-change in the value compared to a reference value, for example, at least 1, 2, 3, 4, 5, 6, 7, 8, 9, 10, 15, 20, 30, 40, 50, 60, 70, 80, 90, 100, 200, 500, 1000-fold, or more, decrease as compared to a reference value.

“Increase” refers to an increase in a particular value of at least 5%, for example, 5, 6, 7, 8, 9, 10, 15, 20, 25, 30, 35, 40, 45, 50, 55, 60, 65, 70, 75, 80, 85, 90, 95, 99, 100, 200, 300, 400, 500% or more as compared to a reference value. An increase in a particular value may also be represented as a fold-change in the value compared to a reference value, for example, at least 1-fold, 2, 3, 4, 5, 6, 7, 8, 9, 10, 15, 20, 30, 40, 50, 60, 70, 80, 90, 100, 200, 500, 1000-fold or more, increase as compared to the level of a reference value.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared.

“Complementary” refers to the capacity for pairing, through base stacking and specific hydrogen bonding, between two sequences comprising naturally or non-naturally occurring (e.g., modified as described above) bases (nucleosides) or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

“Operably linked” refers to a juxtaposition wherein the components so described are in a relationship permitting them to function in their intended manner. For instance, a promoter is operably linked to a polynucleotide sequence if the promoter affects the transcription or expression of the polynucleotide sequence.

The term “subject” includes animals, such as e.g. mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein.

The term “effective amount” refers to the amount of an agent or composition required to result in a particular physiological effect (e.g., an amount required to increase, activate, and/or enhance a particular physiological effect). The effective amount of a particular agent may be represented in a variety of ways based on the nature of the agent, such as mass/volume, # of cells/volume, particles/volume, (mass of the agent)/(mass of the subject), # of cells/(mass of subject), or particles/(mass of subject). The effective amount of a particular agent may be expressed as the half-maximal effective concentration (EC₅₀), which refers to the concentration of an agent that results in a magnitude of a particular physiological response that is half-way between a reference level and a maximum response level.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals.

As used herein, the term “oncolytic virus” refers to a virus that has been modified to, or naturally, preferentially infect cancer cells.

The terms “microRNA,” “miRNA,” and “miR” are used interchangeably herein and refer to small non-coding endogenous RNAs, mostly of about 21-25 nucleotides in length, that regulate gene expression by directing their target messenger RNAs (mRNA) for degradation or translational repression.

“Essential viral gene” as used herein refers to a viral gene that is required for one or more essential viral function, such as viral replication, viral packaging, or viral infectivity.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### Oncolytic Viruses

Examples of oncolytic viruses are known in the art including, but not limited to, herpes simplex virus (HSV), an adenovirus, a polio virus, a vaccinia virus, a measles virus, a vesicular stomatitis virus, an orthomyxovirus, a parvovirus, a maraba virus or a coxsackievirus. In some embodiments, the oncolytic virus of the disclosure is an HSV. In some embodiments, the oncolytic viruses described herein are referred to as recombinant viral vectors or oncolytic vectors.

In certain embodiments, an oncolytic virus described herein is a herpesvirus (for example, herpes simplex virus (e.g., HSV-1 or HSV-2)), an adenovirus, a polio virus, a vaccinia virus, a measles virus, a vesicular stomatitis virus, an orthomyxovirus, a parvovirus, a maraba virus or a coxsackievirus. In particular embodiments, the recombinant viral vector is an HSV capable of tumor-selective vector replication as described in International PCT Publication No. WO 2015/066042, which is incorporated by reference in its entirety.

HSV-based vectors and methods for their construction are described in, for example, U.S. Pat. Nos. 7,078,029, 6,261,552, 5,998,174, 5,879,934, 5,849,572, 5,849,571, 5,837,532, 5,804,413, and 5,658,724, and International Patent Applications WO 91/02788, WO 96/04394, WO 98/15637, and WO 99/06583, which are incorporated herein by reference in their entireties. The sequence of HSV is published (NCBI Accession No. NC_001806; see also McGoech et al., J. Gen. Virol, 69 (PT 7), 1531-1574 (1988)), which may facilitate designing HSV-based vectors of the disclosure. In some cases, the oncolytic virus of the disclosure is a herpes simplex virus (HSV) and comprises a deletion of the internal repeat (joint) region comprising one copy each of the diploid genes ICP0, ICP34.5, LAT, and ICP4 along with the promoter for the ICP47 gene. In some cases, the oncolytic virus of the disclosure is a herpes simplex virus (HSV) that retains the internal repeat (joint) region comprising one copy each of the diploid genes ICP0, ICP34.5, LAT, and ICP4 along with the promoter for the ICP47 gene.

In certain embodiments, the recombinant viral vector of the disclosure is an HSV that exhibits enhanced entry into cells, either through direct infection and/or lateral spread. In one aspect, HSV vectors of the present disclosure can directly infect cells through interaction with cell proteins other than typical mediators of HSV infection (e.g., other than Nectin-1, HVEM, or heparan sulfate/chondroitin sulfate proteoglycans). In certain embodiments, the recombinant viral vector of the disclosure is an HSV and further comprises a mutation of the gB or gH gene that facilitates vector entry through non-canonical receptors. In another aspect, the disclosure provides an HSV vector further comprising mutant gH glycoproteins that exhibit lateral spread in cells typically resistant to HSV lateral spread, such as cells lacking gD receptors. In some embodiments, an HSV vector of the disclosure comprises one or more of the mutant gB or gH proteins as described in U.S. Patent Publication No. 2013/0096186, which is incorporated herein by reference in its entirety. In certain aspects, the mutant entry protein within an HSV vector is a glycoprotein involved with viral entry, such as gB, gH, and the mutant HSV vector can comprise mutated versions of both. However, the mutant entry protein can be any protein effecting entry of the HSV vector into cells. In certain embodiments, the mutant entry protein is other than gD, although the HSV vector can additionally comprise a mutant gD, such as containing a ligand or other desired mutation. Non-limiting mutations of gB or gH glycoprotein for use in the inventive HSV vector occur at one or more of the following residues: gB: D285, gB: A549, gB: S668, gH: N753, and gH: A778. In some embodiments, the inventive HSV vector comprises mutations at both gB: D285 and gB: A549, at both gH: N753 and gH: A778, and/or at each of gB: S668, gH: N753, and gH: A778. In certain embodiments, the HSV vector contains two or more of such mutations (e.g., 3 or more, 4 or more), and the HSV vector can comprise mutations in all five of these residues. In one embodiment, an HSV vector has mutations at gB: 285, gB; 549, gH: 753, and gH: 778. The mutations are referred to herein relative to the codon (amino acid) numbering of the gD, gB, and gH genes of the HSV-1 strain KOS derivative K26GFP. The sequences for gB and gH of K26GFP differ from the sequences for gB as disclosed in GenBank (#AF311740 (incorporated herein by reference)) and for gH (GenBank #X03896 (incorporated herein by reference)) as reflected in Table 1 below.

TABLE 1
Nucleotide Changes in gB and gH Coding Sequences:

| | Amino | | | | | |
|---|---|---|---|---|---|---|
| | acid | | | Nucleotide | | |
| | position | AF311740 | K26GFP | position(s) | AF311740 | K26GFP |
| gB | 313 | T | S | 938-939 | ACG | AGC |
| | 315 | A | T | 943 | GCC | ACC |
| | 515 | H | R | 1,544 | CAC | CGC |

X03896 X03896

| | | | | | | |
|---|---|---|---|---|---|---|
| gH | 12 | I | L | 1,011 | ATT | CTT |
| | 110 | P | S | 1,305 | CCG | TCG |
| | 127 | T | I | 1,357 | ACC | ATC |
| | 138 | S | A | 1,389 | TCG | GCG |
| | 150 | A | T | 1,425 | GCC | ACC |
| | 532 | A | A | 2,573 | GCT | GCG |
| | 633 | R | R | 2,876 | CGT | CGC |

However, K26GFP may contain additional differences in the region of the gene corresponding to nucleotides 2,079-2, 102 of GenBank X03896. Thus, it will be understood that the sequence of either KOS derivative K26GFP or GenBank Accession No. AF311740 can serve as a reference sequence for the gB mutations discussed herein. Also, the sequence of either KOS derivative K26GFP or GenBank Accession No. X03896 can serve as a reference sequence for the gH mutations discussed herein. However, HSV vectors of the disclosure may include homologous mutations in gB and gH of any HSV strain.

In some aspects, the mutation of the entry protein for inclusion in an HSV vector is a substitution mutation; however, mutations are not limited to substitution mutants. In certain embodiments, mutant gB or gH glycoproteins for use in an HSV vector are selected from the group of substitution mutations consisting of gB: D285N, gB: A549T, gB: S668N, gH: N753K, gH: A778V. In certain aspects, an HSV vector includes combinations of these substitutions (such as two or more of such substitutions (e.g., 3 or more, 4 or more, or all)), with the gB: D285N/gB: A549T double mutant, the gH: N753K/gH: A778V double mutant, and the gB: S668N/gH: N753K/gH: A778V triple mutant being examples of embodiments. In one embodiment, an HSV vector comprises gB: D285N/gB: A549T/gH: N753K/gH: A778V.

In certain aspects, an HSV vector comprises a mutant gB and/or a mutant gH glycoprotein, wherein the mutations in the glycoproteins are substitution mutations in at least two residues, wherein, when the vector is HSV-1 K26GFP, the at least two residues are selected from the group consisting of gB: D285, gB: A549, gB: S668, gH: N753, and gH: A778, or wherein when the vector is a homologous HSV, the at least two residues are selected from amino acids that correlate to gB: D285, gB: A549, gB: S668, gH: N753, and gH: A778 wherein the gB: D285 residue correlates to X in VYPYXEFVL (SEQ ID NO: 838), the gB: A549 residue correlates to X in KLNPNXIAS (SEQ ID NO: 839), the gB: S668 residue correlates to X in ITTVXTFID (SEQ ID NO: 840) the gH: N753 residue correlates to X in VDTDXTQQQ (SEQ ID NO: 841), and the gH: A778 residue correlates to X in VPSTXLLLF (SEQ ID NO: 842); and wherein the HSV vector is an HSV-1 or HSV-2 vector.

In some embodiments, the oncolytic HSV viruses described herein comprise one or more mutations in the UL37 gene that reduce HSV infection of neuronal cells, such as those described in International PCT Publication No. WO 2016/141320 and Richard et al., Plos Pathogens, 2017, 13 (12), e1006741.

In some embodiments, the HSV of the disclosure comprises a mutation in the UL37 viral gene, which encodes UL37 inner tegument protein with deamidase activity. The functions of UL37 protein may include: modulation of cytoplasmic secondary envelopment during viral egress; interaction with the capsid via the large tegument protein/LTP for its transportation to the host trans-Golgi network (TGN) where secondary envelopment occurs; modulation of tegumentation and capsid accumulation at the viral assembly complex; deamidation of host DDX58/RIG-I to suppress its function to sense viral dsRNA; and/or deamidation of host cGAS which abolishes cGAMP synthesis and downstream innate immune activation.

In some embodiments, the UL37 viral gene encodes a UL37 protein having at least 70%, at least 75%, at least 80%, at least 85%, at least 90%, at least 95%, at least 97%, at least 98%, at least 99%, or 100% identity to SEQ ID NO: 856.

In some embodiments, the UL37 viral gene encodes a UL37 protein comprising a mutation at 1, 2, 3, 4, or 5 of the amino acid positions corresponding to Q403, E452, Q455, Q511, and R515 of SEQ ID NO: 856. In some embodiments, the mutation is alanine substitution. In some embodiments, the UL37 viral gene encodes a UL37 protein comprising a mutation at all of of the amino acid positions corresponding to Q403, E452, Q455, Q511, and R515 of SEQ ID NO: 856. In some embodiments, the UL37 viral gene encodes a UL37 protein comprising the mutation Q403A/E452A/Q455A/Q511A/R515A according to SEQ ID NO: 856.

In some embodiments, the recombinant HSV is derived from an encephalitic HSV isolate according to SEQ ID NO: 857 (the complete genome sequence of HSV Strain MacIntyre; GenBank Accession Number MN136523.1; American Type Culture Collection (ATCC) Catalog Number VR-39). In some embodiments, the recombinant HSV comprises a polynucleotide sequence having at least 80%, at least 85%, at least 90%, at least 95%, at least 96%, at least 97%, at least 98%, at least 99%, at least 99.5%, or 100% identity to SEQ ID NO: 857 except for the mutation(s) (substitution, insertion and/or deletion) that are explicitly engineered into the recombinant HSV according to the present disclosure.

In some embodiments, the recombinant HSV is defective for anterograde transport.

In some embodiments, the recombinant HSV retains the function of ICP6, ICP34.5, ICP47, or any combination thereof. In some embodiments, the recombinant HSV retains the function of ICP6. In some embodiments, the recombinant HSV retains the function of ICP34.5. In some embodiments, the recombinant HSV retains the function of ICP47. In some embodiments, the recombinant HSV retains the function of the ICP6, ICP34.5, and ICP47 genes.

In some embodiments, the recombinant HSV comprises a bacterial artificial chromosome sequence inserted in the UL40-UL41 intergenic region. In some embodiments, the recombinant HSV comprises a bacterial artificial chromosome sequence inserted in the UL37-UL38 intergenic region.

In some embodiments, the recombinant virus comprises an exogenous expression cassette (e.g., payload molecule expression cassette) inserted in the UL50-UL51 intergenic region. In some embodiments, the recombinant virus comprises an exogenous expression cassette (e.g., payload molecule expression cassette) inserted in the UL3-UL4 intergenic region. In some embodiments, the recombinant virus comprises an exogenous expression cassette (e.g., payload molecule expression cassette) inserted in the UL40-UL41 intergenic region. In some embodiments, the recombinant virus comprises an exogenous expression cassette (e.g., payload molecule expression cassette) inserted in the UL37-UL38 intergenic region.

miRNA-Attenuated Oncolytic Viruses

MicroRNAs (miRNAs or miRs) are small non-coding endogenous RNAs that regulate gene expression by directing their target messenger RNAs for degradation or translational repression. miRs are intimately associated with normal cellular processes and therefore, deregulation of miRNAs contributes to a wide array of diseases including cancer. Many miR genes are located in cancer associated genomic regions, or in fragile sites, further strengthening the evidence that miRs play a pivotal role in cancer. miRs are differentially expressed in cancer tissues compared to normal tissues and can have a causative relationship to tumorigenesis. By exploiting this differential miR expression in diverse tumor types, the cancer therapeutics described herein possess a broad-spectrum safety and efficacy profile, wherein oncolytic viral replication is regulated based on the expression of a particular miR or group of miRs.

In some aspects, the present disclosure utilizes differential miR expression profiles to effectively restrict viral vector replication to tumor cells by incorporating miR target sequences into one or more genes required for viral replication. In some embodiments, the viral vectors described herein comprise two, three, four or more copies of a miR target sequence incorporated into one or more viral genes.

In particular, the present disclosure recognizes that miR-attenuation strategies that protect multiple cell types of the central nervous system from viral lysis enhances the therapeutic efficacy of an oncolytic virus in the treatment of glioblastoma. This is exemplified in the present disclosure by strategies to protect neurons, ependymal cells, oligodendrocytes, astrocytes, hepatocytes and/or endothelial cells from viral lysis while permitting viral replication and lysis in tumor cells. Therefore, in some embodiments, the recombinant viral vector is engineered for safety using a miR attenuation strategy to limit viral replication in the normal CNS cells (e.g., neuron, ependymal cells, oligodendrocytes, astrocytes).

In some embodiments, the present disclosure provides oncolytic viruses, wherein one or more copies of one or more micro-RNA (miRNA) target sequences are inserted into a locus of one or more viral genes. In some embodiments, the one or more viral genes are essential viral genes required for viral replication. In some embodiments, the insertion of the miRNA target sequences can limit viral replication in the normal cells of the central nervous system (CNS), thus provide an enhanced safety profile.

miRs are differentially expressed in a broad array of disease states, including multiple types of cancer. Importantly, miRNAs are differentially expressed in cancer tissues compared to normal tissues, enabling them to serve as a targeting mechanism in a broad variety of cancers. miRNAs that are associated (either positively or negatively) with carcinogenesis, malignant transformation, or metastasis are known as “oncomiRs”.

In some aspects, the expression level of a particular oncomiR is positively associated with the development or maintenance of a particular cancer. Such miRs are referred to herein as “oncogenic miRs.” In some embodiments, the expression of an oncogenic miR is increased in cancerous cells or tissues compared to the expression level observed in non-cancerous controls cells (i.e., normal or healthy controls) or is increased compared to the expression level observed in cancerous cells derived from a different cancer type. In some embodiments, the expression of an oncogenic miR is increased by at least 5%, 10%, 15%, 20%, 25%, 30%, 35%, 40%, 45%, 50%, 60%, 70%, 80%, 90%, 100%, 150%, 200%, 300%, 400%, 500%, 1000% or more compared to the expression of the oncogenic miR in a non-cancerous control cell or a cancerous cell derived from a different cancer type. In some aspects, a cancerous cell or tissue may express an oncogenic miR that is not expressed in non-cancerous control cells or tissues. Examples of oncogenic miRNAs that are frequently over-expressed in cancer tissues include, but are not limited to, miR-21, miR-155 and miR-17-92. Additional examples of oncogenic miRs are listed in Table 13.

In some embodiments, the expression of a particular oncomiR is negatively associated with the development or maintenance of a particular cancer and/or metastasis. Such oncomiRs are referred to herein as “tumor-suppressor miRs” or “tumor-suppressive miRs,” as their expression prevents or suppresses the development of cancer. In some embodiments, the expression of a tumor-suppressor miRNA is decreased in cancerous cells or tissues compared to the expression level observed in non-cancerous control cells (i.e., normal or healthy controls), or is decreased compared to the expression level of the tumor-suppressor miRNA observed in cancerous cells derived from a different cancer type. For example, the expression of a tumor-suppressor miRNA in a cancerous cell may be decreased by at least 5%, 10%, 15%, 20%, 25%, 30%, 35%, 40%, 45%, 50%, 60%, 70%, 80%, 90%, or 100% compared to the expression of the tumor-suppressor miRNA in a non-cancerous control cell or a cancerous cell derived from a different cancer type. In some aspects, a non-cancerous control cell may express a tumor-suppressor miRNA that is not expressed in cancerous cells. Examples of tumor-suppressive miRNAs include, but are not limited to, miR-122, miR-184, miR-34a, let7a, miR-145-5p, miR-199a-5p, miR-451a, miR-125a, miR-125a-5p, miR-126-3p, miR-233-3p, miR-143-3p, miR-1-3p, miR-133a-3p, miR-127a-3p, miR-133b, miR-134-3p, miR-124, miR-101, miR-125b, miR-145, miR-559, miR-213, miR-31-5p, miR-205p, miR-15a, miR-16-1, miR-34, as well as miRNAs of the let-7 family. Additional examples of tumor-suppressive miRs are listed in Table 12 and Table 14.

Cancer pathogenesis is a heterogeneous and multigenic process. As such, activation of particular pathways and the expression of particular genes may lead to cancer development in one context, and result in distinct or opposing results when activated or expressed in a different context. Therefore, the characterization of a particular gene or miR as an “oncogene” or “oncogenic miR” or as a “tumor-suppressor” or “tumor-suppressive miR” is not a binary distinction and will vary according to the type of cancer. For example, the expression of one miRNA may be increased in a particular cancer and associated with the development of that cancer, while the expression of the same miRNA may be decreased in a different cancer and associated with prevention of the development of that cancer. However, some miRNAs may function as oncogenic miRNAs independent of the type of cancer. For example, some miRNAs target mRNA transcripts of tumor suppressor genes for degradation, thereby reducing expression of the tumor suppressor protein. For example, miR-152b functions as an oncogenic miR in the vast majority of hematologic malignancies, but functions as a tumor-suppressive miR in many solid tumors. Further, a particular miR may be highly expressed in both cancerous and non-cancerous cells. For example, miR-155 is highly expressed in normal cells, playing an essential role in macrophage polarization, and is also highly expressed in cancer cells. As such, the development of the miR-attenuated, genome-editing, and microenvironment-remodeling oncolytic viruses described herein is based on the differential expression of a particular miR or group of miRs in one cell population or tissue compared to another cell population or tissue. One of skill in the art will understand that the term tumor-suppressive miR generally refers to a miR that is more highly expressed in a non-cancerous cell or tissue compared to a cancerous cell or tissue, and that the term oncogenic miR generally refers to a miR that is more highly expressed in a cancerous cell or tissue compared to a non-cancerous cell or tissue. One of skill in the art will further understand that a miR characterized as a tumor-suppressive miR in one type of cancer may or more may not function as a tumor-suppressive miR in a different type of cancer, and that a miR characterized as an oncogenic miR in one type of cancer may or more may not function as an oncogenic miR in a different type of cancer.

Table 10 shows the relationship between 12 select oncomiRs (9 tumor suppressors and 3 oncogenic miRNAs) and numerous cancers. A list of 3,410 oncomiR-cancer relationships is shown in Table 11. miRNAs regulate many transcripts of proteins that are involved in the control of cellular proliferation and apoptosis. Regulated proteins include conventional proto-oncoproteins and tumor suppressors such as Ras, Myc, Bcl2, PTEN and p53. Aberrant expression of miRNAs therefore often is involved in development of cancer and can therapeutically be corrected by either inhibiting oncogenic miRNAs or replacing the depleted tumor suppressor miRNA. Further, the differential expression of particular oncomiRs in cancerous vs. non-cancerous cells can be exploited as a means to target cancer therapeutics specifically to cancer cells. As such, in some embodiments, the oncolytic viral vectors described herein can comprise insertion of miRNA target sequences into the viral genome, thereby restricting viral vector replication to cancer or tumor cells, and/or one or more polynucleotides incorporated into the viral genome whose product(s) disrupt the function of an oncogenic miRNA.

One aspect of the disclosure comprises a recombinant oncolytic virus (or viral vector) comprising a plurality of copies of one or more miRNA target sequences inserted into a locus of one or more essential viral genes. In certain embodiments, a recombinant oncolytic virus may comprise miRNA target sequences inserted into a locus of at least one, at least two, at least three, at least four, at least five, at least six, at least seven, at least eight, at least nine, or at least ten essential viral genes. miRNAs expressed in normal (non-cancerous) cells can bind to such target sequences and suppress expression of the viral gene containing the miRNA target sequence, thereby limiting viral replication in healthy, non-cancerous cells. Such recombinant oncolytic viruses are referred to herein as “miR-attenuated” or “replication-restricted” as they demonstrate reduced or attenuated viral replication in cells that express one or more miRNAs capable of binding to the incorporated miR target sequences compared to cells that do not express, or have reduced expression of, the miR. By incorporating miRNA target sequences into key genes required for viral replication, viral replication can be conditionally suppressed in normal diploid cells expressing the miRNAs and can proceed normally in cells that do not express the miRNAs. In such embodiments, normal, non-cancerous cells are protected from lytic effects of infection by the recombinant viral vector.

In certain embodiments, the one or more miRNA target sequences is incorporated into the 5′ untranslated region (UTR) and/or 3′ UTR of one or more essential viral genes. In some embodiments, the oncolytic virus is a herpes simplex virus (HSV), and the viral genes required for viral replication include any of UL1, UL5, UL6, UL7, UL8, UL9, UL11, UL12, UL14, UL15, UL17, UL18, UL19, UL20, UL22, UL25, UL26, UL26.5, UL27, UL28, UL29, UL30, UL31, UL32, UL33, UL34, UL35, UL36, UL37, UL38, UL39, UL40, UL42, UL48, UL49, UL52, UL53, UL54, ICP0, ICP4, ICP22, ICP27, ICP34.5, ICP47, gamma-34.5, US3, US4, US5, US6, US7, US8, US9, US10, US11, and/or US12. In certain embodiments, the oncolytic virus is HSV and comprises one or more miRNA target sequences incorporated into the 5′ or 3′ UTR of one or more essential viral genes. In some embodiments, the oncolytic virus is HSV, and the one or more miRNA target sequences is incorporated into one or more of ICP4, ICP27, UL8, UL42, UL19, and ICP34.5. In some embodiments, the oncolytic virus is HSV, and the one or more miRNA target sequences is incorporated into the 5′ or 3′ UTR of one or more of ICP4, ICP27, UL8, UL42, UL19, and ICP34.5. In some embodiments, the oncolytic virus is HSV, and the one or more miRNA target sequences is incorporated into the 5′ or 3′ UTR of one or more of ICP4, ICP8, ICP27, and UL8.

miRNA Target Sequence Cassettes

In animals, genes for miRNAs are transcribed to a primary miRNA (pri-miRNA), which is then processed in the nucleus by Drosha, a class 2 RNase III enzyme, to form a precursor miRNA (pre-miRNA) hairpin. The pre-miRNA hairpins are transported to the cytoplasm, where they are cleaved by the RNase III enzyme Dicer. This endoribonuclease interacts with 5′ and 3′ ends of the hairpin and cuts away the loop joining the 3′ and 5′ arms, yielding a duplex RNA molecule about 22 nucleotides in length. Although either strand of the duplex may potentially act as a functional miRNA, typically one strand of the miRNA is degraded and only one strand is loaded onto the Argonaute (Ago) protein to produce the effector RNA-induced silencing complex (RISC) where the miRNA and its mRNA target interact (Wahid et al., 1803:11, 2010, 1231-1243).

Herein, the gene encoding a particular miRNA is referenced as “MIR” followed by the miRNA number. The intermediate hairpin pre-miRNA molecules are referenced as “mir-” followed by the miRNA number, while the mature single-stranded miRNA molecule is referenced as “miR-” followed by the miRNA number. For example, “MIR122” refers to the gene encoding a hairpin mir-122 pre-miRNA molecule, which is then processed into a mature miR-122 molecule. Due to the hairpin structure of the pre-miRNA, it is possible that two mature microRNAs can originate from opposite arms of the same pre-miRNA. In some instances, expression data clearly identify one strand as the predominantly expressed miRNA and the other as the minor product. In such instances, the mature miRNA sequences are assigned names of the form miR-##(the predominant product) and miR-##* (minor product from the opposite arm of the precursor). For example, the major and minor products of mir-56 are denoted as miR-56 and miR-56*, respectively. When the existing data are not sufficient to determine which sequence is the predominant one, or when they are found in roughly similar amounts, the two mature miRNA products are denoted as miR-##-5p (from the 5′ arm of the pre-miRNA hairpin) and miR-##-3p (from the 3′ arm of the pre-miRNA hairpin). For example, the two mature miRNA products of mir-142 are denoted as miR-142-5p and miR-142-3p. Because they originate from opposite ends of the pre-miRNA hairpin, the −3p and −5p products of a particular miRNA will comprise different RNA sequences and will therefore recognize different target sequences.

Herein, miRNA target sequences are inserted into the locus of one or more essential viral genes in the form of a “miR target sequence cassette” or “miR-TS cassette.” A miR-TS cassette which refers to a polynucleotide sequence comprising one or more miRNA target sequences and capable of being inserted into a specific locus of a viral gene. When transcribed, the mRNA transcripts of a viral gene comprising a miR-TS cassette will comprise one or more miRNA target sequences. In some embodiments, the miR-TS cassettes described herein comprise at least one miRNA target sequence. In some embodiments, the miR-TS cassettes described herein comprise a plurality of miRNA target sequences. For example, in some embodiments, the miR-TS cassettes described herein comprise 2, 3, 4, 5, 6, 7, 8, 9, 10, 11, 12, 13, 14, 15, 16, 17, 18, 19, 20, or more miRNA target sequences. In some embodiments, the miR-TS cassettes described herein comprise miRNA target sequences for at least 4 miRNAs. In some embodiments, the miR-TS cassettes described herein comprise miRNA target sequences for 4 miRNAs.

In some embodiments, wherein the miR-TS cassettes comprise two or more miRNA target sequences, the two or more target sequences are arranged such that the total length of the miR-TS cassette (m) is less than or equal to the average length of the miRNA target sequences (n) multiplied by the total number of miRNA target sequences in the cassette (γ) plus the average length of a linker sequence (l) multiplied by the total number of miRNA target sequences in the cassette plus 1 (y+1). Thus, the length of a miR-TS cassette (m) can be represented by the formula: m≤(n*y)+ (l*(y+1)), wherein n=the average length of the miRNA target sequences, l=the average length of the linker sequences, and y=the total number of target sequences in the miR-TS cassette). As an illustrative example, if a miR-TS cassettes comprises 4 miRNA target sequences (y) with an average length of 21 nt (n), and the average length of the linker sequences is between 4 and 25 nt (l), the length of the miR-TS cassette (m) is between about 104 nt and about 205 nt.

As used herein, the “length” of a miR-TS cassette is defined as the total number of nucleotides (basepairs for double-stranded polynucleotides) from the 5′ nucleotide of the first miR-TS to the 3′ nucleotide of the last miR-TS in the polynucleotide, inclusive of any intervening sequences. For non-overlapping miR-TSs, the minimum length of a miR-TS cassette will be the sum of the lengths of the miR-TSs. Spacers increase the length. The choice of spacer length determines the number of additional nucleotides in the cassette. Longer spacers increase the length of the cassette more than shorter spacers. By recognizing that shorter spacers (as short as 0, 1, 2, 3, 4, 5, or 6 nt) can be used when miR-TSs are interleaved (minimizing the number of mi-TSs for the same miRNA that are adjacent to one another)—the interleaved miR-TSs serving to increase the space between the other miR-TSs—the present inventors have determined that it is possible to generate shorter miR-TS cassettes than is possible in miR-TS cassettes in which miR-TSs for the same miRNA are arrayed in tandem, e.g. four of one type followed by four of the next type. In some embodiments, the length of the miR-TS cassette is less than 1000 nt. In some embodiments, the length of the miR-TS cassette is less than 900 nt, less than 800 nt, less than 700 nt, less than 600 nt, less than 500 nt, less than 400 nt, less than 300 nt, less than 200 nt, less than 100 nt, or less than 50 nt. In some embodiments, the length of the miR-TS cassette is less than 400 nt, less than 390 nt, less than 380 nt, less than 370 nt, less than 360 nt, less than 350 nt, less than 340 nt, less than 330 nt, or less than 320 nt. In some embodiments, the length of the miR-TS cassette is less than 320 nt.

In some embodiments, the length of the miR-TS cassette is less than 26, 27, 28, 29, or 30 nt times the number of miR-TS sites, less than about 30 nt times the number of miR-TS sites, less than about 35 nt times the number of miR-TS sites, or less than about 40 nt times the number of miR-TS sites.

In some embodiments, the miR-TS cassettes comprise a plurality of miRNA target sequences, wherein each miRNA target sequence in the plurality is a target sequence for the same miRNA. For example, the miR-TS cassettes may comprise 2, 3, 4, 5, 6, 7, 8, 9, 10 or more copies of the same miR target sequence. In some embodiments, the miR-TS cassettes comprise between 2 to 6 copies of the same miR target sequence. In some embodiments, the miR-TS cassettes comprise 4 copies of the same miR target sequence. In some embodiments, the miR-TS cassettes comprise 3 copies of the same miR target sequence.

In some embodiments, the miR-TS cassettes described herein comprise a plurality of miRNA target sequences, wherein the plurality comprises at least two different miRNA target sequences. In some embodiments, the miR-TS cassettes described herein comprise 2, 3, 4, 5, 6, 7, 8, 9, or 10 different miRNA target sequences. For example, in some embodiments, the miR-TS cassette may one or more copies of a first miRNA target sequence and one or more copies of a second miRNA target sequence. In some embodiments, the miR-TS cassette comprises at least 2, 3, 4, 5, 6, 7, 8, 9, 10, or more copies of a first miR target sequence and at least 2, 3, 4, 5, 6, 7, 8, 9, 10, or more copies of a second miR target sequence. In some embodiments, the miR-TS cassette comprises 3 or 4 copies of a first miR target sequence and 3 or 4 copies of a second miR target sequence. In some embodiments, the plurality of miRNA target sequences comprises at least 3 different miRNA target sequences. For example, in some embodiments, the miR-TS cassette comprises one or more copies of a first miR target sequence, one or more copies of a second miR target sequence, and one or more copies of a third miR target sequence. In some embodiments, the miR-TS cassette comprises at least 2, 3, 4, 5, 6, 7, 8, 9, 10, or more copies of a first miR target sequence, at least 2, 3, 4, 5, 6, 7, 8, 9, 10, or more copies of a second miR target sequence, and at least 2, 3, 4, 5, 6, 7, 8, 9, 10, or more copies of a third miR target sequence. In some embodiments, the miR-TS cassette comprises 3 or 4 copies of a first miR target sequence, 3 or 4 copies of a second miR target sequence, and 3 or 4 copies of a third miR target sequence. In some embodiments, the plurality of miRNA target sequences comprises at least 4 different miRNA target sequences. For example, in some embodiments, the miR-TS cassette comprises one or more copies of a first miR target sequence, one or more copies of a second miR target sequence, one or more copies of a third miR target sequence, and one or more copies of a fourth miR target sequence. In some embodiments, the miR-TS cassette comprises at least 2, 3, 4, 5, 6, 7, 8, 9, 10, or more copies of a first miR target sequence, at least 2, 3, 4, 5, 6, 7, 8, 9, 10, or more copies of a second miR target sequence, at least 2, 3, 4, 5, 6, 7, 8, 9, 10, or more copies of a third miR target sequence, and at least 2, 3, 4, 5, 6, 7, 8, 9, 10, or more copies of a fourth miR target sequence. In some embodiments, the miR-TS cassette comprises 3 or 4 copies of a first miR target sequence, 3 or 4 copies of a second miR target sequence, 3 or 4 copies of a third miR target sequence, and 3 or 4 copies of a fourth miR target sequence. In some embodiments, the miR-TS cassette comprises 3 copies of a first miR target sequence, 3 copies of a second miR target sequence, 3 copies of a third miR target sequence, and 3 copies of a fourth miR target sequence.

In some aspects, wherein the miR-TS cassettes comprise a plurality of miRNA target sequences, the plurality of miRNA target sequences may arranged in tandem, without any intervening nucleic acid sequences. In some aspects, the plurality of miRNA target sequences may be separated by a linker sequence. In some embodiments, the linker sequence comprises 2, 3, 4, 5, 6, 7, 8, 9, 10, 15, 20, 25, or more nucleotides. In some embodiments, the linker sequence comprises about 4 to about 20 nucleotides. In some embodiments, the linker sequence comprises about 4 to about 16 nucleotides. In some embodiments, the linker sequence comprises 4 nucleotides. As an illustrative embodiment, a miR-TS cassette may comprise 1, 2, 3, 4, 5, 6, 7, 8, 9, 10, or more of the following subunits: (a) a first miRNA target sequence—linker—a second miRNA target sequence, wherein adjacent subunits are separated by an additional linker sequence. In some embodiments, the first and the second miRNA target sequence are targets of the same miRNA. In some embodiments, the first and the second miRNA target sequence are targets of different miRNAs.

In some embodiments, miR-TS cassettes described herein comprise a miRNA target sequence that is at most 4, at most 3, at most 2, or at most 1 nucleotide(s) different from the reverse complement of a sequence selected from SEQ ID NOs: 1-803 and 861-866. In some embodiments, miR-TS cassettes described herein comprise a miRNA target sequence that comprises or consists of the reverse complement of a sequence selected from SEQ ID NOs: 1-803 and 861-866.

In some embodiments, miR-TS cassettes described herein comprise a miRNA target sequence that comprises or consists of any one of SEQ ID NOs: 804-837 and 867-872.

In some embodiments, the miR-TS cassettes described herein comprise at least 1, at least 2, at least 3, or at least 4 miR-122-5p target sequences. In some embodiments, the miR-122-5p target sequences are at most 4, at most 3, at most 2, or at most 1 nucleotide(s) different from SEQ ID NO: 804. In some embodiments, the miR-122-5p target sequences comprise or consist of SEQ ID NO: 804. In some embodiments, the virus described herein comprises one or more miR-122-5p target sequences in ICP4.

In some embodiments, the miR-TS cassettes described herein comprise at least 1, at least 2, at least 3, or at least 4 miR-124-3p target sequences. In some embodiments, the miR-124-3p target sequences are at most 4, at most 3, at most 2, or at most 1 nucleotide(s) different from SEQ ID NO: 805. In some embodiments, the miR-124-3p target sequences comprise or consist of SEQ ID NO: 805. In some embodiments, the virus described herein comprises one or more miR-124-3p target sequences in ICP4. In some embodiments, the virus described herein comprises one or more miR-124-3p target sequences in ICP8. In some embodiments, the virus described herein comprises one or more miR-124-3p target sequences in ICP27. In some embodiments, the virus described herein comprises one or more miR-124-3p target sequences in both ICP4 and ICP8. In some embodiments, the virus described herein comprises one or more miR-124-3p target sequences in both ICP8 and ICP27. In some embodiments, the virus described herein comprises one or more miR-124-3p target sequences in both ICP4 and ICP27. In some embodiments, the virus described herein comprises one or more miR-124-3p target sequences in ICP4, ICP8, and ICP27.

In some embodiments, the miR-TS cassettes described herein comprise at least 1, at least 2, at least 3, or at least 4 miR-125a-5p target sequences. In some embodiments, the miR-125a-5p target sequences are at most 4, at most 3, at most 2, or at most 1 nucleotide(s) different from SEQ ID NO: 806. In some embodiments, the miR-125a-5p target sequences comprise or consist of SEQ ID NO: 806.

In some embodiments, the miR-TS cassettes described herein comprise at least 1, at least 2, at least 3, or at least 4 miR-126-3p target sequences. In some embodiments, the miR-126-3p target sequences are at most 4, at most 3, at most 2, or at most 1 nucleotide(s) different from SEQ ID NO: 807 or SEQ ID NO: 808. In some embodiments, the miR-126-3p target sequences comprise or consist of SEQ ID NO: 807 or SEQ ID NO: 808.

In some embodiments, the miR-TS cassettes described herein comprise at least 1, at least 2, at least 3, or at least 4 miR-127a-3p target sequences. In some embodiments, the miR-127a-3p target sequences are at most 4, at most 3, at most 2, or at most 1 nucleotide(s) different from SEQ ID NO: 809. In some embodiments, the miR-127a-3p target sequences comprise or consist of SEQ ID NO: 809.

In some embodiments, the miR-TS cassettes described herein comprise at least 1, at least 2, at least 3, or at least 4 miR-128-3p target sequences. In some embodiments, the miR-128-3p target sequences are at most 4, at most 3, at most 2, or at most 1 nucleotide(s) different from SEQ ID NO: 810 or SEQ ID NO: 811. In some embodiments, the miR-128-3p target sequences comprise or consist of SEQ ID NO: 810 or SEQ ID NO: 811.

In some embodiments, the miR-TS cassettes described herein comprise at least 1, at least 2, at least 3, or at least 4 miR-129-3p target sequences. In some embodiments, the miR-129-3p target sequences are at most 4, at most 3, at most 2, or at most 1 nucleotide(s) different from SEQ ID NO: 812. In some embodiments, the miR-129-3p target sequences comprise or consist of SEQ ID NO: 812.

In some embodiments, the miR-TS cassettes described herein comprise at least 1, at least 2, at least 3, or at least 4 miR-129-5p target sequences. In some embodiments, the miR-129-5p target sequences are at most 4, at most 3, at most 2, or at most 1 nucleotide(s) different from SEQ ID NO: 813. In some embodiments, the miR-129-5p target sequences comprise or consist of SEQ ID NO: 813.

In some embodiments, the miR-TS cassettes described herein comprise at least 1, at least 2, at least 3, or at least 4 miR-130b-3p target sequences. In some embodiments, the miR-130b-3p target sequences are at most 4, at most 3, at most 2, or at most 1 nucleotide(s) different from SEQ ID NO: 814. In some embodiments, the miR-130b-3p target sequences comprise or consist of SEQ ID NO: 814.

In some embodiments, the miR-TS cassettes described herein comprise at least 1, at least 2, at least 3, or at least 4 miR-130b-5p target sequences. In some embodiments, the miR-130b-5p target sequences are at most 4, at most 3, at most 2, or at most 1 nucleotide(s) different from SEQ ID NO: 815. In some embodiments, the miR-130b-5p target sequences comprise or consist of SEQ ID NO: 815.

In some embodiments, the miR-TS cassettes described herein comprise at least 1, at least 2, at least 3, or at least 4 miR-133a-3p target sequences. In some embodiments, the miR-133a-3p target sequences are at most 4, at most 3, at most 2, or at most 1 nucleotide(s) different from SEQ ID NO: 816. In some embodiments, the miR-133a-3p target sequences comprise or consist of SEQ ID NO: 816.

In some embodiments, the miR-TS cassettes described herein comprise at least 1, at least 2, at least 3, or at least 4 miR-133b-3p target sequences. In some embodiments, the miR-133b-3p target sequences are at most 4, at most 3, at most 2, or at most 1 nucleotide(s) different from SEQ ID NO: 817. In some embodiments, the miR-133b-3p target sequences comprise or consist of SEQ ID NO: 817.

In some embodiments, the miR-TS cassettes described herein comprise at least 1, at least 2, at least 3, or at least 4 miR-134-3p target sequences. In some embodiments, the miR-134-3p target sequences are at most 4, at most 3, at most 2, or at most 1 nucleotide(s) different from SEQ ID NO: 818. In some embodiments, the miR-134-3p target sequences comprise or consist of SEQ ID NO: 818.

In some embodiments, the miR-TS cassettes described herein comprise at least 1, at least 2, at least 3, or at least 4 miR-137-3p target sequences. In some embodiments, the miR-137-3p target sequences are at most 4, at most 3, at most 2, or at most 1 nucleotide(s) different from SEQ ID NO: 819. In some embodiments, the miR-137-3p target sequences comprise or consist of SEQ ID NO: 819. In some embodiments, the virus described herein comprises one or more miR-137-3p target sequences in ICP4. In some embodiments, the virus described herein comprises one or more miR-137-3p target sequences in ICP27. In some embodiments, the virus described herein comprises one or more miR-137-3p target sequences in ICP4 and ICP27.

In some embodiments, the miR-TS cassettes described herein comprise at least 1, at least 2, at least 3, or at least 4 miR-1-3p target sequences. In some embodiments, the miR-1-3p target sequences are at most 4, at most 3, at most 2, or at most 1 nucleotide(s) different from SEQ ID NO: 820. In some embodiments, the miR-1-3p target sequences comprise or consist of SEQ ID NO: 820.

In some embodiments, the miR-TS cassettes described herein comprise at least 1, at least 2, at least 3, or at least 4 miR-143-3p target sequences. In some embodiments, the miR-143-3p target sequences are at most 4, at most 3, at most 2, or at most 1 nucleotide(s) different from SEQ ID NO: 821. In some embodiments, miR-143-3p target sequences comprise or consist of SEQ ID NO: 821.

In some embodiments, the miR-TS cassettes described herein comprise at least 1, at least 2, at least 3, or at least 4 miR-145-3p target sequences. In some embodiments, the miR-145-3p target sequences are at most 4, at most 3, at most 2, or at most 1 nucleotide(s) different from SEQ ID NO: 822. In some embodiments, the miR-145-3p target sequences comprise or consist of SEQ ID NO: 822.

In some embodiments, the miR-TS cassettes described herein comprise at least 1, at least 2, at least 3, or at least 4 miR-145-5p target sequences. In some embodiments, the miR-145-5p target sequences are at most 4, at most 3, at most 2, or at most 1 nucleotide(s) different from SEQ ID NO: 823. In some embodiments, the miR-145-5p target sequences comprise or consist of SEQ ID NO: 823. In some embodiments, the virus described herein comprises one or more miR-145-5p target sequences in UL8.

In some embodiments, the miR-TS cassettes described herein comprise at least 1, at least 2, at least 3, or at least 4 miR-184-3p target sequences. In some embodiments, the miR-184-3p target sequences are at most 4, at most 3, at most 2, or at most 1 nucleotide(s) different from SEQ ID NO: 824. In some embodiments, the miR-184-3p target sequences comprise or consist of SEQ ID NO: 824.

In some embodiments, the miR-TS cassettes described herein comprise at least 1, at least 2, at least 3, or at least 4 miR-199a-3p target sequences. In some embodiments, the miR-199a-3p target sequences are at most 4, at most 3, at most 2, or at most 1 nucleotide(s) different from SEQ ID NO: 825. In some embodiments, the miR-199a-3p target sequences comprise or consist of SEQ ID NO: 825.

In some embodiments, the miR-TS cassettes described herein comprise at least 1, at least 2, at least 3, or at least 4 miR-199a-5p target sequences. In some embodiments, the miR-199a-5p target sequences are at most 4, at most 3, at most 2, or at most 1 nucleotide(s) different from SEQ ID NO: 826. In some embodiments, the miR-199a-5p target sequences comprise or consist of SEQ ID NO: 826.

In some embodiments, the miR-TS cassettes described herein comprise at least 1, at least 2, at least 3, or at least 4 miR-204-5p target sequences. In some embodiments, the miR-204-5p target sequences are at most 4, at most 3, at most 2, or at most 1 nucleotide(s) different from SEQ ID NO: 827. In some embodiments, the miR-204-5p target sequences comprise or consist of SEQ ID NO: 827.

In some embodiments, the miR-TS cassettes described herein comprise at least 1, at least 2, at least 3, or at least 4 miR-208b-3p target sequences. In some embodiments, the miR-208b-3p target sequences are at most 4, at most 3, at most 2, or at most 1 nucleotide(s) different from SEQ ID NO: 828. In some embodiments, the miR-208b-3p target sequences comprise or consist of SEQ ID NO: 828.

In some embodiments, the miR-TS cassettes described herein comprise at least 1, at least 2, at least 3, or at least 4 miR-214-3p target sequences. In some embodiments, the miR-214-3p target sequences are at most 4, at most 3, at most 2, or at most 1 nucleotide(s) different from SEQ ID NO: 829. In some embodiments, the miR-214-3p target sequences comprise or consist of SEQ ID NO: 829.

In some embodiments, the miR-TS cassettes described herein comprise at least 1, at least 2, at least 3, or at least 4 miR-217-5p target sequences. In some embodiments, the miR-217-5p target sequences are at most 4, at most 3, at most 2, or at most 1 nucleotide(s) different from SEQ ID NO: 830. In some embodiments, the miR-217-5p target sequences comprise or consist of SEQ ID NO: 830.

In some embodiments, the miR-TS cassettes described herein comprise at least 1, at least 2, at least 3, or at least 4 miR-219a-5p target sequences. In some embodiments, the miR-219a-5p target sequences are at most 4, at most 3, at most 2, or at most 1 nucleotide(s) different from SEQ ID NO: 831. In some embodiments, the miR-219a-5p target sequences comprise or consist of SEQ ID NO: 831.

In some embodiments, the miR-TS cassettes described herein comprise at least 1, at least 2, at least 3, or at least 4 miR-223-3p target sequences. In some embodiments, the miR-223-3p target sequences are at most 4, at most 3, at most 2, or at most 1 nucleotide(s) different from SEQ ID NO: 832. In some embodiments, the miR-223-3p target sequences comprise or consist of SEQ ID NO: 832.

In some embodiments, the miR-TS cassettes described herein comprise at least 1, at least 2, at least 3, or at least 4 miR-34a-5p target sequences. In some embodiments, the miR-34a-5p target sequences are at most 4, at most 3, at most 2, or at most 1 nucleotide(s) different from SEQ ID NO: 833. In some embodiments, the miR-34a-5p target sequences comprise or consist of SEQ ID NO: 833.

In some embodiments, the miR-TS cassettes described herein comprise at least 1, at least 2, at least 3, or at least 4 miR-451a target sequences. In some embodiments, the miR-451a target sequences are at most 4, at most 3, at most 2, or at most 1 nucleotide(s) different from SEQ ID NO: 834. In some embodiments, the miR-451a target sequences comprise or consist of SEQ ID NO: 834.

In some embodiments, the miR-TS cassettes described herein comprise at least 1, at least 2, at least 3, or at least 4 miR-559-5p target sequences. In some embodiments, the miR-559-5p target sequences are at most 4, at most 3, at most 2, or at most 1 nucleotide(s) different from SEQ ID NO: 835. In some embodiments, the miR-559-5p target sequences comprise or consist of SEQ ID NO: 835.

In some embodiments, the miR-TS cassettes described herein comprise at least 1, at least 2, at least 3, or at least 4 miR-Let-7a-5p target sequences. In some embodiments, the miR-Let-7a-5p target sequences are at most 4, at most 3, at most 2, or at most 1 nucleotide(s) different from SEQ ID NO: 836. In some embodiments, the miR-Let-7a-5p target sequences comprise or consist of SEQ ID NO: 836.

In some embodiments, the miR-TS cassettes described herein comprise at least 1, at least 2, at least 3, or at least 4 miR-9-5p target sequences. In some embodiments, the miR-9-5p target sequences are at most 4, at most 3, at most 2, or at most 1 nucleotide(s) different from SEQ ID NO: 837. In some embodiments, the miR-9-5p target sequences comprise or consist of SEQ ID NO: 837.

In some embodiments, the miR-TS cassettes described herein comprise at least 1, at least 2, at least 3, or at least 4 miR-34b-5p target sequences. In some embodiments, the miR-34b-5p target sequences are at most 4, at most 3, at most 2, or at most 1 nucleotide(s) different from SEQ ID NO: 867. In some embodiments, the miR-34b-5p target sequences comprise or consist of SEQ ID NO: 867. In some embodiments, the virus described herein comprises one or more miR-34b-5p target sequences in UL8.

In some embodiments, the miR-TS cassettes described herein comprise at least 1, at least 2, at least 3, or at least 4 miR-34b-3p target sequences. In some embodiments, the miR-34b-3p target sequences are at most 4, at most 3, at most 2, or at most 1 nucleotide(s) different from SEQ ID NO: 868. In some embodiments, the miR-34b-3p target sequences comprise or consist of SEQ ID NO: 868.

In some embodiments, the miR-TS cassettes described herein comprise at least 1, at least 2, at least 3, or at least 4 miR-34c-5p target sequences. In some embodiments, the miR-34c-5p target sequences are at most 4, at most 3, at most 2, or at most 1 nucleotide(s) different from SEQ ID NO: 869. In some embodiments, the miR-34c-5p target sequences comprise or consist of SEQ ID NO: 869. In some embodiments, the virus described herein comprises one or more miR-34c-5p target sequences in ICP8. In some embodiments, the virus described herein comprises one or more miR-34c-5p target sequences in ICP27. In some embodiments, the virus described herein comprises one or more miR-34c-5p target sequences in UL8. In some embodiments, the virus described herein comprises one or more miR-34c-5p target sequences in ICP8 and ICP27. In some embodiments, the virus described herein comprises one or more miR-34c-5p target sequences in ICP27 and UL8. In some embodiments, the virus described herein comprises one or more miR-34c-5p target sequences in ICP8 and UL8.

In some embodiments, the miR-TS cassettes described herein comprise at least 1, at least 2, at least 3, or at least 4 miR-128T target sequences. In some embodiments, the miR-128T target sequences are at most 4, at most 3, at most 2, or at most 1 nucleotide(s) different from SEQ ID NO: 870. In some embodiments, the miR-128T target sequences comprise or consist of SEQ ID NO: 870. In some embodiments, the virus described herein comprises one or more miR-128T target sequences in ICP4. In some embodiments, the virus described herein comprises one or more miR-128T target sequences in ICP27. In some embodiments, the virus described herein comprises one or more miR-128T target sequences in ICP4 and ICP27.

In some embodiments, the miR-TS cassettes described herein comprise at least 1, at least 2, at least 3, or at least 4 miR-129-2-3p target sequences. In some embodiments, the miR-129-2-3p target sequences are at most 4, at most 3, at most 2, or at most 1 nucleotide(s) different from SEQ ID NO: 871. In some embodiments, the miR-129-2-3p target sequences comprise or consist of SEQ ID NO: 871. In some embodiments, the virus described herein comprises one or more miR-129-2-3p target sequences in ICP8.

In some embodiments, the miR-TS cassettes described herein comprise at least 1, at least 2, at least 3, or at least 4 miR-132-3p target sequences. In some embodiments, the miR-132-3p target sequences are at most 4, at most 3, at most 2, or at most 1 nucleotide(s) different from SEQ ID NO: 872. In some embodiments, the miR-132-3p target sequences comprise or consist of SEQ ID NO: 872. In some embodiments, the virus described herein comprises one or more miR-132-3p target sequences in ICP8. In some embodiments, the virus described herein comprises one or more miR-132-3p target sequences in UL8. In some embodiments, the virus described herein comprises one or more miR-132-3p target sequences in ICP8 and UL8.

Table 2 below provides sequences of exemplary miRNAs that can bind to the miRNA target sequences in the oncolytic viruses described herein. Additional miRNA sequences are provided in SEQ ID NOs: 33-803.

TABLE 2
Exemplary miRNAs and Target Sequences

| | | SEQ | | SEQ |
|---|---|---|---|---|
| miRNA | miRNA Sequence | ID: | miR-TS | ID: |
| 122-5p | uggagugugacaaugguguuug | 1 | caaacaccattgtcacactcca | 804 |
| 124-3p | uaaggcacgcggugaaugcc | 2 | ggcattcaccgcgtgcctta | 805 |
| 125a-5p | ucccugagacccuuuaaccuguga | 3 | tcacaggttaaagggtctcaggga | 806 |
| 126-3p | ucguaccgugaguaauaaugcg | 4 | cgcattattactcacggtacga | 807 |
| | | | cacattattactcacggtacga | 808 |
| 127a-3p | ucggauccgucugagcuuggcu | 5 | agccaagctcagacggatccga | 809 |
| 128-3p | ucacagugaaccggucucuuu | 6 | aaagagaccggttcactgtga | 810 |
| | | | aaagagaccggttcactgtgg | 811 |
| 129-3p | aagcccuuaccccaaaaaguau | 7 | atactttttggggtaagggctt | 812 |
| 129-5p | cuuuuugggucugggcuugc | 8 | gcaagcccagaccgcaaaaag | 813 |
| 130b-3p | cagugcaaugaugaaagggcau | 9 | atgccctttcatcattgcactg | 814 |
| 130b-5p | acucuuucccuguugcacuac | 10 | gtagtgcaacagggaaagagt | 815 |
| 133a-3p | uuugguccccuucaaccagcug | 11 | cagctggttgaaggggaccaaa | 816 |
| 133b-3p | uuugguccccuucaaccagcua | 12 | tagctggttgaaggggaccaaa | 817 |
| 134-3p | ccugugggccaccuagucaccaa | 13 | ttggtgactaggtggcccacagg | 818 |
| 137-3p | uuauugcuuaagaauacgcguag | 14 | ctacgcgtattcttaagcaataa | 819 |
| 1-3p | uggaauguaaagaaguauguau | 15 | atacatacttctttacattcca | 820 |
| 143-3p | ugagaugaagcacuguagcuc | 16 | gagctacagtgcttcatctca | 821 |
| 145-3p | ggauuccuggaaauacuguucu | 17 | agaacagtatttccaggaatcc | 822 |
| 145-5p | guccaguuuucccaggaaucccu | 18 | agggattcctgggaaaactggac | 823 |
| 184-3p | uggacggagaacugauaagggu | 19 | acccttatcagttctccgtcca | 824 |
| 199a-3p | acaguagucugcacauugguua | 20 | taaccaatgtgcagactactgt | 825 |
| 199a-5p | cccaguguucagacuaccuguuc | 21 | gaacaggtagtctgaacactggg | 826 |
| 204-5p | uucccuuugucauccuaugccu | 22 | aggcataggatgacaaagggaa | 827 |
| 208b-3p | auaagacgaacaaaagguuugu | 23 | acaaaccttttgttcgtcttat | 828 |
| 214-3p | acagcaggcacagacaggcagu | 24 | actgcctgtctgtgcctgctgt | 829 |
| 217-5p | uacugcaucaggaacugauugga | 25 | tccaatcagttcctgatgcagta | 830 |
| 219a-5p | ugauuguccaaacgcaauucu | 26 | agaattgcgtttggacaatca | 831 |
| 223-3p | ugucaguuugucaaauacccca | 27 | tggggtatttgacaaactgaca | 832 |
| 34a-5p | uggcagugucuuagcugguugu | 28 | acaaccagctaagacactgcca | 833 |
| 451a | aaaccguuaccauuacugaguu | 29 | aactcagtaatggtaacggttt | 834 |
| 559-5p | uaaaguaaauaugcaccaaaa | 30 | ttttggtgcatatttacttta | 835 |
| Let7a-5p | ugagguaguagguuguauaguu | 31 | aactatacaacctactacctca | 836 |
| 9-5p | ucuuugguuaucuagcuguauga | 32 | tcatacagctagataaccaaaga | 837 |
| 34b-5p | uaggcagugucauuagcugauug | 861 | caatcagctaatgacactgccta | 867 |
| 34b-3p | caaucacuaacuccacugccau | 862 | atggcagtggagttagtgattg | 868 |
| 34c-5p | aggcaguguaguuagcugauugc | 863 | gcaatcagctaactacactgcct | 869 |
| 128T | CCACAGUGAACCGGUCUCUUU | 864 | AAAGAGACCGGTTCACTGTGG | 870 |
| 129-2-3p | aagcccuuaccccaaaaagcau | 865 | atgctttttggggtaagggctt | 871 |
| 132-3p | uaacagucuacagccauggucg | 866 | cgaccatggctgtagactgtta | 872 |

In some embodiments, the miR-TS cassettes comprise one or more additional polynucleotide sequences that enable the cassette to be inserted into the locus of a viral gene. For example, a miR-TS cassette may further comprise short polynucleotide sequence on the 5′ and 3′ ends that are complementary to a nucleic acid sequence at a desired location in the viral genome. Such sequences are referred to herein as “homology arms” and facilitate the insertion of a miR-TS cassette into a specific location in the viral genome.

In some embodiments, the miR-TS cassettes disclosed comprise two or more pluralities of miR-TSs each corresponding to a different miRNA and the miR-TSs are selected to protect diverse cell types or organs from an oncolytic virus. In some embodiments, the pluralities of miR-TSs are interleaved rather than in tandem to one another. In some embodiments, the miR-TS cassettes have short (e.g., 4-15 nt in length) spacers, resulting in a more compact cassette. In some embodiments, the spacers are 4 nt in length. In some embodiments, the miR-TS cassettes are free from (or have reduced) RNA secondary structures that inhibit activity of the miR-TSs. In some embodiments, the miR-TS cassettes are free from (or have reduced) seed sequences for miRNAs associated with carcinogenesis, malignant transformation, or metastasis (i.e., “oncomiRs”). In some embodiments, the miR-TS cassettes are free from (or have reduced) polyadenylation sites.

Oncolytic Viruses Comprising miR-TS Cassettes

In some embodiments, a recombinant oncolytic virus may comprise one miR-TS cassette incorporated into a locus of one essential viral gene, wherein the miR-TS cassette comprises a plurality of miRNA target sequences, such that the recombinant oncolytic virus comprises a plurality of miRNA target sequences incorporated into a locus of one essential viral gene. In some embodiments, the miR-TS cassette may comprise a plurality of miRNA target sequences, wherein each miRNA target sequence of the plurality is a target for the same miRNA, such that the recombinant oncolytic virus comprises a plurality (e.g., 2, 3, 4, 5, 6, 7, 8, 9, 10 or more) copies of the same miRNA target sequence incorporated into a locus of an essential viral gene. For example, in some embodiments, a recombinant oncolytic HSV may comprise a miR-TS cassette comprising 2, 3, 4, 5, 6 or more target sequences inserted into one of ICP4, ICP27, ICP8, ICP22, ICP34.5, UL5, UL8, UL9, UL30, UL39/40, or UL42. In some embodiments, a recombinant oncolytic HSV may comprise a miR-TS cassette comprising 2, 3, 4, or more target sequences inserted into one of ICP8, ICP22, ICP34.5, UL5, UL8, UL9, UL30, UL39/40, or UL42. In some embodiments, a recombinant oncolytic HSV may comprise a miR-TS cassette comprising 2, 3, 4, 5, 6 or more target sequence inserted into one of ICP4, ICP27, ICP34.5, UL8, or UL9.

In some embodiments, the plurality of miRNA target sequences comprises at least two different miRNA target sequences, at least three different miRNA target sequences, or at least four different miRNA target sequences, such that the recombinant oncolytic virus comprises one or more copies of at least 2, 3, or 4 different miRNA target sequence incorporated into a locus of an essential viral gene.

In some embodiments, a recombinant oncolytic virus may comprise one miR-TS cassette incorporated into the 3′ or 5′ untranslated region (UTR) of the viral genome. In such embodiments, the miR-TS cassette may comprise one copy of a miRNA target sequence, such that the recombinant oncolytic virus comprises one copy of a miRNA target sequence incorporated into the 3′ or 5′ UTR of the viral genome. For example, in some embodiments, a recombinant HSV may comprise a miR-TS cassette inserted into the 3′ or 5′ UTR of a viral gene.

In some aspects, the plurality of miRNA target sequences comprises at least two different miRNA target sequences, at least three different miRNA target sequences, or at least four different miRNA target sequences, such that the recombinant oncolytic virus comprises one or more copies of at least 2, 3, or 4 different miRNA target sequence incorporated into the 3′ or 5′ UTR of the viral gene.

In some embodiments, a recombinant oncolytic virus may comprise a miR-TS cassette incorporated into a locus of two or more essential viral genes. In some embodiments, the recombinant oncolytic virus is an HSV and the two or more essential viral genes are selected from the group consisting of ICP4, ICP27, ICP8, ICP22, ICP34.5, UL5, UL8, UL9, UL30, UL39/40, or UL42. In some embodiments, the recombinant oncolytic virus is an HSV and the two or more essential viral genes are selected from the group consisting of ICP8, ICP22, ICP34.5, UL5, UL8, UL9, UL30, UL39/40, or UL42. In some embodiments, the recombinant oncolytic virus is an HSV and the two or more essential viral genes are selected from the group consisting of ICP4, ICP27, ICP34.5, UL8, or UL9. In some embodiments, the recombinant oncolytic virus is an HSV and the two or more essential viral genes are selected from the group consisting of ICP27, ICP4, ICP34.5, UL8, and UL42.

In some embodiments, a recombinant oncolytic herpesvirus may comprise a miR-TS cassette incorporated into a locus of one or more viral genes selected from ICP4, ICP8, ICP27 and UL8. In some embodiments, a recombinant oncolytic herpesvirus may comprise a miR-TS cassette incorporated into a locus of two or more viral genes selected from ICP4, ICP8, ICP27 and UL8. In some embodiments, a recombinant oncolytic herpesvirus may comprise a miR-TS cassette incorporated into a locus of three or more viral genes selected from ICP4, ICP8, ICP27 and UL8. In some embodiments, a recombinant oncolytic herpesvirus may comprise a miR-TS cassette incorporated into a locus of all of the viral genes ICP4, ICP8, ICP27 and UL8. In some embodiments, a recombinant oncolytic herpesvirus may comprise a miR-TS cassette incorporated into a locus of each of the viral genes ICP4 and ICP8. In some embodiments, a recombinant oncolytic herpesvirus may comprise a miR-TS cassette incorporated into both copies of ICP4. In some embodiments, the recombinant oncolytic herpesvirus is a recombinant herpes simplex virus (HSV).

In some embodiments, the recombinant oncolytic virus comprises a miR-TS cassette comprises one or more miRNA target sequences for miR-34b-5p, miR-34b-3p, miR-34c-5p, miR-122-5p, miR-124-3p, miR-128T, miR-129-5p, miR-129-2-3p, miR-132-3p, miR-137-3p, miR-145-5p, or any combination thereof. In some embodiments, the recombinant oncolytic virus comprises a miR-TS cassette comprises one or more miRNA target sequences for miR-34b-5p, miR-34b-3p, miR-34c-5p, miR-122-5p, miR-124-3p, miR-128T, miR-129-5p, miR-129-2-3p, miR-132-3p, miR-137-3p, and miR-145-5p. In some embodiments, the recombinant oncolytic virus comprises a miR-TS cassette comprises one or more miRNA target sequences for miR-34b-5p, miR-34c-5p, miR-122-5p, miR-124-3p, miR-128T, miR-129-2-3p, miR-132-3p, miR-137-3p, miR-145-5p, or any combination thereof. In some embodiments, the recombinant oncolytic virus comprises a miR-TS cassette comprises one or more miRNA target sequences for miR-34b-5p, miR-34c-5p, miR-122-5p, miR-124-3p, miR-128T, miR-129-2-3p, miR-132-3p, miR-137-3p, and miR-145-5p. In some embodiments, the recombinant oncolytic virus comprises a miR-TS cassette comprises one or more miRNA target sequences for miR-122-5p, miR-124-3p, miR-128T, miR-137-3p, miR-34c-5p, miR-129-2-3p, miR-132-3p, or any combination thereof. In some embodiments, the recombinant oncolytic virus comprises a miR-TS cassette comprises one or more miRNA target sequences for miR-122-5p, miR-124-3p, miR-128T, miR-137-3p, miR-34c-5p, miR-129-2-3p, and miR-132-3p. In some embodiments, the recombinant oncolytic virus is a recombinant herpes simplex virus (HSV).

In some embodiments, the recombinant oncolytic virus comprises one or more miRNA target sequence for a miRNA that has lower expression in a glioblastoma cell than in a normal cell in the brain. Non-limiting examples of such miRNAs are listed in Table 3 below.

TABLE 3
Non-limiting examples of miRNAs with lower
expression in glioblastoma cells

| | miRNA(s) that have lower expression in a |
|---|---|
| Normal cell | glioblastoma cell than in the normal cell |
| Neuron | miR-124-3p, miR-128T, miR-137-3p |
| Ependymal Cells | miR-34b-5p, miR-34b-3p, miR-34c-5p |
| Oligodendrocyte | miR-129-5p, miR-129-2-3p, miR-132-3p |
| Astrocyte | miR-34b-5p, miR-34b-3p, miR-145-5p |
| Endothelial Cells | miR-145-5p |
| Hepatocytes | miR-122-5p |

In some embodiments, the recombinant oncolytic virus comprises one or more miRNA target sequence for a miRNA that has lower expression in glioblastoma cells than in neurons. In some embodiments, such a miRNA is miR-124-3p, miR-128T, miR-137-3p, or any combination thereof. In some embodiments, such miRNAs comprises miR-124-3p, miR-128T, and miR-137-3p.

In some embodiments, the recombinant oncolytic virus comprises one or more miRNA target sequence for a miRNA that has lower expression in glioblastoma cells than in ependymal cells. In some embodiments, such a miRNA is miR-34b-5p, miR-34b-3p, miR-34c-5p, or any combination thereof. In some embodiments, such a miRNA is miR-34b-5p, miR-34c-5p, or any combination thereof. In some embodiments, such miRNAs comprise miR-34b-5p and miR-34c-5p.

In some embodiments, the recombinant oncolytic virus comprises one or more miRNA target sequence for a miRNA that has lower expression in glioblastoma cells than in oligodendrocyte. In some embodiments, such a miRNA is miR-129-5p, miR-129-2-3p, miR-132-3p, or any combination thereof. In some embodiments, such a miRNA is miR-129-2-3p, miR-132-3p, or any combination thereof. In some embodiments, such miRNAs comprise miR-129-2-3p and miR-132-3p.

In some embodiments, the recombinant oncolytic virus comprises one or more miRNA target sequence for a miRNA that has lower expression in glioblastoma cells than in astrocyte. In some embodiments, such a miRNA is miR-34b-5p, miR-34b-3p, miR-145-5p, or any combination thereof.

In some embodiments, the recombinant oncolytic virus comprises one or more miRNA target sequence for a miRNA that has lower expression in glioblastoma cells than in endothelial cells. In some embodiments, such a miRNA comprises miR-145-5p.

In some embodiments, the recombinant oncolytic virus comprises one or more miRNA target sequence for a miRNA that has lower expression in glioblastoma cells than in hepatocytes. In some embodiments, such a miRNA comprises miR-122-5p.

In some embodiments, the recombinant oncolytic virus comprises
(a) one or more miRNA target sequences for miR-122-5p;
(b) one or more miRNA target sequences for miR-145-5p;
(c) one or more miRNA target sequences for miR-124-3p, miR-128T, miR-137-3p, or any combination thereof;
(d) one or more miRNA target sequences for miR-34b-5p, miR-34c-5p, or any combination thereof;
(e) one or more miRNA target sequences for miR-129-2-3p, miR-132-3p, or any combination thereof; or
(f) any combination of (a)-(e).

In some embodiments, the recombinant oncolytic virus comprises
(a) one or more miRNA target sequences for miR-34c-5p;
(b) one or more miRNA target sequences for miR-129-5p, miR-129-2-3p, miR-132-3p, or any combination thereof;
(c) one or more miRNA target sequences for miR-34b-5p, miR-34b-3p, miR-145-5p, or any combination thereof;
(d) one or more miRNA target sequences for miR-124-3p, miR-128T, miR-137-3p, or any combination thereof; or
(e) any combination of (a)-(d).

In some embodiments, the recombinant oncolytic virus comprises
(a) one or more miRNA target sequences for miR-34b-5p, miR-34b-3p, miR-34c-5p, or any combination thereof;
(b) one or more miRNA target sequences for miR-129-5p, miR-129-2-3p, miR-132-3p, or any combination thereof;
(c) one or more miRNA target sequences for miR-124-3p, miR-128T, miR-137-3p, or any combination thereof; or
(d) any combination of (a)-(c).

In some embodiments, the recombinant oncolytic virus comprises
(a) one or more miRNA target sequences for miR-122-5p;
(b) one or more miRNA target sequences for miR-145-5p;
(c) one or more miRNA target sequences for miR-124-3p, miR-128T, miR-137-3p, or any combination thereof;
(d) one or more miRNA target sequences for miR-34b-5p, miR-34c-5p, or any combination thereof; and
(e) one or more miRNA target sequences for miR-129-2-3p, miR-132-3p, or any combination thereof.

In some embodiments, the recombinant oncolytic virus comprises
(a) one or more miRNA target sequences for miR-34c-5p;
(b) one or more miRNA target sequences for miR-129-5p, miR-129-2-3p, miR-132-3p, or any combination thereof;
(c) one or more miRNA target sequences for miR-34b-5p, miR-34b-3p, miR-145-5p, or any combination thereof; and
(d) one or more miRNA target sequences for miR-124-3p, miR-128T, miR-137-3p, or any combination thereof.

In some embodiments, the recombinant oncolytic virus comprises
(a) one or more miRNA target sequences for miR-34b-5p, miR-34b-3p, miR-34c-5p, or any combination thereof;
(b) one or more miRNA target sequences for miR-129-5p, miR-129-2-3p, miR-132-3p, or any combination thereof; and
(d) one or more miRNA target sequences for miR-124-3p, miR-128T, miR-137-3p, or any combination thereof.

In some embodiments, the target sequences are for miRNAs that are expressed in normal brain tissue but not expressed in tumor tissue (e.g., glioblastoma). In some embodiments, the miRNAs are expressed broadly in the brain, for example in neurons, oligodendrocytes, ependymal cells, and/or endothelial cells. In some embodiments, the miRNA target sequences are inserted into one or more viral genes. In some embodiments, viral gene(s) were selected based on their sensitivity to RNA interference. Descriptions of methods for screening and selecting such viral gene(s) can be found, for example, in US 2020/0206285, the content of which is incorporated by reference herein in its entirety for all purposes. In some embodiments, from the candidate viral gene(s) that are sensitive to RNA interference, the viral gene(s) essential for virus replication were selected for inserting the miRNA target sequence(s). In some embodiments, the viral gene(s) were expressed early in the virus life cycle, prior to replication of the virus genome. In some embodiments, the target sequences for one particular miRNA are inserted into more than one viral genes (e.g., two different essential viral genes) to ensure deeper coverage for the protection of corresponding normal cells with high expression of that miRNA.

In some embodiments, the miR-TS cassette comprises one or more miRNA target sequences for miR-34c-5p, miR-124-3p, miR-129-2-3p, miR-132-3p, or any combination thereof. In some embodiments, the miR-TS cassette comprises at least 1, 2, or 3 copies of a target sequence for miR-34c-5p. In some embodiments, the miR-TS cassette comprises at least 1, 2, or 3 copies of a target sequence for miR-124-3p. In some embodiments, the miR-TS cassette comprises at least 1, 2, or 3 copies of a target sequence for miR-129-2-3p. In some embodiments, the miR-TS cassette comprises at least 1, 2, or 3 copies of a target sequence for miR-132-3p. In some embodiments, the miR-TS cassette comprises one or more miRNA target sequences for each of miR-34c-5p, miR-124-3p, miR-129-2-3p, and miR-132-3p. In some embodiments, the miR-TS cassette is incorporated into ICP8 viral gene of the recombinant HSV. In some embodiments, the miRNA target sequences in the miR-TS cassettes are arranged as follows:
(34c-5p)-(124-3p)-(132-3p)-(129-2-3p)-(34c-5p)-(124-3p)-(129-2-3p)-(132-3p)-(124-3p)-(129-2-3p)-(132-3p)-(34c-5p).
   In some embodiments, the miR-TS cassette comprises a polynucleotide sequence having at least 80%, at least 85%, at least 90%, at least 95%, at least 96%, at least 97%, at least 98%, or at least 99% identical to SEQ ID NO: 859. In some embodiments, the miR-TS cassette comprises the polynucleotide sequence of SEQ ID NO: 859.

In some embodiments, the miR-TS cassette comprises one or more miRNA target sequences for miR-122-5p, miR-124-3p, miR-128T, miR-137-3p, or any combination thereof. In some embodiments, the miR-TS cassette comprises at least 1, 2, or 3 copies of a target sequence for miR-122-5p. In some embodiments, the miR-TS cassette comprises at least 1, 2, or 3 copies of a target sequence for miR-124-3p. In some embodiments, the miR-TS cassette comprises at least 1, 2, or 3 copies of a target sequence for miR-128T. In some embodiments, the miR-TS cassette comprises at least 1, 2, or 3 copies of a target sequence for miR-137-3p. In some embodiments, the miR-TS cassette comprises one or more miRNA target sequences for each of miR-122-5p, miR-124-3p, miR-128T, and miR-137-3p. In some embodiments, the miR-TS cassette is incorporated into ICP4 viral gene of the recombinant HSV. In some embodiments, the miR-TS cassette is incorporated into both copies of the ICP4 viral gene in the viral genome. In some embodiments, the miRNA target sequences in the miR-TS cassettes are arranged as follows:
(137-3p)-(128T)-(122-5p)-(124-3p)-(122-5p)-(128T)-(137-3p)-(124-3p)-(128T)-(137-3p)-(124-3p)-(122-5p).

In some embodiments, the miR-TS cassette comprises a polynucleotide sequence having at least 80%, at least 85%, at least 90%, at least 95%, at least 96%, at least 97%, at least 98%, or at least 99% identical to SEQ ID NO: 858. In some embodiments, the miR-TS cassette comprises the polynucleotide sequence of SEQ ID NO: 858.

In some embodiments, the miR-TS cassette comprises one or more miRNA target sequences for miR-34c-5p, miR-124-3p, miR-128T, miR-137-3p, or any combination thereof. In some embodiments, the miR-TS cassette comprises at least 1, 2, or 3 copies of a target sequence for miR-34c-5p. In some embodiments, the miR-TS cassette comprises at least 1, 2, or 3 copies of a target sequence for miR-124-3p. In some embodiments, the miR-TS cassette comprises at least 1, 2, or 3 copies of a target sequence for miR-128T. In some embodiments, the miR-TS cassette comprises at least 1, 2, or 3 copies of a target sequence for miR-137-3p. In some embodiments, the miR-TS cassette comprises one or more miRNA target sequences for each of miR-34c-5p, miR-124-3p, miR-128T, and miR-137-3p. In some embodiments, the miR-TS cassette is incorporated into ICP27 viral gene of the recombinant HSV. In some embodiments, the miRNA target sequences in the miR-TS cassettes are arranged as follows:
(124-3p)-(128T)-(34c-5p)-(137-3p)-(128T)-(34c-5p)-(137-3p)-(124-3p)-(128T)-(137-3p)-(124-3p)-(34c-5p).

In some embodiments, the miR-TS cassette comprises a polynucleotide sequence having at least 80%, at least 85%, at least 90%, at least 95%, at least 96%, at least 97%, at least 98%, or at least 99% identical to SEQ ID NO: 873. In some embodiments, the miR-TS cassette comprises the polynucleotide sequence of SEQ ID NO: 873.

In some embodiments, the miR-TS cassette comprises one or more miRNA target sequences for miR-34b-3p, miR-34c-5p, miR-128T, miR-137-3p, or any combination thereof.

In some embodiments, the miR-TS cassette comprises at least 1, 2, or 3 copies of a target sequence for miR-34b-3p. In some embodiments, the miR-TS cassette comprises at least 1, 2, or 3 copies of a target sequence for miR-34c-5p. In some embodiments, the miR-TS cassette comprises at least 1, 2, or 3 copies of a target sequence for miR-128T. In some embodiments, the miR-TS cassette comprises at least 1, 2, or 3 copies of a target sequence for miR-137-3p. In some embodiments, the miR-TS cassette comprises one or more miRNA target sequences for each of miR-34b-3p, miR-34c-5p, miR-128T, and miR-137-3p. In some embodiments, the miR-TS cassette is incorporated into ICP27 viral gene of the recombinant HSV. In some embodiments, the miRNA target sequences in the miR-TS cassettes are arranged as follows:
(34b-3p)-(128T)-(137-3p)-(34c-5p)-(128T)-(34b-3p)-(137-3p)-(34c-5p)-(137-3p)-(34b-3p)-(128T)-(34c-5p).

In some embodiments, the miR-TS cassette comprises a polynucleotide sequence having at least 80%, at least 85%, at least 90%, at least 95%, at least 96%, at least 97%, at least 98%, or at least 99% identical to SEQ ID NO: 860. In some embodiments, the miR-TS cassette comprises the polynucleotide sequence of SEQ ID NO: 860.

In some embodiments, the miR-TS cassette comprises one or more miRNA target sequences for miR-34b-5p, miR-34c-5p, miR-132-3p, miR-145-5p, or any combination thereof. In some embodiments, the miR-TS cassette comprises at least 1, 2, or 3 copies of a target sequence for miR-34b-5p. In some embodiments, the miR-TS cassette comprises at least 1, 2, or 3 copies of a target sequence for miR-34c-5p. In some embodiments, the miR-TS cassette comprises at least 1, 2, or 3 copies of a target sequence for miR-132-3p. In some embodiments, the miR-TS cassette comprises at least 1, 2, or 3 copies of a target sequence for miR-145-5p. In some embodiments, the miR-TS cassette comprises one or more miRNA target sequences for each of miR-34b-5p, miR-34c-5p, miR-132-3p, and miR-145-5p. In some embodiments, the miR-TS cassette is incorporated into UL8 viral gene of the recombinant HSV. In some embodiments, the miRNA target sequences in the miR-TS cassettes are arranged as follows:
(145-5p)-(34b-5p)-(132-3p)-(34c-5p)-(145-5p)-(34c-5p)-(34b-5p)-(132-3p)-(34b-5p)-(145-5p)-(132-3p)-(34c-5p).

In some embodiments, the miR-TS cassette comprises a polynucleotide sequence having at least 80%, at least 85%, at least 90%, at least 95%, at least 96%, at least 97%, at least 98%, or at least 99% identical to SEQ ID NO: 874. In some embodiments, the miR-TS cassette comprises the polynucleotide sequence of SEQ ID NO: 874.

In some embodiments, the miR-TS cassette comprises one or more miRNA target sequences for miR-34b-5p, miR-34c-5p, miR-129-5p, miR-145-5p, or any combination thereof. In some embodiments, the miR-TS cassette comprises at least 1, 2, or 3 copies of a target sequence for miR-34b-5p. In some embodiments, the miR-TS cassette comprises at least 1, 2, or 3 copies of a target sequence for miR-34c-5p. In some embodiments, the miR-TS cassette comprises at least 1, 2, or 3 copies of a target sequence for miR-129-5p. In some embodiments, the miR-TS cassette comprises at least 1, 2, or 3 copies of a target sequence for miR-145-5p. In some embodiments, the miR-TS cassette comprises one or more miRNA target sequences for each of miR-34b-5p, miR-34c-5p, miR-129-5p, and miR-145-5p. In some embodiments, the miR-TS cassette is incorporated into UL8 viral gene of the recombinant HSV.

In some embodiments, the miR-TS cassette comprises one or more miRNA target sequences for miR-34b-5p, miR-34c-5p, miR-132-3p, miR-145-5p, or any combination thereof. In some embodiments, the miR-TS cassette comprises at least 1, 2, or 3 copies of a target sequence for miR-34b-5p. In some embodiments, the miR-TS cassette comprises at least 1, 2, or 3 copies of a target sequence for miR-34c-5p. In some embodiments, the miR-TS cassette comprises at least 1, 2, or 3 copies of a target sequence for miR-132-3p. In some embodiments, the miR-TS cassette comprises at least 1, 2, or 3 copies of a target sequence for miR-145-5p. In some embodiments, the miR-TS cassette comprises one or more miRNA target sequences for each of miR-34b-5p, miR-34c-5p, miR-132-3p, and miR-145-5p. In some embodiments, the miR-TS cassette is incorporated into UL8 viral gene of the recombinant HSV.

In some embodiments, the miR-TS cassette comprises one or more miRNA target sequences for miR-34b-3p, miR-34c-5p, miR-132-3p, miR-145-5p, or any combination thereof. In some embodiments, the miR-TS cassette comprises at least 1, 2, or 3 copies of a target sequence for miR-34b-3p. In some embodiments, the miR-TS cassette comprises at least 1, 2, or 3 copies of a target sequence for miR-34c-5p. In some embodiments, the miR-TS cassette comprises at least 1, 2, or 3 copies of a target sequence for miR-132-3p. In some embodiments, the miR-TS cassette comprises at least 1, 2, or 3 copies of a target sequence for miR-145-5p. In some embodiments, the miR-TS cassette comprises one or more miRNA target sequences for each of miR-34b-3p, miR-34c-5p, miR-132-3p, and miR-145-5p. In some embodiments, the miR-TS cassette is incorporated into UL8 viral gene of the recombinant HSV.

In some embodiments, the miR-attenuated oncolytic viruses described herein result in reduced viral replication in a cell that expresses a miR capable of binding to one or more of the incorporated miR-target sequences. “Viral replication” refers to the total number of viral replication cycles that occur in a particular cell or population of cells during a given amount of time. In some embodiments, viral replication can be measured directly by assessing the total viral titer present over the course of the given amount of time, or by assessing the number of viral genome copies present (e.g., by sequencing). In some embodiments, the viral vector may additionally comprise a detectable label, such as a fluorescent reporter. In such embodiments, viral replication may be assessed by measuring the fluorescence intensity of the reporter, or the number of cells that express the reporter. In some embodiments, viral replication can be measured indirectly by assessing the number of viable cells over the course of the given amount of time. For example, the level of viral replication would be expected to inversely correlate with the number of viable cells over time.

“Reduced viral replication” as used herein, refers to a level of viral replication that is lower in a first cell or first population of cells compared to a second cell or a second population of cells. In some embodiments, the level of viral replication in the first cell or first population of cells is reduced by at least 5% compared to the level of viral replication in the second cell or population of cells. In some embodiments, the level of viral replication in the first cell or first population of cells is reduced by at least 5%, 6%, 7%, 8%, 9%, 10%, 15%, 20%, 25%, 30%, 35%, 40%, 50%, 60%, 70%, 80%, 90% or more compared to the level of viral replication in the second cell or population of cells. In some embodiments, viral replication in the first cell or first population of cells is completely inhibited compared to the viral replication in the second cell or population of cells.

In some embodiments, the reduced viral replication in the first cell or first population of cells correlates with the expression of a miR capable of binding to the one or more miR-target sequences incorporated into one or more viral genes required for replication. In some embodiments, expression of a miR corresponding to the incorporated miR-target sequence therefore inhibits or reduces the expression of the replication gene, thereby inhibiting or reducing viral replication. In some embodiments, the second cell or second population of cells does not express, or has a reduced expression level, of the miR. In some embodiments, absent or reduced expression of a miR (e.g., in a cancer cell) corresponding to the incorporated miR-target sequence allows for viral replication to proceed. In some embodiments, the expression level of the miR in the second cell or population of cells is at least 5% lower than the expression level of the miR in the first cell or population. In some embodiments, the expression level of the miR in the second cell or population of cells is reduced at least 5%, 6%, 7%, 8%, 9%, 10%, 15%, 20%, 25%, 30%, 35%, 40%, 50%, 60%, 70%, 80%, 90% or more compared to the expression level of the miR in the first cell or population. In some embodiments, the second cell does not express the miR. In particular embodiments, the first cell is a non-cancerous cell and the second cell is a cancerous cell.

In some embodiments, a replication-restricted viral vector (e.g., a miR-attenuated viral vector) is used to treat glioblastoma.

In some embodiments, a recombinant oncolytic virus comprises a plurality of copies of one or more miRNA target sequences for miR-136-3p, miR-432-5p, miR-1-3p, miR-127-3p, miR-379-5p, miR-493-5p, miR-223-5p, miR-223-5p, miR-136-5p, miR-451a, miR-487b-3p, miR-370-3p, miR-410-3p, miR-431-3p, miR-4485-3p, miR-4485-5p, miR-127-5p, miR-409-3p, miR-338-3p, miR-559, miR-411-5p, miR-133a-5p, miR-143-3p, miR-376b-3p, miR-758-3p, miR-1, miR-101, miR-1180, miR-1236, miR-124-3p, miR-125b, miR-126, miR-1280, miR-133a, miR-133b, miR-141, miR-143, miR-144, miR-145, miR-155, miR-16, miR-18a, miR-192, miR-195, miR-200a, miR-200b, miR-200c, miR-203, miR-205, miR-214, miR-218, miR-23b, miR-26a, miR-29c, miR-320c, miR-34a, miR-370, miR-409-3p, miR-429, miR-451, miR-490-5p, miR-493, miR-576-3p, and/or miR-99a inserted into the 5′ UTR or 3′ UTR of one or more viral genes required for viral replication. This oncolytic virus may be used in methods and compositions for treating bladder cancer.

In some embodiments, a recombinant oncolytic virus comprises a plurality of copies of one or more miRNA target sequences for miR-1251-5p, miR-219a-5p, miR-219a-2-3p, miR-124-3p, miR-448, miR-138-2-3p, miR-490-5p, miR-129-1-3p, miR-1264, miR-3943, miR-490-3p, miR-383-5p, miR-133b, miR-129-2-3p, miR-128-2-5p, miR-133a-3p, miR-129-5p, miR-1-3p, miR-885-3p, miR-124-5p, miR-759, miR-7158-3p, miR-770-5p, miR-135a-5p, miR-885-5p, let-7g-5p, miR-100, miR-101, miR-106a, miR-124, miR-124a, miR-125a, miR-125a-5p, miR-125b, miR-127-3p, miR-128, miR-129, miR-136, miR-137, miR-139-5p, miR-142-3p, miR-143, miR-145, miR-146b-5p, miR-149, miR-152, miR-153, miR-195, miR-21, miR-212-3p, miR-219-5p, miR-222, miR-29b, miR-31, miR-3189-3p, miR-320, miR-320a, miR-326, miR-330, miR-331-3p, miR-340, miR-342, miR-34a, miR-376a, miR-449a, miR-483-5p, miR-503, miR-577, miR-663, miR-7, miR-7-5p, miR-873, let-7a, let-7f, miR-107, miR-122, miR-124-5p, miR-139, miR-146a, miR-146b, miR-15b, miR-16, miR-181a, miR-181a-1, miR-181a-2, miR-181b, miR-181b-1, miR-181b-2, miR-181c, miR-181d, miR-184, miR-185, miR-199a-3p, miR-200a, miR-200b, miR-203, miR-204, miR-205, miR-218, miR-23b, miR-26b, miR-27a, miR-29c, miR-328, miR-34c-3p, miR-34c-5p, miR-375, miR-383, miR-451, miR-452, miR-495, miR-584, miR-622, miR-656, miR-98, miR-124-3p, miR-181b-5p, miR-200b, and/or miR-3189-3p inserted into the 5′ UTR or 3′ UTR of one or more viral genes required for viral replication. This oncolytic virus may be used in methods and compositions for treating brain cancer. In certain embodiments, the brain cancer is astrocytoma, glioblastoma, or glioma.

In some embodiments, a recombinant oncolytic virus comprises a plurality of copies of one or more miRNA target sequences for miR-10b-5p, miR-126-3p, miR-145-3p, miR-451a, miR-199b-5p, miR-5683, miR-3195, miR-3182, miR-1271-5p, miR-204-5p, miR-409-5p, miR-136-5p, miR-514a-5p, miR-559, miR-483-3p, miR-1-3p, miR-6080, miR-144-3p, miR-10b-3p, miR-6130, miR-6089, miR-203b-5p, miR-4266, miR-4327, miR-5694, miR-193b, let-7a, let-7a-1, let-7a-2, let-7a-3, let-7b, let-7c, let-7d, let-7e, let-7f-1, let-7f-2, let-7g, let-7i, miR-100, miR-107, miR-10a, miR-10b, miR-122, miR-124, miR-1258, miR-125a-5p, miR-125b, miR-126, miR-127, miR-129, miR-130a, miR-132, miR-133a, miR-143, miR-145, miR-146a, miR-146b, miR-147, miR-148a, miR-149, miR-152, miR-153, miR-15a, miR-16, miR-17-5p, miR-181a, miR-1826, miR-183, miR-185, miR-191, miR-193a-3p, miR-195, miR-199b-5p, miR-19a-3p, miR-200a, miR-200b, miR-200c, miR-205, miR-206, miR-211, miR-216b, miR-218, miR-22, miR-26a, miR-26b, miR-300, miR-30a, miR-31, miR-335, miR-339-5p, miR-33b, miR-34a, miR-34b, miR-34c, miR-374a, miR-379, miR-381, miR-383, miR-425, miR-429, miR-450b-3p, miR-494, miR-495, miR-497, miR-502-5p, miR-517a, miR-574-3p, miR-638, miR-7, miR-720, miR-873, miR-874, miR-92a, miR-98, miR-99a, mmu-miR-290-3p, and/or mmu-miR-290-5p inserted into the 5′ UTR or 3′ UTR of one or more viral genes required for viral replication. This oncolytic virus may be used in methods and compositions for treating breast cancer.

In some embodiments, a recombinant oncolytic virus comprises a plurality of copies of one or more miRNA target sequences for miR-143, miR-145, miR-17-5p, miR-203, miR-214, miR-218, miR-335, miR-342-3p, miR-372, miR-424, miR-491-5p, miR-497, miR-7, miR-99a, miR-99b, miR-100, miR-101, miR-15a, miR-16, miR-34a, miR-886-5p, miR-106a, miR-124, miR-148a, miR-29a, and/or miR-375 inserted into the 5′ UTR or 3′ UTR of one or more viral genes required for viral replication. This oncolytic virus may be used in methods and compositions for treating cervical cancer.

In some embodiments, a recombinant oncolytic virus comprises a plurality of copies of one or more miRNA target sequences for miR-133a-5p, miR-490-5p, miR-124-3p, miR-137, miR-655-3p, miR-376c-3p, miR-369-5p, miR-490-3p, miR-432-5p, miR-487b-3p, miR-342-3p, miR-223-3p, miR-136-3p, miR-136-3p, miR-143-5p, miR-1-3p, miR-214-3p, miR-143-3p, miR-199a-3p, miR-199b-3p, miR-451a, miR-127-3p, miR-133a-3p, miR-145-5p, miR-145-3p, miR-199a-5p, let-7a-1, let-7a-2, let-7a-3, let-7b, let-7c, let-7d, let-7e, let-7f-1, let-7f-2, let-7g, let-7i, miR-100, miR-101, miR-126, miR-142-3p, miR-143, miR-145, miR-192, miR-200c, miR-21, miR-214, miR-215, miR-22, miR-25, miR-302a, miR-320, miR-320a, miR-34a, miR-34c, miR-365, miR-373, miR-424, miR-429, miR-455, miR-484, miR-502, miR-503, miR-93, miR-98, miR-186, miR-30a-5p, miR-627, let-7a, miR-1, miR-124, miR-125a, miR-129, miR-1295b-3p, miR-1307, miR-130b, miR-132, miR-133a, miR-133b, miR-137, miR-138, miR-139, miR-139-5p, miR-140-5p, miR-148a, miR-148b, miR-149, miR-150-5p, miR-154, miR-15a, miR-15b, miR-16, miR-18a, miR-191, miR-193a-5p, miR-194, miR-195, miR-196a, miR-198, miR-199a-5p, miR-203, miR-204-5p, miR-206, miR-212, miR-218, miR-224, miR-24-3p, miR-26b, miR-27a, miR-28-3p, miR-28-5p, miR-29b, miR-30a-3p, miR-30b, miR-328, miR-338-3p, miR-342, miR-345, miR-34a-5p, miR-361-5p, miR-375, miR-378, miR-378a-3p, miR-378a-5p, miR-409-3p, miR-422a, miR-4487, miR-483, miR-497, miR-498, miR-518a-3p, miR-551a, miR-574-5p, miR-625, miR-638, miR-7, miR-96-5p, miR-202-3p, miR-30a, and/or miR-451 inserted into the 5′ UTR or 3′ UTR of one or more viral genes required for viral replication. This oncolytic virus may be used in methods and compositions for treating colon or colorectal cancer.

In some embodiments, a recombinant oncolytic virus comprises a plurality of copies of one or more miRNA target sequences for miR-101, miR-130a, miR-130b, miR-134, miR-143, miR-145, miR-152, miR-205, miR-223, miR-301a, miR-301b, miR-30c, miR-34a, miR-34c, miR-424, miR-449a, miR-543, and/or miR-34b inserted into the 5′ UTR or 3′ UTR of one or more viral genes required for viral replication. This oncolytic virus may be used in methods and compositions for treating endometrial cancer.

In some embodiments, a recombinant oncolytic virus comprises a plurality of copies of one or more miRNA target sequences for miR-125b, miR-138, miR-15a, miR-15b, miR-16, miR-16-1, miR-16-1-3p, miR-16-2, miR-181a, miR-181b, miR-195, miR-223, miR-29b, miR-34b, miR-34c, miR-424, miR-10a, miR-146a, miR-150, miR-151, miR-155, miR-2278, miR-26a, miR-30e, miR-31, miR-326, miR-564, miR-27a, let-7b, miR-124a, miR-142-3p, let-7c, miR-17, miR-20a, miR-29a, miR-30c, miR-720, miR-107, miR-342, miR-34a, miR-202, miR-142-5p, miR-29c, miR-145, miR-193b, miR-199a, miR-214, miR-22, miR-137, and/or miR-197 inserted into the 5′ UTR or 3′ UTR of one or more viral genes required for viral replication. This oncolytic virus may be used in methods and compositions for treating hematologic cancer. In some embodiments, the hematologic cancer is leukemia, lymphoma, or myeloma.

In some embodiments, a recombinant oncolytic virus comprises a plurality of copies of one or more miRNA target sequences for miR-1, miR-145, miR-1826, miR-199a, miR-199a-3p, miR-203, miR-205, miR-497, miR-508-3p, miR-509-3p, let-7a, let-7d, miR-106a*, miR-126, miR-1285, miR-129-3p, miR-1291, miR-133a, miR-135a, miR-138, miR-141, miR-143, miR-182-5p, miR-200a, miR-218, miR-28-5p, miR-30a, miR-30c, miR-30d, miR-34a, miR-378, miR-429, miR-509-5p, miR-646, miR-133b, let-7b, let-7c, miR-200c, miR-204, miR-335, miR-377, and/or miR-506 inserted into the 5′ UTR or 3′ UTR of one or more viral genes required for viral replication. This oncolytic virus may be used in methods and compositions for treating kidney cancer.

In some embodiments, a recombinant oncolytic virus comprises a plurality of copies of one or more miRNA target sequences for let-7a-1, let-7a-2, let-7a-3, let-7b, let-7c, let-7d, let-7e, let-7f, let-7f-1, let-7f-2, let-7g, let-7i, miR-1, miR-100, miR-101, miR-105, miR-122, miR-122a, miR-1236, miR-124, miR-125b, miR-126, miR-127, miR-1271, miR-128-3p, miR-129-5p, miR-130a, miR-130b, miR-133a, miR-134, miR-137, miR-138, miR-139, miR-139-5p, miR-140-5p, miR-141, miR-142-3p, miR-143, miR-144, miR-145, miR-146a, miR-148a, miR-148b, miR-150-5p, miR-15b, miR-16, miR-181a-5p, miR-185, miR-188-5p, miR-193b, miR-195, miR-195-5p, miR-197, miR-198, miR-199a, miR-199a-5p, miR-199b, miR-199b-5p, miR-200a, miR-200b, miR-200c, miR-202, miR-203, miR-204-3p, miR-205, miR-206, miR-20a, miR-21, miR-21-3p, miR-211, miR-212, miR-214, miR-217, miR-218, miR-219-5p, miR-22, miR-223, miR-26a, miR-26b, miR-29a, miR-29b-1, miR-29b-2, miR-29c, miR-302b, miR-302c, miR-30a, miR-30a-3p, miR-335, miR-338-3p, miR-33a, miR-34a, miR-34b, miR-365, miR-370, miR-372, miR-375, miR-376a, miR-377, miR-422a, miR-424, miR-424-5p, miR-433, miR-4458, miR-448, miR-450a, miR-451, miR-485-5p, miR-486-5p, miR-497, miR-503, miR-506, miR-519d, miR-520a, miR-520b, miR-520c-3p, miR-582-5p, miR-590-5p, miR-610, miR-612, miR-625, miR-637, miR-675, miR-7, miR-877, miR-940, miR-941, miR-98, miR-99a, miR-132, and/or miR-31 inserted into the 5′ UTR or 3′ UTR of one or more viral genes required for viral replication. This oncolytic virus may be used in methods and compositions for treating liver cancer. In some embodiments, the liver cancer is hepatocellular carcinoma.

In some embodiments, a recombinant oncolytic virus comprises a plurality of copies of one or more miRNA target sequences for miR-143-3p, miR-126-3p, miR-126-5p, miR-1266-3p, miR-6130, miR-6080, miR-511-5p, miR-143-5p, miR-223-5p, miR-199b-5p, miR-199a-3p, miR-199b-3p, miR-451a, miR-142-5p, miR-144, miR-150-5p, miR-142-3p, miR-214-3p, miR-214-5p, miR-199a-5p, miR-145-3p, miR-145-5p, miR-1297, miR-141, miR-145, miR-16, miR-200a, miR-200b, miR-200c, miR-29b, miR-381, miR-409-3p, miR-429, miR-451, miR-511, miR-99a, let-7a-1, let-7a-2, let-7a-3, let-7b, let-7c, let-7d, let-7e, let-7f-1, let-7f-2, let-7g, let-7i, miR-1, miR-101, miR-133b, miR-138, miR-142-5p, miR-144, miR-1469, miR-146a, miR-153, miR-15a, miR-15b, miR-16-1, miR-16-2, miR-182, miR-192, miR-193a-3p, miR-194, miR-195, miR-198, miR-203, miR-217, miR-218, miR-22, miR-223, miR-26a, miR-26b, miR-29c, miR-33a, miR-34a, miR-34b, miR-34c, miR-365, miR-449a, miR-449b, miR-486-5p, miR-545, miR-610, miR-614, miR-630, miR-660, miR-7515, miR-9500, miR-98, miR-99b, miR-133a, let-7a, miR-100, miR-106a, miR-107, miR-124, miR-125a-3p, miR-125a-5p, miR-126, miR-126*, miR-129, miR-137, miR-140, miR-143, miR-146b, miR-148a, miR-148b, miR-149, miR-152, miR-154, miR-155, miR-17-5p, miR-181a-1, miR-181a-2, miR-181b, miR-181b-1, miR-181b-2, miR-181c, miR-181d, miR-184, miR-186, miR-193b, miR-199a, miR-204, miR-212, miR-221, miR-224, miR-27a, miR-27b, miR-29a, miR-30a, miR-30b, miR-30c, miR-30d, miR-30d-5p, miR-30e-5p, miR-32, miR-335, miR-338-3p, miR-340, miR-342-3p, miR-361-3p, miR-373, miR-375, miR-4500, miR-4782-3p, miR-497, miR-503, miR-512-3p, miR-520a-3p, miR-526b, miR-625*, and/or miR-96 inserted into the 5′ UTR or 3′ UTR of one or more viral genes required for viral replication. This oncolytic virus may be used in methods and compositions for treating lung cancer.

In some embodiments, a recombinant oncolytic virus comprises a plurality of copies of one or more miRNA target sequences for let-7b, miR-101, miR-125b, miR-1280, miR-143, miR-146a, miR-146b, miR-155, miR-17, miR-184, miR-185, miR-18b, miR-193b, miR-200c, miR-203, miR-204, miR-205, miR-206, miR-20a, miR-211, miR-218, miR-26a, miR-31, miR-33a, miR-34a, miR-34c, miR-376a, miR-376c, miR-573, miR-7-5p, miR-9, and/or miR-98 inserted into the 5′ UTR or 3′ UTR of one or more viral genes required for viral replication. This oncolytic virus may be used in methods and compositions for treating melanoma.

In some embodiments, a recombinant oncolytic virus comprises a plurality of copies of one or more miRNA target sequences for let-7d, miR-218, miR-34a, miR-375, miR-494, miR-100, miR-124, miR-1250, miR-125b, miR-126, miR-1271, miR-136, miR-138, miR-145, miR-147, miR-148a, miR-181a, miR-206, miR-220a, miR-26a, miR-26b, miR-29a, miR-32, miR-323-5p, miR-329, miR-338, miR-370, miR-410, miR-429, miR-433, miR-499a-5p, miR-503, miR-506, miR-632, miR-646, miR-668, miR-877, and/or miR-9inserted into the 5′ UTR or 3′ UTR of one or more viral genes required for viral replication. This oncolytic virus may be used in methods and compositions for treating oral cancer.

In some embodiments, a recombinant oncolytic virus comprises a plurality of copies of one or more miRNA target sequences for let-7i, miR-100, miR-124, miR-125b, miR-129-5p, miR-130b, miR-133a, miR-137, miR-138, miR-141, miR-145, miR-148a, miR-152, miR-153, miR-155, miR-199a, miR-200a, miR-200b, miR-200c, miR-212, miR-335, miR-34a, miR-34b, miR-34c, miR-409-3p, miR-411, miR-429, miR-432, miR-449a, miR-494, miR-497, miR-498, miR-519d, miR-655, miR-9, miR-98, miR-101, miR-532-5p, miR-124a, miR-192, miR-193a, and/or miR-7 inserted into the 5′ UTR or 3′ UTR of one or more viral genes required for viral replication. This oncolytic virus may be used in methods and compositions for treating ovarian cancer.

In some embodiments, a recombinant oncolytic virus comprises a plurality of copies of one or more miRNA target sequences for miR-216a-5p, miR-802, miR-217, miR-145-3p, miR-143-3p, miR-451a, miR-375, miR-214-3p, miR-216b-3p, miR-432-5p, miR-216a-3p, miR-199b-5p, miR-199a-5p, miR-136-3p, miR-216b-5p, miR-136-5p, miR-145-5p, miR-127-3p, miR-199a-3p, miR-199b-3p, miR-559, miR-129-2-3p, miR-4507, miR-1-3p, miR-148a-3p, miR-101, miR-1181, miR-124, miR-1247, miR-133a, miR-141, miR-145, miR-146a, miR-148a, miR-148b, miR-150*, miR-150-5p, miR-152, miR-15a, miR-198, miR-203, miR-214, miR-216a, miR-29c, miR-335, miR-34a, miR-34b, miR-34c, miR-373, miR-375, miR-410, miR-497, miR-615-5p, miR-630, miR-96, miR-132, let-7a, let-7a-1, let-7a-2, let-7a-3, let-7b, let-7c, let-7d, let-7e, let-7f-1, let-7f-2, let-7g, let-7i, miR-126, miR-135a, miR-143, miR-144, miR-150, miR-16, miR-200a, miR-200b, miR-200c, miR-217, miR-218, miR-337, miR-494, and/or miR-98 inserted into the 5′ UTR or 3′ UTR of one or more viral genes required for viral replication. This oncolytic virus may be used in methods and compositions for treating pancreatic cancer.

In some embodiments, a recombinant oncolytic virus comprises a plurality of copies of one or more miRNA target sequences for let-7a-3p, let-7c, miR-100, miR-101, miR-105, miR-124, miR-128, miR-1296, miR-130b, miR-133a-1, miR-133a-2, miR-133b, miR-135a, miR-143, miR-145, miR-146a, miR-154, miR-15a, miR-187, miR-188-5p, miR-199b, miR-200b, miR-203, miR-205, miR-212, miR-218, miR-221, miR-224, miR-23a, miR-23b, miR-25, miR-26a, miR-26b, miR-29b, miR-302a, miR-30a, miR-30b, miR-30c-1, miR-30c-2, miR-30d, miR-30e, miR-31, miR-330, miR-331-3p, miR-34a, miR-34b, miR-34c, miR-374b, miR-449a, miR-4723-5p, miR-497, miR-628-5p, miR-642a-5p, miR-765, and/or miR-940 inserted into the 5′ UTR or 3′ UTR of one or more viral genes required for viral replication. This oncolytic virus may be used in methods and compositions for treating prostate cancer.

In some embodiments, a recombinant oncolytic virus comprises a plurality of copies of one or more miRNA target sequences for miR-101, miR-183, miR-204, miR-34a, miR-365b-3p, miR-486-3p, and/or miR-532-5p inserted into the 5′ UTR or 3′ UTR of one or more viral genes required for viral replication. This oncolytic virus may be used in methods and compositions for treating retinoblastoma.

In some embodiments, an oncolytic virus described herein is a herpes simplex virus and wherein the one or more viral genes required for viral replication is selected from the group consisting of UL1, UL5, UL6, UL7, UL8, UL9, UL11, UL12, UL14, UL15, UL17, UL18, UL19, UL20, UL22, UL25, UL26, UL26.5, UL27, UL28, UL29, UL30, UL31, UL32, UL33, UL34, UL35, UL36, UL37, UL38, UL39, UL40, UL42, UL48, UL49, UL52, UL53, UL54, ICP0, ICP4, ICP22, ICP27, ICP47, gamma-34.5, US3, US4, US5, US6, US7, US8, US9, US10, US11, and US12.

### Payload Molecules

In some embodiments, the oncolytic viruses described herein comprise a polynucleotide encoding a payload molecule. As used herein, a “payload molecule” refers to a molecule capable of further enhancing the therapeutic efficacy of a virus. Payload molecules suitable for use in the present disclosure include antigen-binding molecules such as antibodies or antigen binding fragments thereof, cytokines, chemokines, soluble receptors, cell-surface receptor ligands, bipartite peptides, enzymes, and nucleic acids (e.g., shRNAs, siRNAs, antisense RNAs, antagomirs, ribozymes, apatamers, a decoy oligonucleotide, or an antagomir). The nature of the payload molecule will vary with the disease type and desired therapeutic outcome. In some embodiments, one or more miRNA target sequences is incorporated in to the 3′ or 5′ UTR of a polynucleotide sequence encoding a payload molecule. In such embodiments, translation and subsequent expression of the payload does not occur, or is substantially reduced, in cells where the corresponding miRNA is expressed. In some embodiments, one or more miRNA target sequences are inserted into the 3′ and/or 5′ UTR of the polynucleotide sequence encoding the therapeutic polypeptide.

In some embodiments, the payload molecules comprise or consist of payload proteins.

In some embodiments, the oncolytic viruses of the disclosure comprise one or more polynucleotides encoding one or more payload proteins comprising HPGD, ADA2, HYAL1, CHP, CCL21, IL-12, anti-CD47, anti-TGFβ, anti-PD1, anti-TREM2, CTX-BiTE, or any combinations thereof. In some embodiments, the one or more payload molecules comprise 15-hydroxyprostaglandin dehydrogenase [NAD(+)] (HPGD). In some embodiments, the one or more payload molecules comprise Adenosine deaminase 2 (ADA2). In some embodiments, the one or more payload molecules comprise Hyaluronidase-1 (HYAL1). In some embodiments, the one or more payload molecules comprise Chemotaxis inhibitory protein (CHP). In some embodiments, the one or more payload molecules comprise C-C motif chemokine 21 (CCL21). In some embodiments, the one or more payload molecules comprise Interleukin-12 (IL-12). In some embodiments, the one or more payload molecules comprise a chlorotoxin bispecific T-cell engager (CTX-BiTE). In some embodiments, the one or more payload molecules comprise a CD47 antagonist (anti-CD47). In some embodiments, the one or more payload molecules comprise a TGFβ antagonist (anti-TGFβ). In some embodiments, the one or more payload molecules comprise a PD-1 antagonist (anti-PD1). In some embodiments, the one or more payload molecules comprise a TREM2 (Triggering receptor expressed on myeloid cells 2) antagonist (anti-TREM2).

As used herein, the term “antagonist” refers to a molecule that is capable of binding to a target protein and either partially or completely blocks, inhibits, reduces, or neutralizes the activity of the target protein. Non-limiting examples of antagonists include antibodies or antigen binding fragments thereof, aptamers, peptides, and designed ankyrin repeat proteins (DARPins). In some embodiments, the antagonist is an antibody or antigen binding fragment thereof that binds to and inhibit the target protein. In some embodiments, the antibody or antigen binding fragment thereof comprises a full-length immunoglobulin, an scFv, a Fab, a Fab′, an F(ab′)2, an Fv, a diabody, a triabody, a minibody, a single-domain antibody (e.g., VHH), a nanobody, or a multispecific antibody.

In some embodiments, the one or more payload molecules comprise 15-hydroxyprostaglandin dehydrogenase [NAD(+)] (HPGD). Exemplary polypeptide sequences of HPGD can be found in Uniprot Accession Nos. P15428 (for human) and Q8VCCI (for mouse). In some embodiments, the HPGD polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 875. In some embodiments, the HPGD polypeptide comprises, consists essentially of, or consists of the amino acid sequence of SEQ ID NO: 875. In some embodiments, the HPGD polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 876. In some embodiments, the HPGD polypeptide comprises, consists essentially of, or consists of the amino acid sequence of SEQ ID NO: 876.

In some embodiments, the one or more payload molecules comprise Adenosine deaminase 2 (ADA2). Exemplary polypeptide sequence of ADA2 can be found in Uniprot Accession No. Q9NZK5 (for human). In some embodiments, the ADA2 polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 877. In some embodiments, the ADA2 polypeptide comprises, consists essentially of, or consists of the amino acid sequence of SEQ ID NO: 877.

In some embodiments, the one or more payload molecules comprise Hyaluronidase-1 (HYAL1). Exemplary polypeptide sequences of HYAL1 can be found in Uniprot Accession Nos. Q12794 (for human) and Q91ZJ9 (for mouse). In some embodiments, the HYAL1 polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 878. In some embodiments, the HYAL1 polypeptide comprises, consists essentially of, or consists of the amino acid sequence of SEQ ID NO: 878. In some embodiments, the HYAL1 polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 879. In some embodiments, the HYAL1 polypeptide comprises, consists essentially of, or consists of the amino acid sequence of SEQ ID NO: 879.

In some embodiments, the one or more payload molecules comprise Chemotaxis inhibitory protein (CHP). Exemplary polypeptide sequence of CHP can be found in Uniprot Accession No. A6QIG7 (for Staphylococcus aureus). In some embodiments, the CHP polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 880. In some embodiments, the CHP polypeptide comprises, consists essentially of, or consists of the amino acid sequence of SEQ ID NO: 880.

In some embodiments, the one or more payload molecules comprise C-C motif chemokine 21 (CCL21). Exemplary polypeptide sequences of CCL21 can be found in Uniprot Accession Nos. 000585 (for human) and P84444 (for mouse). In some embodiments, the CCL21 polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 881. In some embodiments, the CCL21 polypeptide comprises, consists essentially of, or consists of the amino acid sequence of SEQ ID NO: 881. In some embodiments, the CCL21 polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 882. In some embodiments, the CCL21 polypeptide comprises, consists essentially of, or consists of the amino acid sequence of SEQ ID NO: 882.

In some embodiments, the one or more payload molecules comprise Interleukin-12 (IL-12). IL-12 comprises two disulfide-linked subunits, IL-12 subunit alpha (IL-12a) and IL-12 subunit beta (IL-12b). Exemplary polypeptide sequences of IL-12a can be found in Uniprot Accession Nos. P29459 (for human) and P43431 (for mouse). Exemplary polypeptide sequences of IL-12b can be found in Uniprot Accession Nos. P29460 (for human) and P43432 (for mouse). In some embodiments, the IL-12a polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 883. In some embodiments, the IL-12a polypeptide comprises, consists essentially of, or consists of the amino acid sequence of SEQ ID NO: 883. In some embodiments, the IL-12b polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 884. In some embodiments, the IL-12b polypeptide comprises, consists essentially of, or consists of the amino acid sequence of SEQ ID NO: 884. In some embodiments, the IL-12a polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 885. In some embodiments, the IL-12a polypeptide comprises, consists essentially of, or consists of the amino acid sequence of SEQ ID NO: 885. In some embodiments, the IL-12b polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 886. In some embodiments, the IL-12b polypeptide comprises, consists essentially of, or consists of the amino acid sequence of SEQ ID NO: 886.

In some embodiments, the one or more payload molecules comprise a CD47 antagonist (anti-CD47). In some embodiments, the anti-CD47 comprises an anti-CD47 antibody or an antigen binding fragment thereof. In some embodiments, the anti-CD47 comprises an anti-CD47 VHH domain. In some embodiments, the anti-CD47 polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 887. In some embodiments, the anti-CD47 polypeptide comprises, consists essentially of, or consists of the amino acid sequence of SEQ ID NO: 887. In some embodiments, the anti-CD47 comprises an anti-CD47 VHH domain and an IgG1-Fc. In some embodiments, the anti-CD47 polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 888. In some embodiments, the anti-CD47 polypeptide comprises, consists essentially of, or consists of the amino acid sequence of SEQ ID NO: 888. In some embodiments, the anti-CD47 is an antibody or antigen binding fragment thereof comprising the complementarity-determining regions (CDRs) of SEQ ID NO: 888. In some embodiments, the anti-CD47 antibody or antigen binding fragment thereof comprises heavy chain variable domain or VHH domain CDRs of:

CDR1:
(SEQ ID NO: 895)
GIIFKIND
CDR2:
(SEQ ID NO: 896)
STGGDEA
CDR3:
(SEQ ID NO: 897)
TAVISTDRDGTE

In some embodiments, the anti-CD47 antibody or antigen binding fragment thereof comprises heavy chain variable domain or VHH domain CDRs having at most 1, at most 2, or at most 3 amino acid mutations in CDR1, CDR2 and/or CDR3 according to SEQ ID NOS: 895-897.

In some embodiments, the one or more payload molecules comprise a TGFβ antagonist (anti-TGFβ). In some embodiments, the anti-TGFβ comprises an anti-TGFβ antibody or an antigen binding fragment thereof. In some embodiments, the anti-TGFβ comprises an anti-TGFβ scFv domain. In some embodiments, the anti-TGFβ polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 889. In some embodiments, the anti-TGFβ polypeptide comprises, consists essentially of, or consists of the amino acid sequence of SEQ ID NO: 889. In some embodiments, the anti-TGFβ comprises an anti-TGFβ scFv domain and an IgG1-Fc. In some embodiments, the anti-TGFβ polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 890. In some embodiments, the anti-TGFβ polypeptide comprises, consists essentially of, or consists of the amino acid sequence of SEQ ID NO: 890. In some embodiments, the anti-TGFβ is an antibody or antigen binding fragment thereof comprising the complementarity-determining regions (CDRs) of SEQ ID NO: 890. In some embodiments, the anti-TGFβ antibody or antigen binding fragment thereof comprises heavy chain variable domain CDRs of:

CDR1:
(SEQ ID NO: 898)
GYTFSSNV
CDR2:
(SEQ ID NO: 899)
MGGVIPIVDIAN
CDR3:
(SEQ ID NO: 900)
ASTLGLVLDAMDY

In some embodiments, the anti-TGFβ antibody or antigen binding fragment thereof comprises heavy chain variable domain CDRs having at most 1, at most 2, or at most 3 amino acid mutations in CDR1, CDR2 and/or CDR3 according to SEQ ID NOS: 898-900. In some embodiments, the anti-TGFβ antibody or antigen binding fragment thereof comprises light chain variable domain CDRs of:

CDR1
(SEQ ID NO: 901)
QSLGSSYLA
CDR2
(SEQ ID NO: 902)
GASSRAP
CDR3
(SEQ ID NO: 903)
QQYADSPIT

In some embodiments, the anti-TGFβ antibody or antigen binding fragment thereof comprises light chain variable domain CDRs having at most 1, at most 2, or at most 3 amino acid mutations in CDR1, CDR2 and/or CDR3 according to SEQ ID NOS: 901-903.

In some embodiments, the one or more payload molecules comprise a PD-1 antagonist (anti-PD1). In some embodiments, the anti-PD1 comprises an anti-PD1 antibody or an antigen binding fragment thereof. In some embodiments, the anti-PD1 comprises an anti-PD1 VHH domain. In some embodiments, the anti-PD1 polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 891. In some embodiments, the anti-PD1 polypeptide comprises, consists essentially of, or consists of the amino acid sequence of SEQ ID NO: 891. In some embodiments, the anti-PD1 comprises an anti-PD1 VHH domain and an IgG1-Fc. In some embodiments, the anti-PD1 polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 892. In some embodiments, the anti-PD1 polypeptide comprises, consists essentially of, or consists of the amino acid sequence of SEQ ID NO: 892. In some embodiments, the anti-PD1 is an antibody or antigen binding fragment thereof comprising the complementarity-determining regions (CDRs) of SEQ ID NO: 892. In some embodiments, the anti-PD1 antibody or antigen binding fragment thereof comprises heavy chain variable domain or VHH domain CDRs of:

CDR1:
(SEQ ID NO: 904)
DSIDSLVN
CDR2:
(SEQ ID NO: 905)
IATYITHY
CDR3:
(SEQ ID NO: 906)
YARNIIVDY

In some embodiments, the anti-PD1 antibody or antigen binding fragment thereof comprises heavy chain variable domain or VHH domain CDRs having at most 1, at most 2, or at most 3 amino acid mutations in CDR1, CDR2 and/or CDR3 according to SEQ ID NOS: 904-906.

In some embodiments, the one or more payload molecules comprise a TREM2 antagonist (anti-TREM2). In some embodiments, the anti-TREM2 comprises an anti-TREM2 antibody or an antigen binding fragment thereof. In some embodiments, the anti-TREM2 antibody heavy chain polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 893. In some embodiments, the anti-TREM2 antibody heavy chain polypeptide comprises, consists essentially of, or consists of the amino acid sequence of SEQ ID NO: 893. In some embodiments, the anti-TREM2 antibody light chain polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 894. In some embodiments, the anti-TREM2 antibody light chain polypeptide comprises, consists essentially of, or consists of the amino acid sequence of SEQ ID NO: 894. In some embodiments, the anti-TREM2 is an antibody or antigen binding fragment thereof comprising the complementarity-determining regions (CDRs) of SEQ ID NOs: 893 and 894. In some embodiments, the anti-TREM2 antibody or antigen binding fragment thereof comprises heavy chain variable domain CDRs of:

CDR1:
(SEQ ID NO: 907)
GYTFTDYA
CDR2:
(SEQ ID NO: 908)
ISTYSSNT
CDR3:
(SEQ ID NO: 909)
ARDDGHYVYAMDY

In some embodiments, the anti-TREM2 antibody or antigen binding fragment thereof comprises heavy chain variable domain CDRs having at most 1, at most 2, or at most 3 amino acid mutations in CDR1, CDR2 and/or CDR3 according to SEQ ID NOS: 907-909. In some embodiments, the anti-TREM2 antibody or antigen binding fragment thereof comprises light chain variable domain CDRs of:

CDR1
(SEQ ID NO: 910)
KSLLNSDGFTY
CDR2
(SEQ ID NO: 911)
LVS
CDR3
(SEQ ID NO: 912)
FQSNYLYT

In some embodiments, the anti-TREM2 antibody or antigen binding fragment thereof comprises light chain variable domain CDRs having at most 1, at most 2, or at most 3 amino acid mutations in CDR1, CDR2 and/or CDR3 according to SEQ ID NOS: 910-912.

In some embodiments, the one or more payload molecules comprise a biomolecule comprising a chlorotoxin (CTX). In some embodiments, the CTX is a scorpion CTX. In some embodiments, the CTX polypeptide comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 913. In some embodiments, the CTX polypeptide comprises, consists essentially of, or consists of the amino acid sequence of SEQ ID NO: 913. In some embodiments, the CTX blocks small-conductance chloride channels and binds preferentially to glioma cells. In some embodiments, the biomolecule comprising CTX also comprises an antibody fragment. In some embodiments, the biomolecule comprising CTX also comprises an Fc domain (i.e., CTX-Fc). In some embodiments, the biomolecule comprising CTX also comprises a T-cell engager moiety. In some embodiments, the T-cell engager moiety specifically binds to a protein expressed on the surface of the T-cell. In some embodiments, the T-cell engager moiety specifically binds to CD3. In some embodiments, the CD3-binding T-cell engager moiety comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 914. In some embodiments, the CD3-binding T-cell engager moiety comprises, consists essentially of, or consists of the amino acid sequence of SEQ ID NO: 914. Because CTX binds to chloride channels on the glioma cells, the biomolecule comprising CTX and the T-cell engager moiety is a bispecific T-cell engager (BiTE) (i.e., CTX-BiTE). In some embodiments, the CTX-BiTE comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 915. In some embodiments, the CTX-BiTE comprises, consists essentially of, or consists of the amino acid sequence of SEQ ID NO: 915. In some embodiments, the CTX-BiTE comprises, consists essentially of, or consists of an amino acid sequence that is at least 70%, at least 75%, at least 80%, at least 85%, at least 90%, at least 95%, at least 97%, at least 98%, or at least 99% identical to the sequence of SEQ ID NO: 916. In some embodiments, the CTX-BiTE comprises, consists essentially of, or consists of the amino acid sequence of SEQ ID NO: 916.

In some embodiments, the oncolytic viruses of the disclosure comprise one or more polynucleotides encoding two or more payload molecules comprising any of the combinations listed in Table 4 below.

TABLE 4
Combination of Two Payload Molecules

| Combo # | Payload Molecules |
|---|---|
| 1 | HPGD, ADA2 |
| 2 | HPGD, HYAL1 |
| 3 | HPGD, CHP |
| 4 | HPGD, CCL21 |
| 5 | HPGD, IL-12 |
| 6 | HPGD, anti-CD47 |
| 7 | HPGD, anti-TGFβ |
| 8 | HPGD, anti-PD1 |
| 9 | HPGD, anti-TREM2 |
| 10 | ADA2, HYAL1 |
| 11 | ADA2, CHP |
| 12 | ADA2, CCL21 |
| 13 | ADA2, IL-12 |
| 14 | ADA2, anti-CD47 |
| 15 | ADA2, anti-TGFβ |
| 16 | ADA2, anti-PD1 |
| 17 | ADA2, anti-TREM2 |
| 18 | HYAL1, CHP |
| 19 | HYAL1, CCL21 |
| 20 | HYAL1, IL-12 |
| 21 | HYAL1, anti-CD47 |
| 22 | HYAL1, anti-TGFβ |
| 23 | HYAL1, anti-PD1 |
| 24 | HYAL1, anti-TREM2 |
| 25 | CHP, CCL21 |
| 26 | CHP, IL-12 |
| 27 | CHP, anti-CD47 |
| 28 | CHP, anti-TGFβ |
| 29 | CHP, anti-PD1 |
| 30 | CHP, anti-TREM2 |
| 31 | CCL21, IL-12 |
| 32 | CCL21, anti-CD47 |
| 33 | CCL21, anti-TGFβ |
| 34 | CCL21, anti-PD1 |
| 35 | CCL21, anti-TREM2 |
| 36 | IL-12, anti-CD47 |
| 37 | IL-12, anti-TGFβ |
| 38 | IL-12, anti-PD1 |
| 39 | IL-12, anti-TREM2 |
| 40 | anti-CD47, anti-TGFβ |
| 41 | anti-CD47, anti-PD1 |
| 42 | anti-CD47, anti-TREM2 |
| 43 | anti-TGFβ, anti-PD1 |
| 44 | anti-TGFβ, anti-TREM2 |
| 45 | anti-PD1, anti-TREM2 |
| 46 | CTX-BiTE, ADA2 |
| 47 | CTX-BiTE, HYAL1 |
| 48 | CTX-BiTE, CHP |
| 49 | CTX-BiTE, CCL21 |
| 50 | CTX-BiTE, IL-12 |
| 51 | CTX-BiTE, anti-CD47 |
| 52 | CTX-BiTE, anti-TGFβ |
| 53 | CTX-BiTE, anti-PD1 |
| 54 | CTX-BiTE, anti-TREM2 |
| 55 | CTX-BiTE, HPGD |

In some embodiments, the oncolytic viruses of the disclosure comprise one or more polynucleotides encoding three or more payload molecules comprising any of the combinations listed in Table 5 below.

TABLE 5
Combination of Three Payload Molecules

| Combo # | Payload Molecules |
|---|---|
| 1 | HPGD, ADA2, HYAL1 |
| 2 | HPGD, ADA2, CHP |
| 3 | HPGD, ADA2, CCL21 |
| 4 | HPGD, ADA2, IL-12 |
| 5 | HPGD, ADA2, anti-CD47 |
| 6 | HPGD, ADA2, anti-TGFβ |
| 7 | HPGD, ADA2, anti-PD1 |
| 8 | HPGD, ADA2, anti-TREM2 |
| 9 | HPGD, HYAL1, CHP |
| 10 | HPGD, HYAL1, CCL21 |
| 11 | HPGD, HYAL1, IL-12 |
| 12 | HPGD, HYAL1, anti-CD47 |
| 13 | HPGD, HYAL1, anti-TGFβ |
| 14 | HPGD, HYAL1, anti-PD1 |
| 15 | HPGD, HYAL1, anti-TREM2 |
| 16 | HPGD, CHP, CCL21 |
| 17 | HPGD, CHP, IL-12 |
| 18 | HPGD, CHP, anti-CD47 |
| 19 | HPGD, CHP, anti-TGFβ |
| 20 | HPGD, CHP, anti-PD1 |
| 21 | HPGD, CHP, anti-TREM2 |
| 22 | HPGD, CCL21, IL-12 |
| 23 | HPGD, CCL21, anti-CD47 |
| 24 | HPGD, CCL21, anti-TGFβ |
| 25 | HPGD, CCL21, anti-PD1 |
| 26 | HPGD, CCL21, anti-TREM2 |
| 27 | HPGD, IL-12, anti-CD47 |
| 28 | HPGD, IL-12, anti-TGFβ |
| 29 | HPGD, IL-12, anti-PD1 |
| 30 | HPGD, IL-12, anti-TREM2 |
| 31 | HPGD, anti-CD47, anti-TGFβ |
| 32 | HPGD, anti-CD47, anti-PD1 |
| 33 | HPGD, anti-CD47, anti-TREM2 |
| 34 | HPGD, anti-TGFβ, anti-PD1 |
| 35 | HPGD, anti-TGFβ, anti-TREM2 |
| 36 | HPGD, anti-PD1, anti-TREM2 |
| 37 | ADA2, HYAL1, CHP |
| 38 | ADA2, HYAL1, CCL21 |
| 39 | ADA2, HYAL1, IL-12 |
| 40 | ADA2, HYAL1, anti-CD47 |
| 41 | ADA2, HYAL1, anti-TGFβ |
| 42 | ADA2, HYAL1, anti-PD1 |
| 43 | ADA2, HYAL1, anti-TREM2 |
| 44 | ADA2, CHP, CCL21 |
| 45 | ADA2, CHP, IL-12 |
| 46 | ADA2, CHP, anti-CD47 |
| 47 | ADA2, CHP, anti-TGFβ |
| 48 | ADA2, CHP, anti-PD1 |
| 49 | ADA2, CHP, anti-TREM2 |
| 50 | ADA2, CCL21, IL-12 |
| 51 | ADA2, CCL21, anti-CD47 |
| 52 | ADA2, CCL21, anti-TGFβ |
| 53 | ADA2, CCL21, anti-PD1 |
| 54 | ADA2, CCL21, anti-TREM2 |
| 55 | ADA2, IL-12, anti-CD47 |
| 56 | ADA2, IL-12, anti-TGFβ |
| 57 | ADA2, IL-12, anti-PD1 |
| 58 | ADA2, IL-12, anti-TREM2 |
| 59 | ADA2, anti-CD47, anti-TGFβ |
| 60 | ADA2, anti-CD47, anti-PD1 |
| 61 | ADA2, anti-CD47, anti-TREM2 |
| 62 | ADA2, anti-TGFβ, anti-PD1 |
| 63 | ADA2, anti-TGFβ, anti-TREM2 |
| 64 | ADA2, anti-PD1, anti-TREM2 |
| 65 | HYAL1, CHP, CCL21 |
| 66 | HYAL1, CHP, IL-12 |
| 67 | HYAL1, CHP, anti-CD47 |
| 68 | HYAL1, CHP, anti-TGFβ |
| 69 | HYAL1, CHP, anti-PD1 |
| 70 | HYAL1, CHP, anti-TREM2 |
| 71 | HYAL1, CCL21, IL-12 |
| 72 | HYAL1, CCL21, anti-CD47 |
| 73 | HYAL1, CCL21, anti-TGFβ |
| 74 | HYAL1, CCL21, anti-PD1 |
| 75 | HYAL1, CCL21, anti-TREM2 |
| 76 | HYAL1, IL-12, anti-CD47 |
| 77 | HYAL1, IL-12, anti-TGFβ |
| 78 | HYAL1, IL-12, anti-PD1 |
| 79 | HYAL1, IL-12, anti-TREM2 |
| 80 | HYAL1, anti-CD47, anti-TGFβ |
| 81 | HYAL1, anti-CD47, anti-PD1 |
| 82 | HYAL1, anti-CD47, anti-TREM2 |
| 83 | HYAL1, anti-TGFβ, anti-PD1 |
| 84 | HYAL1, anti-TGFβ, anti-TREM2 |
| 85 | HYAL1, anti-PD1, anti-TREM2 |
| 86 | CHP, CCL21, IL-12 |
| 87 | CHP, CCL21, anti-CD47 |
| 88 | CHP, CCL21, anti-TGFβ |
| 89 | CHP, CCL21, anti-PD1 |
| 90 | CHP, CCL21, anti-TREM2 |
| 91 | CHP, IL-12, anti-CD47 |
| 92 | CHP, IL-12, anti-TGFβ |
| 93 | CHP, IL-12, anti-PD1 |
| 94 | CHP, IL-12, anti-TREM2 |
| 95 | CHP, anti-CD47, anti-TGFβ |
| 96 | CHP, anti-CD47, anti-PD1 |
| 97 | CHP, anti-CD47, anti-TREM2 |
| 98 | CHP, anti-TGFβ, anti-PD1 |
| 99 | CHP, anti-TGFβ, anti-TREM2 |
| 100 | CHP, anti-PD1, anti-TREM2 |
| 101 | CCL21, IL-12, anti-CD47 |
| 102 | CCL21, IL-12, anti-TGFβ |
| 103 | CCL21, IL-12, anti-PD1 |
| 104 | CCL21, IL-12, anti-TREM2 |
| 105 | CCL21, anti-CD47, anti-TGFβ |
| 106 | CCL21, anti-CD47, anti-PD1 |
| 107 | CCL21, anti-CD47, anti-TREM2 |
| 108 | CCL21, anti-TGFβ, anti-PD1 |
| 109 | CCL21, anti-TGFβ, anti-TREM2 |
| 110 | CCL21, anti-PD1, anti-TREM2 |
| 111 | IL-12, anti-CD47, anti-TGFβ |
| 112 | IL-12, anti-CD47, anti-PD1 |
| 113 | IL-12, anti-CD47, anti-TREM2 |
| 114 | IL-12, anti-TGFβ, anti-PD1 |
| 115 | IL-12, anti-TGFβ, anti-TREM2 |
| 116 | IL-12, anti-PD1, anti-TREM2 |
| 117 | anti-CD47, anti-TGFβ, anti-PD1 |
| 118 | anti-CD47, anti-TGFβ, anti-TREM2 |
| 119 | anti-CD47, anti-PD1, anti-TREM2 |
| 120 | anti-TGFβ, anti-PD1, anti-TREM2 |
| 121 | CTX-BiTE, HPGD, ADA2 |
| 122 | CTX-BiTE, HPGD, HYAL1 |
| 123 | CTX-BiTE, HPGD, CHP |
| 124 | CTX-BiTE, HPGD, CCL21 |
| 125 | CTX-BiTE, HPGD, IL-12 |
| 126 | CTX-BiTE, HPGD, anti-CD47 |
| 127 | CTX-BiTE, HPGD, anti-TGFβ |
| 128 | CTX-BiTE, HPGD, anti-PD1 |
| 129 | CTX-BiTE, HPGD, anti-TREM2 |
| 130 | CTX-BiTE, ADA2, HYAL1 |
| 131 | CTX-BiTE, ADA2, CHP |
| 132 | CTX-BiTE, ADA2, CCL21 |
| 133 | CTX-BiTE, ADA2, IL-12 |
| 134 | CTX-BiTE, ADA2, anti-CD47 |
| 135 | CTX-BiTE, ADA2, anti-TGFβ |
| 136 | CTX-BiTE, ADA2, anti-PD1 |
| 137 | CTX-BiTE, ADA2, anti-TREM2 |
| 138 | CTX-BiTE, HYAL1, CHP |
| 139 | CTX-BiTE, HYAL1, CCL21 |
| 140 | CTX-BiTE, HYAL1, IL-12 |
| 141 | CTX-BiTE, HYAL1, anti-CD47 |
| 142 | CTX-BiTE, HYAL1, anti-TGFβ |
| 143 | CTX-BiTE, HYAL1, anti-PD1 |
| 144 | CTX-BiTE, HYAL1, anti-TREM2 |
| 145 | CTX-BiTE, CHP, CCL21 |
| 146 | CTX-BiTE, CHP, IL-12 |
| 147 | CTX-BiTE, CHP, anti-CD47 |
| 148 | CTX-BiTE, CHP, anti-TGFβ |
| 149 | CTX-BiTE, CHP, anti-PD1 |
| 150 | CTX-BiTE, CHP, anti-TREM2 |
| 151 | CTX-BiTE, CCL21, IL-12 |
| 152 | CTX-BiTE, CCL21, anti-CD47 |
| 153 | CTX-BiTE, CCL21, anti-TGFβ |
| 154 | CTX-BiTE, CCL21, anti-PD1 |
| 155 | CTX-BiTE, CCL21, anti-TREM2 |
| 156 | CTX-BiTE, IL-12, anti-CD47 |
| 157 | CTX-BiTE, IL-12, anti-TGFβ |
| 158 | CTX-BiTE, IL-12, anti-PD1 |
| 159 | CTX-BiTE, IL-12, anti-TREM2 |
| 160 | CTX-BiTE, anti-CD47, anti-TGFβ |
| 161 | CTX-BiTE, anti-CD47, anti-PD1 |
| 162 | CTX-BiTE, anti-CD47, anti-TREM2 |
| 163 | CTX-BiTE, anti-TGFβ, anti-PD1 |
| 164 | CTX-BiTE, anti-TGFβ, anti-TREM2 |
| 165 | CTX-BiTE, anti-PD1, anti-TREM2 |

In some embodiments, the oncolytic viruses of the disclosure comprise one or more polynucleotides encoding four or more payload molecules comprising any of the combinations listed in Table 6 below.

TABLE 6
Combination of Four Payload Molecules

| Combo # | Payload Molecules |
|---|---|
| 1 | HPGD, ADA2, HYAL1, CHP |
| 2 | HPGD, ADA2, HYAL1, CCL21 |
| 3 | HPGD, ADA2, HYAL1, IL-12 |
| 4 | HPGD, ADA2, HYAL1, anti-CD47 |
| 5 | HPGD, ADA2, HYAL1, anti-TGFβ |
| 6 | HPGD, ADA2, HYAL1, anti-PD1 |
| 7 | HPGD, ADA2, HYAL1, anti-TREM2 |
| 8 | HPGD, ADA2, CHP, CCL21 |
| 9 | HPGD, ADA2, CHP, IL-12 |
| 10 | HPGD, ADA2, CHP, anti-CD47 |
| 11 | HPGD, ADA2, CHP, anti-TGFβ |
| 12 | HPGD, ADA2, CHP, anti-PD1 |
| 13 | HPGD, ADA2, CHP, anti-TREM2 |
| 14 | HPGD, ADA2, CCL21, IL-12 |
| 15 | HPGD, ADA2, CCL21, anti-CD47 |
| 16 | HPGD, ADA2, CCL21, anti-TGFβ |
| 17 | HPGD, ADA2, CCL21, anti-PD1 |
| 18 | HPGD, ADA2, CCL21, anti-TREM2 |
| 19 | HPGD, ADA2, IL-12, anti-CD47 |
| 20 | HPGD, ADA2, IL-12, anti-TGFβ |
| 21 | HPGD, ADA2, IL-12, anti-PD1 |
| 22 | HPGD, ADA2, IL-12, anti-TREM2 |
| 23 | HPGD, ADA2, anti-CD47, anti-TGFβ |
| 24 | HPGD, ADA2, anti-CD47, anti-PD1 |
| 25 | HPGD, ADA2, anti-CD47, anti-TREM2 |
| 26 | HPGD, ADA2, anti-TGFβ, anti-PD1 |
| 27 | HPGD, ADA2, anti-TGFβ, anti-TREM2 |
| 28 | HPGD, ADA2, anti-PD1, anti-TREM2 |
| 29 | HPGD, HYAL1, CHP, CCL21 |
| 30 | HPGD, HYAL1, CHP, IL-12 |
| 31 | HPGD, HYAL1, CHP, anti-CD47 |
| 32 | HPGD, HYAL1, CHP, anti-TGFβ |
| 33 | HPGD, HYAL1, CHP, anti-PD1 |
| 34 | HPGD, HYAL1, CHP, anti-TREM2 |
| 35 | HPGD, HYAL1, CCL21, IL-12 |
| 36 | HPGD, HYAL1, CCL21, anti-CD47 |
| 37 | HPGD, HYAL1, CCL21, anti-TGFβ |
| 38 | HPGD, HYAL1, CCL21, anti-PD1 |
| 39 | HPGD, HYAL1, CCL21, anti-TREM2 |
| 40 | HPGD, HYAL1, IL-12, anti-CD47 |
| 41 | HPGD, HYAL1, IL-12, anti-TGFβ |
| 42 | HPGD, HYAL1, IL-12, anti-PD1 |
| 43 | HPGD, HYAL1, IL-12, anti-TREM2 |
| 44 | HPGD, HYAL1, anti-CD47, anti-TGFβ |
| 45 | HPGD, HYAL1, anti-CD47, anti-PD1 |
| 46 | HPGD, HYAL1, anti-CD47, anti-TREM2 |
| 47 | HPGD, HYAL1, anti-TGFβ, anti-PD1 |
| 48 | HPGD, HYAL1, anti-TGFβ, anti-TREM2 |
| 49 | HPGD, HYAL1, anti-PD1, anti-TREM2 |
| 50 | HPGD, CHP, CCL21, IL-12 |
| 51 | HPGD, CHP, CCL21, anti-CD47 |
| 52 | HPGD, CHP, CCL21, anti-TGFβ |
| 53 | HPGD, CHP, CCL21, anti-PD1 |
| 54 | HPGD, CHP, CCL21, anti-TREM2 |
| 55 | HPGD, CHP, IL-12, anti-CD47 |
| 56 | HPGD, CHP, IL-12, anti-TGFβ |
| 57 | HPGD, CHP, IL-12, anti-PD1 |
| 58 | HPGD, CHP, IL-12, anti-TREM2 |
| 59 | HPGD, CHP, anti-CD47, anti-TGFβ |
| 60 | HPGD, CHP, anti-CD47, anti-PD1 |
| 61 | HPGD, CHP, anti-CD47, anti-TREM2 |
| 62 | HPGD, CHP, anti-TGFβ, anti-PD1 |
| 63 | HPGD, CHP, anti-TGFβ, anti-TREM2 |
| 64 | HPGD, CHP, anti-PD1, anti-TREM2 |
| 65 | HPGD, CCL21, IL-12, anti-CD47 |
| 66 | HPGD, CCL21, IL-12, anti-TGFβ |
| 67 | HPGD, CCL21, IL-12, anti-PD1 |
| 68 | HPGD, CCL21, IL-12, anti-TREM2 |
| 69 | HPGD, CCL21, anti-CD47, anti-TGFβ |
| 70 | HPGD, CCL21, anti-CD47, anti-PD1 |
| 71 | HPGD, CCL21, anti-CD47, anti-TREM2 |
| 72 | HPGD, CCL21, anti-TGFβ, anti-PD1 |
| 73 | HPGD, CCL21, anti-TGFβ, anti-TREM2 |
| 74 | HPGD, CCL21, anti-PD1, anti-TREM2 |
| 75 | HPGD, IL-12, anti-CD47, anti-TGFβ |
| 76 | HPGD, IL-12, anti-CD47, anti-PD1 |
| 77 | HPGD, IL-12, anti-CD47, anti-TREM2 |
| 78 | HPGD, IL-12, anti-TGFβ, anti-PD1 |
| 79 | HPGD, IL-12, anti-TGFβ, anti-TREM2 |
| 80 | HPGD, IL-12, anti-PD1, anti-TREM2 |
| 81 | HPGD, anti-CD47, anti-TGFβ, anti-PD1 |
| 82 | HPGD, anti-CD47, anti-TGFβ, anti-TREM2 |
| 83 | HPGD, anti-CD47, anti-PD1, anti-TREM2 |
| 84 | HPGD, anti-TGFβ, anti-PD1, anti-TREM2 |
| 85 | ADA2, HYAL1, CHP, CCL21 |
| 86 | ADA2, HYAL1, CHP, IL-12 |
| 87 | ADA2, HYAL1, CHP, anti-CD47 |
| 88 | ADA2, HYAL1, CHP, anti-TGFβ |
| 89 | ADA2, HYAL1, CHP, anti-PD1 |
| 90 | ADA2, HYAL1, CHP, anti-TREM2 |
| 91 | ADA2, HYAL1, CCL21, IL-12 |
| 92 | ADA2, HYAL1, CCL21, anti-CD47 |
| 93 | ADA2, HYAL1, CCL21, anti-TGFβ |
| 94 | ADA2, HYAL1, CCL21, anti-PD1 |
| 95 | ADA2, HYAL1, CCL21, anti-TREM2 |
| 96 | ADA2, HYAL1, IL-12, anti-CD47 |
| 97 | ADA2, HYAL1, IL-12, anti-TGFβ |
| 98 | ADA2, HYAL1, IL-12, anti-PD1 |
| 99 | ADA2, HYAL1, IL-12, anti-TREM2 |
| 100 | ADA2, HYAL1, anti-CD47, anti-TGFβ |
| 101 | ADA2, HYAL1, anti-CD47, anti-PD1 |
| 102 | ADA2, HYAL1, anti-CD47, anti-TREM2 |
| 103 | ADA2, HYAL1, anti-TGFβ, anti-PD1 |
| 104 | ADA2, HYAL1, anti-TGFβ, anti-TREM2 |
| 105 | ADA2, HYAL1, anti-PD1, anti-TREM2 |
| 106 | ADA2, CHP, CCL21, IL-12 |
| 107 | ADA2, CHP, CCL21, anti-CD47 |
| 108 | ADA2, CHP, CCL21, anti-TGFβ |
| 109 | ADA2, CHP, CCL21, anti-PD1 |
| 110 | ADA2, CHP, CCL21, anti-TREM2 |
| 111 | ADA2, CHP, IL-12, anti-CD47 |
| 112 | ADA2, CHP, IL-12, anti-TGFβ |
| 113 | ADA2, CHP, IL-12, anti-PD1 |
| 114 | ADA2, CHP, IL-12, anti-TREM2 |
| 115 | ADA2, CHP, anti-CD47, anti-TGFβ |
| 116 | ADA2, CHP, anti-CD47, anti-PD1 |
| 117 | ADA2, CHP, anti-CD47, anti-TREM2 |
| 118 | ADA2, CHP, anti-TGFβ, anti-PD1 |
| 119 | ADA2, CHP, anti-TGFβ, anti-TREM2 |
| 120 | ADA2, CHP, anti-PD1, anti-TREM2 |
| 121 | ADA2, CCL21, IL-12, anti-CD47 |
| 122 | ADA2, CCL21, IL-12, anti-TGFβ |
| 123 | ADA2, CCL21, IL-12, anti-PD1 |
| 124 | ADA2, CCL21, IL-12, anti-TREM2 |
| 125 | ADA2, CCL21, anti-CD47, anti-TGFβ |
| 126 | ADA2, CCL21, anti-CD47, anti-PD1 |
| 127 | ADA2, CCL21, anti-CD47, anti-TREM2 |
| 128 | ADA2, CCL21, anti-TGFβ, anti-PD1 |
| 129 | ADA2, CCL21, anti-TGFβ, anti-TREM2 |
| 130 | ADA2, CCL21, anti-PD1, anti-TREM2 |
| 131 | ADA2, IL-12, anti-CD47, anti-TGFβ |
| 132 | ADA2, IL-12, anti-CD47, anti-PD1 |
| 133 | ADA2, IL-12, anti-CD47, anti-TREM2 |
| 134 | ADA2, IL-12, anti-TGFβ, anti-PD1 |
| 135 | ADA2, IL-12, anti-TGFβ, anti-TREM2 |
| 136 | ADA2, IL-12, anti-PD1, anti-TREM2 |
| 137 | ADA2, anti-CD47, anti-TGFβ, anti-PD1 |
| 138 | ADA2, anti-CD47, anti-TGFβ, anti-TREM2 |
| 139 | ADA2, anti-CD47, anti-PD1, anti-TREM2 |
| 140 | ADA2, anti-TGFβ, anti-PD1, anti-TREM2 |
| 141 | HYAL1, CHP, CCL21, IL-12 |
| 142 | HYAL1, CHP, CCL21, anti-CD47 |
| 143 | HYAL1, CHP, CCL21, anti-TGFβ |
| 144 | HYAL1, CHP, CCL21, anti-PD1 |
| 145 | HYAL1, CHP, CCL21, anti-TREM2 |
| 146 | HYAL1, CHP, IL-12, anti-CD47 |
| 147 | HYAL1, CHP, IL-12, anti-TGFβ |
| 148 | HYAL1, CHP, IL-12, anti-PD1 |
| 149 | HYAL1, CHP, IL-12, anti-TREM2 |
| 150 | HYAL1, CHP, anti-CD47, anti-TGFβ |
| 151 | HYAL1, CHP, anti-CD47, anti-PD1 |
| 152 | HYAL1, CHP, anti-CD47, anti-TREM2 |
| 153 | HYAL1, CHP, anti-TGFβ, anti-PD1 |
| 154 | HYAL1, CHP, anti-TGFβ, anti-TREM2 |
| 155 | HYAL1, CHP, anti-PD1, anti-TREM2 |
| 156 | HYAL1, CCL21, IL-12, anti-CD47 |
| 157 | HYAL1, CCL21, IL-12, anti-TGFβ |
| 158 | HYAL1, CCL21, IL-12, anti-PD1 |
| 159 | HYAL1, CCL21, IL-12, anti-TREM2 |
| 160 | HYAL1, CCL21, anti-CD47, anti-TGFβ |
| 161 | HYAL1, CCL21, anti-CD47, anti-PD1 |
| 162 | HYAL1, CCL21, anti-CD47, anti-TREM2 |
| 163 | HYAL1, CCL21, anti-TGFβ, anti-PD1 |
| 164 | HYAL1, CCL21, anti-TGFβ, anti-TREM2 |
| 165 | HYAL1, CCL21, anti-PD1, anti-TREM2 |
| 166 | HYAL1, IL-12, anti-CD47, anti-TGFβ |
| 167 | HYAL1, IL-12, anti-CD47, anti-PD1 |
| 168 | HYAL1, IL-12, anti-CD47, anti-TREM2 |
| 169 | HYAL1, IL-12, anti-TGFβ, anti-PD1 |
| 170 | HYAL1, IL-12, anti-TGFβ, anti-TREM2 |
| 171 | HYAL1, IL-12, anti-PD1, anti-TREM2 |
| 172 | HYAL1, anti-CD47, anti-TGFβ, anti-PD1 |
| 173 | HYAL1, anti-CD47, anti-TGFβ, anti-TREM2 |
| 174 | HYAL1, anti-CD47, anti-PD1, anti-TREM2 |
| 175 | HYAL1, anti-TGFβ, anti-PD1, anti-TREM2 |
| 176 | CHP, CCL21, IL-12, anti-CD47 |
| 177 | CHP, CCL21, IL-12, anti-TGFβ |
| 178 | CHP, CCL21, IL-12, anti-PD1 |
| 179 | CHP, CCL21, IL-12, anti-TREM2 |
| 180 | CHP, CCL21, anti-CD47, anti-TGFβ |
| 181 | CHP, CCL21, anti-CD47, anti-PD1 |
| 182 | CHP, CCL21, anti-CD47, anti-TREM2 |
| 183 | CHP, CCL21, anti-TGFβ, anti-PD1 |
| 184 | CHP, CCL21, anti-TGFβ, anti-TREM2 |
| 185 | CHP, CCL21, anti-PD1, anti-TREM2 |
| 186 | CHP, IL-12, anti-CD47, anti-TGFβ |
| 187 | CHP, IL-12, anti-CD47, anti-PD1 |
| 188 | CHP, IL-12, anti-CD47, anti-TREM2 |
| 189 | CHP, IL-12, anti-TGFβ, anti-PD1 |
| 190 | CHP, IL-12, anti-TGFβ, anti-TREM2 |
| 191 | CHP, IL-12, anti-PD1, anti-TREM2 |
| 192 | CHP, anti-CD47, anti-TGFβ, anti-PD1 |
| 193 | CHP, anti-CD47, anti-TGFβ, anti-TREM2 |
| 194 | CHP, anti-CD47, anti-PD1, anti-TREM2 |
| 195 | CHP, anti-TGFβ, anti-PD1, anti-TREM2 |
| 196 | CCL21, IL-12, anti-CD47, anti-TGFβ |
| 197 | CCL21, IL-12, anti-CD47, anti-PD1 |
| 198 | CCL21, IL-12, anti-CD47, anti-TREM2 |
| 199 | CCL21, IL-12, anti-TGFβ, anti-PD1 |
| 200 | CCL21, IL-12, anti-TGFβ, anti-TREM2 |
| 201 | CCL21, IL-12, anti-PD1, anti-TREM2 |
| 202 | CCL21, anti-CD47, anti-TGFβ, anti-PD1 |
| 203 | CCL21, anti-CD47, anti-TGFβ, anti-TREM2 |
| 204 | CCL21, anti-CD47, anti-PD1, anti-TREM2 |
| 205 | CCL21, anti-TGFβ, anti-PD1, anti-TREM2 |
| 206 | IL-12, anti-CD47, anti-TGFβ, anti-PD1 |
| 207 | IL-12, anti-CD47, anti-TGFβ, anti-TREM2 |
| 208 | IL-12, anti-CD47, anti-PD1, anti-TREM2 |
| 209 | IL-12, anti-TGFβ, anti-PD1, anti-TREM2 |
| 210 | anti-CD47, anti-TGFβ, anti-PD1, anti-TREM2 |
| 211 | CTX-BiTE, HPGD, ADA2, HYAL1 |
| 212 | CTX-BiTE, HPGD, ADA2, CHP |
| 213 | CTX-BiTE, HPGD, ADA2, CCL21 |
| 214 | CTX-BiTE, HPGD, ADA2, IL-12 |
| 215 | CTX-BiTE, HPGD, ADA2, anti-CD47 |
| 216 | CTX-BiTE, HPGD, ADA2, anti-TGFβ |
| 217 | CTX-BiTE, HPGD, ADA2, anti-PD1 |
| 218 | CTX-BiTE, HPGD, ADA2, anti-TREM2 |
| 219 | CTX-BiTE, HPGD, HYAL1, CHP |
| 220 | CTX-BiTE, HPGD, HYAL1, CCL21 |
| 221 | CTX-BiTE, HPGD, HYAL1, IL-12 |
| 222 | CTX-BiTE, HPGD, HYAL1, anti-CD47 |
| 223 | CTX-BiTE, HPGD, HYAL1, anti-TGFβ |
| 224 | CTX-BiTE, HPGD, HYAL1, anti-PD1 |
| 225 | CTX-BiTE, HPGD, HYAL1, anti-TREM2 |
| 226 | CTX-BiTE, HPGD, CHP, CCL21 |
| 227 | CTX-BiTE, HPGD, CHP, IL-12 |
| 228 | CTX-BiTE, HPGD, CHP, anti-CD47 |
| 229 | CTX-BiTE, HPGD, CHP, anti-TGFβ |
| 230 | CTX-BiTE, HPGD, CHP, anti-PD1 |
| 231 | CTX-BiTE, HPGD, CHP, anti-TREM2 |
| 232 | CTX-BiTE, HPGD, CCL21, IL-12 |
| 233 | CTX-BiTE, HPGD, CCL21, anti-CD47 |
| 234 | CTX-BiTE, HPGD, CCL21, anti-TGFβ |
| 235 | CTX-BiTE, HPGD, CCL21, anti-PD1 |
| 236 | CTX-BiTE, HPGD, CCL21, anti-TREM2 |
| 237 | CTX-BiTE, HPGD, IL-12, anti-CD47 |
| 238 | CTX-BiTE, HPGD, IL-12, anti-TGFβ |
| 239 | CTX-BiTE, HPGD, IL-12, anti-PD1 |
| 240 | CTX-BiTE, HPGD, IL-12, anti-TREM2 |
| 241 | CTX-BiTE, HPGD, anti-CD47, anti-TGFβ |
| 242 | CTX-BiTE, HPGD, anti-CD47, anti-PD1 |
| 243 | CTX-BiTE, HPGD, anti-CD47, anti-TREM2 |
| 244 | CTX-BiTE, HPGD, anti-TGFβ, anti-PD1 |
| 245 | CTX-BiTE, HPGD, anti-TGFβ, anti-TREM2 |
| 246 | CTX-BiTE, HPGD, anti-PD1, anti-TREM2 |
| 247 | CTX-BiTE, ADA2, HYAL1, CHP |
| 248 | CTX-BiTE, ADA2, HYAL1, CCL21 |
| 249 | CTX-BiTE, ADA2, HYAL1, IL-12 |
| 250 | CTX-BiTE, ADA2, HYAL1, anti-CD47 |
| 251 | CTX-BiTE, ADA2, HYAL1, anti-TGFβ |
| 252 | CTX-BiTE, ADA2, HYAL1, anti-PD1 |
| 253 | CTX-BiTE, ADA2, HYAL1, anti-TREM2 |
| 254 | CTX-BiTE, ADA2, CHP, CCL21 |
| 255 | CTX-BiTE, ADA2, CHP, IL-12 |
| 256 | CTX-BiTE, ADA2, CHP, anti-CD47 |
| 257 | CTX-BiTE, ADA2, CHP, anti-TGFβ |
| 258 | CTX-BiTE, ADA2, CHP, anti-PD1 |
| 259 | CTX-BiTE, ADA2, CHP, anti-TREM2 |
| 260 | CTX-BiTE, ADA2, CCL21, IL-12 |
| 261 | CTX-BiTE, ADA2, CCL21, anti-CD47 |
| 262 | CTX-BiTE, ADA2, CCL21, anti-TGFβ |
| 263 | CTX-BiTE, ADA2, CCL21, anti-PD1 |
| 264 | CTX-BiTE, ADA2, CCL21, anti-TREM2 |
| 265 | CTX-BiTE, ADA2, IL-12, anti-CD47 |
| 266 | CTX-BiTE, ADA2, IL-12, anti-TGFβ |
| 267 | CTX-BiTE, ADA2, IL-12, anti-PD1 |
| 268 | CTX-BiTE, ADA2, IL-12, anti-TREM2 |
| 269 | CTX-BiTE, ADA2, anti-CD47, anti-TGFβ |
| 270 | CTX-BiTE, ADA2, anti-CD47, anti-PD1 |
| 271 | CTX-BiTE, ADA2, anti-CD47, anti-TREM2 |
| 272 | CTX-BiTE, ADA2, anti-TGFβ, anti-PD1 |
| 273 | CTX-BiTE, ADA2, anti-TGFβ, anti-TREM2 |
| 274 | CTX-BiTE, ADA2, anti-PD1, anti-TREM2 |
| 275 | CTX-BiTE, HYAL1, CHP, CCL21 |
| 276 | CTX-BiTE, HYAL1, CHP, IL-12 |
| 277 | CTX-BiTE, HYAL1, CHP, anti-CD47 |
| 278 | CTX-BiTE, HYAL1, CHP, anti-TGFβ |
| 279 | CTX-BiTE, HYAL1, CHP, anti-PD1 |
| 280 | CTX-BiTE, HYAL1, CHP, anti-TREM2 |
| 281 | CTX-BiTE, HYAL1, CCL21, IL-12 |
| 282 | CTX-BiTE, HYAL1, CCL21, anti-CD47 |
| 283 | CTX-BiTE, HYAL1, CCL21, anti-TGFβ |
| 284 | CTX-BiTE, HYAL1, CCL21, anti-PD1 |
| 285 | CTX-BiTE, HYAL1, CCL21, anti-TREM2 |
| 286 | CTX-BiTE, HYAL1, IL-12, anti-CD47 |
| 287 | CTX-BiTE, HYAL1, IL-12, anti-TGFβ |
| 288 | CTX-BiTE, HYAL1, IL-12, anti-PD1 |
| 289 | CTX-BiTE, HYAL1, IL-12, anti-TREM2 |
| 290 | CTX-BiTE, HYAL1, anti-CD47, anti-TGFβ |
| 291 | CTX-BiTE, HYAL1, anti-CD47, anti-PD1 |
| 292 | CTX-BiTE, HYAL1, anti-CD47, anti-TREM2 |
| 293 | CTX-BiTE, HYAL1, anti-TGFβ, anti-PD1 |
| 294 | CTX-BiTE, HYAL1, anti-TGFβ, anti-TREM2 |
| 295 | CTX-BiTE, HYAL1, anti-PD1, anti-TREM2 |
| 296 | CTX-BiTE, CHP, CCL21, IL-12 |
| 297 | CTX-BiTE, CHP, CCL21, anti-CD47 |
| 298 | CTX-BiTE, CHP, CCL21, anti-TGFβ |
| 299 | CTX-BiTE, CHP, CCL21, anti-PD1 |
| 300 | CTX-BiTE, CHP, CCL21, anti-TREM2 |
| 301 | CTX-BiTE, CHP, IL-12, anti-CD47 |
| 302 | CTX-BiTE, CHP, IL-12, anti-TGFβ |
| 303 | CTX-BiTE, CHP, IL-12, anti-PD1 |
| 304 | CTX-BiTE, CHP, IL-12, anti-TREM2 |
| 305 | CTX-BiTE, CHP, anti-CD47, anti-TGFβ |
| 306 | CTX-BiTE, CHP, anti-CD47, anti-PD1 |
| 307 | CTX-BiTE, CHP, anti-CD47, anti-TREM2 |
| 308 | CTX-BiTE, CHP, anti-TGFβ, anti-PD1 |
| 309 | CTX-BiTE, CHP, anti-TGFβ, anti-TREM2 |
| 310 | CTX-BiTE, CHP, anti-PD1, anti-TREM2 |
| 311 | CTX-BiTE, CCL21, IL-12, anti-CD47 |
| 312 | CTX-BiTE, CCL21, IL-12, anti-TGFβ |
| 313 | CTX-BiTE, CCL21, IL-12, anti-PD1 |
| 314 | CTX-BiTE, CCL21, IL-12, anti-TREM2 |
| 315 | CTX-BiTE, CCL21, anti-CD47, anti-TGFβ |
| 316 | CTX-BiTE, CCL21, anti-CD47, anti-PD1 |
| 317 | CTX-BiTE, CCL21, anti-CD47, anti-TREM2 |
| 318 | CTX-BiTE, CCL21, anti-TGFβ, anti-PD1 |
| 319 | CTX-BiTE, CCL21, anti-TGFβ, anti-TREM2 |
| 320 | CTX-BiTE, CCL21, anti-PD1, anti-TREM2 |
| 321 | CTX-BiTE, IL-12, anti-CD47, anti-TGFβ |
| 322 | CTX-BiTE, IL-12, anti-CD47, anti-PD1 |
| 323 | CTX-BiTE, IL-12, anti-CD47, anti-TREM2 |
| 324 | CTX-BiTE, IL-12, anti-TGFβ, anti-PD1 |
| 325 | CTX-BiTE, IL-12, anti-TGFβ, anti-TREM2 |
| 326 | CTX-BiTE, IL-12, anti-PD1, anti-TREM2 |
| 327 | CTX-BiTE, anti-CD47, anti-TGFβ, anti-PD1 |
| 328 | CTX-BiTE, anti-CD47, anti-TGFβ, anti-TREM2 |
| 329 | CTX-BiTE, anti-CD47, anti-PD1, anti-TREM2 |
| 330 | CTX-BiTE, anti-TGFβ, anti-PD1, anti-TREM2 |

In some embodiments, the oncolytic viruses of the disclosure comprise one or more polynucleotides encoding five or more payload molecules comprising any of the combinations listed in Table 7 below.

TABLE 7
Combination of Five Payload Molecules

| Combo # | Payload Molecules |
|---|---|
| 1 | HPGD, ADA2, HYAL1, CHP, CCL21 |
| 2 | HPGD, ADA2, HYAL1, CHP, IL-12 |
| 3 | HPGD, ADA2, HYAL1, CHP, anti-CD47 |
| 4 | HPGD, ADA2, HYAL1, CHP, anti-TGFβ |
| 5 | HPGD, ADA2, HYAL1, CHP, anti-PD1 |
| 6 | HPGD, ADA2, HYAL1, CHP, anti-TREM2 |
| 7 | HPGD, ADA2, HYAL1, CCL21, IL-12 |
| 8 | HPGD, ADA2, HYAL1, CCL21, anti-CD47 |
| 9 | HPGD, ADA2, HYAL1, CCL21, anti-TGFβ |
| 10 | HPGD, ADA2, HYAL1, CCL21, anti-PD1 |
| 11 | HPGD, ADA2, HYAL1, CCL21, anti-TREM2 |
| 12 | HPGD, ADA2, HYAL1, IL-12, anti-CD47 |
| 13 | HPGD, ADA2, HYAL1, IL-12, anti-TGFβ |
| 14 | HPGD, ADA2, HYAL1, IL-12, anti-PD1 |
| 15 | HPGD, ADA2, HYAL1, IL-12, anti-TREM2 |
| 16 | HPGD, ADA2, HYAL1, anti-CD47, anti-TGFβ |
| 17 | HPGD, ADA2, HYAL1, anti-CD47, anti-PD1 |
| 18 | HPGD, ADA2, HYAL1, anti-CD47, anti-TREM2 |
| 19 | HPGD, ADA2, HYAL1, anti-TGFβ, anti-PD1 |
| 20 | HPGD, ADA2, HYAL1, anti-TGFβ, anti-TREM2 |
| 21 | HPGD, ADA2, HYAL1, anti-PD1, anti-TREM2 |
| 22 | HPGD, ADA2, CHP, CCL21, IL-12 |
| 23 | HPGD, ADA2, CHP, CCL21, anti-CD47 |
| 24 | HPGD, ADA2, CHP, CCL21, anti-TGFβ |
| 25 | HPGD, ADA2, CHP, CCL21, anti-PD1 |
| 26 | HPGD, ADA2, CHP, CCL21, anti-TREM2 |
| 27 | HPGD, ADA2, CHP, IL-12, anti-CD47 |
| 28 | HPGD, ADA2, CHP, IL-12, anti-TGFβ |
| 29 | HPGD, ADA2, CHP, IL-12, anti-PD1 |
| 30 | HPGD, ADA2, CHP, IL-12, anti-TREM2 |
| 31 | HPGD, ADA2, CHP, anti-CD47, anti-TGFβ |
| 32 | HPGD, ADA2, CHP, anti-CD47, anti-PD1 |
| 33 | HPGD, ADA2, CHP, anti-CD47, anti-TREM2 |
| 34 | HPGD, ADA2, CHP, anti-TGFβ, anti-PD1 |
| 35 | HPGD, ADA2, CHP, anti-TGFβ, anti-TREM2 |
| 36 | HPGD, ADA2, CHP, anti-PD1, anti-TREM2 |
| 37 | HPGD, ADA2, CCL21, IL-12, anti-CD47 |
| 38 | HPGD, ADA2, CCL21, IL-12, anti-TGFβ |
| 39 | HPGD, ADA2, CCL21, IL-12, anti-PD1 |
| 40 | HPGD, ADA2, CCL21, IL-12, anti-TREM2 |
| 41 | HPGD, ADA2, CCL21, anti-CD47, anti-TGFβ |
| 42 | HPGD, ADA2, CCL21, anti-CD47, anti-PD1 |
| 43 | HPGD, ADA2, CCL21, anti-CD47, anti-TREM2 |
| 44 | HPGD, ADA2, CCL21, anti-TGFβ, anti-PD1 |
| 45 | HPGD, ADA2, CCL21, anti-TGFβ, anti-TREM2 |
| 46 | HPGD, ADA2, CCL21, anti-PD1, anti-TREM2 |
| 47 | HPGD, ADA2, IL-12, anti-CD47, anti-TGFβ |
| 48 | HPGD, ADA2, IL-12, anti-CD47, anti-PD1 |
| 49 | HPGD, ADA2, IL-12, anti-CD47, anti-TREM2 |
| 50 | HPGD, ADA2, IL-12, anti-TGFβ, anti-PD1 |
| 51 | HPGD, ADA2, IL-12, anti-TGFβ, anti-TREM2 |
| 52 | HPGD, ADA2, IL-12, anti-PD1, anti-TREM2 |
| 53 | HPGD, ADA2, anti-CD47, anti-TGFβ, anti-PD1 |
| 54 | HPGD, ADA2, anti-CD47, anti-TGFβ, anti-TREM2 |
| 55 | HPGD, ADA2, anti-CD47, anti-PD1, anti-TREM2 |
| 56 | HPGD, ADA2, anti-TGFβ, anti-PD1, anti-TREM2 |
| 57 | HPGD, HYAL1, CHP, CCL21, IL-12 |
| 58 | HPGD, HYAL1, CHP, CCL21, anti-CD47 |
| 59 | HPGD, HYAL1, CHP, CCL21, anti-TGFβ |
| 60 | HPGD, HYAL1, CHP, CCL21, anti-PD1 |
| 61 | HPGD, HYAL1, CHP, CCL21, anti-TREM2 |
| 62 | HPGD, HYAL1, CHP, IL-12, anti-CD47 |
| 63 | HPGD, HYAL1, CHP, IL-12, anti-TGFβ |
| 64 | HPGD, HYAL1, CHP, IL-12, anti-PD1 |
| 65 | HPGD, HYAL1, CHP, IL-12, anti-TREM2 |
| 66 | HPGD, HYAL1, CHP, anti-CD47, anti-TGFβ |
| 67 | HPGD, HYAL1, CHP, anti-CD47, anti-PD1 |
| 68 | HPGD, HYAL1, CHP, anti-CD47, anti-TREM2 |
| 69 | HPGD, HYAL1, CHP, anti-TGFβ, anti-PD1 |
| 70 | HPGD, HYAL1, CHP, anti-TGFβ, anti-TREM2 |
| 71 | HPGD, HYAL1, CHP, anti-PD1, anti-TREM2 |
| 72 | HPGD, HYAL1, CCL21, IL-12, anti-CD47 |
| 73 | HPGD, HYAL1, CCL21, IL-12, anti-TGFβ |
| 74 | HPGD, HYAL1, CCL21, IL-12, anti-PD1 |
| 75 | HPGD, HYAL1, CCL21, IL-12, anti-TREM2 |
| 76 | HPGD, HYAL1, CCL21, anti-CD47, anti-TGFβ |
| 77 | HPGD, HYAL1, CCL21, anti-CD47, anti-PD1 |
| 78 | HPGD, HYAL1, CCL21, anti-CD47, anti-TREM2 |
| 79 | HPGD, HYAL1, CCL21, anti-TGFβ, anti-PD1 |
| 80 | HPGD, HYAL1, CCL21, anti-TGFβ, anti-TREM2 |
| 81 | HPGD, HYAL1, CCL21, anti-PD1, anti-TREM2 |
| 82 | HPGD, HYAL1, IL-12, anti-CD47, anti-TGFβ |
| 83 | HPGD, HYAL1, IL-12, anti-CD47, anti-PD1 |
| 84 | HPGD, HYAL1, IL-12, anti-CD47, anti-TREM2 |
| 85 | HPGD, HYAL1, IL-12, anti-TGFβ, anti-PD1 |
| 86 | HPGD, HYAL1, IL-12, anti-TGFβ, anti-TREM2 |
| 87 | HPGD, HYAL1, IL-12, anti-PD1, anti-TREM2 |
| 88 | HPGD, HYAL1, anti-CD47, anti-TGFβ, anti-PD1 |
| 89 | HPGD, HYAL1, anti-CD47, anti-TGFβ, anti-TREM2 |
| 90 | HPGD, HYAL1, anti-CD47, anti-PD1, anti-TREM2 |
| 91 | HPGD, HYAL1, anti-TGFβ, anti-PD1, anti-TREM2 |
| 92 | HPGD, CHP, CCL21, IL-12, anti-CD47 |
| 93 | HPGD, CHP, CCL21, IL-12, anti-TGFβ |
| 94 | HPGD, CHP, CCL21, IL-12, anti-PD1 |
| 95 | HPGD, CHP, CCL21, IL-12, anti-TREM2 |
| 96 | HPGD, CHP, CCL21, anti-CD47, anti-TGFβ |
| 97 | HPGD, CHP, CCL21, anti-CD47, anti-PD1 |
| 98 | HPGD, CHP, CCL21, anti-CD47, anti-TREM2 |
| 99 | HPGD, CHP, CCL21, anti-TGFβ, anti-PD1 |
| 100 | HPGD, CHP, CCL21, anti-TGFβ, anti-TREM2 |
| 101 | HPGD, CHP, CCL21, anti-PD1, anti-TREM2 |
| 102 | HPGD, CHP, IL-12, anti-CD47, anti-TGFβ |
| 103 | HPGD, CHP, IL-12, anti-CD47, anti-PD1 |
| 104 | HPGD, CHP, IL-12, anti-CD47, anti-TREM2 |
| 105 | HPGD, CHP, IL-12, anti-TGFβ, anti-PD1 |
| 106 | HPGD, CHP, IL-12, anti-TGFβ, anti-TREM2 |
| 107 | HPGD, CHP, IL-12, anti-PD1, anti-TREM2 |
| 108 | HPGD, CHP, anti-CD47, anti-TGFβ, anti-PD1 |
| 109 | HPGD, CHP, anti-CD47, anti-TGFβ, anti-TREM2 |
| 110 | HPGD, CHP, anti-CD47, anti-PD1, anti-TREM2 |
| 111 | HPGD, CHP, anti-TGFβ, anti-PD1, anti-TREM2 |
| 112 | HPGD, CCL21, IL-12, anti-CD47, anti-TGFβ |
| 113 | HPGD, CCL21, IL-12, anti-CD47, anti-PD1 |
| 114 | HPGD, CCL21, IL-12, anti-CD47, anti-TREM2 |
| 115 | HPGD, CCL21, IL-12, anti-TGFβ, anti-PD1 |
| 116 | HPGD, CCL21, IL-12, anti-TGFβ, anti-TREM2 |
| 117 | HPGD, CCL21, IL-12, anti-PD1, anti-TREM2 |
| 118 | HPGD, CCL21, anti-CD47, anti-TGFβ, anti-PD1 |
| 119 | HPGD, CCL21, anti-CD47, anti-TGFβ, anti-TREM2 |
| 120 | HPGD, CCL21, anti-CD47, anti-PD1, anti-TREM2 |
| 121 | HPGD, CCL21, anti-TGFβ, anti-PD1, anti-TREM2 |
| 122 | HPGD, IL-12, anti-CD47, anti-TGFβ, anti-PD1 |
| 123 | HPGD, IL-12, anti-CD47, anti-TGFβ, anti-TREM2 |
| 124 | HPGD, IL-12, anti-CD47, anti-PD1, anti-TREM2 |
| 125 | HPGD, IL-12, anti-TGFβ, anti-PD1, anti-TREM2 |
| 126 | HPGD, anti-CD47, anti-TGFβ, anti-PD1, anti-TREM2 |
| 127 | ADA2, HYAL1, CHP, CCL21, IL-12 |
| 128 | ADA2, HYAL1, CHP, CCL21, anti-CD47 |
| 129 | ADA2, HYAL1, CHP, CCL21, anti-TGFβ |
| 130 | ADA2, HYAL1, CHP, CCL21, anti-PD1 |
| 131 | ADA2, HYAL1, CHP, CCL21, anti-TREM2 |
| 132 | ADA2, HYAL1, CHP, IL-12, anti-CD47 |
| 133 | ADA2, HYAL1, CHP, IL-12, anti-TGFβ |
| 134 | ADA2, HYAL1, CHP, IL-12, anti-PD1 |
| 135 | ADA2, HYAL1, CHP, IL-12, anti-TREM2 |
| 136 | ADA2, HYAL1, CHP, anti-CD47, anti-TGFβ |
| 137 | ADA2, HYAL1, CHP, anti-CD47, anti-PD1 |
| 138 | ADA2, HYAL1, CHP, anti-CD47, anti-TREM2 |
| 139 | ADA2, HYAL1, CHP, anti-TGFβ, anti-PD1 |
| 140 | ADA2, HYAL1, CHP, anti-TGFβ, anti-TREM2 |
| 141 | ADA2, HYAL1, CHP, anti-PD1, anti-TREM2 |
| 142 | ADA2, HYAL1, CCL21, IL-12, anti-CD47 |
| 143 | ADA2, HYAL1, CCL21, IL-12, anti-TGFβ |
| 144 | ADA2, HYAL1, CCL21, IL-12, anti-PD1 |
| 145 | ADA2, HYAL1, CCL21, IL-12, anti-TREM2 |
| 146 | ADA2, HYAL1, CCL21, anti-CD47, anti-TGFβ |
| 147 | ADA2, HYAL1, CCL21, anti-CD47, anti-PD1 |
| 148 | ADA2, HYAL1, CCL21, anti-CD47, anti-TREM2 |
| 149 | ADA2, HYAL1, CCL21, anti-TGFβ, anti-PD1 |
| 150 | ADA2, HYAL1, CCL21, anti-TGFβ, anti-TREM2 |
| 151 | ADA2, HYAL1, CCL21, anti-PD1, anti-TREM2 |
| 152 | ADA2, HYAL1, IL-12, anti-CD47, anti-TGFβ |
| 153 | ADA2, HYAL1, IL-12, anti-CD47, anti-PD1 |
| 154 | ADA2, HYAL1, IL-12, anti-CD47, anti-TREM2 |
| 155 | ADA2, HYAL1, IL-12, anti-TGFβ, anti-PD1 |
| 156 | ADA2, HYAL1, IL-12, anti-TGFβ, anti-TREM2 |
| 157 | ADA2, HYAL1, IL-12, anti-PD1, anti-TREM2 |
| 158 | ADA2, HYAL1, anti-CD47, anti-TGFβ, anti-PD1 |
| 159 | ADA2, HYAL1, anti-CD47, anti-TGFβ, anti-TREM2 |
| 160 | ADA2, HYAL1, anti-CD47, anti-PD1, anti-TREM2 |
| 161 | ADA2, HYAL1, anti-TGFβ, anti-PD1, anti-TREM2 |
| 162 | ADA2, CHP, CCL21, IL-12, anti-CD47 |
| 163 | ADA2, CHP, CCL21, IL-12, anti-TGFβ |
| 164 | ADA2, CHP, CCL21, IL-12, anti-PD1 |
| 165 | ADA2, CHP, CCL21, IL-12, anti-TREM2 |
| 166 | ADA2, CHP, CCL21, anti-CD47, anti-TGFβ |
| 167 | ADA2, CHP, CCL21, anti-CD47, anti-PD1 |
| 168 | ADA2, CHP, CCL21, anti-CD47, anti-TREM2 |
| 169 | ADA2, CHP, CCL21, anti-TGFβ, anti-PD1 |
| 170 | ADA2, CHP, CCL21, anti-TGFβ, anti-TREM2 |
| 171 | ADA2, CHP, CCL21, anti-PD1, anti-TREM2 |
| 172 | ADA2, CHP, IL-12, anti-CD47, anti-TGFβ |
| 173 | ADA2, CHP, IL-12, anti-CD47, anti-PD1 |
| 174 | ADA2, CHP, IL-12, anti-CD47, anti-TREM2 |
| 175 | ADA2, CHP, IL-12, anti-TGFβ, anti-PD1 |
| 176 | ADA2, CHP, IL-12, anti-TGFβ, anti-TREM2 |
| 177 | ADA2, CHP, IL-12, anti-PD1, anti-TREM2 |
| 178 | ADA2, CHP, anti-CD47, anti-TGFβ, anti-PD1 |
| 179 | ADA2, CHP, anti-CD47, anti-TGFβ, anti-TREM2 |
| 180 | ADA2, CHP, anti-CD47, anti-PD1, anti-TREM2 |
| 181 | ADA2, CHP, anti-TGFβ, anti-PD1, anti-TREM2 |
| 182 | ADA2, CCL21, IL-12, anti-CD47, anti-TGFβ |
| 183 | ADA2, CCL21, IL-12, anti-CD47, anti-PD1 |
| 184 | ADA2, CCL21, IL-12, anti-CD47, anti-TREM2 |
| 185 | ADA2, CCL21, IL-12, anti-TGFβ, anti-PD1 |
| 186 | ADA2, CCL21, IL-12, anti-TGFβ, anti-TREM2 |
| 187 | ADA2, CCL21, IL-12, anti-PD1, anti-TREM2 |
| 188 | ADA2, CCL21, anti-CD47, anti-TGFβ, anti-PD1 |
| 189 | ADA2, CCL21, anti-CD47, anti-TGFβ, anti-TREM2 |
| 190 | ADA2, CCL21, anti-CD47, anti-PD1, anti-TREM2 |
| 191 | ADA2, CCL21, anti-TGFβ, anti-PD1, anti-TREM2 |
| 192 | ADA2, IL-12, anti-CD47, anti-TGFβ, anti-PD1 |
| 193 | ADA2, IL-12, anti-CD47, anti-TGFβ, anti-TREM2 |
| 194 | ADA2, IL-12, anti-CD47, anti-PD1, anti-TREM2 |
| 195 | ADA2, IL-12, anti-TGFβ, anti-PD1, anti-TREM2 |
| 196 | ADA2, anti-CD47, anti-TGFβ, anti-PD1, anti-TREM2 |
| 197 | HYAL1, CHP, CCL21, IL-12, anti-CD47 |
| 198 | HYAL1, CHP, CCL21, IL-12, anti-TGFβ |
| 199 | HYAL1, CHP, CCL21, IL-12, anti-PD1 |
| 200 | HYAL1, CHP, CCL21, IL-12, anti-TREM2 |
| 201 | HYAL1, CHP, CCL21, anti-CD47, anti-TGFβ |
| 202 | HYAL1, CHP, CCL21, anti-CD47, anti-PD1 |
| 203 | HYAL1, CHP, CCL21, anti-CD47, anti-TREM2 |
| 204 | HYAL1, CHP, CCL21, anti-TGFβ, anti-PD1 |
| 205 | HYAL1, CHP, CCL21, anti-TGFβ, anti-TREM2 |
| 206 | HYAL1, CHP, CCL21, anti-PD1, anti-TREM2 |
| 207 | HYAL1, CHP, IL-12, anti-CD47, anti-TGFβ |
| 208 | HYAL1, CHP, IL-12, anti-CD47, anti-PD1 |
| 209 | HYAL1, CHP, IL-12, anti-CD47, anti-TREM2 |
| 210 | HYAL1, CHP, IL-12, anti-TGFβ, anti-PD1 |
| 211 | HYAL1, CHP, IL-12, anti-TGFβ, anti-TREM2 |
| 212 | HYAL1, CHP, IL-12, anti-PD1, anti-TREM2 |
| 213 | HYAL1, CHP, anti-CD47, anti-TGFβ, anti-PD1 |
| 214 | HYAL1, CHP, anti-CD47, anti-TGFβ, anti-TREM2 |
| 215 | HYAL1, CHP, anti-CD47, anti-PD1, anti-TREM2 |
| 216 | HYAL1, CHP, anti-TGFβ, anti-PD1, anti-TREM2 |
| 217 | HYAL1, CCL21, IL-12, anti-CD47, anti-TGFβ |
| 218 | HYAL1, CCL21, IL-12, anti-CD47, anti-PD1 |
| 219 | HYAL1, CCL21, IL-12, anti-CD47, anti-TREM2 |
| 220 | HYAL1, CCL21, IL-12, anti-TGFβ, anti-PD1 |
| 221 | HYAL1, CCL21, IL-12, anti-TGFβ, anti-TREM2 |
| 222 | HYAL1, CCL21, IL-12, anti-PD1, anti-TREM2 |
| 223 | HYAL1, CCL21, anti-CD47, anti-TGFβ, anti-PD1 |
| 224 | HYAL1, CCL21, anti-CD47, anti-TGFβ, anti-TREM2 |
| 225 | HYAL1, CCL21, anti-CD47, anti-PD1, anti-TREM2 |
| 226 | HYAL1, CCL21, anti-TGFβ, anti-PD1, anti-TREM2 |
| 227 | HYAL1, IL-12, anti-CD47, anti-TGFβ, anti-PD1 |
| 228 | HYAL1, IL-12, anti-CD47, anti-TGFβ, anti-TREM2 |
| 229 | HYAL1, IL-12, anti-CD47, anti-PD1, anti-TREM2 |
| 230 | HYAL1, IL-12, anti-TGFβ, anti-PD1, anti-TREM2 |
| 231 | HYAL1, anti-CD47, anti-TGFβ, anti-PD1, anti-TREM2 |
| 232 | CHP, CCL21, IL-12, anti-CD47, anti-TGFβ |
| 233 | CHP, CCL21, IL-12, anti-CD47, anti-PD1 |
| 234 | CHP, CCL21, IL-12, anti-CD47, anti-TREM2 |
| 235 | CHP, CCL21, IL-12, anti-TGFβ, anti-PD1 |
| 236 | CHP, CCL21, IL-12, anti-TGFβ, anti-TREM2 |
| 237 | CHP, CCL21, IL-12, anti-PD1, anti-TREM2 |
| 238 | CHP, CCL21, anti-CD47, anti-TGFβ, anti-PD1 |
| 239 | CHP, CCL21, anti-CD47, anti-TGFβ, anti-TREM2 |
| 240 | CHP, CCL21, anti-CD47, anti-PD1, anti-TREM2 |
| 241 | CHP, CCL21, anti-TGFβ, anti-PD1, anti-TREM2 |
| 242 | CHP, IL-12, anti-CD47, anti-TGFβ, anti-PD1 |
| 243 | CHP, IL-12, anti-CD47, anti-TGFβ, anti-TREM2 |
| 244 | CHP, IL-12, anti-CD47, anti-PD1, anti-TREM2 |
| 245 | CHP, IL-12, anti-TGFβ, anti-PD1, anti-TREM2 |
| 246 | CHP, anti-CD47, anti-TGFβ, anti-PD1, anti-TREM2 |
| 247 | CCL21, IL-12, anti-CD47, anti-TGFβ, anti-PD1 |
| 248 | CCL21, IL-12, anti-CD47, anti-TGFβ, anti-TREM2 |
| 249 | CCL21, IL-12, anti-CD47, anti-PD1, anti-TREM2 |
| 250 | CCL21, IL-12, anti-TGFβ, anti-PD1, anti-TREM2 |
| 251 | CCL21, anti-CD47, anti-TGFβ, anti-PD1, anti-TREM2 |
| 252 | IL-12, anti-CD47, anti-TGFβ, anti-PD1, anti-TREM2 |
| 253 | CTX-BiTE, HPGD, ADA2, HYAL1, CHP |
| 254 | CTX-BiTE, HPGD, ADA2, HYAL1, CCL21 |
| 255 | CTX-BiTE, HPGD, ADA2, HYAL1, IL-12 |
| 256 | CTX-BiTE, HPGD, ADA2, HYAL1, anti-CD47 |
| 257 | CTX-BiTE, HPGD, ADA2, HYAL1, anti-TGFβ |
| 258 | CTX-BiTE, HPGD, ADA2, HYAL1, anti-PD1 |
| 259 | CTX-BiTE, HPGD, ADA2, HYAL1, anti-TREM2 |
| 260 | CTX-BiTE, HPGD, ADA2, CHP, CCL21 |
| 261 | CTX-BiTE, HPGD, ADA2, CHP, IL-12 |
| 262 | CTX-BiTE, HPGD, ADA2, CHP, anti-CD47 |
| 263 | CTX-BiTE, HPGD, ADA2, CHP, anti-TGFβ |
| 264 | CTX-BiTE, HPGD, ADA2, CHP, anti-PD1 |
| 265 | CTX-BiTE, HPGD, ADA2, CHP, anti-TREM2 |
| 266 | CTX-BiTE, HPGD, ADA2, CCL21, IL-12 |
| 267 | CTX-BiTE, HPGD, ADA2, CCL21, anti-CD47 |
| 268 | CTX-BiTE, HPGD, ADA2, CCL21, anti-TGFβ |
| 269 | CTX-BiTE, HPGD, ADA2, CCL21, anti-PD1 |
| 270 | CTX-BiTE, HPGD, ADA2, CCL21, anti-TREM2 |
| 271 | CTX-BiTE, HPGD, ADA2, IL-12, anti-CD47 |
| 272 | CTX-BiTE, HPGD, ADA2, IL-12, anti-TGFβ |
| 273 | CTX-BiTE, HPGD, ADA2, IL-12, anti-PD1 |
| 274 | CTX-BiTE, HPGD, ADA2, IL-12, anti-TREM2 |
| 275 | CTX-BiTE, HPGD, ADA2, anti-CD47, anti-TGFβ |
| 276 | CTX-BiTE, HPGD, ADA2, anti-CD47, anti-PD1 |
| 277 | CTX-BiTE, HPGD, ADA2, anti-CD47, anti-TREM2 |
| 278 | CTX-BiTE, HPGD, ADA2, anti-TGFβ, anti-PD1 |
| 279 | CTX-BiTE, HPGD, ADA2, anti-TGFβ, anti-TREM2 |
| 280 | CTX-BiTE, HPGD, ADA2, anti-PD1, anti-TREM2 |
| 281 | CTX-BiTE, HPGD, HYAL1, CHP, CCL21 |
| 282 | CTX-BiTE, HPGD, HYAL1, CHP, IL-12 |
| 283 | CTX-BiTE, HPGD, HYAL1, CHP, anti-CD47 |
| 284 | CTX-BiTE, HPGD, HYAL1, CHP, anti-TGFβ |
| 285 | CTX-BiTE, HPGD, HYAL1, CHP, anti-PD1 |
| 286 | CTX-BiTE, HPGD, HYAL1, CHP, anti-TREM2 |
| 287 | CTX-BiTE, HPGD, HYAL1, CCL21, IL-12 |
| 288 | CTX-BiTE, HPGD, HYAL1, CCL21, anti-CD47 |
| 289 | CTX-BiTE, HPGD, HYAL1, CCL21, anti-TGFβ |
| 290 | CTX-BiTE, HPGD, HYAL1, CCL21, anti-PD1 |
| 291 | CTX-BiTE, HPGD, HYAL1, CCL21, anti-TREM2 |
| 292 | CTX-BiTE, HPGD, HYAL1, IL-12, anti-CD47 |
| 293 | CTX-BiTE, HPGD, HYAL1, IL-12, anti-TGFβ |
| 294 | CTX-BiTE, HPGD, HYAL1, IL-12, anti-PD1 |
| 295 | CTX-BiTE, HPGD, HYAL1, IL-12, anti-TREM2 |
| 296 | CTX-BiTE, HPGD, HYAL1, anti-CD47, anti-TGFβ |
| 297 | CTX-BiTE, HPGD, HYAL1, anti-CD47, anti-PD1 |
| 298 | CTX-BiTE, HPGD, HYAL1, anti-CD47, anti-TREM2 |
| 299 | CTX-BiTE, HPGD, HYAL1, anti-TGFβ, anti-PD1 |
| 300 | CTX-BiTE, HPGD, HYAL1, anti-TGFβ, anti-TREM2 |
| 301 | CTX-BiTE, HPGD, HYAL1, anti-PD1, anti-TREM2 |
| 302 | CTX-BiTE, HPGD, CHP, CCL21, IL-12 |
| 303 | CTX-BiTE, HPGD, CHP, CCL21, anti-CD47 |
| 304 | CTX-BiTE, HPGD, CHP, CCL21, anti-TGFβ |
| 305 | CTX-BiTE, HPGD, CHP, CCL21, anti-PD1 |
| 306 | CTX-BiTE, HPGD, CHP, CCL21, anti-TREM2 |
| 307 | CTX-BiTE, HPGD, CHP, IL-12, anti-CD47 |
| 308 | CTX-BiTE, HPGD, CHP, IL-12, anti-TGFβ |
| 309 | CTX-BiTE, HPGD, CHP, IL-12, anti-PD1 |
| 310 | CTX-BiTE, HPGD, CHP, IL-12, anti-TREM2 |
| 311 | CTX-BiTE, HPGD, CHP, anti-CD47, anti-TGFβ |
| 312 | CTX-BiTE, HPGD, CHP, anti-CD47, anti-PD1 |
| 313 | CTX-BiTE, HPGD, CHP, anti-CD47, anti-TREM2 |
| 314 | CTX-BiTE, HPGD, CHP, anti-TGFβ, anti-PD1 |
| 315 | CTX-BiTE, HPGD, CHP, anti-TGFβ, anti-TREM2 |
| 316 | CTX-BiTE, HPGD, CHP, anti-PD1, anti-TREM2 |
| 317 | CTX-BiTE, HPGD, CCL21, IL-12, anti-CD47 |
| 318 | CTX-BiTE, HPGD, CCL21, IL-12, anti-TGFβ |
| 319 | CTX-BiTE, HPGD, CCL21, IL-12, anti-PD1 |
| 320 | CTX-BiTE, HPGD, CCL21, IL-12, anti-TREM2 |
| 321 | CTX-BiTE, HPGD, CCL21, anti-CD47, anti-TGFβ |
| 322 | CTX-BiTE, HPGD, CCL21, anti-CD47, anti-PD1 |
| 323 | CTX-BiTE, HPGD, CCL21, anti-CD47, anti-TREM2 |
| 324 | CTX-BiTE, HPGD, CCL21, anti-TGFβ, anti-PD1 |
| 325 | CTX-BiTE, HPGD, CCL21, anti-TGFβ, anti-TREM2 |
| 326 | CTX-BiTE, HPGD, CCL21, anti-PD1, anti-TREM2 |
| 327 | CTX-BiTE, HPGD, IL-12, anti-CD47, anti-TGFβ |
| 328 | CTX-BiTE, HPGD, IL-12, anti-CD47, anti-PD1 |
| 329 | CTX-BiTE, HPGD, IL-12, anti-CD47, anti-TREM2 |
| 330 | CTX-BiTE, HPGD, IL-12, anti-TGFβ, anti-PD1 |
| 331 | CTX-BiTE, HPGD, IL-12, anti-TGFβ, anti-TREM2 |
| 332 | CTX-BiTE, HPGD, IL-12, anti-PD1, anti-TREM2 |
| 333 | CTX-BiTE, HPGD, anti-CD47, anti-TGFβ, anti-PD1 |
| 334 | CTX-BiTE, HPGD, anti-CD47, anti-TGFβ, anti-TREM2 |
| 335 | CTX-BiTE, HPGD, anti-CD47, anti-PD1, anti-TREM2 |
| 336 | CTX-BiTE, HPGD, anti-TGFβ, anti-PD1, anti-TREM2 |
| 337 | CTX-BiTE, ADA2, HYAL1, CHP, CCL21 |
| 338 | CTX-BiTE, ADA2, HYAL1, CHP, IL-12 |
| 339 | CTX-BiTE, ADA2, HYAL1, CHP, anti-CD47 |
| 340 | CTX-BiTE, ADA2, HYAL1, CHP, anti-TGFβ |
| 341 | CTX-BiTE, ADA2, HYAL1, CHP, anti-PD1 |
| 342 | CTX-BiTE, ADA2, HYAL1, CHP, anti-TREM2 |
| 343 | CTX-BiTE, ADA2, HYAL1, CCL21, IL-12 |
| 344 | CTX-BiTE, ADA2, HYAL1, CCL21, anti-CD47 |
| 345 | CTX-BiTE, ADA2, HYAL1, CCL21, anti-TGFβ |
| 346 | CTX-BiTE, ADA2, HYAL1, CCL21, anti-PD1 |
| 347 | CTX-BiTE, ADA2, HYAL1, CCL21, anti-TREM2 |
| 348 | CTX-BiTE, ADA2, HYAL1, IL-12, anti-CD47 |
| 349 | CTX-BiTE, ADA2, HYAL1, IL-12, anti-TGFβ |
| 350 | CTX-BiTE, ADA2, HYAL1, IL-12, anti-PD1 |
| 351 | CTX-BiTE, ADA2, HYAL1, IL-12, anti-TREM2 |
| 352 | CTX-BiTE, ADA2, HYAL1, anti-CD47, anti-TGFβ |
| 353 | CTX-BiTE, ADA2, HYAL1, anti-CD47, anti-PD1 |
| 354 | CTX-BiTE, ADA2, HYAL1, anti-CD47, anti-TREM2 |
| 355 | CTX-BiTE, ADA2, HYAL1, anti-TGFβ, anti-PD1 |
| 356 | CTX-BiTE, ADA2, HYAL1, anti-TGFβ, anti-TREM2 |
| 357 | CTX-BiTE, ADA2, HYAL1, anti-PD1, anti-TREM2 |
| 358 | CTX-BiTE, ADA2, CHP, CCL21, IL-12 |
| 359 | CTX-BiTE, ADA2, CHP, CCL21, anti-CD47 |
| 360 | CTX-BiTE, ADA2, CHP, CCL21, anti-TGFβ |
| 361 | CTX-BiTE, ADA2, CHP, CCL21, anti-PD1 |
| 362 | CTX-BiTE, ADA2, CHP, CCL21, anti-TREM2 |
| 363 | CTX-BiTE, ADA2, CHP, IL-12, anti-CD47 |
| 364 | CTX-BiTE, ADA2, CHP, IL-12, anti-TGFβ |
| 365 | CTX-BiTE, ADA2, CHP, IL-12, anti-PD1 |
| 366 | CTX-BiTE, ADA2, CHP, IL-12, anti-TREM2 |
| 367 | CTX-BiTE, ADA2, CHP, anti-CD47, anti-TGFβ |
| 368 | CTX-BiTE, ADA2, CHP, anti-CD47, anti-PD1 |
| 369 | CTX-BiTE, ADA2, CHP, anti-CD47, anti-TREM2 |
| 370 | CTX-BiTE, ADA2, CHP, anti-TGFβ, anti-PD1 |
| 371 | CTX-BiTE, ADA2, CHP, anti-TGFβ, anti-TREM2 |
| 372 | CTX-BiTE, ADA2, CHP, anti-PD1, anti-TREM2 |
| 373 | CTX-BiTE, ADA2, CCL21, IL-12, anti-CD47 |
| 374 | CTX-BiTE, ADA2, CCL21, IL-12, anti-TGFβ |
| 375 | CTX-BiTE, ADA2, CCL21, IL-12, anti-PD1 |
| 376 | CTX-BiTE, ADA2, CCL21, IL-12, anti-TREM2 |
| 377 | CTX-BiTE, ADA2, CCL21, anti-CD47, anti-TGFβ |
| 378 | CTX-BiTE, ADA2, CCL21, anti-CD47, anti-PD1 |
| 379 | CTX-BiTE, ADA2, CCL21, anti-CD47, anti-TREM2 |
| 380 | CTX-BiTE, ADA2, CCL21, anti-TGFβ, anti-PD1 |
| 381 | CTX-BiTE, ADA2, CCL21, anti-TGFβ, anti-TREM2 |
| 382 | CTX-BiTE, ADA2, CCL21, anti-PD1, anti-TREM2 |
| 383 | CTX-BiTE, ADA2, IL-12, anti-CD47, anti-TGFβ |
| 384 | CTX-BiTE, ADA2, IL-12, anti-CD47, anti-PD1 |
| 385 | CTX-BiTE, ADA2, IL-12, anti-CD47, anti-TREM2 |
| 386 | CTX-BiTE, ADA2, IL-12, anti-TGFβ, anti-PD1 |
| 387 | CTX-BiTE, ADA2, IL-12, anti-TGFβ, anti-TREM2 |
| 388 | CTX-BiTE, ADA2, IL-12, anti-PD1, anti-TREM2 |
| 389 | CTX-BiTE, ADA2, anti-CD47, anti-TGFβ, anti-PD1 |
| 390 | CTX-BiTE, ADA2, anti-CD47, anti-TGFβ, anti-TREM2 |
| 391 | CTX-BiTE, ADA2, anti-CD47, anti-PD1, anti-TREM2 |
| 392 | CTX-BiTE, ADA2, anti-TGFβ, anti-PD1, anti-TREM2 |
| 393 | CTX-BiTE, HYAL1, CHP, CCL21, IL-12 |
| 394 | CTX-BiTE, HYAL1, CHP, CCL21, anti-CD47 |
| 395 | CTX-BiTE, HYAL1, CHP, CCL21, anti-TGFβ |
| 396 | CTX-BiTE, HYAL1, CHP, CCL21, anti-PD1 |
| 397 | CTX-BiTE, HYAL1, CHP, CCL21, anti-TREM2 |
| 398 | CTX-BiTE, HYAL1, CHP, IL-12, anti-CD47 |
| 399 | CTX-BiTE, HYAL1, CHP, IL-12, anti-TGFβ |
| 400 | CTX-BiTE, HYAL1, CHP, IL-12, anti-PD1 |
| 401 | CTX-BiTE, HYAL1, CHP, IL-12, anti-TREM2 |
| 402 | CTX-BiTE, HYAL1, CHP, anti-CD47, anti-TGFβ |
| 403 | CTX-BiTE, HYAL1, CHP, anti-CD47, anti-PD1 |
| 404 | CTX-BiTE, HYAL1, CHP, anti-CD47, anti-TREM2 |
| 405 | CTX-BiTE, HYAL1, CHP, anti-TGFβ, anti-PD1 |
| 406 | CTX-BiTE, HYAL1, CHP, anti-TGFβ, anti-TREM2 |
| 407 | CTX-BiTE, HYAL1, CHP, anti-PD1, anti-TREM2 |
| 408 | CTX-BiTE, HYAL1, CCL21, IL-12, anti-CD47 |
| 409 | CTX-BiTE, HYAL1, CCL21, IL-12, anti-TGFβ |
| 410 | CTX-BiTE, HYAL1, CCL21, IL-12, anti-PD1 |
| 411 | CTX-BiTE, HYAL1, CCL21, IL-12, anti-TREM2 |
| 412 | CTX-BiTE, HYAL1, CCL21, anti-CD47, anti-TGFβ |
| 413 | CTX-BiTE, HYAL1, CCL21, anti-CD47, anti-PD1 |
| 414 | CTX-BiTE, HYAL1, CCL21, anti-CD47, anti-TREM2 |
| 415 | CTX-BiTE, HYAL1, CCL21, anti-TGFβ, anti-PD1 |
| 416 | CTX-BiTE, HYAL1, CCL21, anti-TGFβ, anti-TREM2 |
| 417 | CTX-BiTE, HYAL1, CCL21, anti-PD1, anti-TREM2 |
| 418 | CTX-BiTE, HYAL1, IL-12, anti-CD47, anti-TGFβ |
| 419 | CTX-BiTE, HYAL1, IL-12, anti-CD47, anti-PD1 |
| 420 | CTX-BiTE, HYAL1, IL-12, anti-CD47, anti-TREM2 |
| 421 | CTX-BiTE, HYAL1, IL-12, anti-TGFβ, anti-PD1 |
| 422 | CTX-BiTE, HYAL1, IL-12, anti-TGFβ, anti-TREM2 |
| 423 | CTX-BiTE, HYAL1, IL-12, anti-PD1, anti-TREM2 |
| 424 | CTX-BiTE, HYAL1, anti-CD47, anti-TGFβ, anti-PD1 |
| 425 | CTX-BiTE, HYAL1, anti-CD47, anti-TGFβ, anti-TREM2 |
| 426 | CTX-BiTE, HYAL1, anti-CD47, anti-PD1, anti-TREM2 |
| 427 | CTX-BiTE, HYAL1, anti-TGFβ, anti-PD1, anti-TREM2 |
| 428 | CTX-BiTE, CHP, CCL21, IL-12, anti-CD47 |
| 429 | CTX-BiTE, CHP, CCL21, IL-12, anti-TGFβ |
| 430 | CTX-BiTE, CHP, CCL21, IL-12, anti-PD1 |
| 431 | CTX-BiTE, CHP, CCL21, IL-12, anti-TREM2 |
| 432 | CTX-BiTE, CHP, CCL21, anti-CD47, anti-TGFβ |
| 433 | CTX-BiTE, CHP, CCL21, anti-CD47, anti-PD1 |
| 434 | CTX-BiTE, CHP, CCL21, anti-CD47, anti-TREM2 |
| 435 | CTX-BiTE, CHP, CCL21, anti-TGFβ, anti-PD1 |
| 436 | CTX-BiTE, CHP, CCL21, anti-TGFβ, anti-TREM2 |
| 437 | CTX-BiTE, CHP, CCL21, anti-PD1, anti-TREM2 |
| 438 | CTX-BiTE, CHP, IL-12, anti-CD47, anti-TGFβ |
| 439 | CTX-BiTE, CHP, IL-12, anti-CD47, anti-PD1 |
| 440 | CTX-BiTE, CHP, IL-12, anti-CD47, anti-TREM2 |
| 441 | CTX-BiTE, CHP, IL-12, anti-TGFβ, anti-PD1 |
| 442 | CTX-BiTE, CHP, IL-12, anti-TGFβ, anti-TREM2 |
| 443 | CTX-BiTE, CHP, IL-12, anti-PD1, anti-TREM2 |
| 444 | CTX-BiTE, CHP, anti-CD47, anti-TGFβ, anti-PD1 |
| 445 | CTX-BiTE, CHP, anti-CD47, anti-TGFβ, anti-TREM2 |
| 446 | CTX-BiTE, CHP, anti-CD47, anti-PD1, anti-TREM2 |
| 447 | CTX-BiTE, CHP, anti-TGFβ, anti-PD1, anti-TREM2 |
| 448 | CTX-BiTE, CCL21, IL-12, anti-CD47, anti-TGFβ |
| 449 | CTX-BiTE, CCL21, IL-12, anti-CD47, anti-PD1 |
| 450 | CTX-BiTE, CCL21, IL-12, anti-CD47, anti-TREM2 |
| 451 | CTX-BiTE, CCL21, IL-12, anti-TGFβ, anti-PD1 |
| 452 | CTX-BiTE, CCL21, IL-12, anti-TGFβ, anti-TREM2 |
| 453 | CTX-BiTE, CCL21, IL-12, anti-PD1, anti-TREM2 |
| 454 | CTX-BiTE, CCL21, anti-CD47, anti-TGFβ, anti-PD1 |
| 455 | CTX-BiTE, CCL21, anti-CD47, anti-TGFβ, anti-TREM2 |
| 456 | CTX-BiTE, CCL21, anti-CD47, anti-PD1, anti-TREM2 |
| 457 | CTX-BiTE, CCL21, anti-TGFβ, anti-PD1, anti-TREM2 |
| 458 | CTX-BiTE, IL-12, anti-CD47, anti-TGFβ, anti-PD1 |
| 459 | CTX-BiTE, IL-12, anti-CD47, anti-TGFβ, anti-TREM2 |
| 460 | CTX-BiTE, IL-12, anti-CD47, anti-PD1, anti-TREM2 |
| 461 | CTX-BiTE, IL-12, anti-TGFβ, anti-PD1, anti-TREM2 |
| 462 | CTX-BiTE, anti-CD47, anti-TGFβ, anti-PD1, anti-TREM2 |

In some embodiments, the recombinant oncolytic viruses described herein comprise at least one polynucleotide encoding a payload molecule that that reduces the expression or inhibits the function of an endogenous miRNA, a gene, or a tissue inhibitor of metalloproteinases (TIMP). Such recombinant oncolytic viruses are referred to herein as “genome-editing” or “microenvironment-remodeling” viruses or vectors. The encoded protein or oligonucleotide may reduce expression or inhibit the function of a miRNA, gene, or TIMP in any number of ways including targeting the protein (e.g., a TIMP) for degradation (e.g., by ubiquitination and proteosomal degradation or targeting for lysosomal degradation), blocking interactions with cognate receptors (e.g., blocking antibodies or antigen binding fragments thereof or peptide inhibitors), degrading messenger RNA transcripts (e.g., a short interfering RNA or short hairpin RNA), and/or altering the genomic DNA sequence encoding the specific miRNA, gene, or protein (e.g., by an endonuclease).

In particular embodiments, the protein or oligonucleotide reduces the expression of a miR or a gene involved in carcinogenesis or metastasis (e.g., an oncogenic miR or an oncogene). In some embodiments, a recombinant oncolytic virus comprises at least one polynucleotide encoding a payload molecule that reduces the expression or function of a miRNA that is an oncogenic miRNA (e.g., one or more of the miRNAs listed in Table 13). In some embodiments, the recombinant oncolytic virus comprises at least 1, 2, 3, 4, 5, 6, 7, 8, 9, 10 or more polynucleotides encoding for a protein or oligonucleotide that reduces the expression or function of an oncogenic miRNA. In some embodiments, the recombinant oncolytic virus comprises at least 1, 2, 3, 4, 5, 6, 7, 8, 9, 10 or more polynucleotides encoding for a plurality of proteins or oligonucleotides that reduce the expression or function of a plurality of oncogenic miRNAs. In some embodiments, the protein or oligonucleotide reduces the expression of miR-17-92 and is used to treat lung cancer (e.g., small-cell lung cancer). In other embodiments, the protein or oligonucleotide reduces the expression of miR-221 and/or miR-21 and is used to treat glioblastoma. In certain embodiments, the protein or oligonucleotide reduces the expression of miR-155 and/or miR-17-92 and is used to treat lymphoma (e.g., Burkitt's lymphoma, diffuse large B cell lymphoma, marginal zone lymphoma, or chronic lymphocytic leukemia). In some embodiments, the protein or oligonucleotide reduces the expression of miR-221, miR-222, and/or miR-146 and is used to treat thyroid cancer. In some embodiments, the protein or oligonucleotide reduces the expression of miR-372 and/or miR-373 and is used to treat testicular cancer (e.g., testicular germ cell tumors). In some embodiments, the protein or oligonucleotide reduces the expression of miR-18 and/or miR-224 and is used to treat liver cancer (e.g., hepatocellular carcinoma).

In some embodiments, recombinant viral vectors described herein comprise a polynucleotide encoding a payload molecule that degrades the tumor extracellular matrix (ECM), which in some aspects leads to enhanced viral spread. Matrix metalloproteinases (MMPs) are zinc-dependent proteases that are classified, based on their activity, into collagenases, gelatinases, stromelysins and matrilysins. These proteases are generally secreted as pro-enzymes (zymogens) and are activated by proteolytic removal of the pro-peptide pro-domain. The primary role that MMPs play in cancer is in the degradation of the ECM, which facilitates tumor invasion and metastasis. MMPs are also involved in tumor progression, epithelial to mesenchymal transition (EMT), and angiogenesis. MMPs are regulated by miRs as well as TIMPs, which comprise a family of four protease inhibitors (TIMP1, TIMP2, TIMP3, and TIMP4). A broad array of tumor microenvironments can be degraded by disrupting miRNAs or TIMPs that negatively regulate the MMP family with the recombinant viral vectors of the disclosure. Many of these interactions show that multiple MMPs are regulated by a single miRNA: e.g. let-7 regulates MMP-2, MMP-9, and MMP-14; miR-143 regulates MMP-2, MMP-9, and MMP-13; miR-218 regulates MMP-2, MMP-7, and MMP-9. Furthermore, the vast majority of MMPs may be regulated by a single TIMP master switch: e.g. TIMP1 is known to inhibit most all of the known MMPs and also promotes cell proliferation in a wide range of cell types; TIMP2 interacts with MMP-14 and MMP-2.

In some embodiments, the recombinant oncolytic viruses described herein comprise at least one polynucleotide encoding a protein or an oligonucleotide that reduces the expression or function of a miRNA that is capable of altering the extracellular matrix or capable of modulating a pathway that alters the extracellular matrix, particularly in a tumor microenvironment. A microenvironment remodeling miR, as used herein, refers to a miR. In some embodiments, the protein or oligonucleotide reduces the expression or function of one microenvironment remodeling miR. In some embodiments, the protein or oligonucleotide reduces the expression or function of at least 2, 3, 4, 5, 6, 7, 8, 9, or 10 or more microenvironment remodeling miRs. In some embodiments, the recombinant oncolytic virus comprises a plurality of polynucleotides encoding a plurality of protein or oligonucleotides that reduce the expression or function of a plurality of microenvironment remodeling miRs. In some embodiments, strategies described herein may be utilized by recombinant viral vectors of the present disclosure to knockdown or disrupt expression or function of miRs or TIMPs which negatively regulate MMPs. In some embodiments, a recombinant oncolytic virus reduces the expression of a TIMP selected from TIMP1, TIMP2, TIMP3 and TIMP4.

In some embodiments, the recombinant oncolytic viruses described herein comprise at least one polynucleotide encoding a protein or an oligonucleotide that reduces the expression or function of a gene in the host cell genome. In some aspects, the gene is an oncogenic gene. In some aspects, the gene encodes an oncogenic miR (e.g., a miRNA listed in Table 13), a microenvironment remodeling miR, or a negative regulator of ECM-degradation (e.g., a TIMP). Reduction of gene expression and/or function may be accomplished by at the level of transcription (e.g., mutating, deleting, or silencing the genomic DNA sequence) or at the level of translation (e.g., by inhibiting the production of the gene product through mRNA degradation). In some embodiments, the recombinant oncolytic viruses described herein comprise one or more polynucleotides that encode for nucleases that reduce the expression or function of a gene by enabling the mutation, deletion, or repression of transcription of a gene sequence. In specific embodiments, the nuclease is selected from a Clustered Regulatory Interspaced Short Palindromic Repeats (CRISPR)-associated endonuclease, a zinc-finger nuclease (ZFN) or a Transcription activator-like effector nuclease (TALEN). In non-limiting examples, a CRISPR-associated endonuclease is selected from SpCas9, SpCas9-HF1, SpCas9-HF2, SpCas9-HF3, SpCas9-HF4, SaCas9, FnCpf, FnCas9, eSpCas9, C2C1, C2C3, Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

Recombinant viral vectors of the disclosure may utilize the CRISPR (Clustered Regularly Interspaced Short Palindromic Repeats)/Cas (CRISPR Associated) nuclease system, which is an engineered nuclease system based on a bacterial system that can be used for mammalian genome engineering. Generally, the system comprises a Cas nuclease and a guide RNA (gRNA). The gRNA is comprised of two parts; a crispr-RNA (crRNA) that is specific for a target genomic DNA sequence, and a tracr RNA (trRNA) that facilitates Cas binding. The crRNA and trRNA may be present as separate RNA oligonucleotides, or may be present in the same RNA oligonucleotide, referred to as a single guide-RNA (sgRNA). As used herein, the term “guide RNA” or “gRNA” refers to either the combination of an individual trRNA and an individual crRNA or an sgRNA. See, e.g., Jinek et al. (2012) Science 337:816-821; Cong et al. (2013) Science 339:819-823; Mali et al. (2013) Science 339:823-826; Qi et al. (2013) Cell 152:1173-1183; Jinek et al. (2013), eLife 2: e00471; David Segal (2013) eLife 2: e00563; Ran et al. (2013) Nature Protocols 8 (11): 2281-2308; Zetsche et al. (2015) Cell 163 (3): 759-771; PCT Publication Nos. WO 2007/025097, WO 2008/021207, WO 2010/011961, WO 2010/054108, WO 2010/054154, WO 2012/054726, WO 2012/149470, WO 2012/164565, WO 2013/098244, WO 2013/126794, WO 2013/141680, and WO 2013/142578; U.S. Patent Publication Nos. 2010-0093617, 2013-0011828, 2010-0257638, 2010-0076057, 2011-0217739, 2011-0300538, 2013-0288251, and 2012-0277120; and U.S. Pat. No. 8,546,553, each of which is incorporated herein by reference in its entirety.

Multiple class 1 CRISPR-Cas systems, which include the type I and type III systems, have been identified and functionally characterized in detail, revealing the complex architecture and dynamics of the effector complexes (Brouns et al., 2008, Marraffini and Sontheimer, 2008, Hale et al., 2009, Sinkunas et al., 2013, Jackson et al., 2014, Mulepati et al., 2014). In addition, several class 2-type II CRISPR-Cas systems that employ homologous RNA-guided endonucleases of the Cas9 family as effectors have also been identified and experimentally characterized (Barrangou et al., 2007, Garneau et al., 2010, Deltcheva et al., 2011, Sapranauskas et al., 2011, Jinek et al., 2012, Gasiunas et al., 2012). A second, putative class 2-type V CRISPR-Cas system has been recently identified in several bacterial genomes. The putative type V CRISPR-Cas systems contain a large, ˜1,300 amino acid protein called Cpf1 (CRISPR from Prevotella and Francisella 1).

In some embodiments, an oncolytic virus described herein further comprises at least one polynucleotide encoding a trRNA and crRNA targeted to the miRNA or the TIMP. In some cases, the at least one polynucleotide encoding a trRNA and crRNA is inserted into a locus on the viral genome. In some embodiments, the polynucleotide is an insulated sequence comprising a synthetic insulator or a native viral (e.g., HSV) insulator. In certain embodiments, an oncolytic virus is a herpes simplex virus and the at least one polynucleotide encoding an RNA binding site is inserted into or between one or more loci including the internal repeat joint region (comprising one copy each of the diploid genes ICP0, ICP34.5, LAT, ICP4, and the ICP47 promoter), ICP0, LAT, UL1, UL5, UL6, UL7, UL8, UL9, UL11, UL12, UL14, UL15, UL17, UL18, UL19, UL20, UL22, UL25, UL26, UL26.5, UL27, UL28, UL29, UL30, UL31, UL32, UL33, UL34, UL35, UL36, UL37, UL38, UL39, UL40, UL42, UL48, UL49, UL52, UL53, UL54, ICP0, ICP4, ICP22, ICP27, ICP47, gamma-34.5, US3, US4, US5, US6, US7, US8, US9, US10, US11, and US12. In one embodiment, an oncolytic virus is a herpes simplex virus (HSV) and the at least one polynucleotide encoding an RNA binding site is inserted into a locus between the UL3 and the UL4 open reading frames.

In some embodiments, the recombinant oncolytic virus comprises at least one polynucleotide encoding a payload molecule that activate or enhances an anti-tumor immune response. In some embodiments, the payload molecule is a cytokine, a chemokine, an antibody or antigen binding fragment thereof, a bispecific T-cell engager (BiTE). For example, in some embodiments, the payload molecule is an antibody or antigen binding fragments thereof that bind to and inhibit immune checkpoint receptors (e.g. CTLA4, LAG3, PD1, PDL1, and others). In some embodiments, the payload molecule is an anti-PD1 antibody or antigen-binding fragment thereof, an anti-PDL1 antibody or antigen-binding fragment thereof, or an anti-CTLA4 antibody or antigen-binding fragment thereof.

In some embodiments, the payload molecule comprises a PD1 antagonist. In some embodiments, the PD1 antagonist is an anti-PD1 antibody or antigen-binding fragment thereof. In some embodiments, the PD1 antagonist is an anti-PD1 nanobody.

In some embodiments, the payload molecule comprises IL12.

In some embodiments, the payload molecule is a protein that binds to and activates a cell-surface receptor. For example, in some embodiments, payload molecule comprises an endogenous cell-surface ligand, such as the extracellular domain of 41BBL, the extracellular domain of CD40L, FLT3L. In some embodiments, the payload molecule is a cytokine (e.g., IFNγ, IFNα, IFNβ, TNFα, IL-12, IL-2, IL-6, IL-8, IL-15, GM-CSF, IL-21, IL-35, TGFβ, and others) or chemokine (e.g., CCL4, CXCL10, CCL5, CXCL13, or XCL1).

In some embodiments, the payload molecule is a protein that binding to and activate an activating receptor (e.g., FcγRI, FcγIIa, FcγIIIa, costimulatory receptors, and others). In particular embodiments, the protein is selected from EpCAM, folate, A2A, anti-FGF2, anti-FGFR/FGFR2b, anti-SEMA4D, CD137, CD200, CD38, CD44, CSF-IR, endothelin B Receptor, ISRE7, LFA-1, NG2 (also known as SPEG4), SMADs, STING, and VCAM1.

In certain embodiments, a polynucleotide encoding a protein or an oligonucleotide targeted to reduce expression of an miRNA, a gene, or a TIMP is inserted into a locus on the viral genome of a recombinant oncolytic virus. In some embodiments, the polynucleotide is an insulated sequence comprising a synthetic insulator or a native viral (e.g., HSV) insulator. In certain embodiments, the oncolytic virus is a herpes simplex virus and the at least one polynucleotide encoding an RNA binding site is inserted into or between one or more loci including the internal repeat joint region (comprising one copy each of the diploid genes ICP0, ICP34.5, LAT, ICP4, and the ICP47 promoter), ICP0, LAT, UL1, UL5, UL6, UL7, UL8, UL9, UL11, UL12, UL14, UL15, UL17, UL18, UL19, UL20, UL22, UL25, UL26, UL26.5, UL27, UL28, UL29, UL30, UL31, UL32, UL33, UL34, UL35, UL36, UL37, UL38, UL39, UL40, UL42, UL48, UL49, UL52, UL53, UL54, ICP0, ICP4, ICP22, ICP27, ICP47, gamma-34.5, US3, US4, US5, US6, US7, US8, US9, US10, US11, and US12 . . . . In one embodiment, the virus is a herpes simplex virus (HSV) and the at least one polynucleotide is inserted into a locus between the UL3 and the UL4 open reading frames.

In some embodiments, the recombinant oncolytic virus comprises at least one polynucleotide encoding a payload molecule that inhibits immune suppression by myeloid cells in GBM. In some embodiments, the payload molecule comprises a CD47 antagonist, a TGFβ antagonist, an adenosine deaminase 2 (ADA2), a chemotaxis inhibitory protein (CHP), 15-hydroxyprostaglandin dehydrogenase [NAD(+)] (HPGD), sperm adhesion molecule 1 (SPAM1/HYAL5), or a biomolecule comprising a chlorotoxin (CTX). In some embodiments, the payload molecule comprises a CD47 antagonist. In some embodiments, the CD47 antagonist is an anti-CD47 antibody or antigen-binding fragment thereof. In some embodiments, the payload molecule comprises a TGFβ antagonist. In some embodiments, the TGFβ antagonist is an anti-TGFβ antibody or antigen-binding fragment thereof. In some embodiments, the payload molecule comprises an adenosine deaminase 2 (ADA2). In some embodiments, the payload molecule comprises a chemotaxis inhibitory protein (CHP/CHIPS). In some embodiments, the chemotaxis inhibitory protein is derived from Staphylococcus aureus. In some embodiments, the payload molecule comprises 15-hydroxyprostaglandin dehydrogenase [NAD(+)] (HPGD/PGDH). In some embodiments, the payload molecule comprises sperm adhesion molecule 1 (SPAM1/HYAL5). In some embodiments, the payload molecule comprises a biomolecule comprising a chlorotoxin (CTX). In some embodiments, the CTX is a scorpion CTX. In some embodiments, the biomolecule comprising chlorotoxin also comprises an antibody fragment. In some embodiments, the biomolecule comprising chlorotoxin also comprises an Fc domain (i.e., CTX-Fc). In some embodiments, the biomolecule comprising chlorotoxin also comprises a bispecific T cell engager (BiTE) or a bispecific light T-cell engager (LiTE) (i.e., CTX-BiTE or CTX-LITE).

In some embodiments, the recombinant oncolytic virus comprises at least one protease-activated antibody. Protease-activated antibodies, such as those described by Metz et al. (Protein Eng Des Sel, 25 (10): 571-80, 2012) are activated and bind only to targets following protease cleavage of a protective cap. In some instances, tumor microenvironments possess an array of proteases that are well differentiated from surrounding healthy tissues. For example, the protease cathepsin B is overexpressed in numerous cancers, including breast, cervix, colon, colorectal, gastric, head and neck, liver, lung, melanoma, ovarian, pancreatic, prostate, and thyroid cancer. The human degradome, comprised of a complete list of proteases synthesized by human cells, is made up of at least 569 proteases that are distributed into five broad classes (in order from greatest to least number): metalloproteinases (MMPs), serine, cysteine, threonine, and aspartic proteases (Lopez-Otin et al., Nat Rev Cancer, 7 (10): 800-8, 2007). In particular, protease antibodies specifically cleaved by MMPs can serve as an excellent means of targeting the recombinant viral vectors described herein to the tumor microenvironment, as MMPs are found in the extracellular and pericellular areas of the cell.

In certain embodiments, the protease-activated antibody is incorporated into the viral glycoprotein envelope. Protease-activated antibodies can be incorporated into the glycoprotein envelope of a recombinant viral vector of the disclosure (e.g., an HSV vector) to increase the therapeutic index and reduce off-target infection. In the case of an HSV vector, in some embodiments, the glycoprotein may be gC or gD. In some embodiments, the recombinant oncolytic viruses described herein comprise at least one polynucleotide encoding a protease-activated antibody. In certain embodiments, a protease-activated antibody is activated by a protease selected from a cysteine cathepsin, an aspartic cathepsin, a kallikrein (hK), a serine protease, a caspase, a matrix metalloproteinase (MMP), and a disintegrin and metalloproteinase (ADAM). In some embodiments, a protease is selected from cathepsin K, cathepsin B, cathepsin L, cathepsin E, cathepsin D, hK1, PSA (hK3), hK10, hK15, uPA, uPAR, MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-23A, MMP-23B, MMP-24, MMP-25, MMP-26, MMP-27, or MMP-28.

In some embodiments, the protease-activated antibody binds a protein expressed more highly by cancer cells or in cancer microenvironments than by non-cancer cells or in non-cancer microenvironments. In certain aspects, a protease-activated antibody binds NKG2D, c-met, HGFR, CD8, heparan sulfate, VSPG4 (also known as NG2), EGFR, EGFRvIII, CD133, CXCR4, carcinoembryonic antigen (CEA), CLC-3, annexin II, human transferrin receptor, or EpCAM. In certain instances, multiple protease activated antibodies may be incorporated into a single viral vector particle to ensure that diverse tumor histotypes are targeted. For example, at least 1, 2, 3, 4, 6, 7, 8, 9, 10, or more protease activated antibodies may be incorporated into the viral glycoprotein envelope. In some embodiments, the recombinant oncolytic virus comprises at least 1, 2, 3, 4, 5, 6, 7, 8, 9, 10, or more polynucleotides that encodes for at least 1, 2, 3, 4, 5, 6, 7, 8, 9, 10, or more protease activated antibodies. In some embodiments, an oncolytic virus comprises a first protease-activated antibody that binds a first protein expressed more highly by cancer cells or in cancer microenvironments than by non-cancer cells or in non-cancer microenvironments, and a second protease-activated antibody that binds a second protein expressed more highly by cancer cells or in cancer microenvironments than by non-cancer cells or in non-cancer microenvironments. In further embodiments, an oncolytic virus comprises a plurality of protease-activated antibodies binding a plurality of protein expressed more highly by cancer cells or in cancer microenvironments than by non-cancer cells or in non-cancer microenvironments. An oncolytic virus comprises, for example, a protease-activated antibody that is a human antibody, a humanized antibody or a chimeric antibody. In some embodiments, an oncolytic virus comprises an antibody that is a full-length immunoglobulin, an scFv, a Fab, a Fab′, an F(ab′)2, an Fv, a diabody, a triabody, a minibody, a single-domain antibody, or a multispecific antibody.

In some embodiments, a recombinant oncolytic virus comprises one or more of: one or more micro-RNA (miR) target sequences inserted into a locus of one or more viral genes required for viral replication; one or more polynucleotides encoding one or more proteins or oligonucleotides, wherein the proteins or oligonucleotides reduce the expression or inhibit the function of a miR, a gene, or a TIMP; at least one protease-activated antibody; and/or a polynucleotide encoding at least one protease activated antibody. In some embodiments, a recombinant oncolytic virus comprises: a plurality of copies of one or more miRNA target sequences inserted into a locus of a viral gene required for viral replication in non-cancerous cells; and/or a first polynucleotide encoding a protein or an oligonucleotide targeted to reduce expression of an oncogenic miRNA or an oncogenic gene; and/or a second polynucleotide encoding a protein or an oligonucleotide targeted to reduce expression of a microenvironment remodeling miRNA or a TIMP. In some embodiments, a recombinant oncolytic virus comprises: a plurality of copies of one or more miRNA target sequences inserted into a locus of a viral gene required for viral replication in non-cancerous cells; and/or a polynucleotide encoding a protein or an oligonucleotide targeted to reduce expression of an oncogenic miRNA or an oncogenic gene; and/or at least one protease-activated antibody. In further embodiments, a recombinant oncolytic virus comprises a plurality of copies of one or more miRNA target sequences inserted into a locus of a viral gene required for viral replication in non-cancerous cells; and/or a polynucleotide encoding a protein or an oligonucleotide targeted to reduce expression of a microenvironment remodeling miRNA or a TIMP; and/or at least one protease-activated antibody. In one embodiment, a recombinant oncolytic virus comprises a plurality of copies of one or more miRNA target sequences inserted into a locus of a viral gene required for viral replication in non-cancerous cells; and/or a first polynucleotide encoding a protein or an oligonucleotide targeted to reduce expression of an oncogenic miRNA or an oncogenic gene; and/or a second polynucleotide encoding a protein or an oligonucleotide targeted to reduce expression of a microenvironment remodeling miRNA or a TIMP; and/or at least one protease-activated antibody. In some specific embodiments, an oncolytic virus described in this paragraph is a herpes simplex virus and the viral gene required for viral replication in non-cancerous cells is UL1, UL5, UL6, UL7, UL8, UL9, UL11, UL12, UL14, UL15, UL17, UL18, UL19, UL20, UL22, UL25, UL26, UL26.5, UL27, UL28, UL29, UL30, UL31, UL32, UL33, UL34, UL35, UL36, UL37, UL38, UL39, UL40, UL42, UL48, UL49, UL52, UL53, UL54, ICP0, ICP4, ICP22, ICP27, ICP47, gamma-34.5, US3, US4, US5, US6, US7, US8, US9, US10, US11, and US12.

In certain aspects, the disclosure relates to a recombinant oncolytic virus comprising a first polynucleotide encoding a protein or an oligonucleotide targeted to reduce expression of an oncogenic miRNA or an oncogenic gene; and a second polynucleotide encoding a protein or an oligonucleotide targeted to reduce expression of a microenvironment remodeling miRNA or a TIMP. In other embodiments, a recombinant oncolytic virus comprises a polynucleotide encoding a protein or an oligonucleotide targeted to reduce expression of an oncogenic miRNA or an oncogenic gene; and at least one protease-activated antibody. In some embodiments, a recombinant oncolytic virus comprises a polynucleotide encoding a protein or an oligonucleotide targeted to reduce expression of a microenvironment remodeling miRNA or a TIMP; and at least one protease-activated antibody. In one embodiment, a recombinant oncolytic virus comprises a first polynucleotide encoding a protein or an oligonucleotide targeted to reduce expression of an oncogenic miRNA or an oncogenic gene; and/or a second polynucleotide encoding a protein or an oligonucleotide targeted to reduce expression of a microenvironment remodeling miRNA or a TIMP; and/or at least one protease-activated antibody.

### Codon-Optimization and ORF G/C Content

In some embodiments, the oncolytic virus of the disclosure comprises one or more transgenes having relatively high G/C content.

Without wishing to be bound by any particular theory, it is contemplated that transgene(s) having relatively high G/C content in the open reading frame (ORF; also known as the coding region) have significantly higher expression of the corresponding payload molecule as compared to transgenes having lower G/C content in the ORF when incorporated into the recombinant herpesvirus of the disclosure.

As used herein, the term “guanosine/cytosine content” or “G/C content” refers to the percentage of nitrogenous bases in a DNA or RNA molecule that are either guanine (G) or cytosine (C).

In some embodiments, the G/C content of the ORF of the one or more transgenes described herein is increased compared to the G/C content of the corresponding wild type (unmodified) coding region. The encoded amino acid sequence of the ORF is preferably not modified compared to the encoded amino acid sequence of the corresponding wild type (unmodified) coding region. This can be achieved by codon optimization. Methods for codon optimization are known in the art.

Depending on the amino acid to be encoded by the coding region of the modified RNA as defined herein, there are various possibilities for modification of the ORF to increase its G/C ontent compared to that of the wild type ORF.

In some embodiments, for amino acids that encoded by codons containing exclusively G or C nucleotides, no modification of the codon is necessary. In some embodiments, the codons for Pro (CCC or CCG), Arg (CGC or CGG), Ala (GCC or GCG) and Gly (GGC or GGG) require no modification.

In some embodiments, codons containing A and/or T/U nucleotides can be modified by substitution of other codons which code for the same amino acids but contain no A and/or T/U. For example:
the codons for Pro can be modified from CCT or CCA to CCC or CCG;
the codons for Arg can be modified from CGT or CGA or AGA or AGG to CGC or CGG;
the codons for Ala can be modified from GCT or GCA to GCC or GCG;
the codons for Gly can be modified from GGT or GGA to GGC or GGG.

In some embodiments, although A or T/U nucleotides cannot be eliminated from the codons, it is possible to decrease the A and T/U content by using codons containing a lower content of A and/or T/U nucleotides. For example:
the codons for Phe can be modified from TTT to TTC;
the codons for Leu can be modified from TTA, TTG, CTT or CTA to CTC or CTG;
the codons for Ser can be modified from TCT or TCA or AGT to TCC, TCG or AGC;
the codon for Tyr can be modified from TAT to TAC;
the codon for Cys can be modified from TGT to TGC;
the codon for His can be modified from CAT to CAC;
the codon for Gln can be modified from CAA to CAG;
the codons for Ile can be modified from ATT or ATA to ATC;
the codons for Thr can be modified from ACT or ACA to ACC or ACG;
the codon for Asn can be modified from AAT to AAC;
the codon for Lys can be modified from AAA to AAG;
the codons for Val can be modified from GTT or GTA to GTC or GTG;
the codon for Asp can be modified from GAT to GAC;
the codon for Glu can be modified from GAA to GAG;
the stop codon UAA can be modified to TAG or TGA.

In some embodiments, the codons for Met (ATG) and Trp (TGG) are not modified.

The substitutions listed above can be used either individually or in any possible combination to increase the G/C content of the coding region of the ORF, compared to the starting sequence (e.g., the wild type ORF).

In some embodiments, the G/C content of the ORF of the one or more transgenes described herein is increased by at least 5%, at least 10%, at least 15%, at least 20%, at least 25%, or at least 30%, compared to the G/C content of the wild type coding region or the starting ORF sequence. In some embodiments, at least 5%, at least 10%, at least 20%, at least 30%, at least 40%, at least 50%, at least 60%, at least 70%, at least 80%, at least 90%, at least 95%, or even 100% of the substitutable codons in the ORF of the one or more transgenes are substituted, thereby increasing the G/C content of said ORF.

In some embodiments, the codon optimization further comprises removing the “rare codons” present in the ORF and replacing it by a codon which codes for a tRNA which is relatively frequent in the cell and carries the same amino acid as the relatively rare tRNA. Which tRNAs occur relatively frequently in the cell and which, in contrast, occur relatively rarely is known to a person skilled in the art; see, e.g., Akashi, Curr. Opin. Genet. Dev. 2001, 11 (6): 660-666, which is incorporated by reference in its entirety for all purposes.

In some embodiments, the ORF of at least one of the transgene(s) of the oncolytic virus described herein has G/C content of at least 60%. In some embodiments, the ORF of at least one of the transgene(s) of the oncolytic virus described herein has G/C content of at least 61%. In some embodiments, the ORF of at least one of the transgene(s) of the oncolytic virus described herein has G/C content of at least 62%. In some embodiments, the ORF of at least one of the transgene(s) of the oncolytic virus described herein has G/C content of at least 63%. In some embodiments, the ORF of at least one of the transgene(s) of the oncolytic virus described herein has G/C content of at least 64%. In some embodiments, the ORF of at least one of the transgene(s) of the oncolytic virus described herein has G/C content of at least 59%. In some embodiments, the ORF of at least one of the transgene(s) of the oncolytic virus described herein has G/C content of at least 56%, at least 57%, at least 58%, at least 59%, at least 60%, at least 61%, at least 62%, at least 63%, at least 64%, at least 65%, at least 66%, at least 67%, at least 68%, at least 69%, or at least 70%. In some embodiments, the ORF of at least one of the transgene(s) of the oncolytic virus described herein has G/C content of about 60%, about 61%, about 62%, about 63%, about 64%, about 65%, about 66%, about 67%, about 68%, about 69%, or about 70%. In some embodiments, the ORF of at least one of the transgene(s) of the oncolytic virus described herein has G/C content of between 56%-57%, 57%-58%, 58%-59%, 59%-60%, 60%-61%, 61%-62%, 62%-63%, 63%-64%, 64%-65%, 65%-66%, 66%-67%, 67%-68%, 68%-69%, 69%-70%, 56%-58%, 57%-59%, 58%-60%, 59%-61%, 60%-62%, 61%-63%, 62%-64%, 63%-65%, 64%-66%, 65%-67%, 66%-68%, 67%-69%, 68%-70%, 56%-59%, 57%-60%, 58%-61%, 59%-62%, 60%-63%, 61%-64%, 62%-65%, 63%-66%, 64%-67%, 65%-68%, 66%-69%, 67%-70%, 56%-60%, 57%-61%, 58%-62%, 59%-63%, 60%-64%, 61%-65%, 62%-66%, 63%-67%, 64%-68%, 65%-69%, 66%-70%, 56%-61%, 57%-62%, 58%-63%, 59%-64%, 60%-65%, 61%-66%, 62%-67%, 63%-68%, 64%-69%, 65%-70%, 56%-62%, 57%-63%, 58%-64%, 59%-65%, 60%-66%, 61%-67%, 62%-68%, 63%-69%, 64%-70%, 56%-63%, 57%-64%, 58%-65%, 59%-66%, 60%-67%, 61%-68%, 62%-69%, 63%-70%, 56%-64%, 57%-65%, 58%-66%, 59%-67%, 60%-68%, 61%-69%, 62%-70%, 56%-65%, 57%-66%, 58%-67%, 59%-68%, 60%-69%, 61%-70%, 56%-66%, 57%-67%, 58%-68%, 59%-69%, 60%-70%, 56%-67%, 57%-68%, 58%-69%, 59%-70%, 56%-68%, 57%-69%, 58%-70%, 56%-69%, 57%-70%, or 56%-70%. In some embodiments, the at least one of the transgene(s) of the oncolytic virus are at least 2, at least 3, at least 4, at least 5 transgenes, at least 6 transgenes. In some embodiments, the at least one of the transgene(s) of the oncolytic virus are 1, 2, 3, 4, 5, 6, 7, 8, 9, 10, or 11 transgenes. In some embodiments, the at least one of the transgene(s) of the oncolytic virus are all the transgenes. In some embodiments, the at least one of the transgene(s) of the oncolytic virus comprise one or more transgenes encoding IL-12, a PD1 antagonist, a TREM2 antagonist, HPGD, and/or a biomolecule comprising CTX. In some embodiments, the at least one of the transgene(s) of the oncolytic virus comprise transgenes encoding IL-12, a PD1 antagonist, and a TREM2 antagonist. In some embodiments, the at least one of the transgene(s) of the oncolytic virus comprise transgenes encoding IL-12, a PD1 antagonist, a TREM2 antagonist, and HPGD. In some embodiments, the at least one of the transgene(s) of the oncolytic virus comprise transgenes encoding IL-12, a PD1 antagonist, a TREM2 antagonist, and a biomolecule comprising CTX. In some embodiments, the at least one of the transgene(s) of the oncolytic virus comprise transgenes encoding IL-12, a PD1 antagonist, a TREM2 antagonist, HPGD, and a biomolecule comprising CTX.

In some embodiments, the expression of a payload molecule encoded by the ORF of the transgene in the oncolytic virus described herein is at least 2-fold, at least 3-fold, at least 5-fold, at least 8-fold, or at least 10-fold higher than the expression of the payload protein encoded by a control ORF encoding the same payload molecule in a control oncolytic virus. In some embodiments, the expression of a payload molecule encoded by the ORF of the transgene is at least 2-fold higher than the expression of the payload protein encoded by the control ORF. In some embodiments, the expression of a payload molecule encoded by the ORF of the transgene is at least 3-fold higher than the expression of the payload protein encoded by the control ORF. In some embodiments, the expression of a payload molecule encoded by the ORF of the transgene is at least 5-fold higher than the expression of the payload protein encoded by the control ORF. In some embodiments, the expression of a payload molecule encoded by the ORF of the transgene is at least 8-fold higher than the expression of the payload protein encoded by the control ORF. In some embodiments, the expression of a payload molecule encoded by the ORF of the transgene is at least 10-fold higher than the expression of the payload protein encoded by the control ORF. A skilled person would readily recognize the proper control ORF/control virus. In some embodiments, the control ORF comprises a wildtype polynucleotide sequence encoding the payload protein. In some embodiments, the control ORF is codon optimized based on the codon usage of Homo sapiens. In some embodiments, the control ORF has a G/C content of no more than 55%, no more than 54%, no more than 53%, no more than 52%, no more than 51%, no more than 50%, no more than 49%, no more than 48%, or no more than 47%. In some embodiments, the control ORF has a G/C content of about 55%, about 54%, about 53%, about 52%, about 51%, about 50%, about 49%, about 48%, or about 47%. In some embodiments, the control ORF has a G/C content of about 50%. In some embodiments, the control ORF has a G/C content of about 52%.

In some embodiments, the ORF(s) of the one or more transgene(s) are codon optimized based on the codon usage of an aryl-halorespiring facultative anaerobic myxobacterium. In some embodiments, the ORF(s) of the one or more transgene(s) are codon optimized based on the codon usage of Anaeromyxobacter dehalogenans.

In some embodiments, one or more of the high G/C content ORFs encode an antibody or antigen binding fragment thereof. In some embodiments, the antibody or antigen binding fragment thereof comprises a heavy chain variable domain (VH) and a light chain variable domain (VL). In some embodiments, the antibody or antigen binding fragment thereof comprises a single chain variable fragment (scFv). In some embodiments, the antibody or antigen binding fragment thereof comprises a VHH domain derived from a single domain antibody (sdAb). In some embodiments, the antibody or antigen binding fragment thereof comprises an IgG-Fc. Inn some embodiments, the IgG is IgG1. In some embodiments, the ORF encodes a TREM2 binding antibody or antigen binding fragment thereof and comprises a polynucleotide sequence at least 90%, at least 95%, at least 97%, at least 98%, at least 99%, or 100% identical to SEQ ID NO: 938. In some embodiments, the ORF encodes a PD1 antagonist and comprises a polynucleotide sequence at least 90%, at least 95%, at least 97%, at least 98%, at least 99%, or 100% identical to SEQ ID NO: 937. In some embodiments, the ORF encodes a biomolecule comprising CTX and comprises a polynucleotide sequence at least 90%, at least 95%, at least 97%, at least 98%, at least 99%, or 100% identical to SEQ ID NO: 940 or 941.

In some embodiments, one or more of the high G/C content ORFs encode a cytokine, a chemokine, a receptor, a receptor ligand, an enzyme, and/or a reporter protein. In some embodiments, the ORF encodes IL-12 and comprise a polynucleotide sequence at least 90%, at least 95%, at least 97%, at least 98%, at least 99%, or 100% identical to SEQ ID NO: 936. In some embodiments, the ORF encodes HPGD and comprises a polynucleotide sequence at least 90%, at least 95%, at least 97%, at least 98%, at least 99%, or 100% identical to SEQ ID NO: 939.

### Retargeting

In some embodiments, the recombinant oncolytic viruses described herein comprise a retargeting domain. In some embodiments, the recombinant oncolytic viruses described herein comprise the retargeting domain inserted in one of the virus proteins (e.g., gD). In some embodiments, the recombinant oncolytic viruses described herein comprise a polynucleotide encoding the retargeting domain. In some embodiments, the retargeting domain specifically binds a target protein expressed by a target cell. In some embodiments, the target cell is a cancer cell. In some embodiments, the target cell is a glioblastoma cell. In some embodiments, the retargeting domain enables, or enhances the ability of, the oncolytic virus to infect the target cell.

In some embodiments, the retargeting domain enables the oncolytic virus to infect target cells that are resistant to a control oncolytic virus without the retargeting domain. For example, Nectin-1 is an entry receptor for herpes simplex virus. Cells without (or with minimal) Nectin-1 expression are usually resistant to HSV. More discussion of the Nectin-1 expression and herpes simplex virus infection can be found, for example, in Guzman et al., Acta Virol. 2006; 50 (1): 59-66; Ishino et al., Blood (2019) 134 (Supplement_1): 3242; Friedman et al., Sci. Rep. (2018) 8:13930; and Alayo et al., Sci Rep. (2020); 10:5095, the content of each of which is incorporated by reference in its entirety for all purposes.

In some embodiments, incorporating the retargeting domain into HSV enables the virus to infect cells expressing the target protein but not Nectin-1.

In some embodiments, to insert the retargeting domain into the oncolytic virus, a polynucleotide encoding the retargeting domain is incorporated into the corresponding region of the viral genome of the virus. In some embodiments, the polynucleotide encoding the retargeting domain is inserted into the open reading frame of a US6 gene encoding a glycoprotein D (gD). In some embodiments, the the polynucleotide encoding the retargeting domain replaces the US6 gene region encoding an amino acid sequence corresponding to amino acids 6-24 of SEQ ID NO: 921.

In some embodiments, the target protein expressed by the target cell comprises one or more integrins. In some embodiments, the target protein comprises integrin α5β1, integrin αvβ1, integrin αvβ3, integrin αvβ6, or a combination thereof. In some embodiments, the target protein comprises integrin α5β1. In some embodiments, the target protein comprises integrin αvβ1. In some embodiments, the target protein comprises integrin αvβ3. In some embodiments, the target protein comprises integrin αvβ6.

In some embodiments, the target protein expressed by the target cell comprises epidermal growth factor receptor (EGFR), or a mutant thereof. In some embodiments, the target protein comprises EGFR. In some embodiments, the target protein comprises EGFR variant III (EGFRvIII). Description of EGFR variant III can be found, for example, in Padfield et al., Front Oncol. 2015; 5:5, the content of which is incorporated by reference in its entirety for all purposes.

In some embodiments, the retargeting domain comprises a knottin peptide capable of specifically binding to the target protein expressed by the target cell. Knottins, or inhibitor cystine knots (ICKs), are a family of ultra-stable miniproteins characterized by the presence of at least three interwoven disulfide bridges, which form an intramolecular knot and confer them structural and functional resistance to high temperature, enzymatic degradation, extreme pH and mechanical stress. Typically, knottins are about 30-50 residues in length. Description of knottins, and a database (KNOTTIN) that stores knottin sequences, structures, and functions, can be found, for example, in Postic et al., Nucleic Acids Res. 2018 Jan. 4; 46 (D1): D454-D458, the content of which is incorporated by reference in its entirety for all purposes. In some embodiments, the retargeting domain comprising knottin has no more than 50, no more than 45, no more than 40, or no more than 35 amino acids. In some embodiments, the knottin domain is derived from Ecballium elaterium trypsin inhibitor II (EETI-II trypsin inhibitor). In some embodiments, the knottin domain comprises an amino acid sequence having at least 80%, at least 85%, at least 90%, or 100% identity to SEQ ID NO: 922. In some embodiments, the knottin domain binds to one or more integrins (e.g., integrin α5β1, integrin αvβ1, integrin αvβ3, integrin αvβ6, or a combination thereof).

In some embodiments, the retargeting domain comprises an immunoglobulin domain capable of specifically binding to the target protein expressed by the target cell. In some embodiments, the retargeting domain comprises a binding domain of, or a binding domain derived from, a variable domain of a heavy chain-only antibody (VHH) or a variable fragment of new antigen receptor immunoglobulin (V-NAR). In some embodiments, the retargeting domain comprises no more than 150, no more than 140, or no more than 130 amino acids. In some embodiments, the retargeting domain comprises no more than 150 amino acids. In some embodiments, the retargeting domain comprises between 110-150 amino acids. In some embodiments, the retargeting domain comprises an amino acid sequence having at least 80%, at least 85%, at least 90%, or 100% identity to SEQ ID NO: 923. In some embodiments, the knottin domain binds to EGFR and/or a mutant thereof (e.g., EGFRvIII).

In some embodiments, the retargeting domain comprises no more than 50, no more than 45, no more than 40, or no more than 35 amino acids. In some embodiments, the retargeting domain comprises no more than 150, no more than 140, or no more than 130 amino acids. In some embodiments, the retargeting domain comprises between 110-150 amino acids.

In some embodiments, the oncolytic virus (e.g., HSV) comprising the retargeting domain (and/or a polynucleotide encoding the retargeting domain) is capable of infecting the target cell expressing the target protein. In some embodiments, the target cell is a glioblastoma cell. In some embodiments, the glioblastoma cell has no Nectin-1 expression. In some embodiments, the target cell is a Vero cell with no Nectin-1 expression (e.g., Nectin-1 knock-out cells). In some embodiments, the target cell expresses EGFR. In some embodiments, the target cell expresses an EGFR mutant (e.g., EGFRvIII). In some embodiments, the target cell expresses one or more integrins (e.g., integrin α5β1, integrin αvβ1, integrin αvβ3, integrin αvβ6, or a combination thereof).

### Replication Fidelity and Acyclovir Sensitivity

In some embodiments, the recombinant oncolytic virus described herein comprises a mutation that increases the replication fidelity of the viral genome. In some embodiment, the mutation is located within the DNA polymerase.

The UL30 viral gene of herpesvirus encodes a DNA polymerase catalytic subunit (DPCS). In some embodiments, the encoded DPCS comprises a mutation. In some embodiments, the mutation in DPCS increases the replication fidelity of the herpesvirus. In some embodiments, the mutation in the DPCS increases DNA replication fidelity of the herpesvirus by at least 10%, at least 20%, at least 30%, at least 50%, at least 100%, at least 2-fold, at least 3-fold, or at least 5-fold. In some embodiments, the mutation in the DPCS increases DNA replication fidelity of the herpesvirus by at least 1-fold. In some embodiments, the mutation in the DPCS comprises a mutation at the amino acid position corresponding to L774 of SEQ ID NO: 917. In some embodiments, the mutation is an amino acid substitution. In some embodiments, the mutation comprises the amino acid substitution corresponding to L774F of SEQ ID NO: 917. In some embodiments, the encoded DPCS comprises an amino acid sequence at least 90%, at least 95%, at least 97%, at least 98%, at least 99%, or 100% identical to SEQ ID NO: 917, except for the mutation at the amino acid position corresponding to L774 of SEQ ID NO: 917. Discussion about the DPCS mutation can be found, for example, in Hwang et al., J Virol. 2004 January; 78 (2): 650-657, the content of which is incorporated by reference in its entirety for all purposes.

In some embodiments, the mutation in the DPCS undesirably decreases the herpesvirus' sensitivity to acyclovir and its analogues, even though it increases replication fidelity. Acyclovir and/or its analogues are anti-HSV drugs which can be used as a safety measure to control the unwanted dissemination of oncolytic HSV (oHSV) infection (e.g., to non-cancerous cells in CNS). These drugs may also be used as an imaging tool to locate the HSV infection site in vivo. More description of acyclovir and its analogues can be found, for example, in Klysik et al., Curr Med Chem. 2020; 27 (24): 4118-4137, the content of which is incorporated by reference in its entirety for all purposes.

Accordingly, in some embodiments, the recombinant herpesvirus of the disclosure further comprises a mutation that partially or completely restores, or even enhances, its sensitivity to acyclovir. In some embodiments, the mutation is located in the UL23 viral gene and results in a mutation in the thymidine kinase (TK) encoded by UL23. In some embodiments, the mutation in the TK is at one or more amino acid positions corresponding to L159, 1160, F161, A168 and/or L169 of SEQ ID NO: 918. In some embodiments, the mutation in the TK is at 2, 3, 4, or 5 amino acid positions selected from those corresponding to L159, I160, F161, A168 and/or L169 of SEQ ID NO: 918. In some embodiments, the mutation(s) are amino acid substitution(s). In some embodiments, the mutation(s) in the TK comprise one or more amino acid substitutions of:
(a) L159I or L159L;
(b) I160L or I160F;
(c) F161A, F161V, F161P, or F161L;
(d) A168D, A168Y, A168V or A168F; and/or
(e) L169F, L169Y, L169L, L169I, L169M, L169N, or L169K.

In some embodiments, the mutation in the TK comprises amino acid substitutions corresponding to L159I, 1160F, F161L, A168F and L169M of SEQ ID NO: 918. In some embodiments, the mutation in the TK comprises amino acid substitutions corresponding to I160F, F161A, and A168F of SEQ ID NO: 918. In some embodiments, the mutation in the TK comprises amino acid substitutions corresponding to 1160F, F161L, A168F, and L169N of SEQ ID NO: 918. In some embodiments, the TK comprises an amino acid sequence at least 90%, at least 95%, at least 97%, at least 98%, at least 99%, or 100% identical to SEQ ID NO: 918, except the TK mutation(s) recited in this paragraph. More discussion about the TK mutation(s) can be found, for example, in Black et al., Cancer Res. 2001 Apr. 1; 61 (7): 3022-6, the content of which is incorporated by reference in its entirety for all purposes.

The efficacy of acyclovir may be significantly reduced in cells infected with herpesvirus that are more resistant (e.g., has high IC90/IC50) to acyclovir (e.g., HSV comprising the DPCS L774F mutation). This issue may be more prominent in HSVs that are derived from a strain that is intrinsically less sensitive to acyclovir, such as the HSV-Macintyre strain. The bioavailability of acyclovir, when dosed orally, is about 1.76 ug/ml in the central nervous system of human. Accordingly, in some embodiments, the recombinant herpesvirus of the disclosure has an acyclovir IC50 that is less than 0.5 ug/ml, less than 1.0 ug/ml, less than 1.5 ug/ml, or less than 2.0 ug/ml. In some embodiments, the recombinant herpesvirus of the disclosure has an acyclovir IC50 that is less than 0.5 ug/ml. In some embodiments, the recombinant herpesvirus of the disclosure has an acyclovir IC50 that is less than 0.4 ug/ml. In some embodiments, the recombinant herpesvirus of the disclosure has an acyclovir IC50 that is less than 0.3 ug/ml. In some embodiments, the recombinant herpesvirus of the disclosure has an acyclovir IC50 that is less than 0.2 ug/ml. In some embodiments, the mutation(s) in the TK decrease the IC50 of acyclovir for the herpesvirus by at least 1-fold, at least 2-fold, at least 5-fold, at least 10-fold, at least 20-fold, at least 50-fold, or at least 100-fold. In some embodiments, the mutation(s) in the TK decrease the IC50 of acyclovir for the herpesvirus by at least 10-fold. Determination of acyclovir IC90/IC50 is known in the art and also described in Example 6 below.

This solution for reverting the acyclovir resistance caused by the UL30 mutation(s) relates to the surprising finding that the complementary mutation(s) of TK encoded by UL23 do not compromise the fitness of the virus described herein, even though such mutation(s) of TK likely lower the TK's affinity for the native ligand thymidine (and thus a skilled artisan would expect to instead observe lower virus fitness, similar to what was observed when the TK was knock-out or knock-down). Without wishing to be bound by any particular theory, it is hypothesized that the the complementary mutation(s) introduced into TK lower the availability of the thymidine enzymatic product to a level enough to re-sensitize the mutant DPCS to the presence of low level acyclovir, yet the mutant TK still provides sufficient enzymatic product to maintain the replication fidelity of the mutant DPCS and the overall virus fitness.

In one aspect, the recombinant herpesvirus of the disclosure and a small molecule such as acyclovir can be used together in a method of imaging the infection site of the herpesvirus (e.g., a tumor site) in vivo. In some embodiments, the method comprising administering the recombinant herpesvirus of the disclosure and a small molecule (e.g., acyclovir). In some embodiments, the small molecule is radioisotope labeled acyclovir. In some embodiments, the radioisotope label comprises fluorine-18 (18F) label.

### Syncytial Mutants and Virus Production

In one aspect, the oncolytic virus of the disclosure has enhanced fusogenicity. In some embodiments, the oncolytic virus of the disclosure causes higher degree of syncytial formation upon infecting the target cells compared to a control oncolytic virus without enhanced fusogenicity.

For oncolytic viruses, enhanced fusogenicity induces cell-cell fusion resulting in formation of a syncytium, improves oncolysis and virus spread in tumor cells and enhance immunogenicity through releasing immunostimulatory DAMPs (damage-associated molecular patterns). As used herein, the term “syncytium” refers to a cell-cell fusion which appears in a tissue biopsy or tissue culture sample as a large acellular area with multiple nucleii, i.e., a multinucleate region of cytoplasm.

The term “syncytial mutation” refers to a mutation that increases the ability of the polypeptide to induce syncytium formation (i.e., “enhanced fusogenic activity” or “enhanced fusogenicity”). In some embodiments, the syncytial mutation increases the ability of the polypeptide to induce syncytium formation by at least 10%, at least 20%, at least 30%, at least 50%, at least 100%, at least 2-fold, at least 3-fold, at least 5-fold, at least 10-fold, at least 50-fold, or at least 100-fold, compared to a control polypeptide without the syncytial mutation. In some embodiments, the syncytial mutation increases the ability of the polypeptide to induce syncytium formation by at least 100% compared to a control polypeptide without the syncytial mutation. The induction of syncytium formation may be measured based on the number of cells that are induced to form a syncytium. Methods for measuring the fusogenicity (ability to induce syncytium formation) of a polypeptide are known in the art, for example, see Melancon et al., J Virol. 2005 January; 79 (1): 299-313, the content of which is incorporated by reference in its entirety for all purposes.

For HSV, enhancing fusogenicity through introducing syncytial mutation(s) into gK, gB, UL20 and/or UL24 is described, for example, in US20210386807 and Fan et al., Sci Rep. 2017 Mar. 3; 7:43712, the content of each of which is incorporated by reference in its entirety for all purposes.

However, such enhanced fusogenicity often lowers the viral titer during virus production, thus hampering their clinical applications. To overcome this problem, one aspect of the present disclosure relates to an expression system that limits the expression of the fusogenic protein (the protein with enhanced fusogenicity) and instead favors the expression of a counterpart protein without enhanced fusogenicity during virus production. In some embodiments, limiting the expression of the fusogenic protein (the protein with enhanced fusogenicity) is achieved by inserting a miR-TS cassette into the loci encoding that protein, wherein the miR-TS cassette contains target sequences for one or more miRNAs expressed in the production cells.

In some embodiments, the counterpart protein without enhanced fusogenicity is expressed by a transgene encoded by the oncolytic virus. In some embodiments, the counterpart protein without enhanced fusogenicity is expressed by a transgene in the production cells that is separated from the viral genome.

In the case of herpesviruses such as HSV, in some embodiments this strategy can be applied to gB and/or gK proteins comprising one or more mutations which enhances the ability of the HSV to induce syncytium formation upon infecting the target cells (i.e., syncytial mutation(s)). Without wishing to be bound by any particular theory, it is hypothesized that these syncytial mutations in gB and/or gK act by reducing the receptor engagement threshold required to activate the gB/gK fusogenic proteins, such that during virus production most of the viral particles contain gB/gK proteins in an inactive, post-fusogenic conformation, thus lowering the virus yield.

Accordingly, to improve the yield of the HSV syncytial mutants during virus production (e.g., in Vero cells), an expression system was established to allow co-expression of the non-syncytial version as well as the syncytial mutants of gB and/or gK proteins. In some embodiments, this strategy facilitates the generation of viral envelopes that contain a greater proportion of gB and/or gK proteins in an active conformation.

In some embodiments, the viral genome of the oncolytic virus of the disclosure encodes a first gB and a second gB, wherein the first gB comprises a syncytial mutation, and wherein the second gB comprises no syncytial mutation. In some embodiments, the first gB is encoded by an endogenous gB gene locus and the second gB is encoded by an exogenous expression cassette. In some embodiments, the first gB is encoded by an exogenous expression cassette and the second gB is encoded by an endogenous gB gene locus. In some embodiments, the exogenous expression cassette is located at the UL50-UL51 intergenic locus. In some embodiments, the exogenous expression cassette is located at the UL3-UL4 intergenic locus.

In some embodiments, the viral genome of the herpesvirus of the disclosure encodes a first gK and a second gK, wherein the first gK comprises a syncytial mutation, and wherein the second gK comprises no syncytial mutation. In some embodiments, the first gK is encoded by an endogenous gK gene locus and the second gK is encoded by an exogenous expression cassette. In some embodiments, the first gK is encoded by an exogenous expression cassette and the second gK is encoded by an endogenous gK gene locus. In some embodiments, the exogenous expression cassette is located at the UL50-UL51 intergenic locus. In some embodiments, the exogenous expression cassette is located at the UL3-UL4 intergenic locus.

In some embodiments, the viral genome of the oncolytic virus of the disclosure encodes a first UL20 and a second UL20, wherein the first UL20 comprises a syncytial mutation, and wherein the second UL20 comprises no syncytial mutation. In some embodiments, the first UL20 is encoded by an endogenous UL20 gene locus and the second UL20 is encoded by an exogenous expression cassette. In some embodiments, the first UL20 is encoded by an exogenous expression cassette and the second UL20 is encoded by an endogenous UL20 gene locus. In some embodiments, the exogenous expression cassette is located at the UL50-UL51 intergenic locus. In some embodiments, the exogenous expression cassette is located at the UL3-UL4 intergenic locus.

In some embodiments, the viral genome of the oncolytic virus of the disclosure encodes a first gH and a second gH, wherein the first gH comprises a syncytial mutation, and wherein the second gH comprises no syncytial mutation. In some embodiments, the first gH is encoded by an endogenous gH gene locus and the second gH is encoded by an exogenous expression cassette. In some embodiments, the first gH is encoded by an exogenous expression cassette and the second gH is encoded by an endogenous gH gene locus. In some embodiments, the exogenous expression cassette is located at the UL50-UL51 intergenic locus. In some embodiments, the exogenous expression cassette is located at the UL3-UL4 intergenic locus.

In some embodiments, the viral genome of the oncolytic virus of the disclosure encodes a first UL24 and a second UL24, wherein the first UL24 comprises a syncytial mutation, and wherein the second UL24 comprises no syncytial mutation. In some embodiments, the first UL24 is encoded by an endogenous UL24 gene locus and the second UL24 is encoded by an exogenous expression cassette. In some embodiments, the first UL24 is encoded by an exogenous expression cassette and the second UL24 is encoded by an endogenous UL24 gene locus. In some embodiments, the exogenous expression cassette is located at the UL50-UL51 intergenic locus. In some embodiments, the exogenous expression cassette is located at the UL3-UL4 intergenic locus.

In some embodiments, the oncolytic virus of the disclosure displays enhanced syncytial phenotype in cancer cells.

In one aspect, the disclosure provides cells comprising a recombinant nucleic acid encoding the oncolytic virus of the disclosure (e.g., a virus encoding a first gB and/or gK comprising one or more syncytial mutations and a second gB and/or gK comprising no syncytial mutation).

In one aspect, the disclosure provides cells comprising a first nucleic acid encoding the oncolytic virus of the disclosure and a second nucleic acid, wherein the viral genome of the oncolytic virus encodes a first gB comprising one or more syncytial mutations, and wherein the second nucleic acid encodes a second gB comprising no syncytial mutation. In some embodiments, the oncolytic virus of the disclosure (e.g., HSV) comprises a single copy of gB-encoding viral gene and/or a single copy of gK-encoding viral gene.

In one aspect, the disclosure provides cells comprising a first nucleic acid encoding the oncolytic virus of the disclosure and a second nucleic acid, wherein the viral genome of the oncolytic virus encodes a second gB comprising no syncytial mutation, and wherein the second nucleic acid encodes a first gB comprising one or more syncytial mutations. In some embodiments, the oncolytic virus of the disclosure (e.g., HSV) comprises a single copy of gB-encoding viral gene and/or a single copy of gK-encoding viral gene.

In one aspect, the disclosure provides cells comprising a first nucleic acid encoding the oncolytic virus of the disclosure and a second nucleic acid, wherein the viral genome of the oncolytic virus of the disclosure encodes a first gK comprising one or more syncytial mutations, and wherein the second nucleic acid encodes a second gK comprising no syncytial mutation. In some embodiments, the oncolytic virus of the disclosure (e.g., HSV) comprises a single copy of gB-encoding viral gene and/or a single copy of gK-encoding viral gene.

In one aspect, the disclosure provides cells comprising a first nucleic acid encoding the oncolytic virus of the disclosure and a second nucleic acid, wherein the viral genome of the oncolytic virus of the disclosure encodes a second gK comprising no syncytial mutation, and wherein the second nucleic acid encodes a first gK comprising one or more syncytial mutations. In some embodiments, the oncolytic virus of the disclosure (e.g., HSV) comprises a single copy of gB-encoding viral gene and/or a single copy of gK-encoding viral gene.

In one aspect, the disclosure provides cells comprising a first nucleic acid encoding the oncolytic virus of the disclosure and a second nucleic acid, wherein the viral genome of the oncolytic virus encodes a first gH comprising one or more syncytial mutations, and wherein the second nucleic acid encodes a second gH comprising no syncytial mutation.

In one aspect, the disclosure provides cells comprising a first nucleic acid encoding the oncolytic virus of the disclosure and a second nucleic acid, wherein the viral genome of the oncolytic virus encodes a second gH comprising no syncytial mutation, and wherein the second nucleic acid encodes a first gH comprising one or more syncytial mutations.

In one aspect, the disclosure provides cells comprising a first nucleic acid encoding the oncolytic virus of the disclosure and a second nucleic acid, wherein the viral genome of the oncolytic virus encodes a first UL20 comprising one or more syncytial mutations, and wherein the second nucleic acid encodes a second UL20 comprising no syncytial mutation.

In one aspect, the disclosure provides cells comprising a first nucleic acid encoding the oncolytic virus of the disclosure and a second nucleic acid, wherein the viral genome of the oncolytic virus encodes a second UL20 comprising no syncytial mutation, and wherein the second nucleic acid encodes a first UL20 comprising one or more syncytial mutations.

In one aspect, the disclosure provides cells comprising a first nucleic acid encoding the oncolytic virus of the disclosure and a second nucleic acid, wherein the viral genome of the oncolytic virus encodes a first UL24 comprising one or more syncytial mutations, and wherein the second nucleic acid encodes a second UL24 comprising no syncytial mutation.

In one aspect, the disclosure provides cells comprising a first nucleic acid encoding the oncolytic virus of the disclosure and a second nucleic acid, wherein the viral genome of the oncolytic virus encodes a second UL24 comprising no syncytial mutation, and wherein the second nucleic acid encodes a first UL24 comprising one or more syncytial mutations.

In some embodiments, the first nucleic acid and the second nucleic acid are comprised within a single polynucleotide molecule in the cells. In some embodiments, the first nucleic acid and the second nucleic acid are comprised within two different polynucleotide molecules in the cells.

In some embodiments, the cells are Vero cells. In some embodiments, the cells are for production of the oncolytic virus in vitro.

In some embodiments, the gB syncytial mutation comprises a mutation at one or more amino acid residues corresponding to R796, R800, T813, L817, S854, A855, R858, or A874, an insertion between E816 and L817, a deletion of S869 to C-terminus, a deletion of T877 to C-terminus, or a combination thereof, of SEQ ID NO: 919. In some embodiments, the gB syncytial mutation comprises one or more mutations corresponding to R796C, R800W, T813I, L817H, L817P, S854F, A855V, R858C, R858H, A874P, an insertion of VN or VNVN between E816 and L817, a deletion of S869 to C-terminus, or a deletion of T877 to C-terminus, of SEQ ID NO: 919. In some embodiments, the gB syncytial mutation comprises a mutation corresponding to R796C, R800W, T813I, L817H, L817P, S854F, A855V, R858C, R858H, A874P, an insertion of VN or VNVN between E816 and L817, a deletion of S869 to C-terminus, or a deletion of T877 to C-terminus, of SEQ ID NO: 919. In some embodiments, the gB syncytial mutation comprises a mutation at the amino acid residue corresponding to R796 of SEQ ID NO: 919. In some embodiments, the gB syncytial mutation comprises a mutation corresponding to R796C of SEQ ID NO: 919. In some embodiments, the gB syncytial mutation comprises a mutation at the amino acid residue corresponding to R800 of SEQ ID NO: 919. In some embodiments, the gB syncytial mutation comprises a mutation corresponding to R800W of SEQ ID NO: 919. In some embodiments, the gB syncytial mutation comprises a mutation at the amino acid residue corresponding to T813 of SEQ ID NO: 919. In some embodiments, the gB syncytial mutation comprises a mutation corresponding to T813I of SEQ ID NO: 919. In some embodiments, the gB syncytial mutation comprises a mutation at the amino acid residue corresponding to L817 of SEQ ID NO: 919. In some embodiments, the gB syncytial mutation comprises a mutation corresponding to L817H of SEQ ID NO: 919. In some embodiments, the gB syncytial mutation comprises a mutation corresponding to L817P of SEQ ID NO: 919. In some embodiments, the gB syncytial mutation comprises a mutation at the amino acid residue corresponding to S854 of SEQ ID NO: 919. In some embodiments, the gB syncytial mutation comprises a mutation corresponding to S854F of SEQ ID NO: 919. In some embodiments, the gB syncytial mutation comprises a mutation at the amino acid residue corresponding to A855 of SEQ ID NO: 919. In some embodiments, the gB syncytial mutation comprises a mutation corresponding to A855V of SEQ ID NO: 919. In some embodiments, the gB syncytial mutation comprises a mutation at the amino acid residue corresponding to R858 of SEQ ID NO: 919. In some embodiments, the gB syncytial mutation comprises a mutation corresponding to R858C of SEQ ID NO: 919. In some embodiments, the gB syncytial mutation comprises a mutation corresponding to R858H of SEQ ID NO: 919. In some embodiments, the gB syncytial mutation comprises a mutation at the amino acid residue corresponding to A874 of SEQ ID NO: 919. In some embodiments, the gB syncytial mutation comprises a mutation corresponding to A874P of SEQ ID NO: 919. In some embodiments, the gB syncytial mutation comprises an insertion between amino acid residues corresponding to E816 and L817 of SEQ ID NO: 919. In some embodiments, the gB syncytial mutation comprises a mutation corresponding to an insertion of VN between E816 and L817 of SEQ ID NO: 919. In some embodiments, the gB syncytial mutation comprises a mutation corresponding to an insertion of VNVN between E816 and L817 of SEQ ID NO: 919. In some embodiments, the gB syncytial mutation comprises a deletion of the amino acid residues corresponding to S869 to C-terminus of SEQ ID NO: 919. In some embodiments, the gB syncytial mutation comprises a deletion of the amino acid residues corresponding to T877 to C-terminus of SEQ ID NO: 919.

In some embodiments, the first and the second gB further comprise a mutation corresponding to D285N and/or A549T of SEQ ID NO: 919. In some embodiments, the first gB, but not the second gB, further comprises a mutation corresponding to D285N and/or A549T of SEQ ID NO: 919. In some embodiments, the second gB, but not the first gB, further comprises a mutation corresponding to D285N and/or A549T of SEQ ID NO: 919.

In some embodiments, the open reading frame encoding the first gB is operably linked to a CMV promoter. In some embodiments, the open reading frame encoding the first gB is operably linked to a bGH polyA tail.

In some embodiments, the open reading frame encoding the second gB is operably linked to a CMV promoter. In some embodiments, the open reading frame encoding the second gB is operably linked to a bGH poly A tail.

In some embodiments, the gK syncytial mutation comprises a mutation at one or more amino acid residues corresponding to P33, A40, L86, D99, A111, L118, T121, C243, L304, 1307, or R310 of SEQ ID NO: 920. In some embodiments, the gK syncytial mutation comprises one or more mutations corresponding to P33S, A40V, A40T, L86P, D99N, A111V, L118Q, T121I, C243Y, L304P, 1307N, or R310L of SEQ ID NO: 920. In some embodiments, the gK syncytial mutation comprises a mutation at the amino acid residue corresponding to P33 of SEQ ID NO: 920. In some embodiments, the gK syncytial mutation comprises a mutation corresponding to P33S of SEQ ID NO: 920. In some embodiments, the gK syncytial mutation comprises a mutation at the amino acid residue corresponding to A40 of SEQ ID NO: 920. In some embodiments, the gK syncytial mutation comprises a mutation corresponding to A40V of SEQ ID NO: 920. In some embodiments, the gK syncytial mutation comprises a mutation corresponding to A40T of SEQ ID NO: 920. In some embodiments, the gK syncytial mutation comprises a mutation at the amino acid residue corresponding to L86 of SEQ ID NO: 920. In some embodiments, the gK syncytial mutation comprises a mutation corresponding to L86P of SEQ ID NO: 920. In some embodiments, the gK syncytial mutation comprises a mutation at the amino acid residue corresponding to D99 of SEQ ID NO: 920. In some embodiments, the gK syncytial mutation comprises a mutation corresponding to D99N of SEQ ID NO: 920. In some embodiments, the gK syncytial mutation comprises a mutation at the amino acid residue corresponding to A111 of SEQ ID NO: 920. In some embodiments, the gK syncytial mutation comprises a mutation corresponding to A111V of SEQ ID NO: 920. In some embodiments, the gK syncytial mutation comprises a mutation at the amino acid residue corresponding to L118 of SEQ ID NO: 920. In some embodiments, the gK syncytial mutation comprises a mutation corresponding to L118Q of SEQ ID NO: 920. In some embodiments, the gK syncytial mutation comprises a mutation at the amino acid residue corresponding to T121 of SEQ ID NO: 920. In some embodiments, the gK syncytial mutation comprises a mutation corresponding to T121I of SEQ ID NO: 920. In some embodiments, the gK syncytial mutation comprises a mutation at the amino acid residue corresponding to C243 of SEQ ID NO: 920. In some embodiments, the gK syncytial mutation comprises a mutation corresponding to C243Y of SEQ ID NO: 920. In some embodiments, the gK syncytial mutation comprises a mutation at the amino acid residue corresponding to L304 of SEQ ID NO: 920. In some embodiments, the gK syncytial mutation comprises a mutation corresponding to L304P of SEQ ID NO: 920. In some embodiments, the gK syncytial mutation comprises a mutation at the amino acid residue corresponding to 1307 of SEQ ID NO: 920. In some embodiments, the gK syncytial mutation comprises a mutation corresponding to 1307N of SEQ ID NO: 920. In some embodiments, the gK syncytial mutation comprises a mutation at the amino acid residue corresponding to R310 of SEQ ID NO: 920. In some embodiments, the gK syncytial mutation comprises a mutation corresponding to R310L of SEQ ID NO: 920.

In some embodiments, the open reading frame encoding the first gK is operably linked to a CMV promoter. In some embodiments, the open reading frame encoding the first gK is operably linked to a bGH poly A tail.

In some embodiments, the open reading frame encoding the second gK is operably linked to a CMV promoter. In some embodiments, the open reading frame encoding the second gK is operably linked to a bGH poly A tail.

In some embodiments, the gH syncytial mutation comprises a mutation at one or more amino acid residues corresponding to N753 or A778 of SEQ ID NO: 943. In some embodiments, the gH syncytial mutation comprises one or more mutations corresponding to N753K or A778V of SEQ ID NO: 943. In some embodiments, the gH syncytial mutation comprises mutations corresponding to N753K and A778V of SEQ ID NO: 943. Additional description of the gH mutation can be found, for example, at Uchida et al., J Virol. 2013 February; 87 (3): 1430-1442, the content of which is incorporated by reference in its entirety for all purposes.

In some embodiments, the UL20 syncytial mutation comprises a mutation at one or more amino acid residues corresponding to Y49A, S50A, R51A, R209A, T212A, R213A, or c-terminal deletion after N217, of SEQ ID NO: 944. In some embodiments, the UL20 syncytial mutation comprises one or more mutations corresponding to Y49A, S50A, R51A, R209A, T212A, R213A, or c-terminal deletion after N217, of SEQ ID NO: 944. In some embodiments, the UL20 syncytial mutation comprises the mutations corresponding to Y49A, S50A, and R51A of SEQ ID NO: 944. In some embodiments, the UL20 syncytial mutation comprises the mutations corresponding to R209A, T212A, and R213A of SEQ ID NO: 944.

In some embodiments, the UL24 syncytial mutation comprises a mutation at one or more amino acid residues corresponding to T64, R63, or V64 of SEQ ID NO: 942. In some embodiments, the UL24 syncytial mutation comprises one or more mutations corresponding to T64G, R63V, or V64S of SEQ ID NO: 942. In some embodiments, the UL24 syncytial mutation comprises the mutations corresponding to T64G, R63V and V64S of SEQ ID NO: 942.

This strategy can be extrapolated to other viruses as well. In general, when a syncytial mutation is introduced into a protein encoded by a recombinant virus, so that the recombinant virus has enhanced fusogenicity, the production yield of the recombinant virus in the production cell may be improved by co-expressing a counterpart protein without the syncytial mutation (encoded by the virus or the production cell). Either protein may be encoded by an endogenous viral gene or by an exogenous expression cassette.

In some embodiments, the yield of the recombinant oncolytic virus for the virus or cells comprising the second nucleic acid encoding the counterpart protein without enhanced fusogenicity (e.g., the second gB and/or gK, which comprises no syncytial mutation) is increased by at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold compared to the yield of a control oncolytic virus or control cells that does not encode the counterpart protein without the enhanced fusogenicity. In some embodiments, the yield of the recombinant oncolytic virus is increased by at least 1-fold. In some embodiments, the yield of the recombinant oncolytic virus is increased by at least 2-fold. In some embodiments, the yield of the recombinant oncolytic virus is increased by at least 3-fold. In some embodiments, the yield of the recombinant oncolytic virus is increased by at least 5-fold. In some embodiments, the yield of the recombinant oncolytic virus is increased by at least 8-fold. In some embodiments, the yield of the recombinant oncolytic virus is increased by at least 10-fold. In some embodiments, the yield of the recombinant oncolytic virus is increased by at least 20-fold. In some embodiments, the yield of the recombinant oncolytic virus is increased by at least 50-fold. In some embodiments, the yield of the recombinant oncolytic virus is increased by at least 100-fold. In some embodiments, the yield of the recombinant oncolytic virus is increased by at least 200-fold. In some embodiments, the yield of the recombinant oncolytic virus is increased by at least 500-fold. In some embodiments, the yield of the recombinant oncolytic virus is increased by at least 1000-fold.

In some embodiments, in the oncolytic virus of the disclosure, the gene encoding the fusogenic protein (the protein with enhanced fusogenicity) comprises a miRNA target sequence (miR-TS) cassette, wherein the miR-TS cassette comprises one or more target sequences of one or more miRNAs. In some embodiments, the one or more miRNAs are highly expressed in the cells for virus production in vitro. In some embodiments, the one or more miRNAs have no or minimal expression in the target cells (e.g., cancer cells). In some embodiments, the one or more miRNAs have relatively higher expression in the cells for virus production in vitro compared to the target cells (e.g., cancer cells). In some embodiments, the cells for virus production in vitro express the one or more miRNAs at a level that is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than that of the target cells (e.g., cancer cells).

In some embodiments, the gene encoding the first gB comprises a miRNA target sequence (miR-TS) cassette, wherein the miR-TS cassette comprises one or more target sequences of one or more miRNAs. In some embodiments, the one or more miRNAs are highly expressed in the cells for virus production in vitro. In some embodiments, the one or more miRNAs have no or minimal expression in the target cells (e.g., cancer cells). In some embodiments, the one or more miRNAs have relatively higher expression in the cells for virus production in vitro compared to the target cells (e.g., cancer cells). In some embodiments, the cells for virus production in vitro express the one or more miRNAs at a level that is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than that of the target cells (e.g., cancer cells).

In some embodiments, the gene encoding the first gK comprises a miRNA target sequence (miR-TS) cassette, wherein the miR-TS cassette comprises one or more target sequences of one or more miRNAs. In some embodiments, the one or more miRNAs are highly expressed in the cells for virus production in vitro. In some embodiments, the one or more miRNAs have no or minimal expression in the target cells (e.g., cancer cells). In some embodiments, the one or more miRNAs have relatively higher expression in the cells for virus production in vitro compared to the target cells (e.g., cancer cells). In some embodiments, the cells for virus production in vitro express the one or more miRNAs at a level that is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than that of the target cells (e.g., cancer cells).

In some embodiments, the one or more miRNAs comprise at least one of miR-34c-5p, miR-299-5p, and miR-582-5p. In some embodiments, the one or more miRNAs comprise miR-34c-5p. In some embodiments, the one or more miRNAs comprise miR-299-5p. In some embodiments, the one or more miRNAs comprise miR-582-5p. In some embodiments, the one or more miRNAs comprise at least two of miR-34c-5p, miR-299-5p, and miR-582-5p. In some embodiments, the one or more miRNAs comprise miR-34c-5p and miR-299-5p. In some embodiments, the one or more miRNAs comprise miR-299-5p and miR-582-5p. In some embodiments, the one or more miRNAs comprise miR-34c-5p and miR-582-5p. In some embodiments, the one or more miRNAs comprise miR-34c-5p, miR-299-5p, and miR-582-5p. In some embodiments, the miRNA target sequence comprises or consists of the reverse complement of the miRNA. In some embodiments, the miR-34c-5p target sequences are at most 4, at most 3, at most 2, or at most 1 nucleotide(s) different from SEQ ID NO: 927. In some embodiments, the miR-34c-5p target sequences comprise or consist of SEQ ID NO: 927. In some embodiments, the miR-299-5p target sequences are at most 4, at most 3, at most 2, or at most 1 nucleotide(s) different from SEQ ID NO: 928. In some embodiments, the miR-299-5p target sequences comprise or consist of SEQ ID NO: 928. In some embodiments, the miR-582-5p target sequences are at most 4, at most 3, at most 2, or at most 1 nucleotide(s) different from SEQ ID NO: 929. In some embodiments, the miR-582-5p target sequences comprise or consist of SEQ ID NO: 929. In some embodiments, the miR-TS cassette comprises at least three copies, or at least four copies of the target sequences of each of the miRNA separated by a 4 bp spacer. In some embodiments, the miR-TS cassette is located at the 3′UTR of the gene(s) (e.g., the gene(s) encoding gB and/or gK). In some embodiments, the target sequence of the miRNA comprises or consists of the reverse complement of the miRNA. In some embodiments, the miR-TS cassette comprises at least 1, 2, 3, or 4 copies of a target sequence for miR-34c-5p.

In some embodiments, the miR-TS cassette comprises at least 1, 2, 3, or 4 copies of a target sequence for miR-299-5p. In some embodiments, the miR-TS cassette comprises at least 1, 2, 3, or 4 copies of a target sequence for miR-582-5p. In some embodiments, the miRNA target sequences in the miR-TS cassettes are arranged as follows:
(299-5p)-(34c-5p)-(582-5p)-(34c-5p)-(299-5p)-(582-5p)-(299-5p)-(582-5p)-(34c-5p)-(299-5p)-(582-5p)-(34c-5p).

In some embodiments, the miR-TS cassette comprises a polynucleotide sequence at least 80%, at least 85%, at least 90%, at least 95%, at least 96%, at least 97%, at least 98%, at least 99%, or 100% identical to SEQ ID NO: 930. In some embodiments, the miR-TS cassette comprises the polynucleotide sequence of SEQ ID NO: 930.

In one aspect, the disclosure provides fusogenic oncolytic viruses (e.g., HSV) produced by culturing the cells of the disclosure and recovering the fusogenic oncolytic virus (e.g., HSV) from the cell culture.

In one aspect, the disclosure a fusogenic oncolytic viruses (e.g., HSV) wherein the viral genome of the virus encodes a gK comprising a syncytial mutation corresponding to 1307N of SEQ ID NO: 920. In some embodiments, the gK comprises an amino acid sequence at least 90%, at least 95%, at least 96%, at least 97%, at least 98%, at least 99%, or 100% identical to SEQ ID NO: 920, except the syncytial mutation corresponding to 1307N of SEQ ID NO: 920.

In some embodiments, the presence of the miR-TS cassette decreases the expression of the fusogenic protein (e.g., the first gB or the first gK, which comprises the syncytial mutation) by at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold in the cells for virus production, compared to the expression of a control fusogenic protein encoding gene that does not comprise the miR-TS cassette. In some embodiments, the expression of the fusogenic protein is decreased by at least 1-fold. In some embodiments, the expression of the fusogenic protein is decreased by at least 2-fold. In some embodiments, the expression of the fusogenic protein is decreased by at least 3-fold. In some embodiments, the expression of the fusogenic protein is decreased by at least 5-fold. In some embodiments, the expression of the fusogenic protein is decreased by at least 8-fold. In some embodiments, the expression of the fusogenic protein is decreased by at least 10-fold. In some embodiments, the expression of the fusogenic protein is decreased by at least 20-fold. In some embodiments, the expression of the fusogenic protein is decreased by at least 50-fold. In some embodiments, the expression of the fusogenic protein is decreased by at least 100-fold. In some embodiments, the expression of the fusogenic protein is decreased by at least 200-fold. In some embodiments, the expression of the fusogenic protein is decreased by at least 500-fold. In some embodiments, the expression of the fusogenic protein is decreased by at least 1000-fold.

In some embodiments, the yield of the recombinant oncolytic virus for the virus or the cells comprising the miR-TS cassette in the gene encoding the fusogenic protein (e.g., the first gB or the first gK, which comprises the syncytial mutation) is increased by at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold compared to the yield of a control oncolytic virus or control cells that does not comprise the miR-TS cassette. In some embodiments, the yield of the recombinant oncolytic virus is increased by at least 1-fold. In some embodiments, the yield of the recombinant oncolytic virus is increased by at least 2-fold. In some embodiments, the yield of the recombinant oncolytic virus is increased by at least 3-fold. In some embodiments, the yield of the recombinant oncolytic virus is increased by at least 5-fold. In some embodiments, the yield of the recombinant oncolytic virus is increased by at least 8-fold. In some embodiments, the yield of the recombinant oncolytic virus is increased by at least 10-fold. In some embodiments, the yield of the recombinant oncolytic virus is increased by at least 20-fold. In some embodiments, the yield of the recombinant oncolytic virus is increased by at least 50-fold. In some embodiments, the yield of the recombinant oncolytic virus is increased by at least 100-fold. In some embodiments, the yield of the recombinant oncolytic virus is increased by at least 200-fold. In some embodiments, the yield of the recombinant oncolytic virus is increased by at least 500-fold. In some embodiments, the yield of the recombinant oncolytic virus is increased by at least 1000-fold.

### Particles and Nucleic Acids

In one aspect, the disclosure provides nucleic acid molecules encoding the virus of the disclosure. In some embodiments, the nucleic acid molecule is DNA. In some embodiments, the nucleic acid molecule is RNA. In some embodiments, the type of the nucleic acid molecule (DNA/RNA) is the same as the type of the virus (DNA virus/RNA virus).

In some embodiments, the nucleic acid molecule of the disclosure is comprised within particles. In some embodiments, the particle is a non-viral particle (e.g., LNP). In some embodiments, the particle is a non-tissue derived composition of matter such as liposomes, lipoplexes, nanoparticles, nanocapsules, microparticles, microspheres, lipid particles, exosomes, vesicles, and the like. In some embodiments, the particles are non-proteinaceous and non-immunogenic. In some embodiments, the particles are inorganic particles. In some embodiments, the inorganic particles are gold nanoparticles (GNP), gold nanorods (GNR), magnetic nanoparticles (MNP), magnetic nanotubes (MNT), carbon nanohorns (CNH), carbon fullerenes, carbon nanotubes (CNT), calcium phosphate nanoparticles (CPNP), mesoporous silica nanoparticles (MSN), silica nanotubes (SNT), or a starlike hollow silica nanoparticle (SHNP).

In some embodiments, encapsulation of the nucleic acid molecules of the disclosure allows for delivery of a viral genome without the induction of a systemic, anti-viral immune response and mitigates the effects of neutralizing anti-viral antibodies. Further, encapsulation of the nucleic acid molecules of the disclosure shields the genomes from degradation and facilitates the introduction into target host cells. In some embodiments, the particles are nanoparticles. In some embodiments, the particles are lipid nanoparticles. In some embodiments, the particles are exosomes. In some embodiments, the particle comprises no additional nucleic acid molecule. In some embodiments, the particles comprises no viral protein.

In some embodiments, the particles of the disclosure are nanoscopic in size, in order to enhance solubility, avoid possible complications caused by aggregation in vivo and to facilitate pinocytosis. In some embodiments, the particle has an average diameter of about less than about 1000 nm. In some embodiments, the particle has an average diameter of less than about 500 nm. In some embodiments, the particle has an average diameter of between about 30 and about 100 nm, between about 50 and about 100 nm, or between about 75 and about 100 nm. In some embodiments, the particle has an average diameter of between about 30 and about 75 nm or between about 30 and about 50 nm. In some embodiments, the particle has an average diameter between about 100 and about 500 nm. In some embodiments, the particle has an average diameter between about 200 and 400 nm. In some embodiments, the particle has an average size of about 350 nm.

In some embodiments, the particles are lipid nanoparticles (LNPs). In some embodiments, the LNP comprises one or more lipids such as such as triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate). In some embodiments, the LNP comprises one or more cationic lipids and one or more helper lipids. In some embodiments, the LNP comprises one or more cationic lipids, a cholesterol, and one or more neutral lipids.

### Compositions and Methods of Use

Certain aspects of the disclosure relate to stocks and compositions comprising the oncolytic viruses described herein. In some aspects, the disclosure relates to a viral stock comprising an oncolytic virus described herein. In some embodiments, a viral stock is a homogeneous stock. The preparation and analysis of viral stocks is well known in the art. For example, a viral stock can be manufactured in roller bottles containing cells transduced with the viral vector. The viral stock can then be purified on a continuous nycodenze gradient, and aliquotted and stored until needed. Viral stocks vary considerably in titer, depending largely on viral genotype and the protocol and cell lines used to prepare them.

In particular embodiments, the titer of a viral stock (e.g., an HSV-based vector viral stock) contemplated herein is at least about 10⁵ plaque-forming units (pfu), such as at least about 10⁶ pfu or even more preferably at least about 10⁷ pfu. In certain embodiments, the titer can be at least about 10⁸ pfu, or at least about 10⁹ pfu, and high titer stocks of at least about 10¹⁰ pfu or at least about 10¹¹ pfu are most preferred.

The disclosure further contemplates a composition comprising an oncolytic virus or a nucleic acid molecule described herein and a pharmaceutically acceptable carrier. The phrase “pharmaceutically-acceptable” refers to molecular entities and compositions that do not produce an allergic or similar untoward reaction when administered to a subject (e.g., a human). The term “composition” as used herein refers to a formulation of one or more oncolytic virus or a nucleic acid molecules described herein that is capable of being administered or delivered to a subject and/or a cell. Typically, formulations include all physiologically acceptable compositions including derivatives and/or prodrugs, solvates, stereoisomers, racemates, or tautomers thereof with any physiologically acceptable carriers, diluents, and/or excipients. A “therapeutic composition” or “pharmaceutical composition” (used interchangeably herein) is a composition of one or more agents capable of being administered or delivered to a patient and/or subject and/or cell for the treatment of a particular disease or disorder.

The compositions disclosed herein may be formulated in a neutral or salt form. “Pharmaceutically acceptable salt” includes both acid and base addition salts. Pharmaceutically-acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

As used herein “pharmaceutically acceptable carrier” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, dispersion media, coatings, antibacterial and antifungal agents, isotonic and absorption delaying agents, and the like that are physiologically compatible, including pharmaceutically acceptable cell culture media and/or emulsifier which has been approved by the United States Food and Drug Administration as being acceptable for use in humans and/or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations. Except insofar as any conventional media and/or agent is incompatible with the agents of the present disclosure, its use in therapeutic compositions is contemplated. Supplementary active ingredients also can be incorporated into the compositions.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In one embodiment, a composition comprising a carrier is suitable for parenteral administration, e.g., intravascular (intravenous or intraarterial), intraperitoneal or intramuscular administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with a viral vector or nucleic acid molecule, use thereof in the pharmaceutical compositions of the disclosure is contemplated.

The compositions of the disclosure may comprise one or more polypeptides, polynucleotides, vectors comprising same, infected cells, etc., as described herein, formulated in pharmaceutically-acceptable or physiologically-acceptable solutions for administration to a cell or an animal, either alone, or in combination with one or more other modalities of therapy. It will also be understood that, if desired, the compositions of the disclosure may be administered in combination with other agents as well, such as, e.g., cytokines, growth factors, hormones, small molecules or various pharmaceutically-active agents. There is virtually no limit to other components that may also be included in the compositions, provided that the additional agents do not adversely affect the ability of the composition to deliver the intended therapy.

In the pharmaceutical compositions of the disclosure, formulation of pharmaceutically-acceptable excipients and carrier solutions is well-known to those of skill in the art, as is the development of suitable dosing and treatment regimens for using the particular compositions described herein in a variety of treatment regimens. Upon formulation, solutions are administered in a manner compatible with the dosage formulation and in such amount as is therapeutically effective to result in an improvement or remediation of the symptoms. The formulations are easily administered in a variety of dosage forms such as ingestible solutions, drug release capsules and the like. Some variation in dosage can occur depending on the condition of the subject being treated. The person responsible for administration can, in any event, determine the appropriate dose for the individual subject. Moreover, for human administration, preparations meet sterility, general safety and purity standards as required by FDA Center for Biologics Evaluation and Research standards. The route of administration will vary, naturally, with the location and nature of the disease being treated, and may include, for example intradermal, transdermal, subdermal, parenteral, nasal, intravenous, intramuscular, intranasal, subcutaneous, percutaneous, intratracheal, intraperitoneal, intratumoral, perfusion, lavage, direct injection, and oral administration.

In certain circumstances it will be desirable to deliver the compositions, recombinant viral vectors, and nucleic acid molecules disclosed herein parenterally, intravenously, intramuscularly, or even intraperitoneally as described, for example, in U.S. Pat. Nos. 5,543,158; 5,641,515 and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabenes, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, MD: Lippincott Williams & Wilkins, 2000). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions can be prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain embodiments, the compositions may be delivered by intranasal sprays, inhalation, and/or other aerosol delivery vehicles. Methods for delivering polynucleotides and peptide compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In certain embodiments, the delivery may occur by use of liposomes, nanocapsules, microparticles, microspheres, lipid particles, vesicles, optionally mixing with CPP polypeptides, and the like, for the introduction of the compositions of the present disclosure into suitable host cells. In particular, the compositions of the present disclosure may be formulated for delivery either encapsulated in a lipid particle, a liposome, a vesicle, a nanosphere, a nanoparticle or the like. The formulation and use of such delivery vehicles can be carried out using known and conventional techniques. The formulations and compositions of the disclosure may comprise one or more polypeptides, polynucleotides, and small molecules, as described herein, formulated in pharmaceutically-acceptable or physiologically-acceptable solutions (e.g., culture medium) for administration to a cell or an animal, either alone, or in combination with one or more other modalities of therapy. It will also be understood that, if desired, the compositions of the disclosure may be administered in combination with other agents as well, such as, e.g., cells, other proteins or polypeptides or various pharmaceutically-active agents.

In a particular embodiment, a formulation or composition according to the present disclosure comprises a cell contacted with a combination of any number of polynucleotides or viral vectors, as contemplated herein.

In certain aspects, the present disclosure provides formulations or compositions suitable for the delivery of viral vector systems.

Exemplary formulations for ex vivo delivery may also include the use of various transfection agents known in the art, such as calcium phosphate, electroporation, heat shock and various liposome formulations (i.e., lipid-mediated transfection). Liposomes are lipid bilayers entrapping a fraction of aqueous fluid. DNA spontaneously associates to the external surface of cationic liposomes (by virtue of its charge) and these liposomes will interact with the cell membrane.

Particular embodiments of the disclosure may comprise other formulations, such as those that are well known in the pharmaceutical art, and are described, for example, in Remington: The Science and Practice of Pharmacy, 20th Edition. Baltimore, MD: Lippincott Williams & Wilkins, 2000.

In certain aspects, the present disclosure provides pharmaceutically acceptable compositions which comprise a therapeutically effective amount of one or more viral vectors or polynucleotides, as described herein, formulated together with one or more pharmaceutically acceptable carriers (additives) and/or diluents (e.g., pharmaceutically acceptable cell culture medium). As used herein, a “therapeutically effective amount” refers to the amount of a composition or recombinant virus described herein required to achieve a desired physiologic and/or biological outcome. A “therapeutically effective amount” of a virus, a viral stock, or a composition may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient). The therapeutically effective amount may be quantified by the total number of plaque forming units (pfu) (e.g. at least 1e¹ to at least 1e²⁰, particularly about 1e⁴ to about 1e¹⁵, more particularly about 1e⁶ to about 1e¹² pfu), or number of viral genomes (e.g. at least 1e¹ to at least 1e²⁰, particularly about 1e⁴ to about 1e¹⁵, more particularly about 1e⁶ to about 1e¹² viral genomes). One of skill in the art will understand that the therapeutically effective amount will vary based on the type of virus being administered, nature of the formulation, route of administration, nature and/or severity of the disease to be treated, and/or general health and well-being of the subject.

Some aspects of the disclosure encompass a method of killing a cancerous cell, comprising exposing the cancerous cell to an oncolytic virus described herein or compositions thereof under conditions sufficient for the oncolytic virus to infect and replicate within said cancerous cell, and wherein replication of the oncolytic virus within the cancerous cell results in cell death. In certain embodiments, the cancerous cell has a reduced expression of a miR compared to a non-cancerous cell. In some embodiments, a cancerous cell killed by this method is in vivo. In certain embodiments, a cancerous cell killed by this method is within a tumor.

The disclosure relates to a method of treating cancer in a subject in need thereof, comprising administering a prophylactically effective amount or a therapeutically effective amount of an oncolytic virus, a viral stock, a particle, or a composition as described herein to the subject. A “subject,” as used herein, includes any animal that exhibits a symptom of a disease, disorder, or condition that can be treated with the recombinant viral vectors, compositions, and methods disclosed herein. Suitable subjects (e.g., patients) include laboratory animals (such as mouse, rat, rabbit, or guinea pig), farm animals (such as horse or cow), and domestic animals or pets (such as cat or dog). Non-human primates and, preferably, human patients, are included.

“Administration” refers herein to introducing an oncolytic virus, a viral stock, or a composition thereof into a subject or contacting an oncolytic virus, a viral stock, or a composition thereof with a cell and/or tissue. Administration can occur by injection, irrigation, inhalation, consumption, electro-osmosis, hemodialysis, iontophoresis, and other methods known in the art. The route of administration will vary, naturally, with the location and nature of the disease being treated, and may include, for example auricular, buccal, conjunctival, cutaneous, dental, endocervical, endosinusial, endotracheal, enteral, epidural, interstitial, intra-articular, intra-arterial, intra-abdominal, intraauricular, intrabiliary, intrabronchial, intrabursal, intracavernous, intracerebral, intracisternal, intracorneal, intracronal, intracoronary, intracranial, intradermal, intradiscal, intraductal, intraduodenal, intraduodenal, intradural, intraepicardial, intraepidermal, intraesophageal, intragastric, intragingival, intrahepatic, intraileal, intralesional, intralingual, intraluminal, intralymphatic, intramammary, intramedulleray, intrameningeal, instramuscular, intranasal, intranodal, intraocular, intraomentum, intraovarian, intraperitoneal, intrapericardial, intrapleural, intraprostatic, intrapulmonary, intraruminal, intrasinal, intraspinal, intrasynovial, intratendinous, intratesticular, intratracheal, intrathecal, intrathoracic, intratubular, intratumoral, intratympanic, intrauterine, intraperitoneal, intravascular, intraventricular, intravesical, intravestibular, intravenous, intravitreal, larangeal, nasal, nasogastric, oral, ophthalmic, oropharyngeal, parenteral, percutaneous, periarticular, peridural, perineural, periodontal, respiratory, retrotubular, rectal, spinal, subarachnoid, subconjunctival, subcutaneous, subdermal, subgingival, sublingual, submucosal, subretinal, topical, transdermal, transendocardial, transmucosal, transplacental, trantracheal, transtympanic, ureteral, urethral, and/or vaginal perfusion, lavage, direct injection, and oral administration.

The term “treating” and “treatment” as used herein refers to administering to a subject a therapeutically effective amount of a recombinant virus or composition thereof as described herein so that the subject has an improvement in a disease or condition, or a symptom of the disease or condition. The improvement is any improvement or remediation of the disease or condition, or symptom of the disease or condition. The improvement is an observable or measurable improvement, or may be an improvement in the general feeling of well-being of the subject. Thus, one of skill in the art realizes that a treatment may improve the disease condition, but may not be a complete cure for the disease. A “prophylactically effective amount” refers to an amount of a virus, a viral stock, or a composition effective to achieve the desired prophylactic result. As used herein, “prophylaxis” can mean complete prevention of the symptoms of a disease, a delay in onset of the symptoms of a disease, or a lessening in the severity of subsequently developed disease symptoms. Typically, but not necessarily, since a prophylactic dose is used in subjects prior to or at an earlier stage of disease, the prophylactically effective amount is less than the therapeutically effective amount.

“Cancer” herein refers to or describes the physiological condition in mammals that is typically characterized by unregulated cell growth. Examples of cancer include but are not limited to carcinoma, lymphoma, blastoma, sarcoma (including liposarcoma, osteogenic sarcoma, angiosarcoma, endotheliosarcoma, leiomyosarcoma, chordoma, lymphangiosarcoma, lymphangioendotheliosarcoma, rhabdomyosarcoma, fibrosarcoma, myxosarcoma, chondrosarcoma), neuroendocrine tumors, mesothelioma, synovioma, schwannoma, meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwannoma, and other carcinomas, as well as head and neck cancer.

In certain embodiments, an oncolytic virus (e.g., an HSV), a viral stock, or a composition as described herein are used to treat a cancer selected from lung cancer (e.g., small cell lung cancer or non-small cell lung cancer), breast cancer, ovarian cancer, cervical cancer, prostate cancer, testicular cancer, colorectal cancer, colon cancer, pancreatic cancer, liver cancer (e.g., hepatocellular carcinoma (HCC)), gastric cancer, head and neck cancer, thyroid cancer, malignant glioma, glioblastoma, melanoma, non-melanoma skin cancer, B-cell chronic lymphocytic leukemia, diffuse large B-cell lymphoma (DLBCL), and marginal zone lymphoma (MZL). In some embodiments, the cancer is glioblastoma.

In some embodiments, the cancer is a non-melanoma skin cancer. Non-melanoma skin cancer refers to all the types of cancer that occur in the skin that are not melanoma. In some embodiments, the non-melanoma skin cancer is angiosarcoma, basal cell carcinoma, cutaneous B-cell lymphoma, cutaneous T-cell lymphoma, dermatofibrosarcoma protuberans, merkel cell carcinoma, sebaceous carcinoma, or squamous cell carcinoma of the skin.

In certain aspects, the disclosure relates to an oncolytic viral vector as shown in any one of the figures or embodiments disclosed herein.

### Further Numbered Embodiments

Further numbered embodiments of the present disclosure are provided as follows:

Embodiment 1. A recombinant herpesvirus, wherein the viral genome of the recombinant herpesvirus:
(a) comprises one or more transgenes, optionally wherein the open reading frame(s) (ORFs) of the transgene(s) have a G/C content of at least 60%, at least 61%, at least 62%, at least 63%, or at least 64%;
(b) comprises one or more miRNA target sequences;
(c) comprises a polynucleotide encoding a retargeting domain;
(d) comprises a UL30 viral gene encoding a DNA polymerase catalytic subunit (DPCS) and a UL23 viral gene encoding a thymidine kinase (TK), wherein the DPCS comprises a mutation, and wherein the TK comprises a mutation;
(e) encodes a first gB and/or a second gB, wherein the first gB comprises a syncytial mutation, and wherein the second gB comprises no syncytial mutation;
(f) encodes a first gK and/or a second gK, wherein the first gK comprises a syncytial mutation, and wherein the second gK comprises no syncytial mutation;
(g) encodes a first gH and/or a second gH, wherein the first gH comprises a syncytial mutation, and wherein the second gH comprises no syncytial mutation;
(h) encodes a first UL20 and/or a second UL20, wherein the first UL20 comprises a syncytial mutation, and wherein the second UL20 comprises no syncytial mutation; and/or
(i) encodes a first UL24 and/or a second UL24, wherein the first UL24 comprises a syncytial mutation, and wherein the second UL24 comprises no syncytial mutation.

Embodiment 2. The recombinant herpesvirus of Embodiment 1, wherein the viral genome of the recombinant herpesvirus comprises the one or more transgenes, wherein the one or more transgenes encode one or more payload proteins selected from 15-hydroxyprostaglandin dehydrogenase [NAD(+)] (HPGD), adenosine deaminase 2 (ADA2), hyaluronidase-1 (HYAL1), hemotaxis inhibitory protein (CHP), C-C motif chemokine 21 (CCL21), interleukin-12 (IL-12), a CD47 antagonist, a transforming growth factor beta (TGFβ) antagonist, a programmed death-1 (PD1) antagonist, a triggering receptor expressed on myeloid cells-2 (TREM2) antagonist, a biomolecule comprising chlorotoxin (CTX), or any combinations thereof.

Embodiment 3. The recombinant herpesvirus of Embodiment 2, wherein the one or more payload proteins comprise or consist of IL-12, a PD1 antagonist, and a TREM2 antagonist.

Embodiment 4. The recombinant herpesvirus of Embodiment 3, wherein the one or more payload proteins comprise HPGD.

Embodiment 5. The recombinant herpesvirus of Embodiment 3 or 4, wherein the one or more payload proteins comprise a biomolecule comprising CTX.

Embodiment 6. The recombinant herpesvirus of Embodiment 2, wherein the one or

more payload proteins comprise or consist of one of the combinations of payload proteins listed in Tables 4-7.

Embodiment 7. The recombinant herpesvirus of any one of Embodiments 2-6, wherein the one or more payload proteins comprise HPGD.

Embodiment 8. The recombinant herpesvirus of any one of Embodiments 2-7, wherein the one or more payload proteins comprise ADA2.

Embodiment 9. The recombinant herpesvirus of any one of Embodiments 2-8, wherein the one or more payload proteins comprise HYAL1.

Embodiment 10. The recombinant herpesvirus of any one of Embodiments 2-9, wherein the one or more payload proteins comprise CHP.

Embodiment 11. The recombinant herpesvirus of any one of Embodiments 2-10, wherein the one or more payload proteins comprise CCL21.

Embodiment 12. The recombinant herpesvirus of any one of Embodiments 2-11, wherein the one or more payload proteins comprise IL-12.

Embodiment 13. The recombinant herpesvirus of any one of Embodiments 2-12, wherein the one or more payload proteins comprise the CD47 antagonist.

Embodiment 14. The recombinant herpesvirus of any one of Embodiments 2-13, wherein the one or more payload proteins comprise the TGFβ antagonist.

Embodiment 15. The recombinant herpesvirus of any one of Embodiments 2-14, wherein the one or more payload proteins comprise the PD1 antagonist.

Embodiment 16. The recombinant herpesvirus of any one of Embodiments 2-15, wherein the one or more payload proteins comprise the TREM2 antagonist.

Embodiment 17. The recombinant herpesvirus of any one of Embodiments 2-16, wherein the antagonist comprises an antibody or antigen binding fragment thereof.

Embodiment 18. The recombinant herpesvirus of any one of Embodiments 2-17, wherein the one or more payload proteins comprise the biomolecule comprising CTX.

Embodiment 19. The recombinant herpesvirus of Embodiment 5 or 18, wherein the biomolecule comprising CTX further comprises a T-cell engager moiety specifically binding to a protein expressed on the surface of the T-cell.

Embodiment 20. The recombinant herpesvirus of Embodiment 19, wherein the protein expressed on the surface of the T-cell is CD3.

Embodiment 21. The recombinant herpesvirus of Embodiment 20, wherein the T-cell engager moiety comprises an amino acid sequence at least 90%, at least 95%, at least 97%, at least 98%, at least 99%, or 100% identical to SEQ ID NO: 914.

Embodiment 22. The recombinant herpesvirus of any one of Embodiments 18-21, wherein the CTX comprises or consists of an amino acid sequence at least 95% identical to SEQ ID NO: 913.

Embodiment 23. The recombinant herpesvirus of any one of Embodiments 2-22, wherein:
(i) the HPGD comprises an amino acid sequence at least 90%, at least 95%, at least 97%, at least 98%, at least 99%, or 100% identical to SEQ ID NO: 875;
(ii) the ADA2 comprises an amino acid sequence at least 90%, at least 95%, at least 97%, at least 98%, at least 99%, or 100% identical to SEQ ID NO: 877;
(iii) the HYAL1 comprises an amino acid sequence at least 90%, at least 95%, at least 97%, at least 98%, at least 99%, or 100% identical to SEQ ID NO: 878;
(iv) the CHP comprises an amino acid sequence at least 90%, at least 95%, at least 97%, at least 98%, at least 99%, or 100% identical to SEQ ID NO: 880;
(v) the CCL21 comprises an amino acid sequence at least 90%, at least 95%, at least 97%, at least 98%, at least 99%, or 100% identical to SEQ ID NO: 881;
(vi) the IL-12 comprises a subunit alpha comprising an amino acid sequence at least 90%, at least 95%, at least 97%, at least 98%, at least 99%, or 100% identical to SEQ ID NO: 883 and a subunit beta comprising an amino acid sequence at least 90%, at least 95%, at least 97%, at least 98%, at least 99%, or 100% identical to SEQ ID NO: 884;
(vii) the CD47 antagonist comprises VHH CDR1 of SEQ ID NO: 895, VHH CDR2 of SEQ ID NO: 896, VHH CDR3 of SEQ ID NO: 897, and/or an amino acid sequence at least 90%, at least 95%, at least 97%, at least 98%, at least 99%, or 100% identical to SEQ ID NO:

887 or 888;
(viii) the TGFβ antagonist comprises a heavy chain variable domain (VH) comprising CDR1 of SEQ ID NO: 898, CDR2 of SEQ ID NO: 899, and CDR3 of SEQ ID NO: 900, and/or a light chain variable domain (VL) comprising CDR1 of SEQ ID NO: 901, CDR2 of SEQ ID NO: 902, and CDR3 of SEQ ID NO: 903; optionally wherein the TGFβ antagonist comprises an amino acid sequence at least 90%, at least 95%, at least 97%, at least 98%, at least 99%, or 100% identical to SEQ ID NO: 889 or 890;
(ix) the PD1 antagonist comprises VHH CDR1 of SEQ ID NO: 904, VHH CDR2 of SEQ ID NO: 905, VHH CDR3 of SEQ ID NO: 906, and/or an amino acid sequence at least 90%, at least 95%, at least 97%, at least 98%, at least 99%, or 100% identical to SEQ ID NO: 891 or 892;
(x) the biomolecule comprising CTX comprises an amino acid sequence at least 90%, at least 95%, at least 97%, at least 98%, at least 99%, or 100% identical to SEQ ID NO: 915 or 916; and/or 891 or 892;
(xi) the TREM2 antagonist comprises a heavy chain variable domain (VH) comprising CDR1 of SEQ ID NO: 907, CDR2 of SEQ ID NO: 908, CDR3 of SEQ ID NO: 909, and/or an amino acid sequence at least 90%, at least 95%, at least 97%, at least 98%, at least 99%, or 100% identical to SEQ ID NO: 893; and/or a light chain variable domain (VL) comprising CDR1 of SEQ ID NO: 910, CDR2 of SEQ ID NO: 911, CDR3 of SEQ ID NO: 912, and/or an amino acid sequence at least 90%, at least 95%, at least 97%, at least 98%, at least 99%, or 100% identical to SEQ ID NO: 894.

Embodiment 24. The recombinant herpesvirus of any one of Embodiments 1-23, wherein the ORF of at least one of the transgene(s) has the G/C content of at least 60%, at least 61%, at least 62%, at least 63%, or at least 64%.

Embodiment 25. The recombinant herpesvirus of Embodiment 24, wherein the ORFs of all of the transgene(s) have the G/C content of at least 60%, at least 61%, at least 62%, at least 63%, or at least 64%.

Embodiment 26. The recombinant herpesvirus of Embodiment 24, wherein the ORFs of the transgene(s) encoding IL-12, the PD1 antagonist, the TREM2 antagonist, HPGD, and/or the biomolecule comprising CTX have the G/C content of at least 60%, at least 61%, at least 62%, at least 63%, or at least 64%.

Embodiment 27. The recombinant herpesvirus of any one of Embodiments 24-26, wherein the expression of a payload protein encoded by the ORF of the transgene is at least 2-fold, at least 3-fold, at least 5-fold, at least 8-fold, or at least 10-fold higher than the expression of the payload protein encoded by a control ORF having a G/C content of about 52% in a control recombinant herpesvirus; optionally wherein the control ORF is codon optimized based on the codon usage of Homo sapiens.

Embodiment 28. The recombinant herpesvirus of any one of Embodiments 24-27, wherein the ORF(s) of the transgene(s) are codon optimized based on the codon usage of Anaeromyxobacter dehalogenans.

Embodiment 29. The recombinant herpesvirus of any one of Embodiments 24-28, wherein the transgene(s) encode an antibody or antigen binding fragment thereof.

Embodiment 30. The recombinant herpesvirus of Embodiment 29, wherein the antibody or antigen binding fragment thereof comprises a heavy chain variable domain (VH) and a light chain variable domain (VL).

Embodiment 30.1 The recombinant herpesvirus of any one of Embodiments 23, 29 and 30, wherein the transgene encoding the TREM2 antagonist comprises a polynucleotide sequence at least 90%, at least 95%, at least 97%, at least 98%, at least 99%, or 100% identical to SEQ ID NO: 938.

Embodiment 31. The recombinant herpesvirus of Embodiment 29 or 30, wherein the antibody or antigen binding fragment thereof comprises a VHH domain derived from a single domain antibody (sdAb).

Embodiment 32. The recombinant herpesvirus of Embodiment 30 or 31, wherein the antibody or antigen binding fragment thereof comprises an IgG-Fc, optionally wherein the IgG is IgG1.

Embodiment 33. The recombinant herpesvirus of any one of Embodiments 23 and 29-32, wherein the transgene encoding the PD1 antagonist comprises a polynucleotide sequence at least 90%, at least 95%, at least 97%, at least 98%, at least 99%, or 100% identical to SEQ ID NO: 937.

Embodiment 34. The recombinant herpesvirus of any one of Embodiments 23 and 29-33, wherein the transgene encoding the biomolecule comprising CTX comprises a polynucleotide sequence at least 90%, at least 95%, at least 97%, at least 98%, at least 99%, or 100% identical to SEQ ID NO: 940 or 941.

Embodiment 35. The recombinant herpesvirus of any one of Embodiments 24-34, comprising the transgene(s) encoding a cytokine, a chemokine, a receptor, a receptor ligand, an enzyme, and/or a reporter protein.

Embodiment 36. The recombinant herpesvirus of Embodiment 23 or 35, wherein the transgene encoding IL-12 comprise a polynucleotide sequence at least 90%, at least 95%, at least 97%, at least 98%, at least 99%, or 100% identical to SEQ ID NO: 936.

Embodiment 37. The recombinant herpesvirus of any one of Embodiments 23 and 35-36, wherein the transgene encoding HPGD comprises a polynucleotide sequence at least 90%, at least 95%, at least 97%, at least 98%, at least 99%, or 100% identical to SEQ ID NO: 939.

Embodiment 38. The recombinant herpesvirus of any one of Embodiments 1-37, comprising the miRNA target sequences for miR-34b-5p, miR-34b-3p, miR-34c-5p, miR-122-5p, miR-124-3p, miR-128T, miR-129-5p, miR-129-2-3p, miR-132-3p, miR-137-3p, miR-145-5p, or any combination thereof.

Embodiment 39. The recombinant herpesvirus of Embodiment 38, comprising the miRNA target sequences for miR-34b-5p, miR-34c-5p, miR-122-5p, miR-124-3p, miR-128T, miR-129-2-3p, miR-132-3p, miR-137-3p, miR-145-5p, or any combination thereof.

Embodiment 40. The recombinant herpesvirus of Embodiment 38, comprising the miRNA target sequences for miR-34b-5p, miR-34c-5p, miR-122-5p, miR-124-3p, miR-128T, miR-129-2-3p, miR-132-3p, miR-137-3p, and miR-145-5p.

Embodiment 41. The recombinant herpesvirus of any one of Embodiments 38-40, comprising:
(a) one or more miRNA target sequences for miR-122-5p;
(b) one or more miRNA target sequences for miR-145-5p;
(c) one or more miRNA target sequences for miR-124-3p, miR-128T, miR-137-3p, or any combination thereof;
(d) one or more miRNA target sequences for miR-34b-5p, miR-34c-5p, or any combination thereof; and
(e) one or more miRNA target sequences for miR-129-2-3p, miR-132-3p, or any combination thereof.

Embodiment 42. The recombinant herpesvirus of any one of Embodiments 38-41, comprising a first miR-TS cassette inserted into a first viral gene, wherein the first miR-TS cassette comprises one or more miRNA target sequences for each of miR-34c-5p, miR-124-3p, miR-129-2-3p, and miR-132-3p.

Embodiment 43. The recombinant herpesvirus of Embodiment 42, wherein the miRNA target sequences in the first miR-TS cassette are arranged as (34c-5p)-(124-3p)-(132-3p)-(129-2-3p)-(34c-5p)-(124-3p)-(129-2-3p)-(132-3p)-(124-3p)-(129-2-3p)-(132-3p)-(34c-5p).

Embodiment 44. The recombinant herpesvirus of Embodiment 42 or 43, wherein the first miR-TS cassette comprises a nucleic acid sequence at least 90%, at least 95%, at least 96%, at least 97%, at least 98%, at least 99%, or 100% identical to SEQ ID NO: 859.

Embodiment 45. The recombinant herpesvirus of any one of Embodiments 42-44, wherein the first viral gene is ICP8.

Embodiment 46. The recombinant herpesvirus of any one of Embodiments 38-45, comprising a second miR-TS cassette inserted into a second viral gene, wherein the second miR-TS cassette comprises one or more miRNA target sequences for each of miR-122-5p, miR-124-3p, miR-128T, and miR-137-3p.

Embodiment 47. The recombinant herpesvirus of Embodiment 46, wherein the miRNA target sequences in the second miR-TS cassette are arranged as (137-3p)-(128T)-(122-5p)-(124-3p)-(122-5p)-(128T)-(137-3p)-(124-3p)-(128T)-(137-3p)-(124-3p)-(122-5p).

Embodiment 48. The recombinant herpesvirus of Embodiment 46 or 47, wherein the second miR-TS cassette comprises a nucleic acid sequence at least 90%, at least 95%, at least 96%, at least 97%, at least 98%, at least 99%, or 100% identical to SEQ ID NO: 858.

Embodiment 49. The recombinant herpesvirus of any one of Embodiments 46-48, wherein the second viral gene is ICP4.

Embodiment 50. The recombinant herpesvirus of Embodiments 49, comprising the second miR-TS cassette in both ICP4 viral genes of the viral genome.

Embodiment 51. The recombinant herpesvirus of any one of Embodiments 1-50, comprising one or more miRNA target sequences in both ICP4 viral genes of the viral genome; optionally wherein the miRNA target sequences are the same in both said ICP4 viral genes.

Embodiment 52. The recombinant herpesvirus of any one of Embodiments 38-51, comprising a third miR-TS cassette inserted into a third viral gene, wherein the third miR-TS cassette comprises one or more miRNA target sequences for each of miR-34c-5p, miR-124-3p, miR-128T, and miR-137-3p.

Embodiment 53. The recombinant herpesvirus of Embodiment 52, wherein the miRNA target sequences in the third miR-TS cassette are arranged as (124-3p)-(128T)-(34c-5p)-(137-3p)-(128T)-(34c-5p)-(137-3p)-(124-3p)-(128T)-(137-3p)-(124-3p)-(34c-5p).

Embodiment 54. The recombinant herpesvirus of Embodiment 52 or 53, wherein the third miR-TS cassette comprises a nucleic acid sequence at least 90%, at least 95%, at least 96%, at least 97%, at least 98%, at least 99%, or 100% identical to SEQ ID NO: 873.

Embodiment 55. The recombinant herpesvirus of any one of Embodiments 38-51, comprising a third miR-TS cassette inserted into a third viral gene, wherein the third miR-TS cassette comprises one or more miRNA target sequences for each of miR-34b-3p, miR-34c-5p, miR-128T, miR-137-3p.

Embodiment 56. The recombinant herpesvirus of any one of Embodiments 52-55, wherein the third viral gene is ICP27.

Embodiment 57. The recombinant herpesvirus of any one of Embodiments 38-56, comprising a fourth miR-TS cassette inserted into a fourth viral gene, wherein:
(i) the fourth miR-TS cassette comprises one or more miRNA target sequences for each of miR-34b-5p, miR-34c-5p, miR-129-5p, and miR-145-5p;
(ii) the fourth miR-TS cassette comprises one or more miRNA target sequences for each of miR-34b-5p, miR-34c-5p, miR-132-3p, and miR-145-5p; or
(iii) the fourth miR-TS cassette comprises one or more miRNA target sequences for each of miR-34b-3p, miR-34c-5p, miR-132-3p, and miR-145-5p.

Embodiment 58. The recombinant herpesvirus of any one of Embodiments 38-56, comprising a fourth miR-TS cassette inserted into a fourth viral gene, wherein the fourth miR-TS cassette comprises one or more miRNA target sequences for each of miR-34b-5p, miR-34c-5p, miR-132-3p, and miR-145-5p.

Embodiment 59. The recombinant herpesvirus of Embodiment 58, wherein the miRNA target sequences in the fourth miR-TS cassette are arranged as (145-5p)-(34b-5p)-(132-3p)-(34c-5p)-(145-5p)-(34c-5p)-(34b-5p)-(132-3p)-(34b-5p)-(145-5p)-(132-3p)-(34c-5p).

Embodiment 60. The recombinant herpesvirus of Embodiment 58 or 59, wherein the fourth miR-TS cassette comprises a nucleic acid sequence at least 90%, at least 95%, at least 96%, at least 97%, at least 98%, at least 99%, or 100% identical to SEQ ID NO: 874.

Embodiment 61. The recombinant herpesvirus of any one of Embodiments 57-60, wherein the fourth viral gene is UL8.

Embodiment 62. The recombinant herpesvirus of any one of Embodiments 42-61, wherein each of the miR-TS cassettes comprises at least 2, at least 3, or at least 4 copies of each of the miRNA target sequences.

Embodiment 63. The recombinant herpesvirus of any one of Embodiments 42-61, wherein each of the miR-TS cassettes comprises 3 copies of each of the miRNA target sequences.

Embodiment 64. The recombinant herpesvirus of any one of Embodiments 38-63, wherein the replication of the recombinant HSV is reduced in a non-cancerous cell compared to the replication of the recombinant HSV in a cancerous cell; optionally wherein the cancerous cell is a glioblastoma cell.

Embodiment 65. The recombinant herpesvirus of Embodiment 64, wherein the non-cancerous cell is selected from the group consisting of a neuron, an ependymal cell, an oligodendrocyte, an endothelial cell, a hepatocyte, an astrocyte, and any combination thereof.

Embodiment 66. The recombinant herpesvirus of Embodiment 64, wherein the non-cancerous cell is an astrocyte.

Embodiment 67. The recombinant herpesvirus of any one of Embodiments 38-66, wherein:
(a) the one or more miRNA target sequences for miR-34b-5p comprise or consist of SEQ ID NO: 867;
(b) the one or more miRNA target sequences for miR-34b-3p comprise or consist of SEQ ID NO: 868;
(c) the one or more miRNA target sequences for miR-34c-5p comprise or consist of SEQ ID NO: 869;
(d) the one or more miRNA target sequences for miR-122-5p comprise or consist of SEQ ID NO: 804;
(e) the one or more miRNA target sequences for miR-124-3p comprise or consist of SEQ ID NO: 805;
(f) the one or more miRNA target sequences for miR-128T comprise or consist of SEQ ID NO: 870;
(g) the one or more miRNA target sequences for miR-129-5p comprise or consist of SEQ ID NO: 813;
(h) the one or more miRNA target sequences for miR-129-2-3p comprise or consist of SEQ ID NO: 871;
(i) the one or more miRNA target sequences for miR-132-3p comprise or consist of SEQ ID NO: 872;
(j) the one or more miRNA target sequences for miR-137-3p comprise or consist of SEQ ID NO: 819; and/or
(k) the one or more miRNA target sequences for miR-145-5p comprise or consist of SEQ ID NO: 823.

Embodiment 68. The recombinant herpesvirus of any one of Embodiments 1-67, comprising the polynucleotide encoding the retargeting domain, wherein the retargeting domain specifically binds a target protein expressed by a target cell.

Embodiment 69. The recombinant herpesvirus of Embodiment 68, wherein the polynucleotide encoding the retargeting domain is inserted into the open reading frame of a US6 gene encoding a glycoprotein D (gD).

Embodiment 70. The recombinant herpesvirus of Embodiment 69, wherein the polynucleotide encoding the retargeting domain replaces the US6 gene region encoding an amino acid sequence corresponding to amino acids 6-24 of SEQ ID NO: 921.

Embodiment 71. The recombinant herpesvirus of any one of Embodiments 68-70, wherein the target protein expressed by the target cell comprises integrin α5β1, integrin αvβ1, integrin αvβ3, integrin αvβ6, or a combination thereof.

Embodiment 72. The recombinant herpesvirus of any one of Embodiments 68-71, wherein the target protein expressed by the target cell comprises epidermal growth factor receptor (EGFR).

Embodiment 73. The recombinant herpesvirus of any one of Embodiments 68-72, wherein the retargeting domain comprises a knottin peptide capable of specifically binding to the target protein expressed by the target cell.

Embodiment 74. The recombinant herpesvirus of Embodiment 73, wherein the retargeting domain comprises no more than 50, no more than 45, no more than 40, or no more than 35 amino acids.

Embodiment 75. The recombinant herpesvirus of Embodiment 73 or 74, wherein the retargeting domain comprises an amino acid sequence having at least 80%, at least 85%, at least 90%, or 100% identity to SEQ ID NO: 922.

Embodiment 76. The recombinant herpesvirus of any one of Embodiments 68-75, wherein the retargeting domain comprises an immunoglobulin domain capable of specifically binding to the target protein expressed by the target cell.

Embodiment 77. The recombinant herpesvirus of any one of Embodiments 68-76, wherein the retargeting domain comprises a binding domain of, or a binding domain derived from, a variable domain of a heavy chain-only antibody (VHH) or a variable domain of new antigen receptor immunoglobulin (V-NAR).

Embodiment 78. The recombinant herpesvirus of Embodiment 76 or 77, wherein the retargeting domain comprises no more than 150, no more than 140, or no more than 130 amino acids.

Embodiment 79. The recombinant herpesvirus of any one of Embodiments 76-78, wherein the retargeting domain comprises an amino acid sequence having at least 80%, at least 85%, at least 90%, or 100% identity to SEQ ID NO: 923.

Embodiment 80. The recombinant herpesvirus of any one of Embodiments 68-79, wherein the herpesvirus is capable of infecting the target cell expressing the target protein.

Embodiment 81. The recombinant herpesvirus of any one of Embodiments 68-80, wherein the herpesvirus is capable of infecting cells without Nectin-1 expression; optionally the cells are Vero cells.

Embodiment 82. The recombinant herpesvirus of any one of Embodiments 1-81, comprising the UL30 viral gene encoding the DPCS comprising the mutation and the UL23 viral gene encoding the TK comprising the mutation.

Embodiment 83. The recombinant herpesvirus of Embodiment 82, wherein the mutation in the DPCS increases DNA replication fidelity of the herpesvirus by at least 10%, at least 20%, at least 30%, at least 50%, at least 100%, at least 2-fold, at least 3-fold, or at least 5-fold.

Embodiment 84. The recombinant herpesvirus of Embodiment 82 or 83, wherein the mutation in the DPCS is at an amino acid position corresponding to L774 of SEQ ID NO: 917; preferably, the mutation is an amino acid substitution.

Embodiment 85. The recombinant herpesvirus of Embodiment 84, wherein the mutation in the DPCS is the amino acid substitution corresponding to L774F of SEQ ID NO: 917.

Embodiment 86. The recombinant herpesvirus of any one of Embodiments 82-85, wherein the DPCS comprises an amino acid sequence at least 90%, at least 95%, at least 97%, at least 98%, at least 99%, or 100% identical to SEQ ID NO: 917, except for the mutation in the DPCS.

Embodiment 87. The recombinant herpesvirus of any one of Embodiments 82-86, wherein the IC50 of acyclovir is less than 0.5 ug/ml, less than 1.0 ug/ml, less than 1.5 ug/ml, or less than 2.0 ug/ml for the herpesvirus.

Embodiment 88. The recombinant herpesvirus of any one of Embodiments 82-87, wherein the mutation in the TK decreases the IC50 of acyclovir for the herpesvirus by at least 1-fold, at least 2-fold, at least 5-fold, at least 10-fold, at least 20-fold, at least 50-fold, or at least 100-fold.

Embodiment 89. The recombinant herpesvirus of any one of Embodiments 82-88, wherein the mutation in the TK is at one or more amino acid positions corresponding to L159, 1160, F161, A168 and/or L169 of SEQ ID NO: 918; preferably, the mutation is amino acid substitution.

Embodiment 90. The recombinant herpesvirus of Embodiment 89, wherein the mutation in the TK comprises one or more amino acid substitutions of:
(a) L159I or L159L;
(b) I160L or I160F;
(c) F161A, F161V, F161P, or F161L;
(d) A168D, A168Y, A168V or A168F; and/or
(e) L169F, L169Y, L169L, L169I, L169M, L169N, or L169K.

Embodiment 91. The recombinant herpesvirus of any one of Embodiments 82-90, wherein the mutation in the TK comprises amino acid substitutions corresponding to L159I, I160F, F161L, A168F and L169M of SEQ ID NO: 918.

Embodiment 92. The recombinant herpesvirus of any one of Embodiments 82-90, wherein the mutation in the TK comprises amino acid substitutions corresponding to I160F, F161A, and A168F of SEQ ID NO: 918.

Embodiment 93. The recombinant herpesvirus of any one of Embodiments 82-90, wherein the mutation in the TK comprises amino acid substitutions corresponding to I160F, F161L, A168F, and L169N of SEQ ID NO: 918.

Embodiment 94. The recombinant herpesvirus of any one of Embodiments 82-93, wherein the TK comprises an amino acid sequence at least 90%, at least 95%, at least 97%, at least 98%, at least 99%, or 100% identical to SEQ ID NO: 918, except the mutation in the TK.

Embodiment 95. The recombinant herpesvirus of any one of Embodiments 1-94, wherein the viral genome of the herpesvirus encodes:
(i) the first gB and/or the second gB, wherein the first gB comprises the syncytial mutation, and wherein the second gB comprises no syncytial mutation;
(ii) the first gK and/or the second gK, wherein the first gK comprises the syncytial mutation, and wherein the second gK comprises no syncytial mutation;
(iii) the first gH and/or the second gH, wherein the first gH comprises the syncytial mutation, and wherein the second gH comprises no syncytial mutation;
(iv) the first UL20 and/or the second UL20, wherein the first UL20 comprises the syncytial mutation, and wherein the second UL20 comprises no syncytial mutation; and/or
(v) the first UL24 and/or the second UL24, wherein the first UL24 comprises the syncytial mutation, and wherein the second UL24 comprises no syncytial mutation.

Embodiment 96. The recombinant herpesvirus of Embodiment 95, wherein the first gB is encoded by an endogenous gB-encoding gene locus and the second gB is encoded by an exogenous expression cassette.

Embodiment 97. The recombinant herpesvirus of Embodiment 95, wherein the first gB is encoded by an exogenous expression cassette and the second gB is encoded by an endogenous gB-encoding gene locus.

Embodiment 98. The recombinant herpesvirus of any one of Embodiments 95-97, wherein the first gK is encoded by an endogenous gK-encoding gene locus and the second gK is encoded by an exogenous expression cassette.

Embodiment 99. The recombinant herpesvirus of any one of Embodiments 95-97, wherein the first gK is encoded by an exogenous expression cassette and the second gK is encoded by an endogenous gK-encoding gene locus.

Embodiment 100. The recombinant herpesvirus of any one of Embodiments 95-99, wherein the first gH is encoded by an endogenous gH-encoding gene locus and the second gH is encoded by an exogenous expression cassette.

Embodiment 101. The recombinant herpesvirus of any one of Embodiments 95-99, wherein the first gH is encoded by an exogenous expression cassette and the second gH is encoded by an endogenous gH-encoding gene locus.

Embodiment 102. The recombinant herpesvirus of any one of Embodiments 95-101, wherein the first UL20 is encoded by an endogenous UL20 gene locus and the second UL20 is encoded by an exogenous expression cassette.

Embodiment 103. The recombinant herpesvirus of any one of Embodiments 95-101, wherein the first UL20 is encoded by an exogenous expression cassette and the second UL20 is encoded by an endogenous UL20 gene locus.

Embodiment 104. The recombinant herpesvirus of any one of Embodiments 95-103, wherein the first UL24 is encoded by an endogenous UL24 gene locus and the second UL24 is encoded by an exogenous expression cassette.

Embodiment 105. The recombinant herpesvirus of any one of Embodiments 95-103, wherein the first UL24 is encoded by an exogenous expression cassette and the second UL24 is encoded by an endogenous UL24 gene locus.

Embodiment 106. The recombinant herpesvirus of any one of Embodiments 95-105, wherein the viral genome of the herpesvirus encodes the first gB but not the second gB.

Embodiment 107. The recombinant herpesvirus of any one of Embodiments 95-106, wherein the viral genome of the herpesvirus encodes the first gK but not the second gK.

Embodiment 108. The recombinant herpesvirus of any one of Embodiments 95-107, wherein the viral genome of the herpesvirus encodes the first gH but not the second gH.

Embodiment 109. The recombinant herpesvirus of any one of Embodiments 95-108, wherein the viral genome of the herpesvirus encodes the first UL20 but not the second UL20

Embodiment 110. The recombinant herpesvirus of any one of Embodiments 95-109, wherein the viral genome of the herpesvirus encodes the first UL24 but not the second UL24.

Embodiment 111. The recombinant herpesvirus of any one of Embodiments 95-110, wherein the viral genome of the herpesvirus encodes the first gB and the first gK; optionally, wherein the viral genome of the herpesvirus further encodes the first gH and the first UL24.

Embodiment 112. The recombinant herpesvirus of any one of Embodiments 96-111, wherein the exogenous expression cassette is located at UL3-UL4 intergenic region.

Embodiment 113. The recombinant herpesvirus of any one of Embodiments 96-111, wherein the exogenous expression cassette is located at UL50-UL51 intergenic region.

Embodiment 114. The recombinant herpesvirus of any one of Embodiments 95-113, wherein the recombinant herpesvirus displays syncytial phenotype in cancer cells.

Embodiment 115. A cell, comprising a recombinant nucleic acid encoding the recombinant herpesvirus of any one of Embodiments 95-114.

Embodiment 116. A cell, comprising a first nucleic acid encoding a recombinant herpesvirus and a second nucleic acid, wherein:
(i) the viral genome of the herpesvirus encodes a first gB, wherein the first gB comprises a syncytial mutation, the second nucleic acid encodes a second gB, wherein the second gB comprises no syncytial mutation; or, the viral genome of the herpesvirus encodes a second gB, wherein the second gB comprises no syncytial mutation, the second nucleic acid encodes a first gB, wherein the first gB comprises a syncytial mutation;
(ii) the viral genome of the herpesvirus encodes a first gK, wherein the first gK comprises a syncytial mutation, the second nucleic acid encodes a second gK, wherein the second gK comprises no syncytial mutation; or the viral genome of the herpesvirus encodes a second gK, wherein the second gK comprises no syncytial mutation, the second nucleic acid encodes a first gK, wherein the first gK comprises a syncytial mutation;
(iii) the viral genome of the herpesvirus encodes a first gH, wherein the first gH comprises a syncytial mutation, the second nucleic acid encodes a second gH, wherein the second gH comprises no syncytial mutation; or the viral genome of the herpesvirus encodes a second gH, wherein the second gH comprises no syncytial mutation, the second nucleic acid encodes a first gH, wherein the first gH comprises a syncytial mutation;
(iv) the viral genome of the herpesvirus encodes a first UL20, wherein the first UL20 comprises a syncytial mutation, the second nucleic acid encodes a second UL20, wherein the second UL20 comprises no syncytial mutation; or the viral genome of the herpesvirus encodes a second UL20, wherein the second UL20 comprises no syncytial mutation, the second nucleic acid encodes a first UL20, wherein the first UL20 comprises a syncytial mutation; and/or
(v) the viral genome of the herpesvirus encodes a first UL24, wherein the first UL24 comprises a syncytial mutation, the second nucleic acid encodes a second UL24, wherein the second UL24 comprises no syncytial mutation; or the viral genome of the herpesvirus encodes a second UL24, wherein the second UL24 comprises no syncytial mutation, the second nucleic acid encodes a first UL24, wherein the first UL24 comprises a syncytial mutation.

Embodiment 117. The cell of Embodiment 116, wherein the viral genome of the herpesvirus encodes the first gB, wherein the first gB comprises the syncytial mutation, wherein the second nucleic acid encodes the second gB, wherein the second gB comprises no syncytial mutation.

Embodiment 118. The cell of Embodiment 116, wherein the viral genome of the herpesvirus encodes the second gB, wherein the second gB comprises no syncytial mutation, wherein the second nucleic acid encodes the first gB, wherein the first gB comprises the syncytial mutation.

Embodiment 119. The cell of any one of Embodiments 116-118, wherein the viral genome of the herpesvirus encodes the first gK, wherein the first gK comprises the syncytial mutation, wherein the second nucleic acid encodes the second gK, wherein the second gK comprises no syncytial mutation.

Embodiment 120. The cell of any one of Embodiments 116-118, wherein the viral genome of the herpesvirus encodes the second gK, wherein the second gK comprises no syncytial mutation, wherein the second nucleic acid encodes the first gK, wherein the first gK comprises the syncytial mutation.

Embodiment 121. The cell of any one of Embodiments 116-120, wherein the viral genome of the herpesvirus encodes the first gH, wherein the first gH comprises the syncytial mutation, wherein the second nucleic acid encodes the second gH, wherein the second gH comprises no syncytial mutation.

Embodiment 122. The cell of any one of Embodiments 116-120, wherein the viral genome of the herpesvirus encodes the second gH, wherein the second gH comprises no syncytial mutation, wherein the second nucleic acid encodes the first gH, wherein the first gH comprises the syncytial mutation.

Embodiment 123. The cell of any one of Embodiments 116-122, wherein the viral genome of the herpesvirus encodes the first UL20, wherein the first UL20 comprises the syncytial mutation, wherein the second nucleic acid encodes the second UL20, wherein the second UL20 comprises no syncytial mutation.

Embodiment 124. The cell of any one of Embodiments 116-122, wherein the viral genome of the herpesvirus encodes the second UL20, wherein the second UL20 comprises no syncytial mutation, wherein the second nucleic acid encodes the first UL20, wherein the first UL20 comprises the syncytial mutation.

Embodiment 125. The cell of any one of Embodiments 116-124, wherein the viral genome of the herpesvirus encodes the first UL24, wherein the first UL24 comprises the syncytial mutation, wherein the second nucleic acid encodes the second UL24, wherein the second UL24 comprises no syncytial mutation.

Embodiment 126. The cell of any one of Embodiments 116-124, wherein the viral genome of the herpesvirus encodes the second UL24, wherein the second UL24 comprises no syncytial mutation, wherein the second nucleic acid encodes the first UL24, wherein the first UL24 comprises the syncytial mutation.

Embodiment 127. The cell of any of Embodiments 116-126, wherein the recombinant herpesvirus comprises a single copy of gB-encoding viral gene, a single copy of gK-encoding viral gene, a single copy of gH-encoding viral gene, a single copy of UL20 viral gene, and/or a single copy of UL24 viral gene.

Embodiment 128. The cell of any of Embodiments 116-127, wherein the first nucleic acid and the second nucleic acid are comprised within a single polynucleotide molecule.

Embodiment 129. The cell of any of Embodiments 116-127, wherein the first nucleic acid and the second nucleic acid are comprised within two different polynucleotide molecules.

Embodiment 130. The cell of any of Embodiments 115-129, wherein the cell is a Vero cell.

Embodiment 131. The recombinant herpesvirus of any of Embodiments 95-114 or the cell of any of Embodiments 115-130, wherein the gB syncytial mutation comprises a mutation at one or more amino acid residues corresponding to R796, R800, T813, L817, S854, A855, R858, or A874, an insertion between E816 and L817, a deletion of S869 to C-terminus, a deletion of T877 to C-terminus, or a combination thereof, of SEQ ID NO: 919.

Embodiment 132. The recombinant herpesvirus of any of Embodiments 95-114 or the cell of any of Embodiments 115-130, wherein the gB syncytial mutation comprises one or more mutations corresponding to R796C, R800W, T813I, L817H, L817P, S854F, A855V, R858C, R858H, A874P, an insertion of VN or VNVN between E816 and L817, a deletion of S869 to C-terminus, or a deletion of T877 to C-terminus, of SEQ ID NO: 919.

Embodiment 133. The recombinant herpesvirus of any of Embodiments 95-114 or the cell of any of Embodiments 115-130, wherein the gB syncytial mutation comprises a deletion of T877 to C-terminus according to SEQ ID NO: 919.

Embodiment 134. The recombinant herpesvirus of any of Embodiments 95-114 and 131-133, or the cell of any of Embodiments 115-133, wherein the first and/or the second gB comprise a mutation corresponding to D285N and/or A549T of SEQ ID NO: 919.

Embodiment 135. The recombinant herpesvirus of any of Embodiments 95-114 and 131-134, or the cell of any of Embodiments 115-134, wherein the gK syncytial mutation comprises a mutation at one or more amino acid residues corresponding to P33, A40, L86, D99, A111, L118, T121, C243, L304, 1307, or R310 of SEQ ID NO: 920.

Embodiment 136. The recombinant herpesvirus of any of Embodiments 95-114 and 131-134, or the cell of any of Embodiments 115-134, wherein the gK syncytial mutation comprises one or more mutations corresponding to P33S, A40V, A40T, L86P, D99N, A111V, L118Q, T121I, C243Y, L304P, 1307N, or R310L of SEQ ID NO: 920.

Embodiment 137. The recombinant herpesvirus of any of Embodiments 95-114 and 131-134, or the cell of any of Embodiments 115-134, wherein the gK syncytial mutation comprises 1307N according to SEQ ID NO: 920.

Embodiment 138. The recombinant herpesvirus of any of Embodiments 95-114 and 131-137, or the cell of any of Embodiments 115-137, wherein the gH syncytial mutation comprises a mutation at one or more amino acid residues corresponding to N753 or A778 of SEQ ID NO: 943.

Embodiment 139. The recombinant herpesvirus of any of Embodiments 95-114 and 131-137, or the cell of any of Embodiments 115-137, wherein the gH syncytial mutation comprises one or more mutations corresponding to N753K or A778V of SEQ ID NO: 943.

Embodiment 140. The recombinant herpesvirus of any of Embodiments 95-114 and 131-139, or the cell of any of Embodiments 115-139, wherein the UL20 syncytial mutation comprises a mutation at one or more amino acid residues corresponding to Y49, S50, R51, R209, T212, R213, or C-terminal deletion after N217, of SEQ ID NO: 944.

Embodiment 141. The recombinant herpesvirus of any of Embodiments 95-114 and 131-139, or the cell of any of Embodiments 115-139, wherein the UL20 syncytial mutation comprises one or more mutations corresponding to Y49A, S50A, R51A, R209A, T212A, R213A, or C-terminal deletion after N217, of SEQ ID NO: 944.

Embodiment 142. The recombinant herpesvirus of any of Embodiments 95-114 and 131-141, or the cell of any of Embodiments 115-141, wherein the UL24 syncytial mutation comprises a mutation at one or more amino acid residues corresponding to T64, R63, or V64 of SEQ ID NO: 942.

Embodiment 143. The recombinant herpesvirus of any of Embodiments 95-114 and 131-141, or the cell of any of Embodiments 115-141, wherein the UL24 syncytial mutation comprises one or more mutations corresponding to T64G, R63V, or V64S of SEQ ID NO: 942.

Embodiment 144. The recombinant herpesvirus of any of Embodiments 95-114 and 131-143, or the cell of any of Embodiments 115-143, wherein the open reading frame encoding the first gB is operably linked to a CMV promoter and/or a bGH poly A tail.

Embodiment 145. The recombinant herpesvirus of any of Embodiments 95-114 and 131-143, or the cell of any of Embodiments 115-143, wherein the open reading frame encoding the second gB is operably linked to a CMV promoter and/or a bGH poly A tail.

Embodiment 146. The recombinant herpesvirus of any of Embodiments 95-114 and 131-145, or the cell of any of Embodiments 115-145, wherein the open reading frame encoding the first gK is operably linked to a CMV promoter and/or a bGH poly A tail.

Embodiment 147. The recombinant herpesvirus of any of Embodiments 95-114 and 131-145, or the cell of any of Embodiments 115-145, wherein the open reading frame encoding the second gK is operably linked to a CMV promoter and/or a bGH polyA tail.

Embodiment 148. The recombinant herpesvirus of any of Embodiments 95-114 and 131-147, or the cell of any of Embodiments 115-147, wherein the yield of the recombinant herpesvirus is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than the yield of a control herpesvirus or a control cell that does not encode the second gB, the second gK, the second gH, the second UL20, or the second UL24.

Embodiment 149. The recombinant herpesvirus of any of Embodiments 95-114 and 131-148, or the cell of any of Embodiments 115-148, wherein the gene encoding the first gB comprises a miRNA target sequence (miR-TS) cassette, wherein the miR-TS cassette comprises one or more target sequences of one or more miRNAs.

Embodiment 150. The recombinant herpesvirus of any of Embodiments 95-114 and 131-149, or the cell of any of Embodiments 115-149, wherein the gene encoding the first gK comprises a miRNA target sequence (miR-TS) cassette, wherein the miR-TS cassette comprises one or more target sequences of one or more miRNAs.

Embodiment 151. The recombinant herpesvirus of any of Embodiments 95-114 and 131-150, or the cell of any of Embodiments 115-150, wherein the gene encoding the first gH comprises a miRNA target sequence (miR-TS) cassette, wherein the miR-TS cassette comprises one or more target sequences of one or more miRNAs.

Embodiment 152. The recombinant herpesvirus of any of Embodiments 95-114 and 131-151, or the cell of any of Embodiments 115-151, wherein the gene encoding the first UL20 comprises a miRNA target sequence (miR-TS) cassette, wherein the miR-TS cassette comprises one or more target sequences of one or more miRNAs.

Embodiment 153. The recombinant herpesvirus of any of Embodiments 95-114 and 131-152, or the cell of any of Embodiments 115-152, wherein the gene encoding the first UL24 comprises a miRNA target sequence (miR-TS) cassette, wherein the miR-TS cassette comprises one or more target sequences of one or more miRNAs.

Embodiment 154. The recombinant herpesvirus of any one of Embodiments 149-153, or the cell of any one of Embodiments 149-153, wherein the one or more miRNAs comprise at least one of miR-34c-5p, miR-299-5p, and miR-582-5p.

Embodiment 155. The recombinant herpesvirus of any one of Embodiments 149-153, or the cell of any one of Embodiments 149-153, wherein the one or more miRNAs comprise at least two of miR-34c-5p, miR-299-5p, and miR-582-5p.

Embodiment 156. The recombinant herpesvirus of any one of Embodiments 149-153, or the cell of any one of Embodiments 149-153, wherein the one or more miRNAs comprise miR-34c-5p, miR-299-5p, and miR-582-5p.

Embodiment 157. The recombinant herpesvirus or the cell of any of Embodiments 149-156, wherein the miR-TS cassette comprises at least three copies, or at least four copies of the target sequences of each of the miRNA separated by a 4 bp spacer.

Embodiment 158. The recombinant herpesvirus or the cell of any of Embodiments 149-157, wherein the miR-TS cassette is located at the 3′UTR of the gene.

Embodiment 159. The recombinant herpesvirus or the cell of any of Embodiments 149-158, wherein the target sequence of the miRNA comprises or consists of the reverse complement of the miRNA.

Embodiment 160. The recombinant herpesvirus or the cell of any of Embodiments 149-159, wherein the miR-TS cassette comprises the polynucleotide sequence of SEQ ID NO: 930.

Embodiment 161. The recombinant herpesvirus or the cell of any of Embodiments 149-160, wherein the yield of the recombinant herpesvirus is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than the yield of a control herpesvirus or a control cell that does not comprise the miR-TS cassette.

Embodiment 162. A recombinant herpesvirus produced by culturing the cell of any of Embodiments 115-161 and recovering the recombinant herpesvirus from the cell culture.

Embodiment 163. A recombinant herpesvirus, wherein the viral genome of the herpesvirus encodes a gK comprising a syncytial mutation corresponding to 1307N of SEQ ID NO: 920.

Embodiment 164. The recombinant herpesvirus of any one of Embodiments 1-114 and 131-163, or the cell of any of Embodiments 115-161, wherein the herpesvirus is an alphaherpesvirus.

Embodiment 165. The recombinant herpesvirus or the cell of Embodiment 164, wherein the alphaherpesvirus is a herpes simplex virus.

Embodiment 166. The recombinant herpesvirus or the cell of Embodiment 165, wherein the herpes simplex virus is a herpes simplex virus-1 (HSV-1).

Embodiment 167. The recombinant herpesvirus of any one of Embodiments 1-114 and 131-166, or the cell of any of Embodiments 115-161 and 164-166, wherein the recombinant herpesvirus is oncolytic.

Embodiment 168. The recombinant herpesvirus or the cell of any one of Embodiments 164-167, wherein the recombinant herpesvirus is derived from an encephalitic HSV isolate according to SEQ ID NO: 857; optionally wherein the recombinant herpesvirus comprises a polynucleotide sequence having at least 80%, at least 85%, at least 90%, at least 95%, at least 96%, at least 97%, at least 98%, at least 99%, at least 99.5%, or 100% identity to SEQ ID NO: 857.

Embodiment 169. The recombinant herpesvirus or the cell of any one of Embodiments 164-168, wherein the recombinant herpesvirus is defective for anterograde transport.

Embodiment 170. The recombinant herpesvirus of any one of Embodiments 1-114 and 131-169, comprising a mutation in the UL37 viral gene.

Embodiment 171. The recombinant herpesvirus of Embodiment 170, wherein the UL37 viral gene encodes a UL37 protein comprising a mutation at at least 1, at least 2, at least 3, at least 4, or all 5 amino acid positions corresponding to Q403, E452, Q455, Q511, and R515 of SEQ ID NO: 856.

Embodiment 172. The recombinant herpesvirus of Embodiment 171, where the mutation in the UL37 viral gene comprises Q403A, E452A, Q455A, Q511A, and R515A according to SEQ ID NO: 856.

Embodiment 173. The recombinant herpesvirus of any one of Embodiments 1-114 and 131-172, encoding a gB comprising the mutations corresponding to A549T/D285N of SEQ ID NO: 919.

Embodiment 174. The recombinant herpesvirus of any one of Embodiments 1-114 and 131-173, wherein the recombinant herpesvirus retains the function of ICP6, ICP34.5, and/or ICP47.

Embodiment 175. The recombinant herpesvirus of any one of Embodiments 1-114 and 131-174, wherein the one or more transgenes are inserted in the UL50-UL51 intergenic region.

Embodiment 176. A recombinant virus comprising one or more transgenes encoding one or more payload proteins selected from HPGD, ADA2, HYAL1, CHP, CCL21, IL-12, a CD47 antagonist, a TGFβ antagonist, a PD1 antagonist, a TREM2 antagonist, a biomolecule comprising chlorotoxin (CTX), or any combinations thereof.

Embodiment 177. The recombinant virus of Embodiment 176, wherein the one or more payload proteins comprise or consist of IL-12, a PD1 antagonist, and a TREM2 antagonist.

Embodiment 178. The recombinant virus of Embodiment 177, wherein the one or more payload proteins comprise HPGD.

Embodiment 179. The recombinant virus of Embodiment 177 or 178, wherein the one or more payload proteins comprise a biomolecule comprising CTX.

Embodiment 180. The recombinant virus of Embodiment 176, wherein the one or more payload proteins comprise or consist of one of the combinations of payload proteins listed in Tables 4-7.

Embodiment 181. The recombinant virus of any one of Embodiments 176-180, wherein the one or more payload proteins comprise HPGD.

Embodiment 182. The recombinant virus of any one of Embodiments 176-181, wherein the one or more payload proteins comprise ADA2.

Embodiment 183. The recombinant virus of any one of Embodiments 176-182, wherein the one or more payload proteins comprise HYAL1.

Embodiment 184. The recombinant virus of any one of Embodiments 176-183, wherein the one or more payload proteins comprise CHP.

Embodiment 185. The recombinant virus of any one of Embodiments 176-184, wherein the one or more payload proteins comprise CCL21.

Embodiment 186. The recombinant virus of any one of Embodiments 176-185, wherein the one or more payload proteins comprise IL-12.

Embodiment 187. The recombinant virus of any one of Embodiments 176-186, wherein the one or more payload proteins comprise the CD47 antagonist.

Embodiment 188. The recombinant virus of any one of Embodiments 176-187, wherein the one or more payload proteins comprise the TGFβ antagonist.

Embodiment 189. The recombinant virus of any one of Embodiments 176-188, wherein the one or more payload proteins comprise the PD1 antagonist.

Embodiment 190. The recombinant virus of any one of Embodiments 176-189, wherein the one or more payload proteins comprise the TREM2 antagonist.

Embodiment 191. The recombinant virus of any one of Embodiments 176-190, wherein the antagonist comprises an antibody or antigen binding fragment thereof.

Embodiment 192. The recombinant virus of any one of Embodiments 176-191, wherein the one or more payload proteins comprise the biomolecule comprising CTX.

Embodiment 193. The recombinant virus of Embodiment 179 or 192, wherein the biomolecule comprising CTX further comprises a T-cell engager moiety specifically binding to a protein expressed on the surface of the T-cell.

Embodiment 194. The recombinant virus of Embodiment 193, wherein the protein expressed on the surface of the T-cell is CD3.

Embodiment 195. A recombinant virus comprising:
(a) one or more miRNA target sequences for miR-122-5p;
(b) one or more miRNA target sequences for miR-145-5p;
(c) one or more miRNA target sequences for miR-124-3p, miR-128T, miR-137-3p, or any combination thereof;
(d) one or more miRNA target sequences for miR-34b-5p, miR-34c-5p, or any combination thereof; and
(e) one or more miRNA target sequences for miR-129-2-3p, miR-132-3p, or any combination thereof.

Embodiment 196. The recombinant virus of Embodiment 195, comprising the miRNA target sequences for miR-34b-5p, miR-34c-5p, miR-122-5p, miR-124-3p, miR-128T, miR-129-2-3p, miR-132-3p, miR-137-3p, and miR-145-5p.

Embodiment 197. A recombinant virus, wherein the viral genome of the recombinant virus encodes a protein comprising a syncytial mutation and a counterpart protein without the syncytial mutation.

Embodiment 198. The recombinant virus of Embodiment 197, wherein the protein comprising the syncytial mutation and the counterpart protein without the syncytial mutation share at least 90%, at least 95%, at least 96%, at least 97%, at least 98%, at least 99%, or 100% sequence identity, except the syncytial mutation.

Embodiment 199. The recombinant virus of Embodiment 197 or 198, wherein the protein comprising the syncytial mutation is encoded by an endogenous viral gene and the counterpart protein without the syncytial mutation is encoded by an exogenous expression cassette.

Embodiment 200. The recombinant virus of Embodiment 197 or 198, wherein the protein comprising the syncytial mutation is encoded by an exogenous expression cassette and the counterpart protein without the syncytial mutation is encoded by an endogenous viral gene.

Embodiment 201. The recombinant virus of Embodiment 197 or 198, wherein both the protein comprising the syncytial mutation and the counterpart protein without the syncytial mutation are encoded by one exogenous expression cassette or by different exogenous expression cassettes.

Embodiment 202. A cell, comprising a recombinant nucleic acid encoding the recombinant virus of any one of Embodiments 197-201.

Embodiment 203. A cell, comprising a first nucleic acid encoding a recombinant virus and a second nucleic acid, wherein the viral genome of the recombinant virus encodes a protein comprising a syncytial mutation, wherein the second nucleic acid encodes a a counterpart protein without the syncytial mutation.

Embodiment 204. The cell of Embodiment 203, wherein the protein comprising the syncytial mutation and the counterpart protein without the syncytial mutation share at least 90%, at least 95%, at least 96%, at least 97%, at least 98%, at least 99%, or 100% sequence identity, except the syncytial mutation.

Embodiment 205. The recombinant virus of any one of Embodiments 197-201 or the cell of any one of Embodiments 202-204, wherein the yield of the virus is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than the yield of a control virus or a control cell that does not encodes the counterpart protein without the syncytial mutation.

Embodiment 206. The recombinant virus of any one of Embodiments 197-201 and 205, or the cell of any one of Embodiments 202-205, wherein the gene encoding the protein comprising the syncytial mutation comprises a miRNA target sequence (miR-TS) cassette, wherein the miR-TS cassette comprises one or more target sequences of one or more miRNAs.

Embodiment 207. The recombinant virus or the cell of Embodiment 206, wherein the one or more miRNAs comprise at least one, at least two, or all of miRNAs selected from miR-34c-5p, miR-299-5p, and miR-582-5p.

Embodiment 208. The recombinant virus or the cell of Embodiment 206 or 207, wherein the yield of the recombinant virus is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than the yield of a control virus or a control cell that does not comprise the miR-TS cassette.

Embodiment 209. The recombinant virus produced by culturing the cell of any of Embodiments 202-208 and recovering the recombinant herpesvirus from the cell culture.

Embodiment 210. The recombinant virus of any one of Embodiments 176-201 and 205-209, or the cell of any of Embodiments 202-208, wherein the recombinant virus is derived from a herpes simplex virus, an adenovirus, a polio virus, a vaccinia virus, a measles virus, a vesicular stomatitis virus, an orthomyxovirus, a parvovirus, a maraba virus, or a coxsackievirus.

Embodiment 211. The recombinant virus of any one of Embodiments 176-201 and 205-210, or the cell of any of Embodiments 202-208, wherein the recombinant virus is oncolytic.

Embodiment 212. A nucleic acid molecule encoding the recombinant herpesvirus of any one of Embodiments 1-114 and 131-175, or the recombinant virus of any one of Embodiments 176-201 and 205-211.

Embodiment 213. The nucleic acid molecule of Embodiment 212, wherein the nucleic acid molecule is DNA.

Embodiment 214. The nucleic acid molecule of Embodiment 212, wherein the nucleic acid molecule is RNA.

Embodiment 215. A viral stock comprising the recombinant herpesvirus of any one of Embodiments 1-114 and 131-175, or the recombinant virus of any one of Embodiments 176-201 and 205-211.

Embodiment 216. A particle comprising the nucleic acid molecule of any one of Embodiments 212-214.

Embodiment 217. The particle of Embodiment 216, wherein the particle is selected from the group consisting of a nanoparticle, an exosome, a liposome, and a lipoplex.

Embodiment 218. The particle of Embodiment 216, wherein the particle is a lipid nanoparticle.

Embodiment 219. The particle of any one of Embodiments 216-218, wherein contacting a eukaryotic cell with the particle results in production of infectious virus particles by the eukaryotic cell.

Embodiment 220. A pharmaceutical composition comprising:
(i) the recombinant herpesvirus of any one of Embodiments 1-114 and 131-175, the recombinant virus of any one of Embodiments 176-201 and 205-211, the nucleic acid molecule of any one of Embodiments 212-214, or the particle of any one of Embodiments 216-219; and
(ii) a pharmaceutically-acceptable carrier.

Embodiment 221. A method of killing a cancerous cell, comprising exposing the cancerous cell to the recombinant herpesvirus of any one of Embodiments 1-114 and 131-175, the recombinant virus of any one of Embodiments 176-201 and 205-211, the particle of any one of Embodiments 216-219, or the pharmaceutical composition of Embodiment 220, under conditions sufficient for the virus or particle to infect and the virus to replicate within said cancerous cell, and wherein replication of the virus within the cancerous cell results in cell death.

Embodiment 222. The method of Embodiment 221, wherein the cell is in vitro or in vivo.

Embodiment 223. The method of Embodiment 221 or 222, wherein the cancerous cell has a reduced expression of a miRNA capable of binding to the one or more miRNA target sequences compared to the expression of the miRNA in a non-cancerous cell.

Embodiment 224. The method of any one of Embodiments 221-223, wherein replication of the virus is increased or maintained in the cancerous cell with a reduced expression of the miR capable of binding to the one or more miRNA target sequences.

Embodiment 225. The method of any one of Embodiments 221-224, wherein the cancerous cell is a cell of lung cancer, breast cancer, ovarian cancer, cervical cancer, prostate cancer, testicular cancer, colorectal cancer, colon cancer, pancreatic cancer, liver cancer, gastric cancer, head and neck cancer, thyroid cancer, malignant glioma, glioblastoma, melanoma, non-melanoma skin cancer, B-cell chronic lymphocytic leukemia, diffuse large B-cell lymphoma (DLBCL), or marginal zone lymphoma (MZL).

Embodiment 226. The method of any one of Embodiments 221-224, wherein the cancerous cell is a glioblastoma cell.

Embodiment 227. A method of treating cancer in a subject in need thereof, comprising administering the recombinant herpesvirus of any one of Embodiments 1-114 and 131-175, the recombinant virus of any one of Embodiments 176-201 and 205-211, the particle of any one of Embodiments 216-219, or the pharmaceutical composition of Embodiment 220 to the subject.

Embodiment 228. The method of Embodiment 227, wherein the virus, the particle, or the composition is administered intravenously, subcutaneously, intratumorally, intramuscularly, or intranasally.

Embodiment 229. The method of Embodiment 227, wherein the virus, the particle, or the composition is administered intratumorally.

Embodiment 230. The method of Embodiment 227, wherein the virus, the particle, or the composition is administered intravenously.

Embodiment 231. The method of any one of Embodiments 227-230, wherein the virus, the particle, or the composition is administered only once.

Embodiment 232. The method of any one of Embodiments 227-231, wherein the cancer is selected from the group consisting of lung cancer, breast cancer, ovarian cancer, cervical cancer, prostate cancer, testicular cancer, colorectal cancer, colon cancer, pancreatic cancer, liver cancer, gastric cancer, head and neck cancer, thyroid cancer, malignant glioma, glioblastoma, melanoma, non-melanoma skin cancer, B-cell chronic lymphocytic leukemia, diffuse large B-cell lymphoma (DLBCL), and marginal zone lymphoma (MZL).

Embodiment 233. The method of any one of Embodiments 227-231, wherein the cancer is glioblastoma.

Embodiment 234. A cell line, comprising the cell of any of Embodiments 115-160 and 202-208.

Embodiment 235. A method of producing a recombinant herpesvirus, comprising culturing the cell of any of Embodiments 115-160 and 202-208, or the cell line of Embodiment 234, and recovering the recombinant herpesvirus from the cell culture.

Embodiment 236. The recombinant herpesvirus of any one of Embodiments 82-114 and 131-175 for use in combination with a small molecule for imaging the infection site of the herpesvirus.

Embodiment 237. A method of imaging the infection site of an herpesvirus in vivo, comprising administering the recombinant herpesvirus of any one of Embodiments 82-114 and 131-175 and a small molecule.

Embodiment 238. The recombinant virus for use of Embodiment 236 or the method of Embodiment 237, wherein the small molecule is radioisotope labeled acyclovir; optionally wherein the radioisotope label comprises fluorine-18 (18F) label.

### EXAMPLES

The following examples for the purpose of illustrating various embodiments of the disclosure and are not meant to limit the present disclosure in any fashion. The present examples, along with the methods described herein, are exemplary, and are not intended as limitations on the scope of the disclosure. Alterations, modifications, and other changes to the described embodiments which are encompassed within the spirit of the disclosure as defined by the scope of the claims are specifically contemplated.

### Example 1—Construction of HSV Backbone for Glioblastoma Treatment

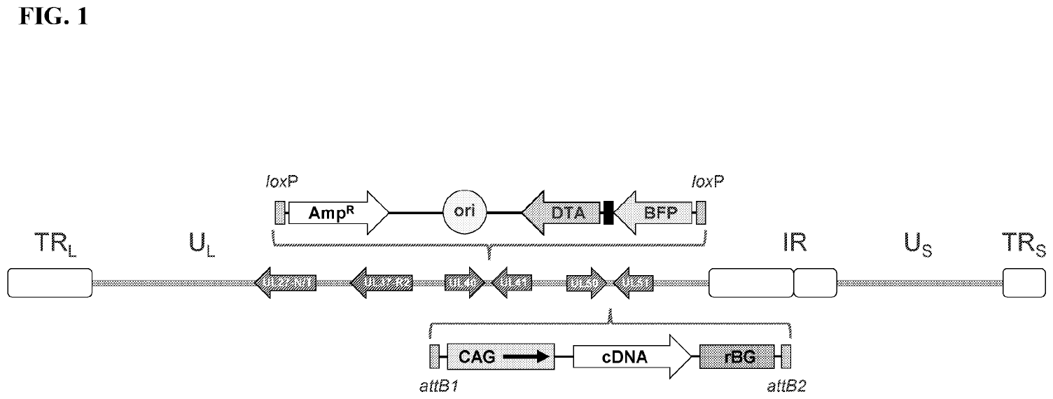

An HSV backbone vector (termed “ONCR-GBM”) was engineered based on an encephalitic HSV isolate (SEQ ID NO: 857) that is defective for anterograde transport. As illustrated in FIG. 1, the modifications included: a bacterial artificial chromosome (BAC) sequence inserted in the intergenic region between UL40-UL41 viral genes; D285N/A549T double mutations (“NT” mutations) introduced into the UL27 viral gene encoding gB to enhance cell entry; and the Q403A/E452A/Q455A/Q511A/R515A combo mutations (“R2” mutations) introduced into the UL37 viral gene to disrupt retrograde transport. The ICP47, ICP34.5, and ICP6 genes were unaltered. cDNA encoding the payload molecule(s) may be inserted into the intergenic region between UL50-UL51 viral genes. Based on this HSV backbone, several miR-attenuated recombinant HSV vector were generated: the “ONCR-GBM ICP4-miRT” HSV vector comprises a miR-TS cassette of SEQ ID NO: 858 inserted into ICP4; the “ONCR-GBM ICP4-miRT ICP8-miRT” HSV vector comprises the miR-TS cassette of SEQ ID NO: 858 inserted into ICP4 and an additional miR-TS cassette of SEQ ID NO: 859 inserted into ICP8.

### Example 2—Engineering of miRNA Target Sequence Cassettes

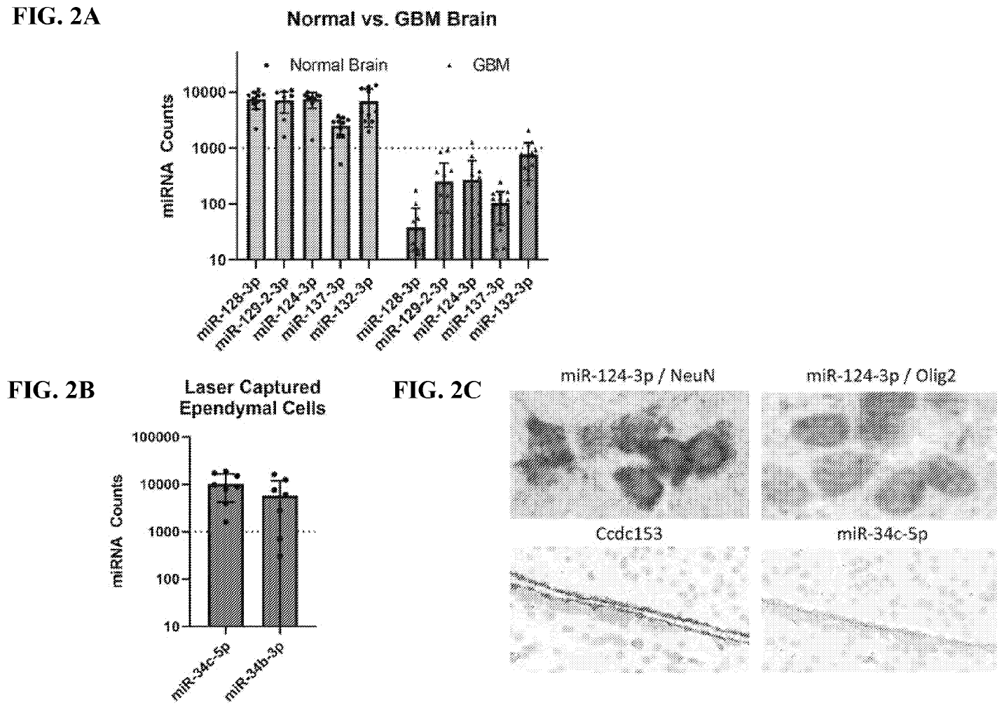

Several miRNAs with reduced expression in glioblastoma cells compared to normal brain cells were identified using purified cell populations. As shown in FIG. 2A, such miRNAs include miR-128-3p, miR-129-2-3p, miR-124-3p, miR-137-3p, and miR-132-3p. FIG. 2B shows that miR-34c-5p and miR-34b-3p were highly expressed in ependymal cells so the corresponding miR target sequences could be used to inhibit the replication of the oncolytic virus in ependymal cells. Such findings were then validated using in situ hybridization and/or antibody staining to localize the miRNA expression to specific cell types. For example, FIG. 2C upper row shows that miR-124-3p was highly expressed in neurons but not oligodendrocytes, and FIG. 2C lower row shows that miR-34c-5p was highly expressed in ependymal cells (which was characterized with high expression of biomarker Ccdc153).

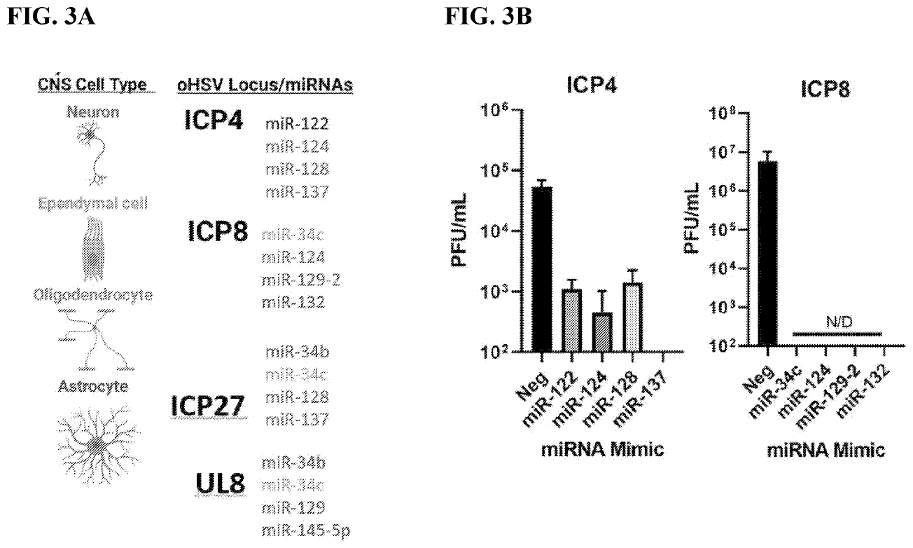

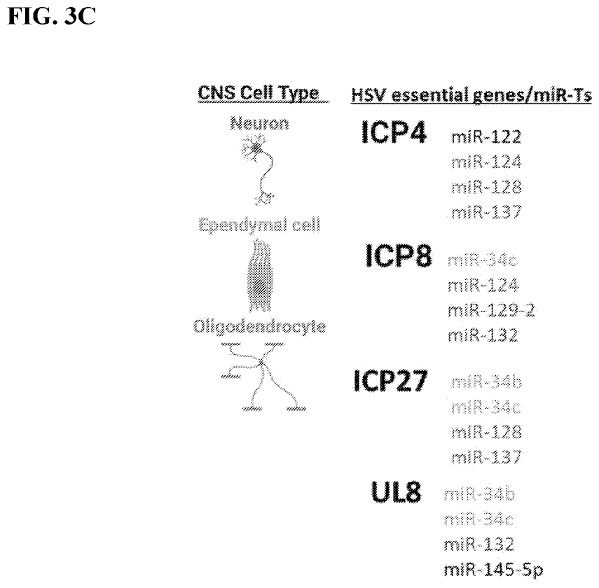

In vitro cell experiments were used to test whether the replication of the “ONCR-GBM ICP4-miRT ICP8-miRT” HSV vector could be attenuated by the corresponding miRNAs. This HSV vector comprises miRNA target sequences for miR-122-5p, miR-124-3p, miR-128T, and miR-137-3p in ICP4 and miRNA target sequences for miR-34c-5p, miR-124-3p, miR-129-2-3p, miR-132-3p in ICP27. To evaluate miRNA mediated attenuation, each of the corresponding miRNA mimic was added to the supernatant of cells infected with the HSV vector, and the replication capability of the HSV in each condition was analyzed based on the final count of plaque forming units. As shown in FIG. 3B, insertion of the miRNA target sequences into ICP4 or ICP8 effectively suppressed viral replication of the HSV vector at the presence of each corresponding miRNA.

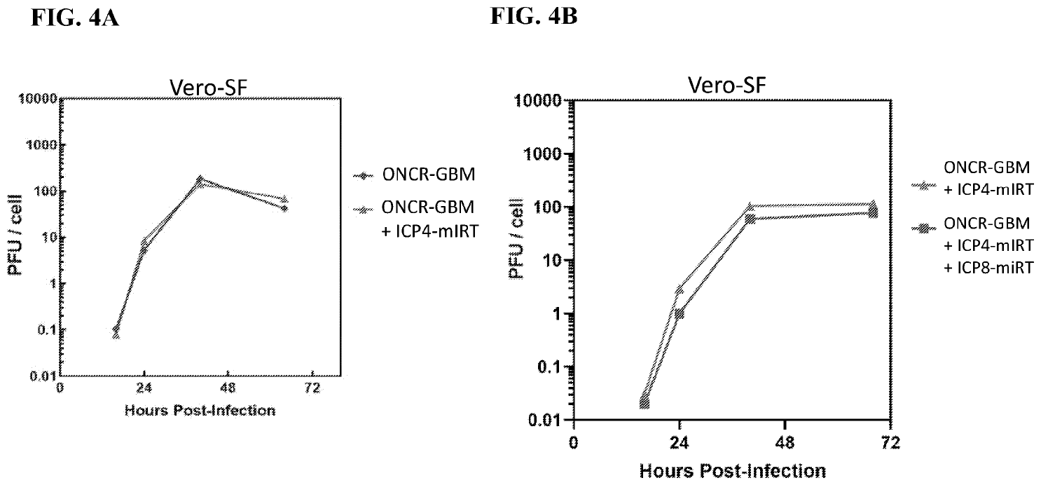

Next, growth kinetics study was performed to test the effect of miR-TS cassettes on the propagation of the engineered HSV vectors “ONCR-GBM ICP4-miRT” and “ONCR-GBM ICP4-miRT ICP8-miRT” as described in Example 1 above. These vectors were tested for propagation in Vero cells and GBM cells. The results show that insertion of the miR-TS cassette(s) had minimal effects on growth kinetics during propagation in Vero cells (FIG. 4A and FIG. 4B) and GBM tumor cell lines (data not shown).

### Example 3—In Vivo Studies of miR-Attenuated HSV Demonstrates Superior Safety Profile

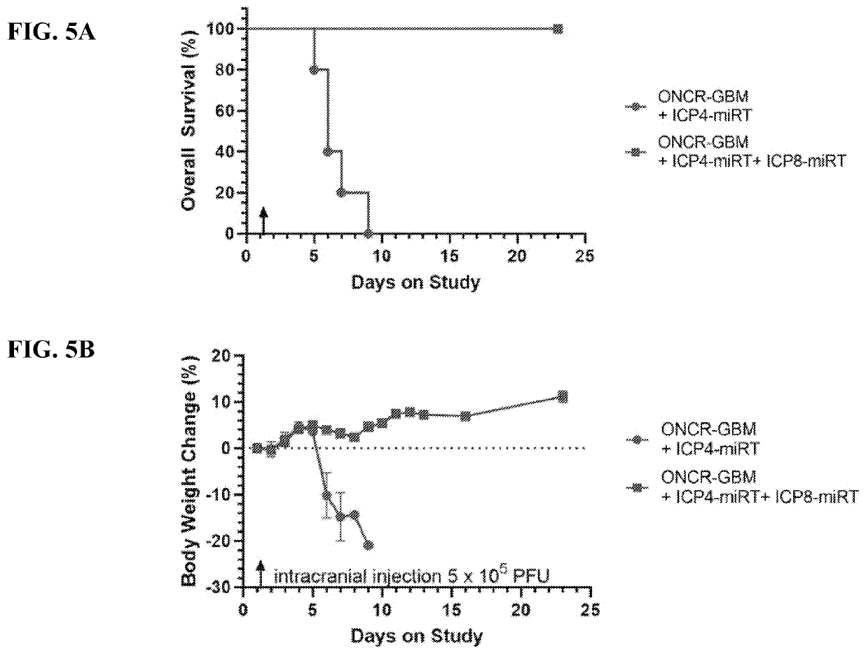

In vivo experiments were conducted to test the safety profile of the miR-attenuated HSV oncolytic virus. The ONCR-GBM based virus in Example 2 was intracranially injected into adult C57BL/6 mice and the overall survival (FIG. 5A) and body weight change (FIG. 5B) were monitored. The results showed that ONCR-GBM virus with CNS-specific miR target sequences inserted in ICP4 and ICP8 is well tolerated at a dose of 5×10⁵ pfu. As a comparison, intracranial injection of ˜20 pfu of non-attenuated ONCR-GBM was lethal (data not shown). Therefore, the insertion of miR-TS cassettes into both ICP4 and ICP8 loci resulted in remarkably reduced neurotoxicity and a >10,000-fold increase in safety/tolerability.

### Example 4—Engineering of miR-TS Cassettes

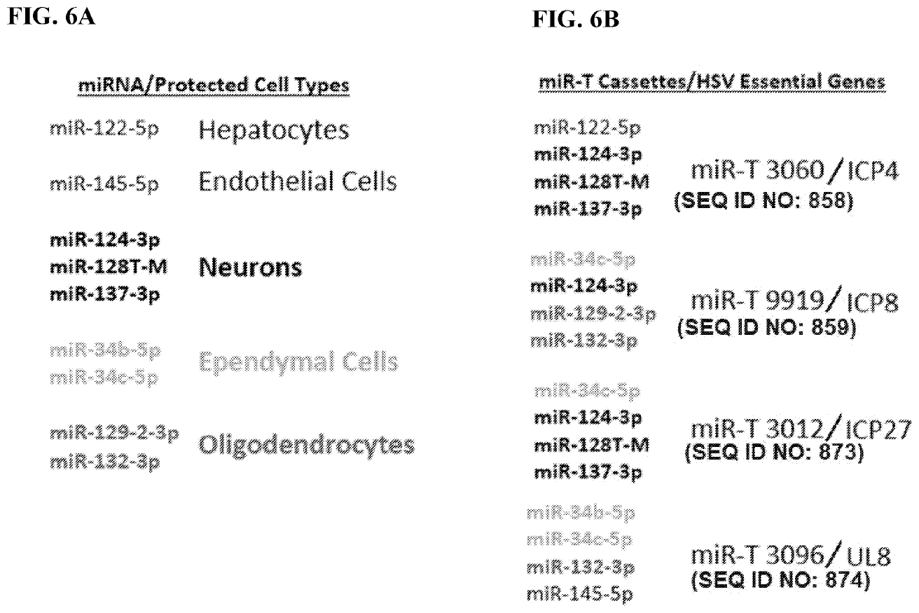

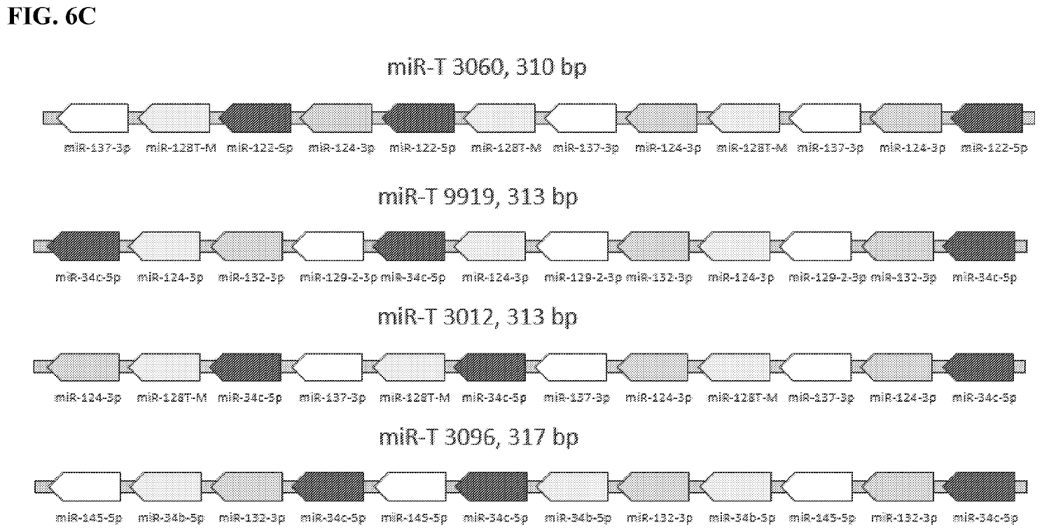

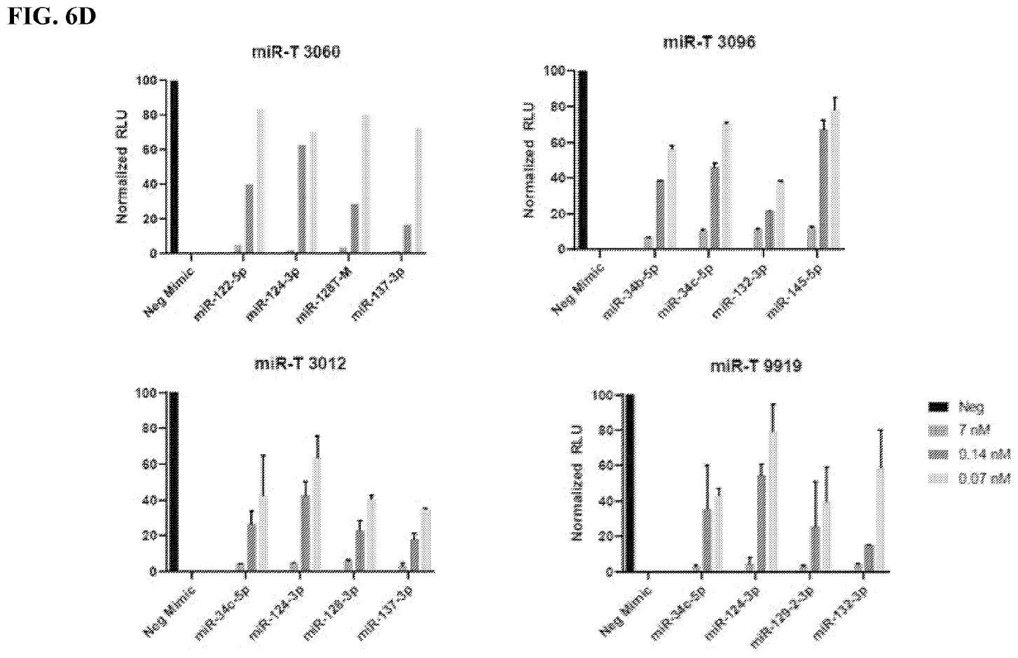

Design of miR-TS Cassettes: MicroRNAs for protecting the desired cell types (hepatocytes, endothelial cells, neurons, ependymal cells, and oligodendrocytes) were identified by direct quantification using a fluorescent hybridization assay (nCounter miRNA Expression kit, nanoString Inc) (FIG. 6A). The corresponding miRNA target sequences for each miRNA were assembled into four individual miR-TS cassettes: miR-T 3060 (SEQ ID NO: 858), miR-T 9919 (SEQ ID NO: 859), miR-T 3012 (SEQ ID NO: 873), and miR-T 3096 (SEQ ID NO: 874). Each miR-TS cassette contained 3 copies of miRNA target sequences for 4 different miRNAs (FIG. 6B), and adjacent miRNA target sequences were separated by a 4 bp spacer. The miR-TS configuration within each cassette are shown in FIG. 6C.

Characterization of miR-TS cassettes: Each of the miR-TS cassettes were subcloned into the psiCheck2 dual Firefly/Renilla luciferase reporter vector (Promega Inc., Madison WI) such that they controlled the expression of the Renilla luciferase, but not the Firefly luciferase. Each construct was co-transfected into HEK293 cells along with the indicated miRNA mimic (mirVana Mimics, ThermoFisher Inc, Waltham MA), incubated for 2 days, and then the activity of each reporter gene was assayed using a homogeneous assay (Dual-Glo Luciferase Assay System, Promega, Madison WI). The attenuation conferred by each miR-TS cassette was calculated as the ratio of Renilla/Firefly luciferase activity and was normalized relative to that of the negative miRNA mimic control. As shown in FIG. 6D, expression of the Renilla luciferase was suppressed by the presence of each corresponding microRNA of the indicated miR-TS cassette.

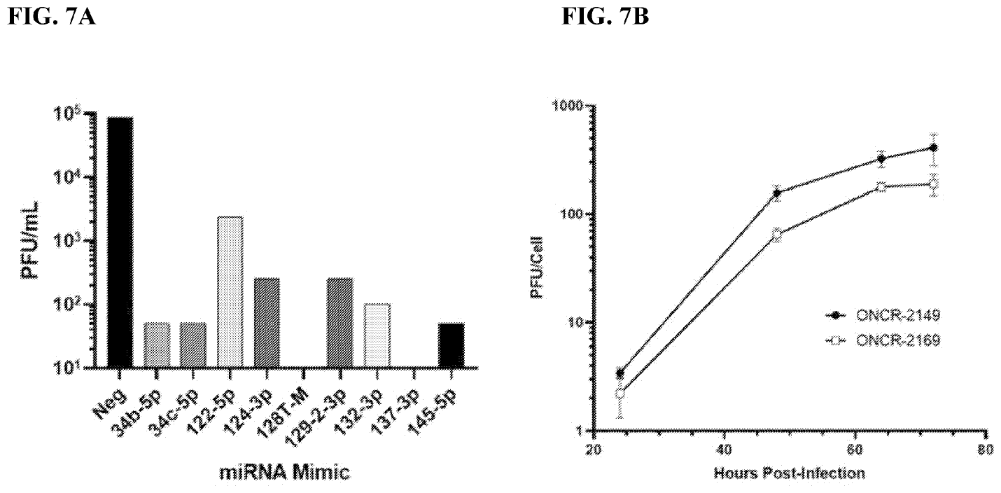

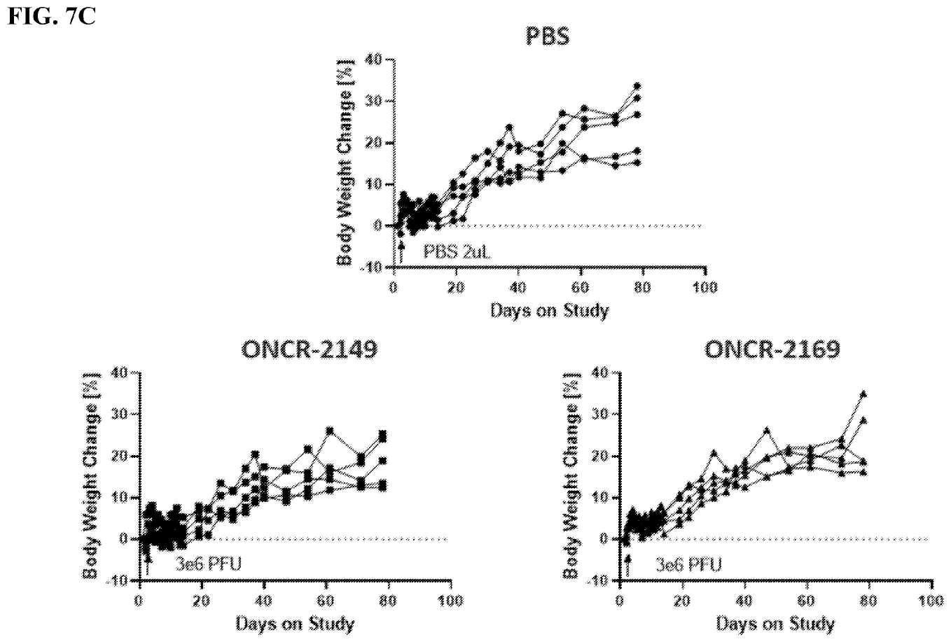

In vitro Characterization of miR-TS Attenuated HSV: All four miR-TS cassettes were inserted into the corresponding HSV viral gene as shown in Table 8 below and FIG. 6B to generate the HSV virus ONCR-2169. A431 cells were transfected with 7 nM of the indicted miRNA mimic (mirVana Mimics, ThermoFisher, Waltham MA), inoculated with ONCR-2169 at a multiplicity of infection of 0.03, then incubated for 5 days to allow for virus outgrowth. The cells were harvested and then lysed through three rapid freeze-thaw cycles to release the virus. Lysates were analyzed for virus yield by plaque assay of serial dilutions on Vero cell monolayers. As shown in FIG. 7A, by targeting at least one of the miR-TS cassettes in ONCR-2169, each of the indicated miRNA mimics resulted in significant decrease of virus yield (mostly >100-fold), demonstrating successful miRNA-medicated growth attenuation.

TABLE 8
Design of miR-TS Cassettes in ONCR-2169

| | | | | | Cell Type |
|---|---|---|---|---|---|
| | | | | | Protected by |
| Viral Gene | ICP4 | ICP8 | ICP27 | UL8 | mir-TS*: |
| Cassette | miR-T 3060 | miR-T 9919 | miR-T 3012 | miR-T 3096 | Neurons |
| miR-TS | miR-122-5p | miR-34c-5p | miR-34c-5p | miR-34b-5p | Ependymal cells |
| | miR-124-3p | miR-124-3p | miR-124-3p | miR-34c-5p | Oligodendrocytes |
| | miR-128T-M | miR-129-2-3p | miR-128T-M | miR-132-3p | Endothelial cells |
| | miR-137-3p | miR-132-3p | miR-137-3p | miR-145-5p | Hepatocytes |

*Font (bold, italic, and/or underline) matches the corresponding miR-TS.

The viral growth was also analyzed in the absence of microRNA mimics. Two HSV constructs were analyzed. ONCR-2149 has a miR-T3060 cassette in both copies of ICP4 and a miR-T9919 cassette in ICP8. ONCR-2169 has a miR-T3012 in ICP27 and a miR-T3096 cassette in UL8 but is otherwise identical to ONCR-2149. Vero cell monolayers were infected with the viruses at a multiplicity of infection of 0.03, then incubated for 3 days to allow for virus outgrowth. The cells were harvested and then lysed through three rapid freeze-thaw cycles to release the virus. Lysates were analyzed for virus yield by plaque assay of serial dilutions on Vero cell monolayers. The result shows that both viruses had similar yield in the absence of corresponding microRNA mimics (FIG. 7B).

In vivo Characterization of miR-TS Attenuated HSV: ten-week old female BALB/c mice were injected intra-cranially with 2 ul of the indicated viruses (ONCR-2149 or ONCR-2169), then monitored daily for signs of neurotoxicity and changes in their overall body weight. As shown FIG. 7C, no statistically significant difference was detected between any treatment groups, demonstrating that the miR-TS cassettes protect normal cells in the brain from the toxicity of the corresponding HSV constructs. According to prior reports, the lethal dosage of the HSV MacIntyre strain was about 20 PFU in some mouse strains. Therefore, the results here suggest that these engineered HSV comprising miR-TS cassettes can be administered at a dose that is at least 5 orders of magnitude higher that the original HSV strain. It is also worth noting that astrocytes and oligodendrocytes in mice are more resistant to HSV then those in human, and therefore additional safety margin can be beneficial for an oncolytic HSV that will be used for treating human subjects. Accordingly, even higher doses (e.g., 5e⁶ PFU) of the ONCR-2149 and ONCR-2169 viruses will be used to test the additional protection offered by the inclusion of the miR-TS cassettes in ICP27 and UL8 of ONCR-2169.

### Example 5—Engineering of Retargeted HSV

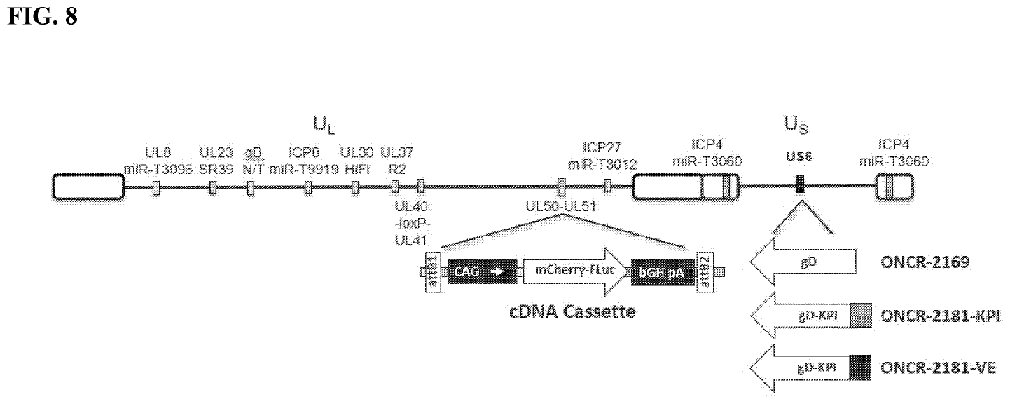

Two HSV constructs were engineered to retarget the HSV for alternative cell receptors, as shown in FIG. 8. Virus backbones were based on ONCR-2169, the cloned derivative of the HSV MacIntyre strain comprising miR-TS cassettes in ICP4, ICP8, IP27, and UL8, as well as the indicated mutations in UL23, UL30, UL37, and gB. As indicated on the right side of the figure, retargeting mutations were incorporated into the US6/gD gene by replacing the coding region for amino acid residues 6-24 of gD, the HVEM and 3-O-S-Heparan Sulfate binding domain, with a sequence encoding either i) a Knottin peptide that binds integrins (KPI); or ii) a VHH nanobody that binds the extracellular domain of EGFR (VE).

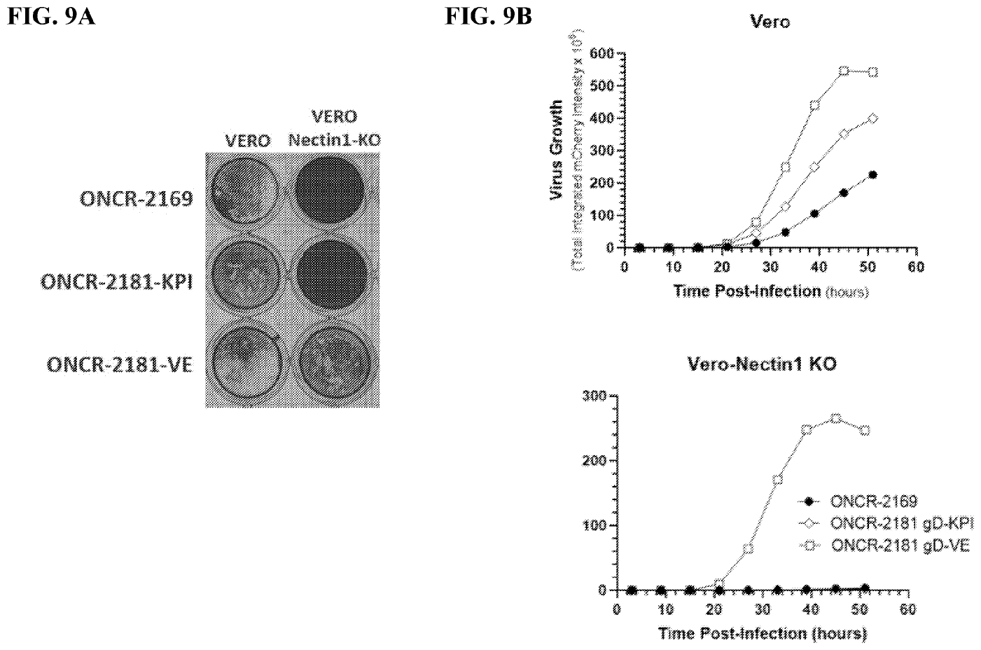

The engineered retargeting HSVs were tested for their growth properties in different Vero cells: regular Vero cells, and Vero cells with Nectin-1 knockout (KO). Cell monolayers were infected with the indicated viruses at a multiplicity of infection of 1.0. After incubation for 48 hours the monolayers were fixed and stained with crystal violet to visualize plaques associated with virus growth. As shown in FIG. 9A, the ONCR-2181-KPI mutant lost its infectivity for Vero-Nectin-1 knockout cells just like the parental HSV, whereas the ONCR-2181-VE mutant retained the infectivity even in Vero-Nectin-1 knockout cells. In another proliferation assay, Vero and Vero-Nectin-1 knockout cell monolayers were infected with the indicated viruses at a multiplicity of infection of 0.1. Growth of the viruses was assayed by quantifying mCherry reporter gene expression over the course of ˜48 hours using an automated inverted microscope (IncuCyte S3, Sartorius Inc). As shown in FIG. 9B, both gD-KPI and gD-VE mutations conferred improved growth kinetics on Vero cells, but only the gD-VE mutant facilitated growth on the Vero-Nectin-1 knockout cell line. Therefore, the gD-VE mutant gained the ability to infect and grow in cells with no Nectin-1 expression.

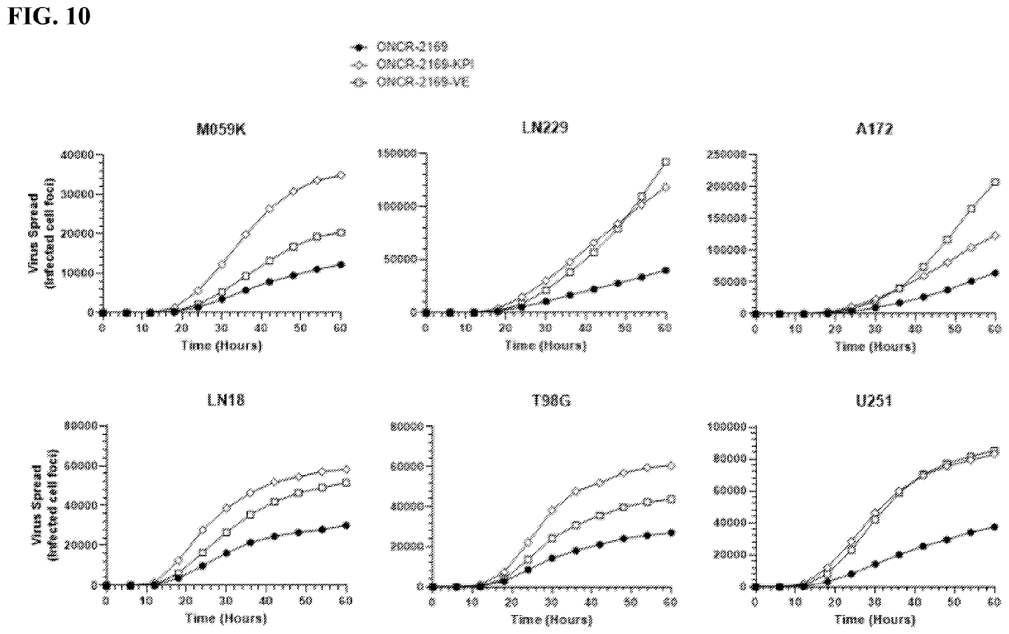

The engineered retargeting HSVs were then analyzed for their growth properties in glioblastoma cell lines. Monolayers of the indicated glioblastoma cancer cell lines were infected with the indicated viruses at a multiplicity of infection of 0.1. Growth of the viruses was assayed by quantifying mCherry reporter gene expression over the course of ˜48 hours using an automated inverted microscope (IncuCyte S3, Sartorius Inc). As shown in FIG. 10, the gD-KPI and gD-VE mutations conferred improved growth kinetics in most of the assayed cell lines.

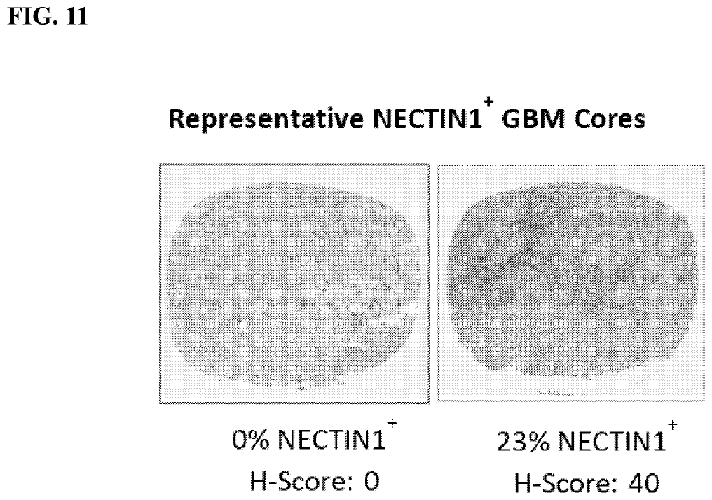

The expressions of Nectin-1 and epidermal growth factor receptor (EGFR) in primary human glioblastoma (GBM) tumors were analyzed by microarray. As shown in Table 9 below, the prevalence of GBM cores with high Nectin-1 expression is much lower than that of GBM cores with high EGFR expression. Representative Nectin-1+GBM Cores are shown in FIG. 11. Based on the expression data, the EGFR-retargeted HSV will be more applicable to a wilder range of GBM tumors.

TABLE 9
Expression of Nectin-1 and EGFR in Glioblastoma Samples

| | No. of | | | Average H- |
|---|---|---|---|---|
| | core | Prevalence- | Average | score** of |
| | tumors | % of cores | % of tumor | Biomarker⁺ |
| Biomarker | (N)* | Biomarker+ ≥1% | cells | cores (0-300) |
| Nectin-1 | 41 | 2.4% | 23% | 39.6 |
| EGFR | 41 | 85% | 62% | 161 |

*primary GBM IV, including 1 IDHmut, 5 recurrent GBM
**H-score is a measure of expression intensity (1+, 2+, 3+) and % tumor area = 1 × (% cells 1+) + 2 × (% cells 2+) + 3 × (% cells 3+)

### Example 6—Improving Replication Fidelity and Acyclovir Sensitivity of the HSV

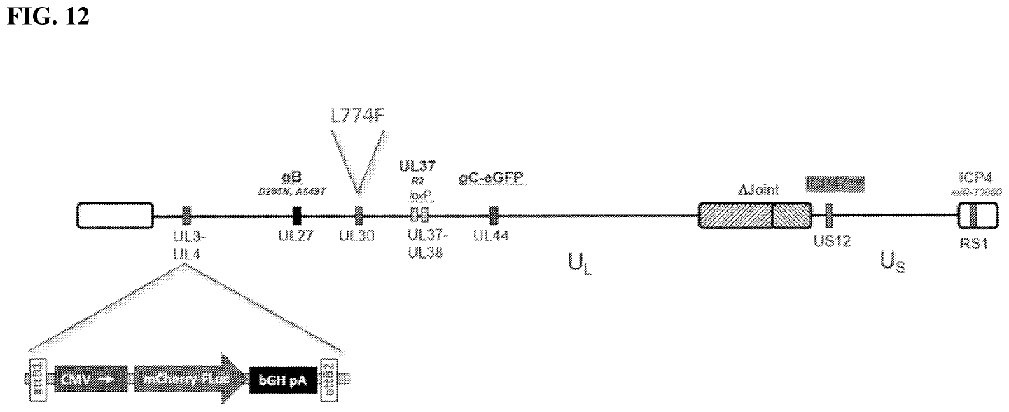

The L774F mutation was introduced into the UL30 gene of ONCR-1010 viral construct to generate ONCR-1012 viral construct (FIG. 12). Both viruses expressed the GFP reporter gene from UL44/gC promoter and the mCherry and FLuc reporter genes from the CMV promoter in the payload cassette. ONCR-1010 (UL30-WT) and ONCR-1012 (UL30-L774F) viruses were triple plaque purified, then serially propagated on Vero cells every 3-5 days. Viral DNA was purified at passage number 1, 5, 10, 15, and 20 for analysis by next gen sequencing (NGS). Sequencing data was analyzed by removing all variants that had <1000 total reads at that position and that occurred at <1% in the data set, and then removing all variant calls that were already present at passage number 1.

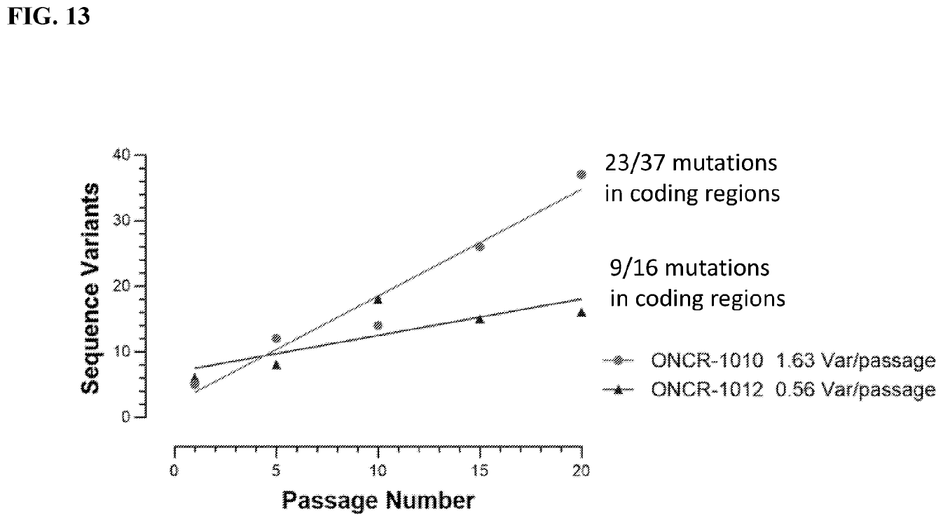

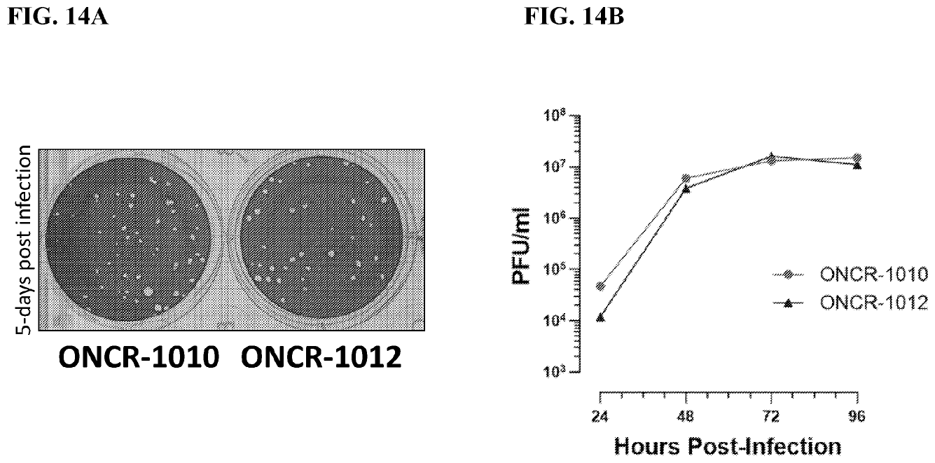

As shown in FIG. 13, the UL30-L774F mutation significantly reduced the mutation frequency of the virus, from 1.63 mutation/passage on average in the ONCR-1010 control to 0.56 mutation/passage on average in the ONCR-1012 construct. In the ONCR-1010 group, 23 of the 37 mutations were in the coding regions; in the ONCR-1012 group, 9 of the 16 mutations were in the coding regions. And, as shown in FIG. 14A and FIG. 14B, the UL30-L774F mutation had little effect on virus production.

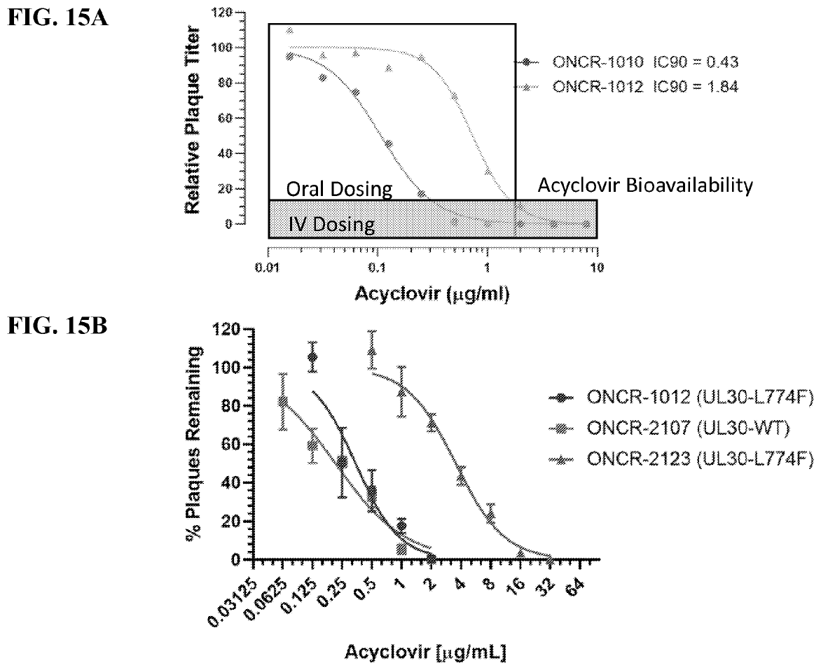

The UL30-L774F mutation, however, lowered acyclovir sensitivity of the HSV construct and thus might compromise its treatment efficacy. Acyclovir can be used as a prodrug in HSV-TK mediated suicide gene therapy of cancer, which requires its phosphorylation by HSV thymidine kinase (TK). Virus sensitivity to acyclovir was assayed by adding increasing concentrations of the drug to intact Vero cell monolayers and then infecting with a standard dose of virus. The Vero cell monolayers were incubated for 4 days to allow for virus growth, and then the virus was harvested and the corresponding lysates assayed for virus by plaque assay. The IC90 was calculated as the amount of acyclovir that produced a 90% reduction in plaque titer compared to the untreated control. As shown in FIG. 15A, when introduced into ONCR-1010, the UL30-L774F mutation increased the IC90 of acyclovir from 0.43 ug/ml to 1.84 ug/ml, indicating lower acyclovir sensitivity. Because the bioavailability of acyclovir, when dosed orally, was about 1.76 ug/ml in the CNS, the efficacy of acyclovir may be significantly reduced in cells infected with HSV comprising the UL30-L774F mutation. This issue may be more prominent in an HSV strain that is intrinsically less sensitive to acyclovir, such as the HSV-Macintyre strain. As shown in FIG. 15B, when introduced into ONCR-2107 derived from HSV-MacIntyre, the UL30-L774F mutation rendered acyclovir resistance to the resultant ONCR-2123 construct.

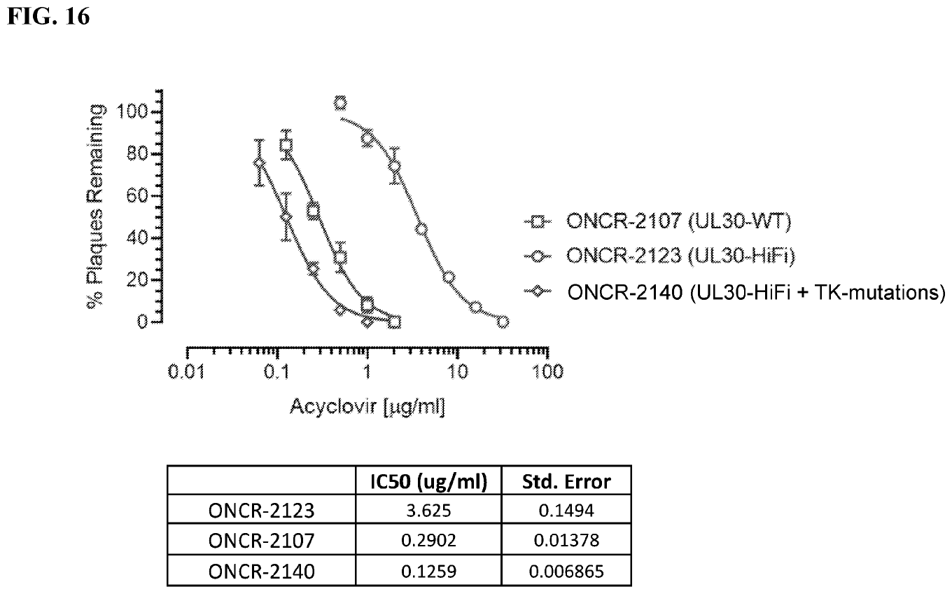

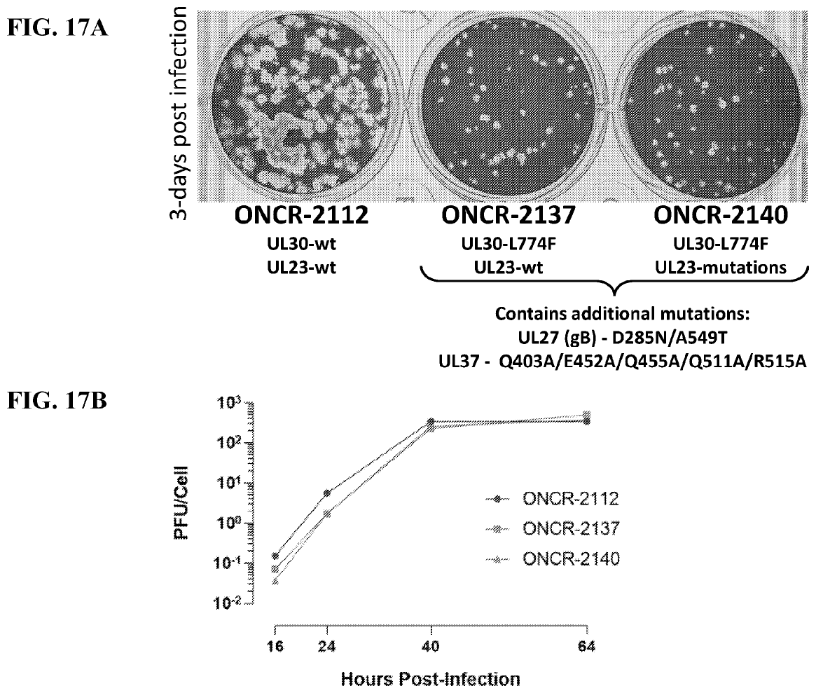

To improve the acyclovir sensitivity of HSV variants carrying the UL30-L774F mutation, additional mutations were introduced into the UL23 viral gene encoding HSV thymidine kinase (HSV-TK) of ONCR-02123, resulting in the amino acid substitutions corresponding to L159I, 1160F, F161L, A168F and L169M of SEQ ID NO: 918. As shown in FIG. 16, these UL23 mutations restored acyclovir sensitivity of the HSV-MacIntyre derived ONCR-2140 virus carrying the UL30-L774F mutation. These HSV-TK mutations did not significantly affect virus production, as shown in FIG. 17A and FIG. 17B. Thus, combining the UL30-L774F and these UL23 mutations results in improved replication fidelity without compromising the acyclovir sensitivity.

### Example 7—Improving the Titer of HSV Syncytial Mutants During Virus Production

HSV syncytial mutations improve oncolysis and virus spread in tumor cells and enhance immunogenicity through releasing immunostimulatory DAMPs. However, these syncytial mutations often lower the viral titer during virus production, thus hampering their clinical applications.

Accordingly, to improve the yield of the HSV syncytial mutants during virus production (e.g., in Vero cells), an expression system was established to allow co-expression of non-syncytial version and syncytial mutant of either gB or gK protein, which facilitated the generation of viral envelopes that contained a greater proportion of gB or gK in an active conformation.

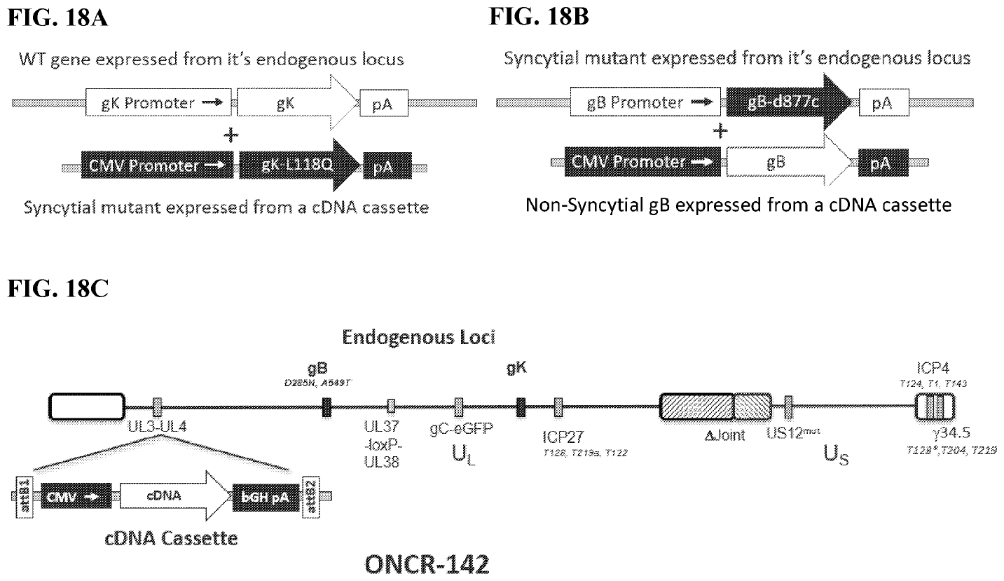

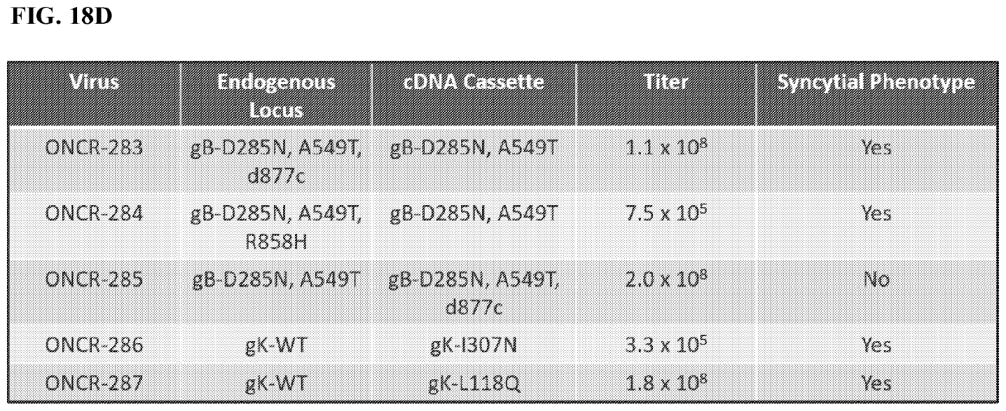

FIG. 18A-18C show multiple viral vector designs that allowed co-expression of both non-syncytial and syncytial variants of either gB or gK during virus production. The viral vector design in FIG. 18A allowed co-expression of wildtype gK from its endogenous locus and a mammalian codon optimized syncytial gK-L118Q mutant from a cDNA expression cassette. Other pairs generated in this configuration included “endogenous wildtype gK+mammalian codon optimized syncytial gK-1307N mutant in the cDNA cassette” and “endogenous non-syncytial gB+mammalian codon optimized syncytial gB-d877c mutant in the cDNA cassette.” (Here, the syncytial gB-d877c mutation refers to a gB protein comprising the deletion of all the C-terminal residues starting from amino acid T877.) On the other hand, the viral vector design in FIG. 18B allowed co-expression of a syncytial gB-d877c mutant from its endogenous locus in the virus genome and a mammalian codon-optimized gB from a cDNA expression cassette. Other pairs generated in this configuration include “syncytial gB-R858H mutant in the endogenous locus+mammalian codon optimized non-syncytial gB in the cDNA cassette.” In all these designs, the encoded gB protein comprised D285N and A549T mutations, regardless of whether the gB protein comprised the additional syncytial mutation (R858H or d877c). The ONCR-142 HSV backbone was used to evaluate the performance of all these viral vector designs. FIG. 18C shows the relative locations of the endogenous gB and gK loci as well as the cDNA cassette, which is inserted in the UL3-UL4 intergenic locus.

To test the production yield of each HSV construct, Vero cells were inoculated with the indicated viruses at a multiplicity of infection of 0.03 then incubated for 5 days to allow for virus outgrowth. The cells were harvested and then lysed through 3 rapid freeze-thaw cycles to release the virus. Lysates were analyzed for virus yield and syncytia formation by plaque assay of serial dilutions on Vero cell monolayers. As shown in FIG. 18D, simultaneous expression of both wild-type and syncytial mutants during virus growth increased the production yield of multiple vectors, including ONCR-283, ONCR-285 and ONCR-287. In comparison, HSV comprising either gB-d877c or gK-L118Q syncytial mutation without the corresponding non-syncytial copy of gB or gK had much lower virus yield (data not shown). And, other than ONCR-285, the other four HSV constructs all displayed syncytial phenotype.

To further improve virus production yield, a miRNA target sequences cassette (miR-TS cassette) was inserted into the gene loci encoding the gB or gK syncytial mutant to regulate the expression of the gB or gK syncytial mutant during virus production. The miR-TS cassette comprised target sequences for three miRNAs (miR-34c-5p, miR-299-5p, and miR-582-5p), which are highly expressed in the Vero production cell line but not in tumor cells. These three microRNAs were identified by direct quantification of RNA sample preparations of Vero cells using a fluorescent hybridization assay (nCounter miRNA Expression kit, NanoString Inc). As a result, the expression of the non-syncytial gB or gK was favored in Vero production cells but the expression of the syncytial mutant was favored in tumor cells.

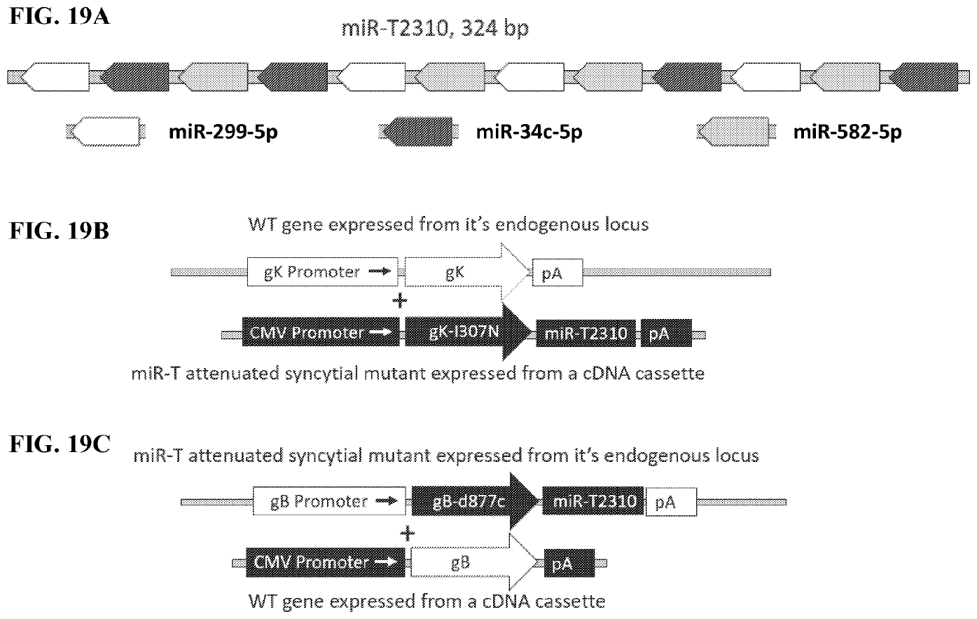

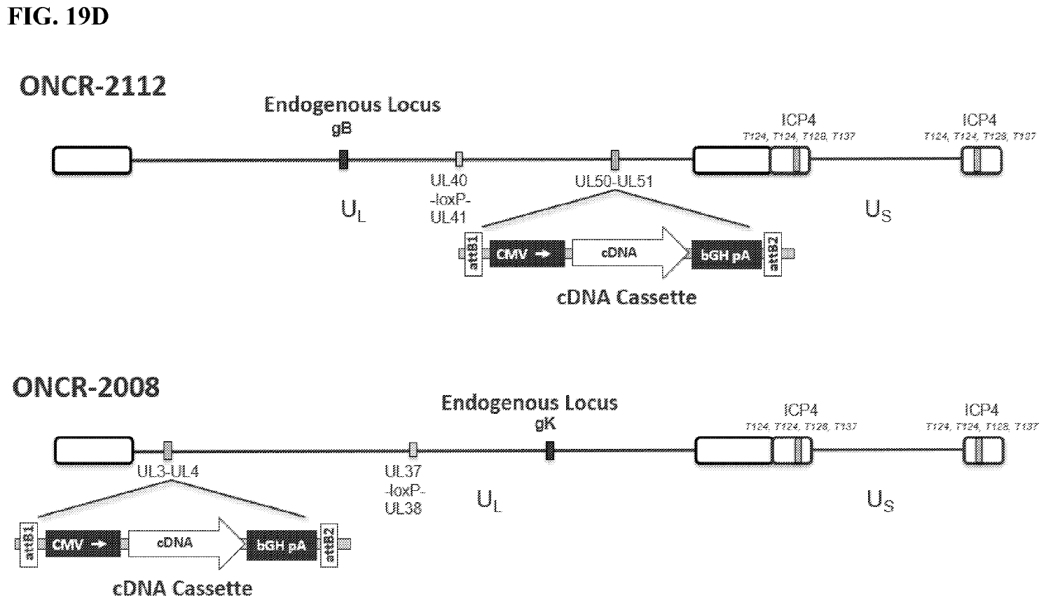

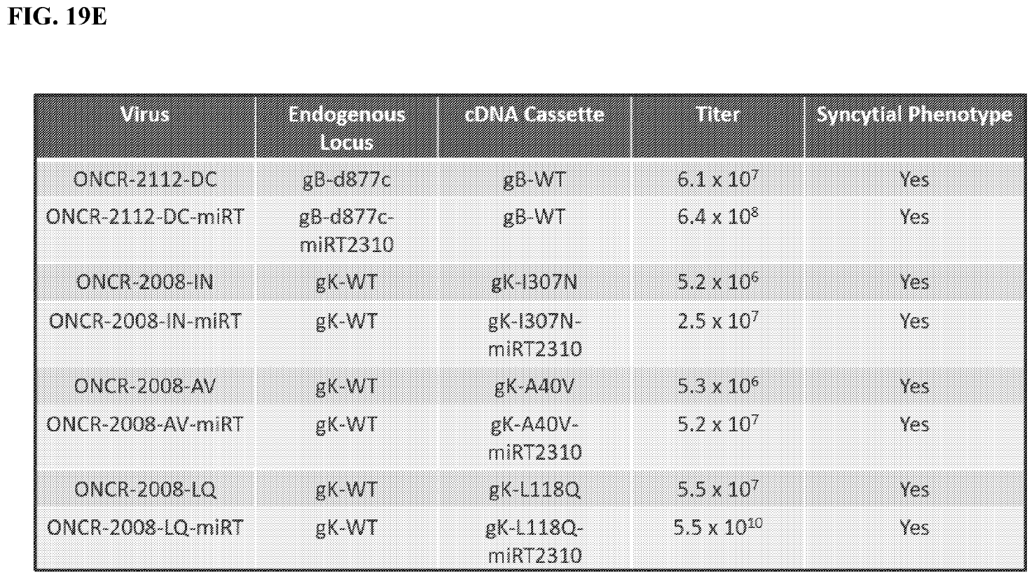

As shown in FIG. 19A, this miR-TS cassette, termed miR-T2310, consisted of 4 copies of each miRNA target sequence separated by a 4 bp spacer (SEQ ID NO: 930). The corresponding miRNA target sequences (SEQ ID NO: 927-929) are the reverse complement of miR-34c-5p (SEQ ID NO: 924), miR-299-5p (SEQ ID NO: 925), and miR-582-5p (SEQ ID NO: 926), respectively. As shown in FIG. 19B and FIG. 19C, the miR-T2310 cassette was inserted in the 3′UTR of the gene locus encoding the gK or gB syntytial mutant, located either at the cDNA cassette or the endogenous HSV locus. In the configuration of FIG. 19B, the HSV comprised unmodified gK at its endogenous locus and a miR-T2310 attenuated, mammalian codon optimized syncytial gK-1307N mutant in a cDNA expression cassette. Other gK mutants generated in this configuration included mammalian codon optimized gK-1307N with no miR-T2310 attenuation, mammalian codon optimized gK-A40V with or without miR-T2310 attenuation, and mammalian codon optimized gK-L118Q with or without miR-T2310 attenuation. In the configuration of FIG. 19C, the HSV comprised a miR-T2310 attenuated syncytial gB-d877c mutant at its endogenous locus and a mammalian codon-optimized non-syncytial gB in a cDNA expression cassette. Similarly, another HSV construct comprising syncytial gB-d877c mutant at its endogenous locus but without the miR-T2310 attenuation was also generated in this configuration.

The HSV backbones used in the miR-TS cassette studies were ONCR-2112 and ONCR-2008, which differed by the cDNA cassette location and the loxP location but were otherwise identical (FIG. 19D). The ONCR-2112 HSV backbone was used to evaluate the performance of the gB syncytial mutant constructs, whereas the ONCR-2008 HSV backbone was used to evaluate the performance of the gK syncytial mutant constructs.

To test the production yield of each HSV construct, Vero cells were inoculated with the indicated viruses at a multiplicity of infection of 0.03, then incubated for 5 days to allow for virus outgrowth. The cells were harvested and then lysed through 3 rapid freeze-thaw cycles to release the virus. Lysates were analyzed for virus yield and syncytia formation by plaque assay of serial dilutions on Vero cell monolayers. As shown in FIG. 19E, for all of the HSV constructs tested, miR-T attenuation of syncytial mutants during virus growth significantly increased virus production yield and they all displayed syncytial phenotype

### Example 8—Codon Optimization of Transgene

Experiments were conducted to test the effect of G/C content on the protein expression level of codon optimized transgenes in the HSV constructs. Several ORFs encoding mouse IL-12 (mIL12) and anti-PD1 antibody were tested. These ORFs have different G/C contents ranging from 52% to 64%:
SEQ ID NO: 931: G/C content: 64%;
SEQ ID NO: 932: G/C content: 63%;
SEQ ID NO: 933: G/C content: 60%;
SEQ ID NO: 934: G/C content: 52%;
SEQ ID NO: 935: G/C content: 52% (T2A);

In these ORFs, the coding sequences for mIL12 and anti-PD1 antibody were separated by a furin-T2A cleavage peptide coding sequence and the two subunits of mIL12 were linked by a 15-mer GS linker peptide, except the 52% GC (T2A) construct (SEQ ID NO: 935) in which the two subunits of mIL12 were linked by a furin-T2A cleavage peptide.

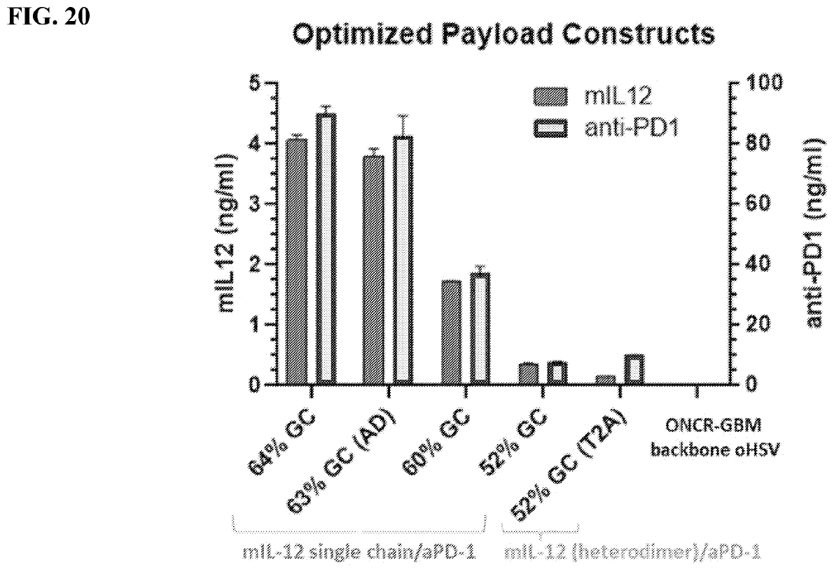

Each of the ORFs was cloned into the HSV viral construct at the UL50-UL51 intergenic region and tested for protein expression. As shown in FIG. 20, higher G/C content in the transgene ORF enhanced payload expression: the protein expression levels of IL-12 and anti-PD1 antibody were increased by more than 10-fold by switching from ˜52% G/C content in the mammalian codon-optimized version to ˜63% G/C content in the A. dehalogenans codon-optimized version. Similar correlations of higher G/C content and increased protein expression level were also observed in codon optimized ORFs encoding two reporter proteins. Thus, higher ORF G/C content (e.g., those optimized based on the codon usage of A. dehalogenans) increases the expression level of the transgene(s) in the HSV construct.

### Example 9—Oncolytic HSV Viruses with Payloads for Treating Glioblastoma

In vivo experiments were conducted to test the efficacy of the HSV oncolytic virus encoding different payload molecules against glioblastoma. Two orthotopic mouse models were used. One orthotopic model used CT2A cells modified to express FLuc which enables imaging post-implantation. The other orthotopic model used GL261 cells modified to express mNectin1, which enabled HSV infection, and also to express FLuc which enabled imaging post-implantation. Cells were implanted intracranially in 12-week-old C57B16 mice by stereotactic injection, and one week later the tumors were injected with the indicated viruses. A brief description of the protocols for implantation is provided in Takenaka et al. Nat Neurosci 22, 729-740 (2019).

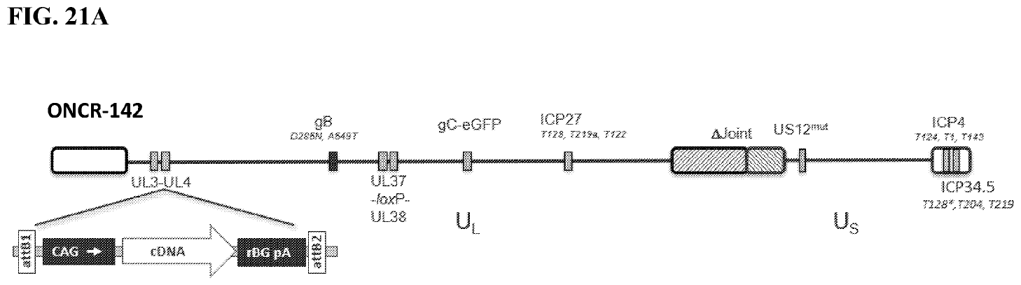

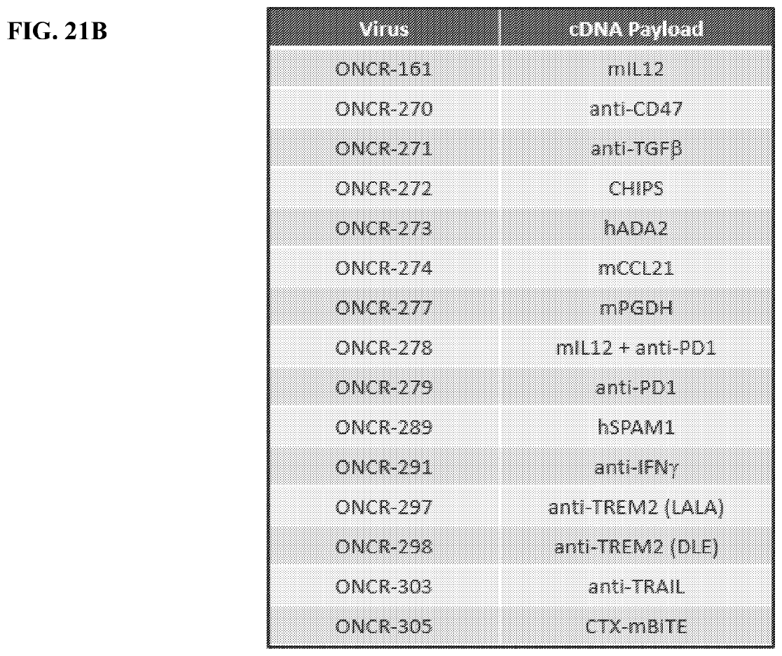

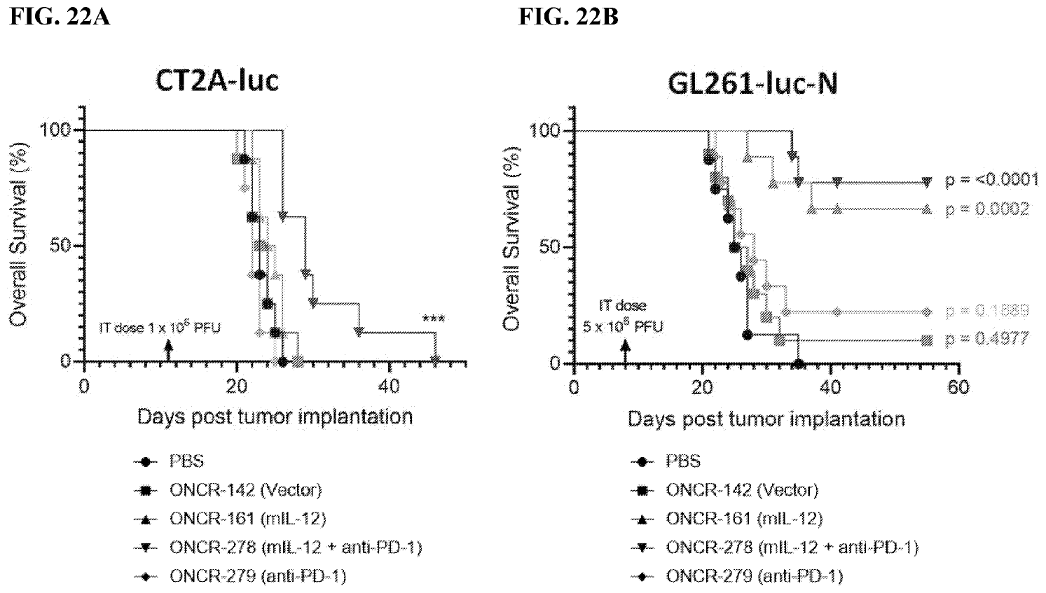

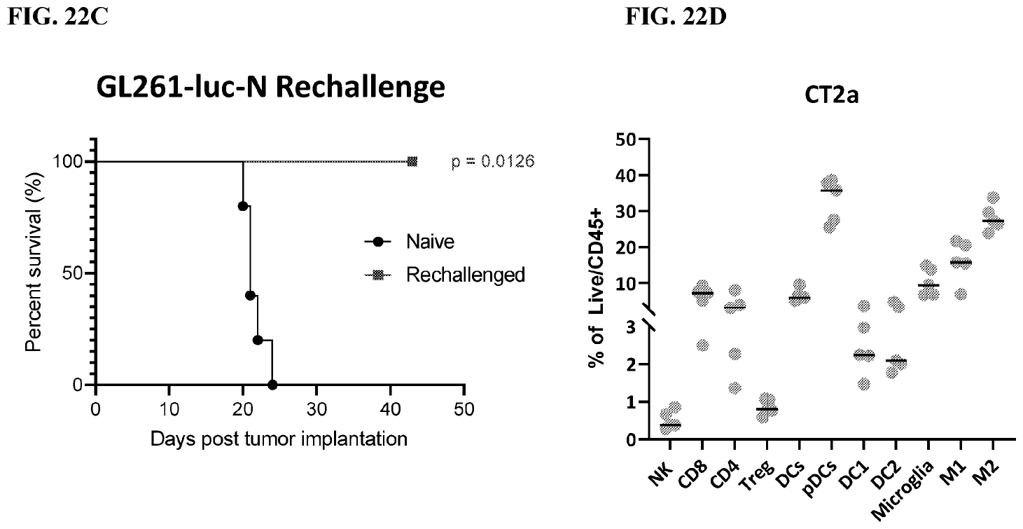

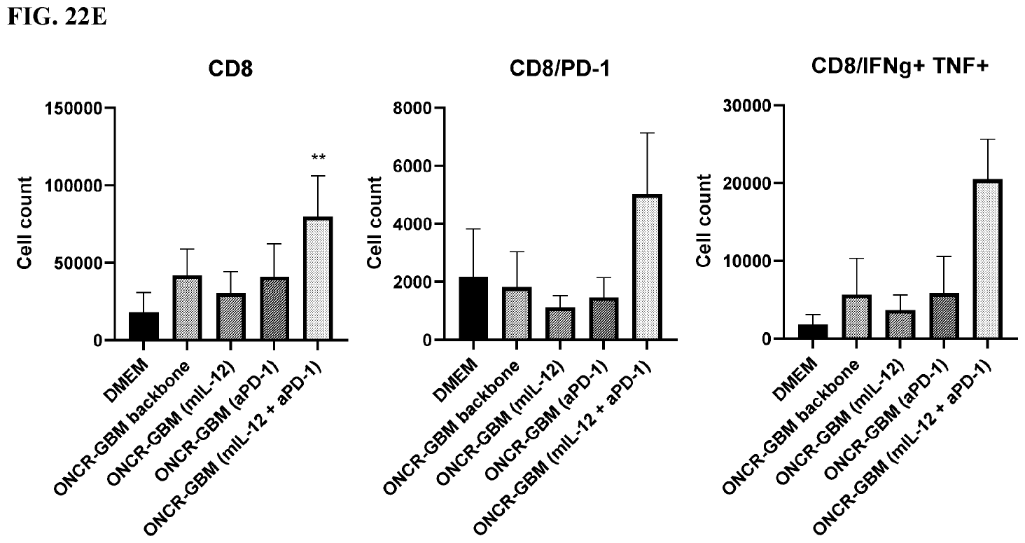

HSV oncolytic viruses encoding the payload molecule(s) were constructed by inserting the payload expression cassette into the UL3-UL4 intergenic region, and inserting the BAC sequences into the UL37-UL38 intergenic region. The virus backbone (control virus) is ONCR-142, which was a cloned derivative of the HSV KOS strain that (i) had miR-T attenuation cassettes in ICP4, ICP27, and ICP34.5, (ii) had the entire internal repeat region deleted (DJoint), and (iii) comprised the indicated mutations in US12, gB, and gC. The control virus contained only the BAC sequences but not the payload sequence. See FIG. 21A. The payload molecule(s) in various HSV constructs are shown in FIG. 21B. In the first set of experiments, viruses encoding mouse IL-12 (mIL-12) and/or anti-PD-1 antibody and the control virus were each injected intratumorally at the indicated time and dosage according to FIG. 22A and FIG. 22B. The results show that the HSV oncolytic virus expressing both mIL12 and the anti-PD1 antibody displayed synergistic effect at prolonging the survival of the animal models, compared to either payload alone. The HSV oncolytic virus expressing mIL12 also significantly prolonged the survival of the animals although it was less effective than the virus expressing both mIL12 and the anti-PD1 antibody. As shown in FIG. 22C, the the animals were also protected from tumor cell re-challenge study in the GL261 model. FIG. 22D shows the percentage of various immune cells in the CT2A model. And FIG. 22E shows that ONCR-GBM with both mIL12 and anti-PD1 payloads stimulated CD8 T cells recruitment and activation.

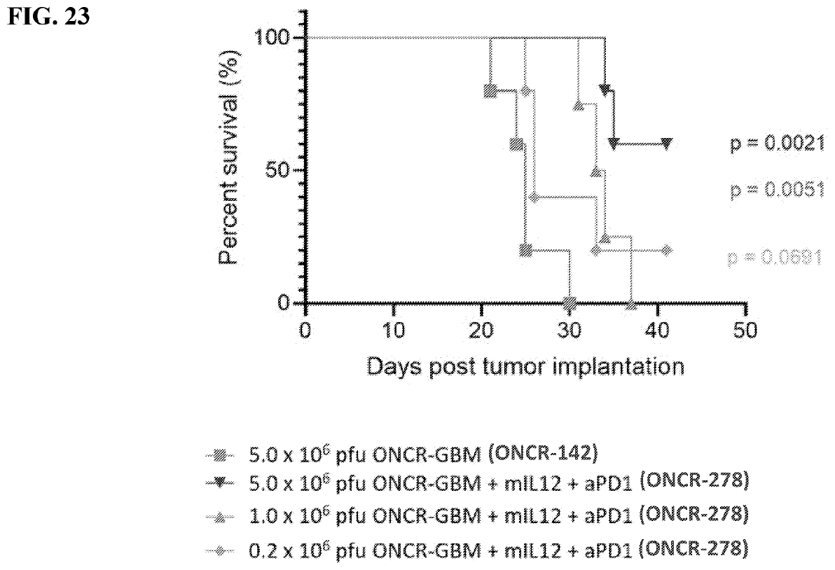

Additional experiments were then conducted to analyze the dose-response relationship of the HSV oncolytic virus expressing both mIL12 and anti-PD1 antibody (ONCR-278) in the GL261 orthotopic mouse model. As shown in FIG. 23, the results demonstrated clear dose response for the HSV oncolytic virus expressing both mIL12 and anti-PD1 antibody. Higher doses were more efficacies than lower doses. And the intermediate, suboptimal dose of 1×10⁶ pfu was selected for use in combination screens by co-dosing with viruses encoding alternative payloads (e.g., antibodies, cytokines, and/or enzymes) based on their potential to modulate the immune-suppressive tumor microenvironment of GBM. The combination of payloads could then be incorporated into the same HSV oncolytic virus.

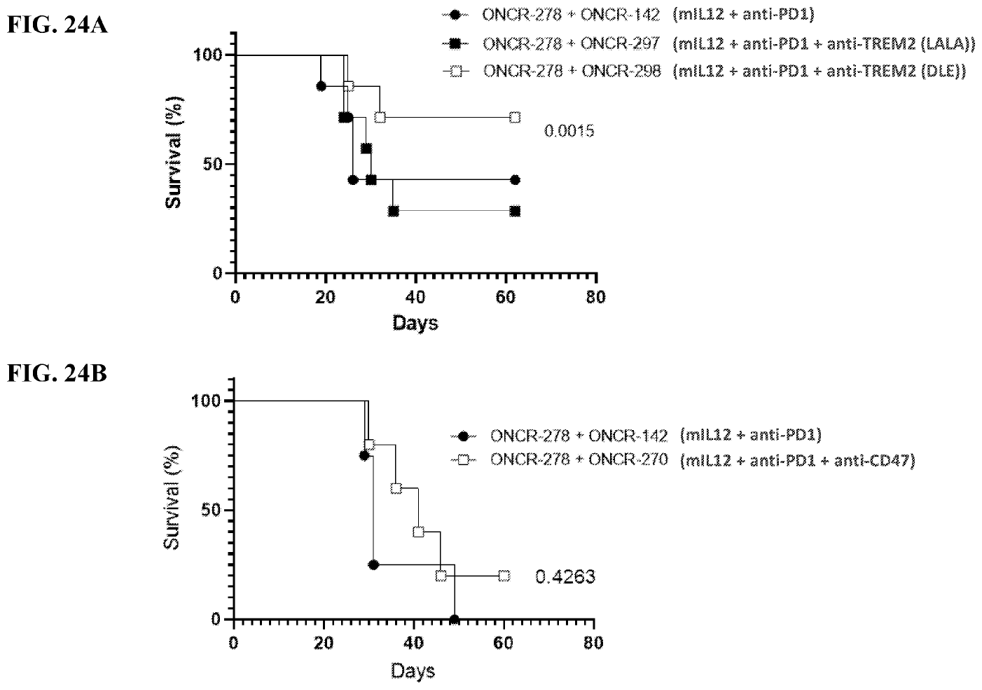

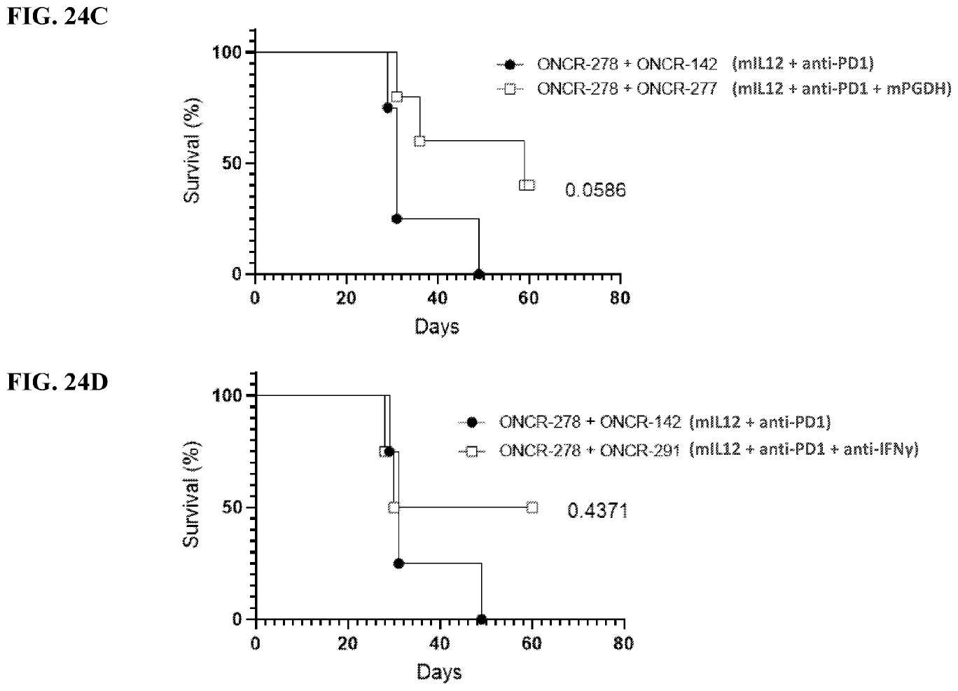

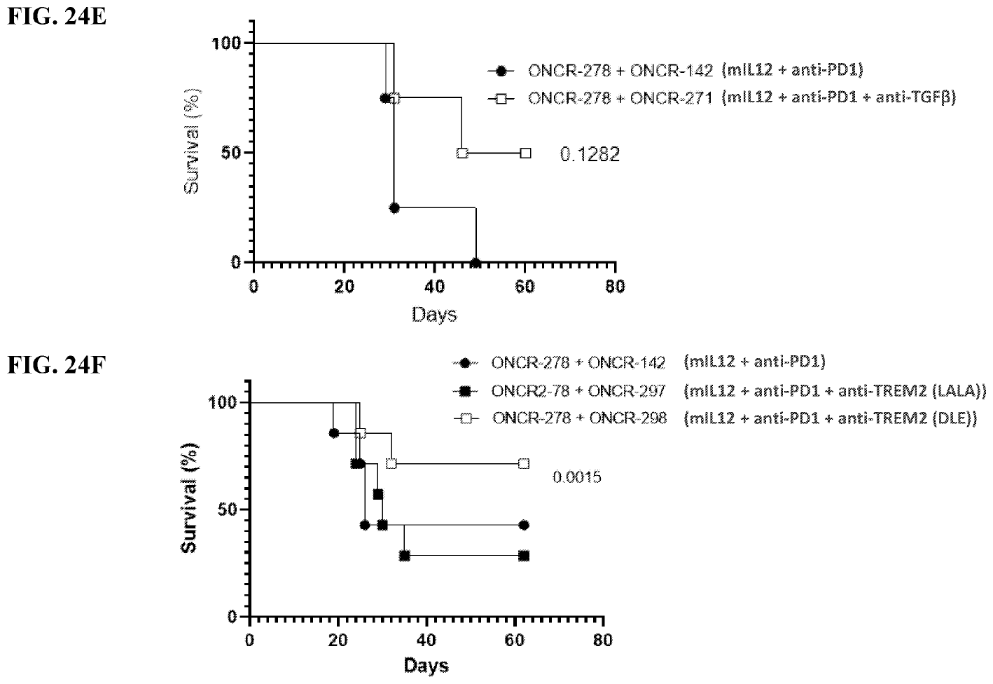

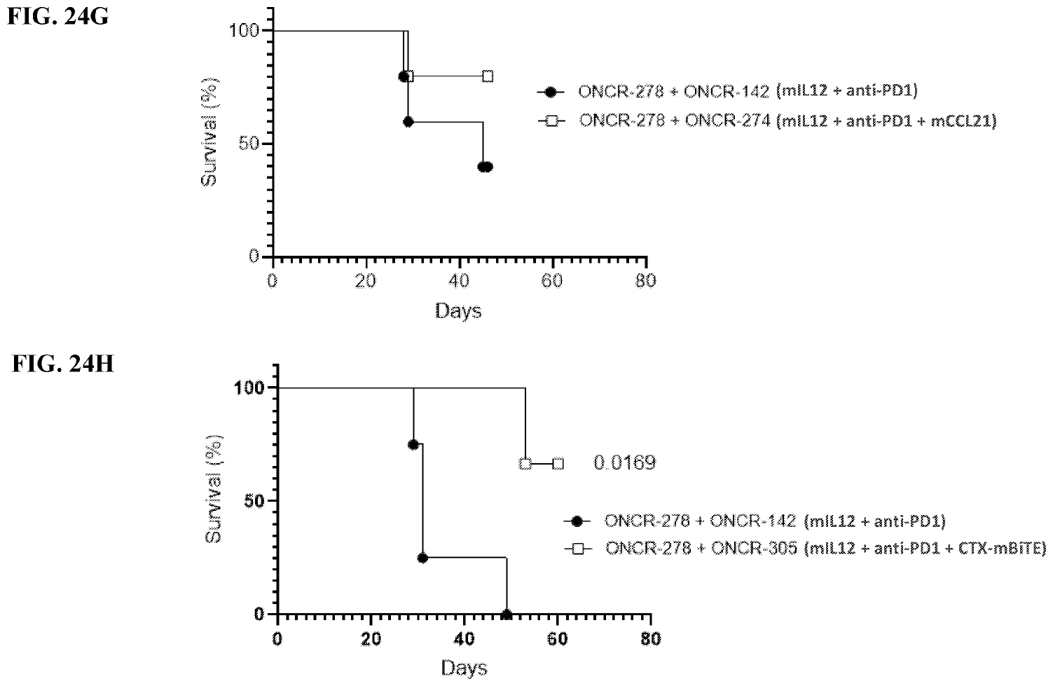

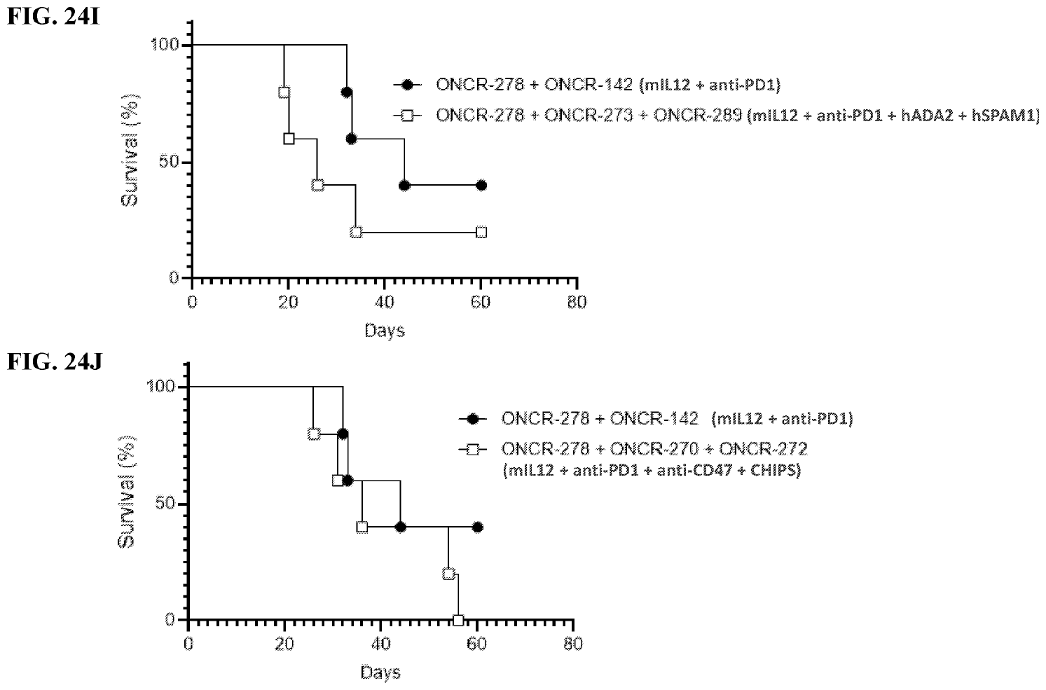

For the combination screens, GL261-luc-N1 cells were intracranially implanted in 12-week-old C57B16 mice, then 1 week later the tumors were injected with the combination of viruses encoding various candidate payloads. The results are shown in FIGS. 24A-24J. Each of the anti-TREM2-DLE, PGDH, and CTX-mBiTE payloads were identified as providing improved efficacy when co-expressed with the mIL12 and anti-PD1 payload.

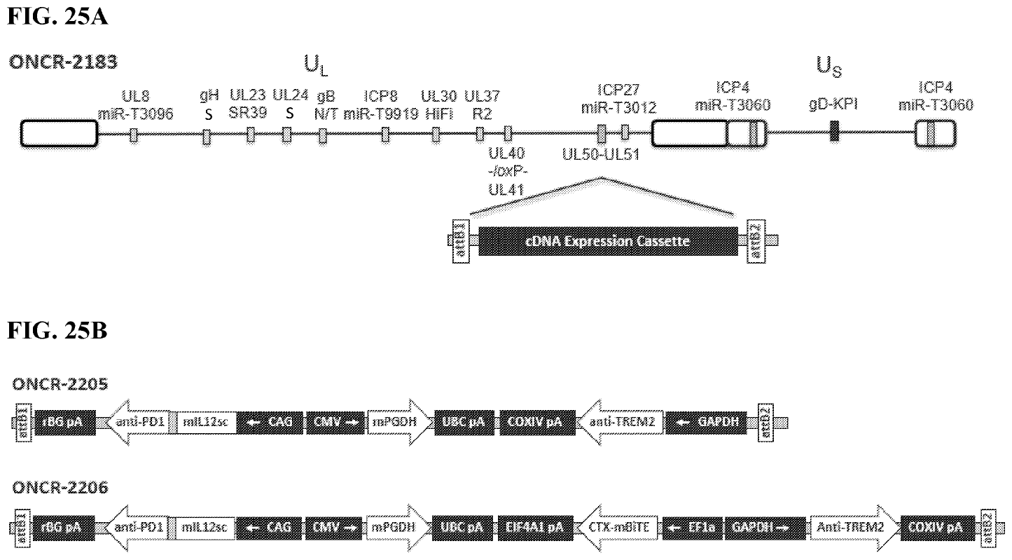

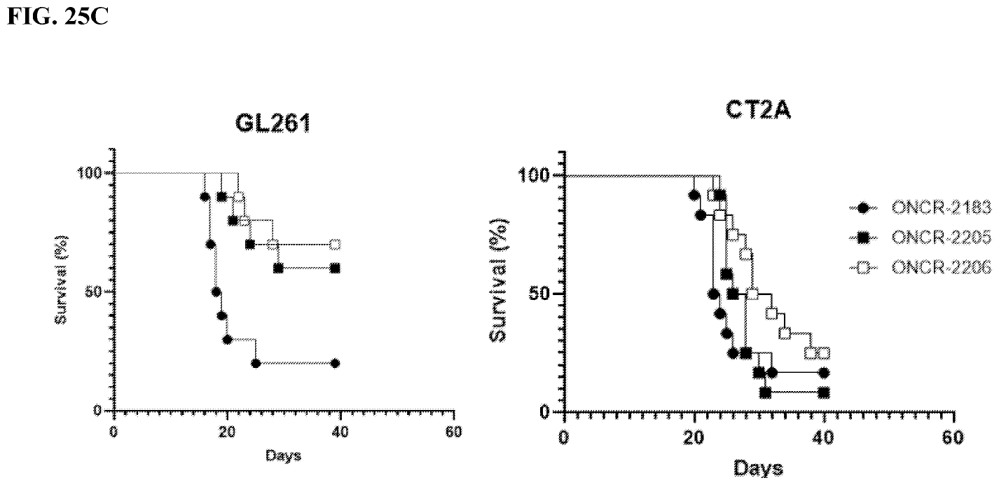

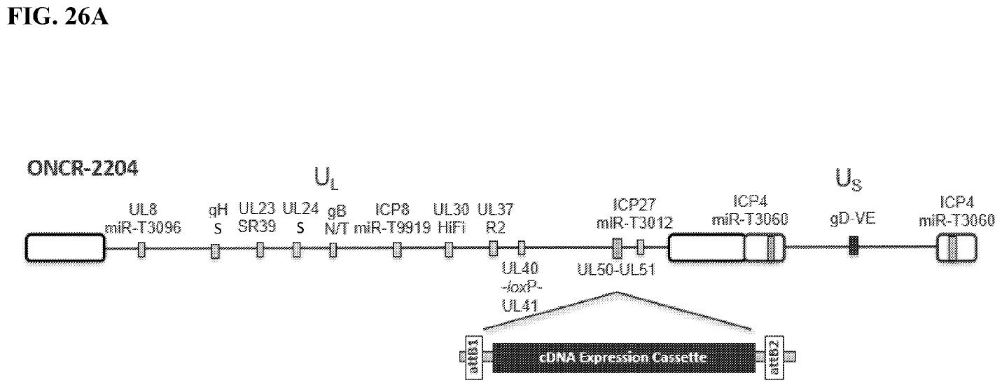

The efficacy of payload combinations was also tested in the ONCR-2183 HSV backbone (FIG. 25A), which is a cloned derivative of the HSV MacIntyre strain containing:
miR-T attenuation cassettes miR-T 3060 (SEQ ID NO: 858), miR-T 9919 (SEQ ID NO: 859), miR-T 3012 (SEQ ID NO: 873), and miR-T 3096 (SEQ ID NO: 874) in ICP4 (both loci), ICP8, ICP27, and UL8, respectively;
UL23 encoding a thymidine kinase comprising L159I, 1160F, F161L, A168F and L169M combo mutations;
UL27 encoding a glycoprotein B (gB) comprising D285N/A549T mutations;
UL30 encoding a DNA polymerase catalytic subunit (DPCS) comprising L774F mutation;
UL37 encoding a tegument protein comprising the “R2” combo mutations (Q403A/E452A/Q455A/Q511A/R515A).
US6 encoding a mutant gD with the native amino acid residues 6-24 of SEQ ID NO: 921 replaced with a knottin peptide that binds to integrins (SEQ ID NO: 922);
the loxP-BAC replicon located in the UL40-UL41 intergenic region; and
a cDNA expression cassette located in the UL50-UL51 intergenic region.

The HSV construct further contains syncytial mutations in the viral genes encoding gH and UL24. Exemplary cDNA expression cassettes are provided in FIG. 25B, each of which can be inserted into the UL50-UL51 intergenic locus of ONCR-2183 to create a recombinant HSV for expressing the corresponding payloads combination of i) IL-12, anti-PD1, PGDH, and anti-TREM2 (ONCR-2205); or ii) IL-12, anti-PD1, PGDH, CTX-BITE, and anti-TREM2 (ONCR-2206). Each of these HSV constructs were tested for tumor inhibition efficacy using the GL261 and CT2A Model Systems. GL261-luc-N1 or CT2A-luc-N1 cells were intracranially implanted in 12-week-old C57B16 mice, then 1 week later the tumors were injected with the indicated viruses. As shown in FIG. 25C, the payload combination in either ONCR-2205 or ONCR-2206 provides superior tumor inhibition efficacy compared to the no payload control ONCR-2183 in GL261 and CT2A models.

Based on the examples above, an HSV backbone derived from the HSV MacIntyre strain, ONCR-2204, was constructed to incorporate various design features (FIG. 26A), including:
miR-T attenuation cassettes miR-T 3060 (SEQ ID NO: 858), miR-T 9919 (SEQ ID NO: 859), miR-T 3012 (SEQ ID NO: 873), and miR-T 3096 (SEQ ID NO: 874) in ICP4 (both loci), ICP8, ICP27, and UL8, respectively;
UL23 encoding a thymidine kinase comprising L159I, I160F, F161L, A168F and L169M combo mutations;
UL27 encoding a glycoprotein B (gB) comprising D285N/A549T mutations;
UL30 encoding a DNA polymerase catalytic subunit (DPCS) comprising L774F mutation;
UL37 encoding a tegument protein comprising the “R2” combo mutations (Q403A/E452A/Q455A/Q511A/R515A).
US6 encoding a mutant gD with the native amino acid residues 6-24 of SEQ ID NO: 921 replaced with a VHH nanobody that binds the extracellular domain of EGFR (SEQ ID NO: 923);
the loxP-BAC replicon located in the UL40-UL41 intergenic region; and
a cDNA expression cassette located in the UL50-UL51 intergenic region.

The HSV construct further contains syncytial mutations in the viral genes encoding gH and UL24. Exemplary cDNA expression cassettes are provided in FIG. 26B, each of which can be inserted into the UL50-UL51 intergenic locus of ONCR-2204 to create a recombinant HSV for expressing the corresponding payloads combination of i) IL-12, anti-PD1, PGDH, and anti-TREM2 (ONCR-2218); ii) IL-12, anti-PD1, PGDH, CTX-BITE, and anti-TREM2 (ONCR-2219); or iii) IL-12, anti-PD1, CTX-BiTE, and anti-TREM2 (ONCR-2233). Each of these HSV constructs are tested for tumor inhibition efficacy using the GL261-luc-N1 and CT2A-luc-N1 intracranial syngeneic GBM Model Systems. GL261-luc-N1 or CT2A-luc-N1 cells are intracranially implanted in 12-week-old C57B16 mice, then about 7 days later the tumors are injected with the indicated viruses, and the survival benefit of these HSV constructs is compared, similar to the analysis performed for the ONCR-2183 backbone based HSV constructs.

While preferred embodiments of the present disclosure are shown and described herein, it will be obvious to those skilled in the art that such embodiments are provided by way of example only. Numerous variations, changes, and substitutions can be implemented by those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.

TABLE 10
Summary of relationships between 12 select oncomiRs (9 tumor
suppressors and 3 oncogenic miRNAs) and various cancers

| | Down-regulated | | | | | | | | | Up-regulated | | |
|---|---|---|---|---|---|---|---|---|---|---|---|---|
| | | miR- | miR- | miR- | miR- | miR- | miR- | miR- | miR- | miR- | miR- | miR- |
| Malignancy | let-7 | 15a | 16 | 29a | 34a | 98 | 101 | 124 | 202 | 17 | 21 | 155 |
| acute lymphoblastic leukemia | X | | | | | | | X | | | | |
| acute myeloid leukemia | X | | | X | | | | | | X | | X |
| acute promyelocytic leukemia | X | | | | | | | | | | | |
| adrenal cortical carcinoma | | | | | | | | | | | X | |
| anaplastic astrocytoma | | | | | | | | X | | | | |
| anaplastic large-cell lymphoma | | | | | | | | | | | | X |
| astrocytoma | | | | | | | | X | | | | |
| B cell lymphoma | | | | | X | | | | | X | | |
| bladder cancer | | | X | | X | | X | X | | X | | X |
| breast cancer | X | X | X | X | X | X | | X | | X | | X |
| breast carcinoma | | | | | | | | | | X | | |
| bronchioloalveolar carcinoma | X | | | | | X | | | | | | |
| cervical cancer | | | | | | | | X | | X | | X |
| cervical carcinoma | | X | X | | X | | X | | | | | |
| cervical squamous cell | | | | X | | | | X | | | | |
| carcinoma | | | | | | | | | | | | |
| cholangiocarcinoma | | | | | X | | X | | | X | | |
| chondrosarcoma | X | | | | | | | | | | | |
| chordoma | | | | | X | | | | | | | |
| choriocarcinoma | | | | | X | | | | | | | |
| chronic lymphocytic leukemia | | X | X | | | | | | | | | X |
| chronic myelogenous leukemia | | | X | | | | | | | | | X |
| clear cell renal cell cancer | | | | | X | | | | | | | X |
| colon cancer | X | | | | X | X | X | | | | | X |
| colorectal cancer | X | X | X | X | X | | X | X | X | X | | X |
| colorectal carcinoma | | | | | | | | | | X | | X |
| cutaneous T cell lymphoma | | | | | | | | | | | | X |
| diffuse large B cell lymphoma | | | | | | | | | | | | X |
| endometrial cancer | | | | | X | | X | | | | | X |
| epithelial ovarian cancer | | | | | | | | X | | | | |
| esophageal cancer | | X | | | | | X | X | | | | |
| esophageal squamous cell | X | | X | | X | X | X | | | X | | |
| carcinoma | | | | | | | | | | | | |
| extrahepatic cholangiocarcinoma | | | | | X | | | | | | | |
| follicular lymphoma | | | | | | | | | X | | | |
| gallbladder carcinoma | | | | | | | | | | | | X |
| gastric cancer | X | | | X | X | X | X | X | X | X | | X |
| glioblastoma | X | | | | X | | X | X | | | | |
| glioma | X | | X | | X | X | | X | | X | | X |
| head and neck cancer | | | | | | | | | | | | |
| head and neck squamous cell | X | | X | X | X | | | | | | X | |
| carcinoma | | | | | | | | | | | | |
| hepatocellular carcinoma | X | | X | X | X | X | X | X | X | X | X | X |
| hypopharyngeal squamous | | | | | | | | | | | X | |
| cell carcinoma | | | | | | | | | | | | |
| kidney cancer | | | | | | | | | | | X | |
| laryngeal carcinoma | | | X | | | | | | | | X | |
| laryngeal squamous cell | | | | | | | X | | | | X | |
| carcinoma | | | | | | | | | | | | |
| liver cancer | | | | | | | X | | | | X | X |
| lung adenocarcinoma | | | X | | | | | | | | | X |
| lung cancer | X | X | X | | X | X | X | | | X | X | X |
| malignant melanoma | X | | | | X | X | X | | | X | X | X |
| malt lymphoma | | | | | | | | | | | | X |
| mantle cell lymphoma | | | | X | | | | X | | X | | X |
| medulloblastoma | | | | | | | | X | | X | | |
| mesenchymal cancer | | | | X | | | | | | | | |
| monocytic leukemia | | | | X | | | | | | | | |
| multiple myeloma | | | | | | | | | | | X | |
| nasopharyngeal cancer | | | | | | | | | | X | | |
| nasopharyngeal carcinoma | X | | | | | X | X | X | | | X | X |
| neuroblastoma | X | X | X | X | X | X | | X | | | | |
| non-small cell lung cancer | X | X | X | X | X | | X | X | | X | X | X |
| oral cancer | X | | | | X | | | | | | X | |
| oral squamous cell carcinoma | | | | X | | | | X | | | X | X |
| osteosarcoma | X | X | X | | X | | X | X | X | X | X | |
| ovarian cancer | X | | | | X | X | | X | | | X | X |
| ovarian carcinoma | | | | | | | X | | | | | |
| pancreatic adenocarcinoma | | | | | X | | | | | | X | |
| pancreatic cancer | | X | | | | | X | X | | X | X | |
| pancreatic ductal | X | X | X | | X | X | | | | | X | |
| adenocarcinoma | | | | | | | | | | | | |
| papillary thyroid carcinoma | X | | X | | X | | X | | | | X | X |
| pituitary carcinoma | | | | | | | | | | X | | |
| prostate cancer | X | X | X | | X | | X | X | | | X | |
| rectal cancer | | | | | X | | | | | | X | X |
| renal cell carcinoma | X | | X | | X | | | | | | X | |
| renal clear cell carcinoma | X | | | | | | | | | | | X |
| retinoblastoma | | | | | X | | X | | | | X | |
| squamous carcinoma | | X | X | | X | | | | | | X | X |
| T cell lymphoblastic lymphoma | | | | | | | | | | X | | |
| uveal melanoma | | | | | X | | | | | | | |

TABLE 11
Summary of miRNA expression in cancer

| Malignancy | Down-regulated miRs | Up-regulated miRs |
|---|---|---|
| breast cancer | let-7a, let-7a-1, let-7a-2, let-7a-3, let-7b, | mir-10b, mir-125a, mir-135a, |
| | let-7c, let-7d, let-7e, let-7f-1, let-7f-2, | mir-140, mir-141, mir-142, mir- |
| | let-7g, let-7i, mir-100, mir-107, mir-10a, | 150, mir-155, mir-181a, mir- |
| | mir-10b, mir-122, mir-124, mir-1258, | 181b, mir-182, mir-18a, mir-18b, |
| | mir-125a-5p, mir-125b, mir-126, mir- | mir-191, mir-196a, mir-197, mir- |
| | 127, mir-129, mir-130a, mir-132, mir- | 19a, mir-19b, mir-200a, mir- |
| | 133a, mir-143, mir-145, mir-146a, mir- | 200b, mir-200c, mir-203, mir- |
| | 146b, mir-147, mir-148a, mir-149, mir- | 205, mir-20a, mir-20b, mir-21, |
| | 152, mir-153, mir-15a, mir-16, mir-17- | mir-217, mir-221, mir-224, mir- |
| | 5p, mir-181a, mir-1826, mir-183, mir- | 23a, mir-24, mir-24-2-5p, mir-24- |
| | 185, mir-191, mir-193a-3p, mir-193b, | 3p, mir-27a, mir-29a, mir-29b-1, |
| | mir-195, mir-199b-5p, mir-19a-3p, mir- | mir-29b-2, mir-29c, mir-373, |
| | 200a, mir-200b, mir-200c, mir-205, mir- | mir-378, mir-423, mir-429, mir- |
| | 206, mir-211, mir-216b, mir-218, mir-22, | 495, mir-503, mir-510, mir-520c, |
| | mir-26a, mir-26b, mir-300, mir-30a, mir- | mir-526b, mir-96 |
| | 31, mir-335, mir-339-5p, mir-33b, mir- | |
| | 34a, mir-34b, mir-34c, mir-374a, mir- | |
| | 379, mir-381, mir-383, mir-425, mir-429, | |
| | mir-450b-3p, mir-494, mir-495, mir-497, | |
| | mir-502-5p, mir-517a, mir-574-3p, mir- | |
| | 638, mir-7, mir-720, mir-7515, mir-92a, | |
| | mir-98, mir-99a, mmu-mir-290-3p, | |
| | mmu-mir-290-5p | |
| chondrosarcoma | let-7a, mir-100, mir-136, mir-145, mir- | |
| | 199a, mir-222, mir-30a, mir-335, mir- | |
| | 376a | |
| colorectal cancer | let-7a, mir-1, mir-100, mir-101, mir-124, | let-7a, mir-103, mir-106a, mir- |
| | mir-125a, mir-126, mir-129, mir-1295b- | 10b, mir-1179, mir-1229, mir- |
| | 3p, mir-1307, mir-130b, mir-132, mir- | 1246, mir-125b-2*, mir-1269a, |
| | 133a, mir-133b, mir-137, mir-138, mir- | mir-130b, mir-133b, mir-135a, |
| | 139, mir-139-5p, mir-140-5p, mir-143, | mir-135a-1, mir-135a-2, mir- |
| | mir-145, mir-148a, mir-148b, mir-149, | 135b, mir-139-3p, mir-145, mir- |
| | mir-150-5p, mir-154, mir-15a, mir-15b, | 150, mir-150*, mir-155, mir-17, |
| | mir-16, mir-18a, mir-191, mir-192, mir- | mir-181a, mir-182, mir-183, mir- |
| | 193a-5p, mir-194, mir-195, mir-196a, | 18a, mir-191, mir-196a, mir- |
| | mir-198, mir-199a-5p, mir-200c, mir- | 196b, mir-19a, mir-19b, mir- |
| | 203, mir-204-5p, mir-206, mir-212, mir- | 200b, mir-200c, mir-203, mir- |
| | 215, mir-218, mir-22, mir-224, mir-24- | 204-5p, mir-20a, mir-20a-5p, |
| | 3p, mir-26b, mir-27a, mir-28-3p, mir-28- | mir-21, mir-210, mir-211, mir- |
| | 5p, mir-29b, mir-30a-3p, mir-30b, mir- | 221, mir-223, mir-224, mir-23a, |
| | 320a, mir-328, mir-338-3p, mir-342, mir- | mir-25, mir-27a, mir-29a, mir- |
| | 345, mir-34a, mir-34a-5p, mir-361-5p, | 301a, mir-31, mir-32, mir-320b, |
| | mir-375, mir-378, mir-378a-3p, mir- | mir-326, mir-424, mir-429, mir- |
| | 378a-5p, mir-409-3p, mir-422a, mir- | 494, mir-497, mir-499-5p, mir- |
| | 4487, mir-483, mir-497, mir-498, mir- | 592, mir-630, mir-7-5p, mir- |
| | 518a-3p, mir-551a, mir-574-5p, mir-625, | 892a, mir-92, mir-92a, mir-93, |
| | mir-638, mir-7, mir-96-5p | mir-95, mir-96 |
| esophageal squamous | let-7a, let-7a-1, let-7a-2, let-7a-3, let-7b, | mir-100, mir-1179, mir-1290, |
| cell carcinoma | let-7c, let-7d, let-7e, let-7f-1, let-7f-2, | mir-130b, mir-145, mir-16, mir- |
| | let-7g, let-7i, mir-1, mir-100, mir-101, | 17, mir-183, mir-18a, mir-19a, |
| | mir-126, mir-1294, mir-133a, mir-133b, | mir-19b, mir-208, mir-20a, mir- |
| | mir-138, mir-143, mir-145, mir-150, mir- | 21, mir-218, mir-223, mir-25, |
| | 185, mir-195, mir-200b, mir-203, mir-21, | mir-30a-5p, mir-31, mir-330-3p, |
| | mir-210, mir-214, mir-218, mir-22, mir- | mir-373, mir-9, mir-92a, mir-942 |
| | 27a, mir-29b, mir-29c, mir-302b, mir- | |
| | 34a, mir-375, mir-494, mir-518b, mir- | |
| | 655, mir-98, mir-99a | |
| gastric cancer | let-7a, let-7b, let-7g, mir-1, mir-101, mir- | mir-100, mir-103, mir-106a, mir- |
| | 103a, mir-10a, mir-10b, mir-1207-5p, | 106b, mir-107, mir-10a, mir-10b, |
| | mir-122, mir-1228*, mir-124, mir-124- | mir-1259, mir-125b, mir-126, |
| | 3p, mir-125a-3p, mir-126, mir-1266, mir- | mir-1274a, mir-1303, mir-130b*, |
| | 1271, mir-129-1-3p, mir-129-2-3p, mir- | mir-135a-5p, mir-135b, mir-138, |
| | 129-3p, mir-129-5p, mir-133a, mir-133b, | mir-143, mir-146a, mir-147, mir- |
| | mir-137, mir-141, mir-143, mir-144, mir- | 148a, mir-150, mir-17, mir-17- |
| | 145, mir-146a, mir-146a-5p, mir-148a, | 5p, mir-181a, mir-181a-2*, mir- |
| | mir-148b, mir-149, mir-152, mir-155, | 181a-5p, mir-181c, mir-183, mir- |
| | mir-155-5p, mir-181a, mir-181b, mir- | 185, mir-18a, mir-191, mir-192, |
| | 182, mir-183, mir-185, mir-194, mir-195, | mir-196a, mir-196a*, mir-196a- |
| | mir-197, mir-199a-3p, mir-200b, mir- | 5p, mir-196b, mir-199a, mir- |
| | 200c, mir-202-3p, mir-204, mir-204-5p, | 199a-3p, mir-199a-5p, mir-19a, |
| | mir-205, mir-206, mir-210, mir-212, mir- | mir-19b, mir-200b, mir-20a, mir- |
| | 217, mir-218, mir-22, mir-23b, mir-24, | 21, mir-214, mir-215, mir-221, |
| | mir-26a, mir-29a, mir-29a-3p, mir-29b, | mir-221*, mir-222, mir-223, mir- |
| | mir-29b-1, mir-29b-2, mir-29c, mir-30a- | 224, mir-23a, mir-23b, mir-27a, |
| | 5p, mir-30b, mir-31, mir-328, mir-329, | mir-27b, mir-296-5p, mir-301a, |
| | mir-331-3p, mir-335-5p, mir-338, mir- | mir-302f, mir-337-3p, mir-340*, |
| | 338-3p, mir-34a, mir-34b, mir-34c, mir- | mir-34a, mir-362-3p, mir-370, |
| | 361-5p, mir-367, mir-375, mir-378, mir- | mir-374a, mir-377, mir-421, mir- |
| | 409-3p, mir-410, mir-429, mir-433, mir- | 425, mir-500, mir-520c-3p, mir- |
| | 449, mir-449a, mir-490-3p, mir-494, mir- | 544, mir-575, mir-601, mir-616*, |
| | 497, mir-503, mir-506, mir-513b, mir- | mir-650, mir-92, mir-98, mir-99a |
| | 520d-3p, mir-542-3p, mir-622, mir-625, | |
| | mir-638, mir-663, mir-7, mir-765, mir-9 | |
| glioma | let-7a, let-7f, mir-106a, mir-107, mir- | mir-106b, mir-106b-5p, mir-10b, |
| | 122, mir-124, mir-124-5p, mir-124a, mir- | mir-125b, mir-132, mir-155, mir- |
| | 125b, mir-128, mir-136, mir-137, mir- | 17, mir-181a, mir-182, mir-183, |
| | 139, mir-143, mir-145, mir-146a, mir- | mir-193b, mir-19a, mir-19b, mir- |
| | 146b, mir-146b-5p, mir-152, mir-15b, | 20a, mir-210, mir-214, mir-221, |
| | mir-16, mir-181a, mir-181a-1, mir-181a- | mir-222, mir-224, mir-23a, mir- |
| | 2, mir-181b, mir-181b-1, mir-181b-2, | 24, mir-24-3p, mir-25, mir-26a, |
| | mir-181c, mir-181d, mir-184, mir-185, | mir-27a-3p, mir-27b, mir-30a-5p, |
| | mir-195, mir-199a-3p, mir-200a, mir- | mir-30e, mir-30e*, mir-328, mir- |
| | 200b, mir-203, mir-204, mir-205, mir- | 335, mir-33a, mir-372, mir-486, |
| | 218, mir-219-5p, mir-23b, mir-26b, mir- | mir-494, mir-497, mir-566, mir- |
| | 27a, mir-29c, mir-320, mir-326, mir-328, | 603, mir-650, mir-675, mir-9, |
| | mir-34a, mir-34c-3p, mir-34c-5p, mir- | mir-92b, mir-93, mir-96 |
| | 375, mir-383, mir-451, mir-452, mir- | |
| | 483-5p, mir-495, mir-584, mir-622, mir- | |
| | 656, mir-7, mir-98 | |
| nasopharyngeal | let-7a, let-7a-1, let-7a-2, let-7a-3, let-7b, | mir-10b, mir-144, mir-149, mir- |
| carcinoma | let-7c, let-7d, let-7e, let-7f-1, let-7f-2, | 155, mir-18a, mir-21, mir-214, |
| | let-7g, let-7i, mir-1, mir-101, mir-124, | mir-24, mir-421, mir-663, mir-7- |
| | mir-138, mir-143, mir-145, mir-148a, | 5p, mir-93 |
| | mir-200b, mir-204, mir-216b, mir-29c, | |
| | mir-320a, mir-324-3p, mir-34c, mir-375, | |
| | mir-378, mir-451, mir-506, mir-9, mir-98 | |
| non-small cell lung | let-7a, let-7c, mir-1, mir-100, mir-101, | mir-10b, mir-125a-5p, mir-1280, |
| cancer | mir-106a, mir-107, mir-124, mir-125a- | mir-136, mir-140, mir-141, mir- |
| | 3p, mir-125a-5p, mir-126*, mir-129, mir- | 142-3p, mir-145, mir-146a, mir- |
| | 133a, mir-137, mir-138, mir-140, mir- | 150, mir-18a, mir-196a, mir-19a, |
| | 143, mir-145, mir-146a, mir-146b, mir- | mir-200a, mir-200c, mir-205, |
| | 148a, mir-148b, mir-149, mir-152, mir- | mir-205-5p, mir-21, mir-212, |
| | 153, mir-154, mir-155, mir-15a, mir-16, | mir-22, mir-221, mir-222, mir- |
| | mir-17-5p, mir-181a-1, mir-181a-2, mir- | 24, mir-25, mir-29c, mir-31, mir- |
| | 181b, mir-181b-1, mir-181b-2, mir-181c, | 328, mir-330-3p, mir-339, mir- |
| | mir-181d, mir-184, mir-186, mir-193b, | 34a, mir-375, mir-494, mir-675- |
| | mir-195, mir-199a, mir-204, mir-212, | 5p, mir-9, mir-92b, mir-93, mir- |
| | mir-221, mir-224, mir-26b, mir-27a, mir- | 95 |
| | 27b, mir-29a, mir-29b, mir-29c, mir-30a, | |
| | mir-30b, mir-30c, mir-30d, mir-30d-5p, | |
| | mir-30e-5p, mir-32, mir-335, mir-338- | |
| | 3p, mir-340, mir-342-3p, mir-34a, mir- | |
| | 34b, mir-361-3p, mir-365, mir-373, mir- | |
| | 375, mir-429, mir-449a, mir-4500, mir- | |
| | 451, mir-4782-3p, mir-497, mir-503, | |
| | mir-512-3p, mir-520a-3p, mir-526b, mir- | |
| | 625*, mir-96, mir-99a | |
| osteosarcoma | let-7a, mir-1, mir-100, mir-101, mir-122, | mir-128, mir-151-3p, mir-17, |
| | mir-124, mir-125b, mir-126, mir-127-3p, | mir-181a, mir-181b, mir-181c, |
| | mir-132, mir-133a, mir-141, mir-142-3p, | mir-18a, mir-191, mir-195-5p, |
| | mir-142-5p, mir-143, mir-144, mir-145, | mir-199a-3p, mir-19a, mir-19b, |
| | mir-153, mir-16, mir-183, mir-194, mir- | mir-20a, mir-21, mir-210, mir- |
| | 195, mir-199a-3p, mir-204, mir-212, mir- | 214, mir-221, mir-27a, mir-300, |
| | 217, mir-218, mir-22, mir-23a, mir-24, | mir-320a, mir-374a-5p, mir-720, |
| | mir-26a, mir-26b, mir-29b, mir-32, mir- | mir-9, mir-92a |
| | 320, mir-335, mir-33b, mir-340, mir-34a, | |
| | mir-34b, mir-34c, mir-375, mir-376c, | |
| | mir-382, mir-3928, mir-424, mir-429, | |
| | mir-449a, mir-451, mir-454, mir-503, | |
| | mir-519d, mir-646 | |
| pancreatic ductal | let-7a, let-7a-1, let-7a-2, let-7a-3, let-7b, | mir-10b, mir-186, mir-18a, mir- |
| adenocarcinoma | let-7c, let-7d, let-7e, let-7f-1, let-7f-2, | 192, mir-194, mir-196a, mir-198, |
| | let-7g, let-7i, mir-126, mir-135a, mir- | mir-203, mir-21, mir-212, mir- |
| | 143, mir-144, mir-145, mir-148a, mir- | 30b-5p, mir-31, mir-34a, mir- |
| | 150, mir-15a, mir-16, mir-200a, mir- | 369-5p, mir-376a, mir-541 |
| | 200b, mir-200c, mir-217, mir-218, mir- | |
| | 337, mir-375, mir-494, mir-615-5p, mir- | |
| | 98 | |
| renal cell carcinoma | let-7a, let-7d, mir-1, mir-106a*, mir-126, | mir-100, mir-1233, mir-1260b, |
| | mir-1285, mir-129-3p, mir-1291, mir- | mir-146a, mir-146b, mir-16, mir- |
| | 133a, mir-133b, mir-135a, mir-138, mir- | 193a-3p, mir-203a, mir-21, mir- |
| | 141, mir-143, mir-145, mir-182-5p, mir- | 210, mir-27a, mir-362, mir-572, |
| | 199a-3p, mir-200a, mir-205, mir-218, | mir-7 |
| | mir-28-5p, mir-30a, mir-30c, mir-30d, | |
| | mir-34a, mir-378, mir-429, mir-509-3p, | |
| | mir-509-5p, mir-646 | |
| bronchioloalveolar | let-7a-1, let-7a-2, let-7a-3, let-7b, let-7c, | |
| carcinoma | let-7d, let-7e, let-7f-1, let-7f-2, let-7g, | |
| | let-7i, mir-98 | |
| colon cancer | let-7a-1, let-7a-2, let-7a-3, let-7b, let-7c, | mir-1290, mir-145, mir-155, mir- |
| | let-7d, let-7e, let-7f-1, let-7f-2, let-7g, | 181a, mir-18a, mir-200c, mir-31, |
| | let-7i, mir-100, mir-101, mir-126, mir- | mir-675 |
| | 142-3p, mir-143, mir-145, mir-192, mir- | |
| | 200c, mir-21, mir-214, mir-215, mir-25, | |
| | mir-302a, mir-320, mir-320a, mir-34a, | |
| | mir-34c, mir-365, mir-373, mir-424, mir- | |
| | 429, mir-455, mir-484, mir-502, mir-503, | |
| | mir-93, mir-98 | |
| hepatocellular | let-7a-1, let-7a-2, let-7a-3, let-7b, let-7c, | mir-106b, mir-10b, mir-122, mir- |
| carcinoma | let-7d, let-7e, let-7f, let-7f-1, let-7f-2, let- | 1228, mir-1269, mir-128a, mir- |
| | 7g, let-7i, mir-1, mir-100, mir-101, mir- | 130a, mir-130b, mir-146a, mir- |
| | 105, mir-122, mir-122a, mir-1236, mir- | 153, mir-155, mir-17-5p, mir- |
| | 124, mir-125b, mir-126, mir-127, mir- | 181a, mir-181a-1, mir-181a-2, |
| | 1271, mir-128-3p, mir-129-5p, mir-130a, | mir-181b, mir-181b-1, mir-181b- |
| | mir-130b, mir-133a, mir-134, mir-137, | 2, mir-181c, mir-181d, mir-182, |
| | mir-138, mir-139, mir-139-5p, mir-140- | mir-183, mir-184, mir-190b, mir- |
| | 5p, mir-141, mir-142-3p, mir-143, mir- | 191, mir-20a, mir-20b, mir-21, |
| | 144, mir-145, mir-146a, mir-148a, mir- | mir-210, mir-214, mir-215, mir- |
| | 148b, mir-150-5p, mir-15b, mir-16, mir- | 216a, mir-217, mir-221, mir-222, |
| | 181a-5p, mir-185, mir-188-5p, mir-193b, | mir-223, mir-224, mir-23a, mir- |
| | mir-195, mir-195-5p, mir-197, mir-198, | 24, mir-25, mir-27a, mir-301a, |
| | mir-199a, mir-199a-5p, mir-199b, mir- | mir-30d, mir-31, mir-3127, mir- |
| | 199b-5p, mir-200a, mir-200b, mir-200c, | 32, mir-331-3p, mir-362-3p, mir- |
| | mir-202, mir-203, mir-204-3p, mir-205, | 371-5p, mir-372, mir-373, mir- |
| | mir-206, mir-20a, mir-21, mir-21-3p, | 423, mir-429, mir-452, mir-483- |
| | mir-211, mir-212, mir-214, mir-217, mir- | 3p, mir-483-5p, mir-485-3p, mir- |
| | 218, mir-219-5p, mir-22, mir-26a, mir- | 490-3p, mir-494, mir-495, mir- |
| | 26b, mir-29a, mir-29b-1, mir-29b-2, mir- | 500, mir-501-5p, mir-519d, mir- |
| | 29c, mir-302b, mir-302c, mir-30a, mir- | 520g, mir-574-3p, mir-590-5p, |
| | 30a-3p, mir-335, mir-338-3p, mir-33a, | mir-630, mir-650, mir-657, mir- |
| | mir-34a, mir-34b, mir-365, mir-370, mir- | 664, mir-885-5p, mir-9, mir-92a, |
| | 372, mir-375, mir-376a, mir-377, mir- | mir-96 |
| | 422a, mir-424, mir-424-5p, mir-433, mir- | |
| | 4458, mir-448, mir-450a, mir-451, mir- | |
| | 485-5p, mir-486-5p, mir-497, mir-503, | |
| | mir-506, mir-519d, mir-520a, mir-520b, | |
| | mir-520c-3p, mir-582-5p, mir-590-5p, | |
| | mir-610, mir-612, mir-625, mir-637, mir- | |
| | 675, mir-7, mir-877, mir-940, mir-941, | |
| | mir-98, mir-99a | |
| lung cancer | let-7a-1, let-7a-2, let-7a-3, let-7b, let-7c, | mir-10b, mir-135b, mir-150, mir- |
| | let-7d, let-7e, let-7f-1, let-7f-2, let-7g, | 155, mir-17, mir-182, mir-183- |
| | let-7i, mir-1, mir-101, mir-133b, mir- | 3p, mir-18a, mir-197, mir-19a, |
| | 138, mir-142-5p, mir-144, mir-145, mir- | mir-19b, mir-205, mir-20a, mir- |
| | 1469, mir-146a, mir-153, mir-15a, mir- | 21, mir-210, mir-24, mir-30d, |
| | 15b, mir-16-1, mir-16-2, mir-182, mir- | mir-4423, mir-5100, mir-570, |
| | 192, mir-193a-3p, mir-194, mir-195, mir- | mir-663, mir-7, mir-92a |
| | 198, mir-203, mir-217, mir-218, mir-22, | |
| | mir-223, mir-26a, mir-26b, mir-29c, mir- | |
| | 33a, mir-34a, mir-34b, mir-34c, mir-365, | |
| | mir-449a, mir-449b, mir-486-5p, mir- | |
| | 545, mir-610, mir-614, mir-630, mir-660, | |
| | mir-7-5p, mir-9500, mir-98, mir-99b | |
| neuroblastoma | let-7a-1, let-7a-2, let-7a-3, let-7b, let-7c, | mir-125b, mir-15a, mir-15b, mir- |
| | let-7d, let-7e, let-7f-1, let-7f-2, let-7g, | 16-1, mir-16-2, mir-18a, mir-195, |
| | let-7i, mir-124, mir-137, mir-145, mir- | mir-19a, mir-23a, mir-421, mir- |
| | 181c, mir-184, mir-200a, mir-29a, mir- | 92 |
| | 335, mir-338-3p, mir-34a, mir-449a, mir- | |
| | 885-5p, mir-98 | |
| prostate cancer | let-7a-3p, let-7c, mir-100, mir-101, mir- | mir-125b, mir-141, mir-153, mir- |
| | 105, mir-124, mir-128, mir-1296, mir- | 155, mir-181a-1, mir-181a-2, |
| | 130b, mir-133a-1, mir-133a-2, mir-133b, | mir-181b, mir-181b-1, mir-181b- |
| | mir-135a, mir-143, mir-145, mir-146a, | 2, mir-181c, mir-181d, mir-182, |
| | mir-154, mir-15a, mir-187, mir-188-5p, | mir-182-5p, mir-183, mir-18a, |
| | mir-199b, mir-200b, mir-203, mir-205, | mir-204, mir-20a, mir-21, mir- |
| | mir-212, mir-218, mir-221, mir-224, mir- | 221, mir-223-3p, mir-31, mir- |
| | 23a, mir-23b, mir-25, mir-26a, mir-26b, | 429, mir-96 |
| | mir-29b, mir-302a, mir-30a, mir-30b, | |
| | mir-30c-1, mir-30c-2, mir-30d, mir-30e, | |
| | mir-31, mir-330, mir-331-3p, mir-34a, | |
| | mir-34b, mir-34c, mir-374b, mir-449a, | |
| | mir-4723-5p, mir-497, mir-628-5p, mir- | |
| | 642a-5p, mir-720, mir-940 | |
| acute lymphoblastic | let-7b, mir-124a, mir-142-3p | mir-128 |
| leukemia | | |
| malignant melanoma | let-7b, mir-101, mir-125b, mir-1280, | mir-126, mir-141, mir-15b, mir- |
| | mir-143, mir-146a, mir-146b, mir-155, | 17, mir-17-5p, mir-182, mir-18a, |
| | mir-17, mir-184, mir-185, mir-18b, mir- | mir-193b, mir-200a, mir-200b, |
| | 193b, mir-200c, mir-203, mir-204, mir- | mir-200c, mir-20a, mir-21, mir- |
| | 205, mir-206, mir-20a, mir-211, mir-218, | 210, mir-214, mir-221, mir-222, |
| | mir-26a, mir-31, mir-33a, mir-34a, mir- | mir-429, mir-455-5p, mir-532-5p, |
| | 34c, mir-376a, mir-376c, mir-573, mir-7, | mir-638, mir-92a |
| | mir-9, mir-98 | |
| renal clear cell | let-7b, let-7c, mir-138, mir-141, mir- | mir-122, mir-155, mir-630 |
| carcinoma | 200c, mir-204, mir-218, mir-335, mir- | |
| | 377, mir-506 | |
| acute myeloid | let-7c, mir-17, mir-181a, mir-20a, mir- | mir-125b, mir-126-5p, mir-128, |
| leukemia | 223, mir-26a, mir-29a, mir-30c, mir-7 | mir-155, mir-29a, mir-32, mir- |
| | | 331, mir-370, mir-378 |
| acute promyelocytic | let-7c, mir-107, mir-342 | mir-181a, mir-181b, mir-92a |
| leukemia | | |
| head and neck | let-7d, mir-1, mir-107, mir-128, mir- | mir-106b, mir-134, mir-16, mir- |
| squamous cell | 133a, mir-138, mir-149, mir-200c, mir- | 184, mir-196a, mir-21, mir-25, |
| carcinoma | 205, mir-218, mir-27a*, mir-29a, mir- | mir-30a-5p, mir-31, mir-372, |
| | 29b-1, mir-29b-2, mir-29c, mir-300, mir- | mir-93 |
| | 34a, mir-363, mir-375, mir-874 | |
| oral cancer | let-7d, mir-218, mir-34a, mir-375, mir- | mir-10b, mir-196a-1, mir-196a-2, |
| | 494 | mir-196b, mir-21 |
| papillary thyroid | mir-101, mir-130b, mir-138, mir-146a, | let-7e, mir-146b, mir-146b-5p, |
| carcinoma | mir-16, mir-195, mir-199a-3p, mir-204- | mir-151-5p, mir-155, mir-181a-1, |
| | 5p, mir-219-5p, mir-26a, mir-34b, mir- | mir-181a-2, mir-181b-1, mir- |
| | 613 | 181b-2, mir-181c, mir-181d, mir- |
| | | 182, mir-183, mir-199b-5p, mir- |
| | | 21, mir-221, mir-222, mir-339- |
| | | 5p, mir-34a |
| glioblastoma | let-7g-5p, mir-100, mir-101, mir-106a, | mir-10b, mir-125b, mir-127-3p, |
| | mir-124, mir-124a, mir-125a, mir-125a- | mir-148a, mir-18a, mir-196a, |
| | 5p, mir-125b, mir-127-3p, mir-128, mir- | mir-196a-1, mir-196a-2, mir- |
| | 129, mir-136, mir-137, mir-139-5p, mir- | 196b, mir-21, mir-210, mir-210- |
| | 142-3p, mir-143, mir-145, mir-146b-5p, | 3p, mir-223, mir-340, mir-576- |
| | mir-149, mir-152, mir-153, mir-195, mir- | 5p, mir-626, mir-92b |
| | 21, mir-212-3p, mir-219-5p, mir-222, | |
| | mir-29b, mir-31, mir-3189-3p, mir-320, | |
| | mir-320a, mir-326, mir-330, mir-331-3p, | |
| | mir-340, mir-342, mir-34a, mir-376a, | |
| | mir-449a, mir-483-5p, mir-503, mir-577, | |
| | mir-663, mir-7, mir-744 | |
| ovarian cancer | let-7i, mir-100, mir-124, mir-125b, mir- | mir-106a, mir-141, mir-148b, |
| | 129-5p, mir-130b, mir-133a, mir-137, | mir-181b, mir-182, mir-200a, |
| | mir-138, mir-141, mir-145, mir-148a, | mir-200c, mir-205, mir-20a, mir- |
| | mir-152, mir-153, mir-155, mir-199a, | 21, mir-210, mir-214, mir-221, |
| | mir-200a, mir-200b, mir-200c, mir-212, | mir-224-5p, mir-23b, mir-25, |
| | mir-335, mir-34a, mir-34b, mir-34c, mir- | mir-26a, mir-27a, mir-27b, mir- |
| | 409-3p, mir-411, mir-429, mir-432, mir- | 346, mir-378, mir-424, mir-503, |
| | 449a, mir-494, mir-497, mir-498, mir- | mir-572, mir-9, mir-96 |
| | 519d, mir-655, mir-9, mir-98 | |
| bladder cancer | mir-1, mir-101, mir-1180, mir-1236, mir- | mir-103a-3p, mir-10b, mir-135a, |
| | 124-3p, mir-125b, mir-126, mir-1280, | mir-137, mir-141, mir-155, mir- |
| | mir-133a, mir-133b, mir-141, mir-143, | 17-5p, mir-182, mir-182-5p, mir- |
| | mir-144, mir-145, mir-155, mir-16, mir- | 183, mir-185, mir-19a, mir-203, |
| | 18a, mir-192, mir-195, mir-200a, mir- | mir-205, mir-210, mir-221, mir- |
| | 200b, mir-200c, mir-203, mir-205, mir- | 222, mir-223, mir-23a, mir-23b, |
| | 214, mir-218, mir-23b, mir-26a, mir-29c, | mir-26b, mir-639, mir-96 |
| | mir-320c, mir-34a, mir-370, mir-409-3p, | |
| | mir-429, mir-451, mir-490-5p, mir-493, | |
| | mir-576-3p, mir-99a | |
| chordoma | mir-1, mir-222, mir-31, mir-34a, mir-608 | mir-140-3p, mir-148a |
| kidney cancer | mir-1, mir-145, mir-1826, mir-199a, mir- | mir-183, mir-21, mir-210, mir- |
| | 199a-3p, mir-203, mir-205, mir-497, mir- | 223 |
| | 508-3p, mir-509-3p | |
| cervical carcinoma | mir-100, mir-101, mir-15a, mir-16, mir- | mir-133b, mir-21, mir-25, mir- |
| | 34a, mir-886-5p, mir-99a, mir-99b | 373 |
| mesenchymal cancer | mir-100, mir-141, mir-199b-5p, mir- | mir-125b-1-3p, mir-182 |
| | 200a, mir-200b, mir-200c, mir-29a, mir- | |
| | 29b-1, mir-29b-1-5p, mir-29b-2, mir-29c, | |
| | mir-335, mir-429, mir-99a | |
| oral squamous cell | mir-100, mir-124, mir-1250, mir-125b, | mir-125b, mir-126, mir-146a, |
| carcinoma | mir-126, mir-1271, mir-136, mir-138, | mir-146b, mir-155, mir-181b, |
| | mir-145, mir-147, mir-148a, mir-181a, | mir-196a-1, mir-196a-2, mir- |
| | mir-206, mir-220a, mir-26a, mir-26b, | 196b, mir-21, mir-221, mir-222, |
| | mir-29a, mir-32, mir-323-5p, mir-329, | mir-24, mir-27b, mir-31, mir-345 |
| | mir-338, mir-370, mir-410, mir-429, mir- | |
| | 433, mir-499a-5p, mir-503, mir-506, mir- | |
| | 632, mir-646, mir-668, mir-877, mir-9 | |
| ovarian carcinoma | mir-100, mir-101, mir-34b, mir-34c, mir- | mir-148b, mir-182 |
| | 532-5p | |
| cholangiocarcinoma | mir-101, mir-144, mir-200b, mir-200c | mir-17, mir-18a, mir-19a, mir- |
| | | 19b, mir-20a, mir-21, mir-26a, |
| | | mir-92a |
| endometrial cancer | mir-101, mir-130a, mir-130b, mir-134, | mir-106a, mir-145, mir-155, mir- |
| | mir-143, mir-145, mir-152, mir-205, mir- | 182, mir-200b, mir-200c, mir- |
| | 223, mir-301a, mir-301b, mir-30c, mir- | 205, mir-21, mir-222-3p, mir-25, |
| | 34a, mir-34c, mir-424, mir-449a, mir- | mir-93 |
| | 543 | |
| esophageal cancer | mir-124, mir-126, mir-140, mir-197, mir- | mir-101, mir-10b, mir-130a, mir- |
| | 203, mir-218, mir-223, mir-30b, mir-375, | 141, mir-143, mir-146b, mir-15a, |
| | mir-454, mir-486, mir-574-3p | mir-183, mir-196b, mir-200a, |
| | | mir-203, mir-205, mir-21, mir- |
| | | 210, mir-221, mir-27a, mir-28- |
| | | 3p, mir-31, mir-452, mir-96, mir- |
| | | 99b |
| liver cancer | mir-101, mir-122, mir-132, mir-140-5p, | mir-1301, mir-155, mir-21, mir- |
| | mir-145, mir-148b, mir-31, mir-338-3p, | 221, mir-27a, mir-525-3p |
| | mir-433 | |
| pancreatic cancer | mir-101, mir-1181, mir-124, mir-1247, | mir-10a, mir-10b, mir-132, mir- |
| | mir-133a, mir-141, mir-145, mir-146a, | 15a, mir-17-5p, mir-181a, mir- |
| | mir-148a, mir-148b, mir-150*, mir-150- | 18a, mir-191, mir-196a, mir-21, |
| | 5p, mir-152, mir-15a, mir-198, mir-203, | mir-212, mir-214, mir-222, mir- |
| | mir-214, mir-216a, mir-29c, mir-335, | 27a, mir-301a, mir-301a-3p, mir- |
| | mir-34a, mir-34b, mir-34c, mir-373, mir- | 367, mir-424-5p, mir-7, mir-92, |
| | 375, mir-410, mir-497, mir-615-5p, mir- | mir-99a |
| | 630, mir-96 | |
| retinoblastoma | mir-101, mir-183, mir-204, mir-34a, mir- | mir-181b, mir-21 |
| | 365b-3p, mir-486-3p, mir-532-5p | |
| cervical squamous cell | mir-106a, mir-124, mir-148a, mir-214, | mir-205 |
| carcinoma | mir-218, mir-29a, mir-375 | |
| clear cell renal cell | mir-106a-5p, mir-135a-5p, mir-206 | mir-142-5p, mir-155, mir-21-5p |
| cancer | | |
| laryngeal carcinoma | | mir-106b, mir-16, mir-21, mir- |
| | | 27a, mir-423-3p |
| medulloblastoma | mir-124, mir-128a, mir-199b-5p, mir- | mir-106b, mir-17, mir-18a, mir- |
| | 206, mir-22, mir-31, mir-383 | 19a, mir-19b, mir-20a, mir-30b, |
| | | mir-30d, mir-92 |
| pituitary carcinoma | | mir-106b, mir-122, mir-20a, mir- |
| | | 493 |
| prostate carcinoma | mir-107 | |
| cervical cancer | mir-143, mir-145, mir-17-5p, mir-203, | mir-10a, mir-155, mir-181a, mir- |
| | mir-214, mir-218, mir-335, mir-342-3p, | 181b, mir-196a, mir-19a, mir- |
| | mir-372, mir-424, mir-491-5p, mir-497, | 19b, mir-205, mir-20a, mir-21, |
| | mir-7, mir-99a, mir-99b | mir-215, mir-224, mir-31, mir- |
| | | 494, mir-590-5p, mir-92a, mir- |
| | | 944 |
| chronic myelogenous | mir-10a, mir-146a, mir-150, mir-151, | mir-424, mir-96 |
| leukemia | mir-155, mir-2278, mir-26a, mir-30e, | |
| | mir-31, mir-326, mir-564 | |
| gastrointestinal cancer | mir-122a, mir-148a, mir-152 | |
| anaplastic astrocytoma | mir-124, mir-137 | |
| astrocytoma | mir-124-3p, mir-181b-5p, mir-200b, mir- | mir-335 |
| | 3189-3p | |
| epithelial ovarian | mir-124a, mir-192, mir-193a, mir-7 | mir-372, mir-373 |
| cancer | | |
| mantle cell lymphoma | mir-142-3p, mir-142-5p, mir-150, mir- | mir-124a, mir-155, mir-17, mir- |
| | 223, mir-29a, mir-29b, mir-29c | 18a, mir-19a, mir-19b, mir-20a, |
| | | mir-92a |
| chronic lymphocytic | mir-125b, mir-138, mir-15a, mir-15b, | mir-150, mir-155 |
| leukemia | mir-16, mir-16-1, mir-16-1-3p, mir-16-2, | |
| | mir-181a, mir-181b, mir-195, mir-223, | |
| | mir-29b, mir-34b, mir-34c, mir-424 | |
| follicular cancer | NA | mir-125b |
| malignant | mir-126 | |
| mesothelioma | | |
| small cell lung cancer | mir-126, mir-138, mir-27a | mir-25 |
| meningioma | mir-128, mir-200a | mir-224, mir-335 |
| laryngeal squamous | mir-129-5p, mir-203, mir-205, mir-206, | mir-21, mir-9, mir-93 |
| cell carcinoma | mir-24, mir-370, mir-375 | |
| medullary thyroid | mir-129-5p | mir-183 |
| carcinoma | | |
| lung adenocarcinoma | mir-1297, mir-141, mir-145, mir-16, mir- | mir-150, mir-155, mir-31 |
| | 200a, mir-200b, mir-200c, mir-29b, mir- | |
| | 381, mir-409-3p, mir-429, mir-451, mir- | |
| | 511, mir-99a | |
| pancreatic carcinoma | mir-132, mir-375 | mir-301b |
| lung squamous cell | mir-133a, mir-218 | |
| carcinoma | | |
| multiple myeloma | mir-137, mir-197, mir-214 | mir-21 |
| squamous carcinoma | mir-15a, mir-16, mir-203, mir-205, mir- | mir-137, mir-155, mir-184, mir- |
| | 375 | 196a, mir-203, mir-21, mir-221, |
| | | mir-27a, mir-34a |
| uveal melanoma | mir-137, mir-144, mir-145, mir-182, mir- | NA |
| | 34a, mir-34b, mir-34c, mir-9 | |
| anaplastic thyroid | mir-138 | mir-146b, mir-221, mir-222 |
| carcinoma | | |
| colorectal carcinoma | mir-139, mir-143, mir-145, mir-202-3p, | mir-17, mir-182, mir-191, mir- |
| | mir-30a, mir-338-3p, mir-429, mir-451, | 21, mir-95 |
| | mir-93 | |
| malt lymphoma | | mir-142-5p, mir-155 |
| thyroid cancer | mir-144, mir-886-3p | |
| primary cns | mir-145, mir-193b, mir-199a, mir-214 | |
| lymphomas | | |
| follicular thyroid | mir-199b | mir-146b, mir-183, mir-197, mir- |
| carcinoma | | 221, mir-346 |
| gallbladder carcinoma | mir-146b-5p | mir-155, mir-182 |
| adult t-cell leukemia | | mir-150 |
| anaplastic large-cell | | mir-155 |
| lymphoma | | |
| cutaneous t-cell | | mir-155 |
| lymphoma | | |
| diffuse large B-cell | | mir-155, mir-21 |
| lymphoma | | |
| rectal cancer | | mir-155, mir-200c, mir-21-5p, |
| | | mir-34a |
| tongue cancer | mir-15b, mir-200b | |
| b-cell lymphoma | mir-34a | mir-17, mir-18a, mir-19a, mir- |
| | | 19b, mir-20a, mir-92a |
| breast carcinoma | | mir-17, mir-18a, mir-19a, mir- |
| | | 19b, mir-20a, mir-24, mir-92a |
| nasopharyngeal cancer | mir-218, mir-223, mir-29c | mir-17, mir-20a |
| gastric | mir-181b, mir-182, mir-200a, mir-302b, | mir-23a, mir-27a, mir-373 |
| adenocarcinoma | mir-449a, mir-9 | |
| colorectal | | mir-182 |
| adenocarcinoma | | |
| colon carcinoma | mir-186, mir-30a-5p | mir-221, mir-23a |
| adrenal cortical | mir-195, mir-1974, mir-335, mir-497 | mir-21, mir-210, mir-483-3p, |
| carcinoma | | mir-483-5p |
| esophageal | mir-203 | mir-196a, mir-199a-3p, mir- |
| adenocarcinoma | | 199a-5p, mir-199b-3p, mir-200a, |
| | | mir-223 |
| gastrointestinal | mir-218, mir-221, mir-222 | mir-196a |
| stromal tumor | | |
| uterine leiomyoma | mir-197 | |
| choriocarcinoma | mir-199b, mir-218, mir-34a | |
| follicular lymphoma | mir-202 | |
| basal cell carcinoma | mir-203 | |
| hypopharyngeal | | mir-203 |
| cancer | | |
| pancreatic | | mir-203, mir-301a |
| adenocarcinoma | | |
| rhabdomyosarcoma | mir-203 | |
| head and neck cancer | NA | mir-21 |
| hypopharyngeal | mir-451a, mir-504 | mir-21 |
| squamous cell | | |
| carcinoma | | |
| t-cell lymphoma | mir-22 | |
| thyroid carcinoma | | mir-221, mir-222 |
| splenic marginal zone | mir-223 | |
| lymphoma | | |
| laryngeal cancer | | mir-23a |
| primary thyroid | mir-26a | |
| lymphoma | | |
| acute leukemia | mir-27a | |
| monocytic leukemia | mir-29a, mir-29b | |
| oral carcinoma | mir-375 | mir-31 |
| primary gallbladder | mir-335 | |
| carcinoma | | |
| endometrial serous | mir-34b | |
| adenocarcinoma | | |
| esophageal carcinoma | mir-451 | |
| hepatoblastoma | | mir-492 |
| colonic | mir-627 | |
| adenocarcinoma | | |

TABLE 12
Exemplary tumor suppressive miRs

| Cancer | Down regulated tumor suppressive miR |
|---|---|
| acute leukemia | mir-27a |
| acute lymphoblastic leukemia | let-7b, mir-124a, mir-142-3p |
| acute myeloid leukemia | let-7c, mir-17, mir-181a, mir-20a, mir-223, mir-26a, mir-29a, mir-30c, |
| | mir-720 |
| acute promyelocytic leukemia | let-7c, mir-107, mir-342 |
| adrenal cortical carcinoma | mir-195, mir-1974, mir-335, mir-497 |
| anaplastic astrocytoma | mir-124, mir-137 |
| anaplastic thyroid carcinoma | mir-138 |
| astrocytoma | mir-124-3p, mir-181b-5p, mir-200b, mir-3189-3p |
| basal cell carcinoma | mir-203 |
| b-cell lymphoma | mir-34a |
| bladder cancer | mir-1, mir-101, mir-1180, mir-1236, mir-124-3p, mir-125b, mir-126, mir- |
| | 1280, mir-133a, mir-133b, mir-141, mir-143, mir-144, mir-145, mir-155, |
| | mir-16, mir-18a, mir-192, mir-195, mir-200a, mir-200b, mir-200c, mir- |
| | 203, mir-205, mir-214, mir-218, mir-23b, mir-26a, mir-29c, mir-320c, |
| | mir-34a, mir-370, mir-409-3p, mir-429, mir-451, mir-490-5p, mir-493, |
| | mir-576-3p, mir-99a |
| breast cancer | let-7a, let-7a-1, let-7a-2, let-7a-3, let-7b, let-7c, let-7d, let-7e, let-7f-1, let- |
| | 7f-2, let-7g, let-7i, mir-100, mir-107, mir-10a, mir-10b, mir-122, mir-124, |
| | mir-1258, mir-125a-5p, mir-125b, mir-126, mir-127, mir-129, mir-130a, |
| | mir-132, mir-133a, mir-143, mir-145, mir-146a, mir-146b, mir-147, mir- |
| | 148a, mir-149, mir-152, mir-153, mir-15a, mir-16, mir-17-5p, mir-181a, |
| | mir-1826, mir-183, mir-185, mir-191, mir-193a-3p, mir-193b, mir-195, |
| | mir-199b-5p, mir-19a-3p, mir-200a, mir-200b, mir-200c, mir-205, mir- |
| | 206, mir-211, mir-216b, mir-218, mir-22, mir-26a, mir-26b, mir-300, |
| | mir-30a, mir-31, mir-335, mir-339-5p, mir-33b, mir-34a, mir-34b, mir- |
| | 34c, mir-374a, mir-379, mir-381, mir-383, mir-425, mir-429, mir-450b- |
| | 3p, mir-494, mir-495, mir-497, mir-502-5p, mir-517a, mir-574-3p, mir- |
| | 638, mir-7, mir-720, mir-873, mir-874, mir-92a, mir-98, mir-99a, mmu- |
| | mir-290-3p, mmu-mir-290-5p |
| bronchioloalveolar carcinoma | let-7a-1, let-7a-2, let-7a-3, let-7b, let-7c, let-7d, let-7e, let-7f-1, let-7f-2, |
| | let-7g, let-7i, mir-98 |
| cervical cancer | mir-143, mir-145, mir-17-5p, mir-203, mir-214, mir-218, mir-335, mir- |
| | 342-3p, mir-372, mir-424, mir-491-5p, mir-497, mir-7, mir-99a, mir-99b |
| cervical carcinoma | mir-100, mir-101, mir-15a, mir-16, mir-34a, mir-886-5p, mir-99a, mir- |
| | 99b |
| cervical squamous cell carcinoma | mir-106a, mir-124, mir-148a, mir-214, mir-218, mir-29a, mir-375 |
| cholangiocarcinoma | mir-101, mir-144, mir-200b, mir-200c |
| chondrosarcoma | let-7a, mir-100, mir-136, mir-145, mir-199a, mir-222, mir-30a, mir-335, |
| | mir-376a |
| chordoma | mir-1, mir-222, mir-31, mir-34a, mir-608 |
| choriocarcinoma | mir-199b, mir-218, mir-34a |
| chronic lymphocytic leukemia | mir-125b, mir-138, mir-15a, mir-15b, mir-16, mir-16-1, mir-16-1-3p, |
| | mir-16-2, mir-181a, mir-181b, mir-195, mir-223, mir-29b, mir-34b, mir- |
| | 34c, mir-424 |
| chronic myelogenous leukemia | mir-10a, mir-138, mir-146a, mir-150, mir-151, mir-155, mir-16, mir- |
| | 2278, mir-26a, mir-30e, mir-31, mir-326, mir-564 |
| clear cell renal cell cancer | mir-106a-5p, mir-135a-5p, mir-206 |
| colon cancer | let-7a-1, let-7a-2, let-7a-3, let-7b, let-7c, let-7d, let-7e, let-7f-1, let-7f-2, |
| | let-7g, let-7i, mir-100, mir-101, mir-126, mir-142-3p, mir-143, mir-145, |
| | mir-192, mir-200c, mir-21, mir-214, mir-215, mir-22, mir-25, mir-302a, |
| | mir-320, mir-320a, mir-34a, mir-34c, mir-365, mir-373, mir-424, mir- |
| | 429, mir-455, mir-484, mir-502, mir-503, mir-93, mir-98 |
| colon carcinoma | mir-186, mir-30a-5p |
| colonic adenocarcinoma | mir-627 |
| colorectal cancer | let-7a, mir-1, mir-100, mir-101, mir-124, mir-125a, mir-126, mir-129, |
| | mir-1295b-3p, mir-1307, mir-130b, mir-132, mir-133a, mir-133b, mir- |
| | 137, mir-138, mir-139, mir-139-5p, mir-140-5p, mir-143, mir-145, mir- |
| | 148a, mir-148b, mir-149, mir-150-5p, mir-154, mir-15a, mir-15b, mir-16, |
| | mir-18a, mir-191, mir-192, mir-193a-5p, mir-194, mir-195, mir-196a, |
| | mir-198, mir-199a-5p, mir-200c, mir-203, mir-204-5p, mir-206, mir-212, |
| | mir-215, mir-218, mir-22, mir-224, mir-24-3p, mir-26b, mir-27a, mir-28- |
| | 3p, mir-28-5p, mir-29b, mir-30a-3p, mir-30b, mir-320a, mir-328, mir- |
| | 338-3p, mir-342, mir-345, mir-34a, mir-34a-5p, mir-361-5p, mir-375, |
| | mir-378, mir-378a-3p, mir-378a-5p, mir-409-3p, mir-422a, mir-4487, |
| | mir-483, mir-497, mir-498, mir-518a-3p, mir-551a, mir-574-5p, mir-625, |
| | mir-638, mir-7, mir-96-5p |
| colorectal carcinoma | mir-139, mir-143, mir-145, mir-202-3p, mir-30a, mir-338-3p, mir-429, |
| | mir-451, mir-93 |
| endometrial cancer | mir-101, mir-130a, mir-130b, mir-134, mir-143, mir-145, mir-152, mir- |
| | 205, mir-223, mir-301a, mir-301b, mir-30c, mir-34a, mir-34c, mir-424, |
| | mir-449a, mir-543 |
| endometrial serous adenocarcinoma | mir-34b |
| epithelial ovarian cancer | mir-124a, mir-192, mir-193a, mir-7 |
| esophageal adenocarcinoma | mir-203 |
| esophageal cancer | mir-124, mir-126, mir-140, mir-197, mir-203, mir-218, mir-223, mir-30b, |
| | mir-375, mir-454, mir-486, mir-574-3p |
| esophageal carcinoma | mir-451 |
| esophageal squamous cell | let-7a, let-7a-1, let-7a-2, let-7a-3, let-7b, let-7c, let-7d, let-7e, let-7f-1, let- |
| carcinoma | 7f-2, let-7g, let-7i, mir-1, mir-100, mir-101, mir-126, mir-1294, mir-133a, |
| | mir-133b, mir-138, mir-143, mir-145, mir-150, mir-185, mir-195, mir- |
| | 200b, mir-203, mir-21, mir-210, mir-214, mir-218, mir-22, mir-27a, mir- |
| | 29b, mir-29c, mir-302b, mir-34a, mir-375, mir-494, mir-518b, mir-655, |
| | mir-98, mir-99a |
| follicular lymphoma | mir-202 |
| follicular thyroid carcinoma | mir-199b |
| gallbladder carcinoma | mir-146b-5p |
| gastric adenocarcinoma | mir-181b, mir-182, mir-200a, mir-302b, mir-449a, mir-9 |
| gastric cancer | let-7a, let-7b, let-7g, mir-1, mir-101, mir-103a, mir-10a, mir-10b, mir- |
| | 1207-5p, mir-122, mir-1228*, mir-124, mir-124-3p, mir-125a-3p, mir- |
| | 126, mir-1266, mir-127, mir-1271, mir-129-1-3p, mir-129-2-3p, mir-129- |
| | 3p, mir-129-5p, mir-133a, mir-133b, mir-137, mir-141, mir-143, mir-144, |
| | mir-145, mir-146a, mir-146a-5p, mir-148a, mir-148b, mir-149, mir-152, |
| | mir-155, mir-155-5p, mir-181a, mir-181b, mir-182, mir-183, mir-185, |
| | mir-194, mir-195, mir-197, mir-199a-3p, mir-200b, mir-200c, mir-202- |
| | 3p, mir-204, mir-204-5p, mir-205, mir-206, mir-210, mir-212, mir-217, |
| | mir-218, mir-22, mir-23b, mir-24, mir-26a, mir-29a, mir-29a-3p, mir-29b, |
| | mir-29b-1, mir-29b-2, mir-29c, mir-30a-5p, mir-30b, mir-31, mir-328, |
| | mir-329, mir-331-3p, mir-335-5p, mir-338, mir-338-3p, mir-34a, mir- |
| | 34b, mir-34c, mir-361-5p, mir-367, mir-375, mir-378, mir-409-3p, mir- |
| | 410, mir-429, mir-433, mir-449, mir-449a, mir-490-3p, mir-494, mir-497, |
| | mir-503, mir-506, mir-513b, mir-520d-3p, mir-542-3p, mir-622, mir-625, |
| | mir-638, mir-663, mir-7, mir-874, mir-9 |
| gastrointestinal cancer | mir-122a, mir-148a, mir-152 |
| gastrointestinal stromal tumor | mir-218, mir-221, mir-222 |
| glioblastoma | let-7g-5p, mir-100, mir-101, mir-106a, mir-124, mir-124a, mir-125a, mir- |
| | 125a-5p, mir-125b, mir-127-3p, mir-128, mir-129, mir-136, mir-137, mir- |
| | 139-5p, mir-142-3p, mir-143, mir-145, mir-146b-5p, mir-149, mir-152, |
| | mir-153, mir-195, mir-21, mir-212-3p, mir-219-5p, mir-222, mir-29b, |
| | mir-31, mir-3189-3p, mir-320, mir-320a, mir-326, mir-330, mir-331-3p, |
| | mir-340, mir-342, mir-34a, mir-376a, mir-449a, mir-483-5p, mir-503, |
| | mir-577, mir-663, mir-7, mir-7-5p, mir-873 |
| glioma | let-7a, let-7f, mir-106a, mir-107, mir-122, mir-124, mir-124-5p, mir- |
| | 124a, mir-125b, mir-128, mir-136, mir-137, mir-139, mir-143, mir-145, |
| | mir-146a, mir-146b, mir-146b-5p, mir-152, mir-15b, mir-16, mir-181a, |
| | mir-181a-1, mir-181a-2, mir-181b, mir-181b-1, mir-181b-2, mir-181c, |
| | mir-181d, mir-184, mir-185, mir-195, mir-199a-3p, mir-200a, mir-200b, |
| | mir-203, mir-204, mir-205, mir-218, mir-219-5p, mir-23b, mir-26b, mir- |
| | 27a, mir-29c, mir-320, mir-326, mir-328, mir-34a, mir-34c-3p, mir-34c- |
| | 5p, mir-375, mir-383, mir-451, mir-452, mir-483-5p, mir-495, mir-584, |
| | mir-622, mir-656, mir-7, mir-98 |
| head and neck squamous cell | let-7d, mir-1, mir-107, mir-128, mir-133a, mir-138, mir-149, mir-200c, |
| carcinoma | mir-205, mir-218, mir-27a*, mir-29a, mir-29b-1, mir-29b-2, mir-29c, |
| | mir-300, mir-34a, mir-363, mir-375, mir-874 |
| hepatocellular carcinoma | let-7a-1, let-7a-2, let-7a-3, let-7b, let-7c, let-7d, let-7e, let-7f, let-7f-1, let- |
| | 7f-2, let-7g, let-7i, mir-1, mir-100, mir-101, mir-105, mir-122, mir-122a, |
| | mir-1236, mir-124, mir-125b, mir-126, mir-127, mir-1271, mir-128-3p, |
| | mir-129-5p, mir-130a, mir-130b, mir-133a, mir-134, mir-137, mir-138, |
| | mir-139, mir-139-5p, mir-140-5p, mir-141, mir-142-3p, mir-143, mir- |
| | 144, mir-145, mir-146a, mir-148a, mir-148b, mir-150-5p, mir-15b, mir- |
| | 16, mir-181a-5p, mir-185, mir-188-5p, mir-193b, mir-195, mir-195-5p, |
| | mir-197, mir-198, mir-199a, mir-199a-5p, mir-199b, mir-199b-5p, mir- |
| | 200a, mir-200b, mir-200c, mir-202, mir-203, mir-204-3p, mir-205, mir- |
| | 206, mir-20a, mir-21, mir-21-3p, mir-211, mir-212, mir-214, mir-217, |
| | mir-218, mir-219-5p, mir-22, mir-223, mir-26a, mir-26b, mir-29a, mir- |
| | 29b-1, mir-29b-2, mir-29c, mir-302b, mir-302c, mir-30a, mir-30a-3p, |
| | mir-335, mir-338-3p, mir-33a, mir-34a, mir-34b, mir-365, mir-370, mir- |
| | 372, mir-375, mir-376a, mir-377, mir-422a, mir-424, mir-424-5p, mir- |
| | 433, mir-4458, mir-448, mir-450a, mir-451, mir-485-5p, mir-486-5p, mir- |
| | 497, mir-503, mir-506, mir-519d, mir-520a, mir-520b, mir-520c-3p, mir- |
| | 582-5p, mir-590-5p, mir-610, mir-612, mir-625, mir-637, mir-675, mir-7, |
| | mir-877, mir-940, mir-941, mir-98, mir-99a |
| hypopharyngeal squamous cell | mir-451a, mir-504 |
| carcinoma | |
| kidney cancer | mir-1, mir-145, mir-1826, mir-199a, mir-199a-3p, mir-203, mir-205, mir- |
| | 497, mir-508-3p, mir-509-3p |
| laryngeal squamous cell carcinoma | mir-129-5p, mir-203, mir-205, mir-206, mir-24, mir-370, mir-375 |
| liver cancer | mir-101, mir-122, mir-132, mir-140-5p, mir-145, mir-148b, mir-31, mir- |
| | 338-3p, mir-433 |
| lung adenocarcinoma | mir-1297, mir-141, mir-145, mir-16, mir-200a, mir-200b, mir-200c, mir- |
| | 29b, mir-381, mir-409-3p, mir-429, mir-451, mir-511, mir-99a |
| lung cancer | let-7a-1, let-7a-2, let-7a-3, let-7b, let-7c, let-7d, let-7e, let-7f-1, let-7f-2, |
| | let-7g, let-7i, mir-1, mir-101, mir-133b, mir-138, mir-142-5p, mir-144, |
| | mir-145, mir-1469, mir-146a, mir-153, mir-15a, mir-15b, mir-16-1, mir- |
| | 16-2, mir-182, mir-192, mir-193a-3p, mir-194, mir-195, mir-198, mir- |
| | 203, mir-217, mir-218, mir-22, mir-223, mir-26a, mir-26b, mir-29c, mir- |
| | 33a, mir-34a, mir-34b, mir-34c, mir-365, mir-449a, mir-449b, mir-486- |
| | 5p, mir-545, mir-610, mir-614, mir-630, mir-660, mir-7515, mir-9500, |
| | mir-98, mir-99b |
| lung squamous cell carcinoma | mir-133a, mir-218 |
| malignant melanoma | let-7b, mir-101, mir-125b, mir-1280, mir-143, mir-146a, mir-146b, mir- |
| | 155, mir-17, mir-184, mir-185, mir-18b, mir-193b, mir-200c, mir-203, |
| | mir-204, mir-205, mir-206, mir-20a, mir-211, mir-218, mir-26a, mir-31, |
| | mir-33a, mir-34a, mir-34c, mir-376a, mir-376c, mir-573, mir-7-5p, mir-9, |
| | mir-98 |
| malignant mesothelioma | mir-126 |
| mantle cell lymphoma | mir-142-3p, mir-142-5p, mir-150, mir-223, mir-29a, mir-29b, mir-29c |
| medullary thyroid carcinoma | mir-129-5p |
| medulloblastoma | mir-124, mir-128a, mir-199b-5p, mir-206, mir-22, mir-31, mir-383 |
| meningioma | mir-128, mir-200a |
| mesenchymal cancer | mir-100, mir-141, mir-199b-5p, mir-200a, mir-200b, mir-200c, mir-29a, |
| | mir-29b-1, mir-29b-1-5p, mir-29b-2, mir-29c, mir-335, mir-429, mir-99a |
| monocytic leukemia | mir-29a, mir-29b |
| multiple myeloma | mir-137, mir-197, mir-214 |
| nasopharyngeal cancer | mir-218, mir-223, mir-29c |
| nasopharyngeal carcinoma | let-7a, let-7a-1, let-7a-2, let-7a-3, let-7b, let-7c, let-7d, let-7e, let-7f-1, let- |
| | 7f-2, let-7g, let-7i, mir-1, mir-101, mir-124, mir-138, mir-143, mir-145, |
| | mir-148a, mir-200b, mir-204, mir-216b, mir-223, mir-29c, mir-320a, mir- |
| | 324-3p, mir-34c, mir-375, mir-378, mir-451, mir-506, mir-9, mir-98 |
| neuroblastoma | let-7a-1, let-7a-2, let-7a-3, let-7b, let-7c, let-7d, let-7e, let-7f-1, let-7f-2, |
| | let-7g, let-7i, mir-124, mir-137, mir-145, mir-181c, mir-184, mir-200a, |
| | mir-29a, mir-335, mir-338-3p, mir-34a, mir-449a, mir-885-5p, mir-98 |
| non-small cell lung cancer | let-7a, let-7c, mir-1, mir-100, mir-101, mir-106a, mir-107, mir-124, mir- |
| | 125a-3p, mir-125a-5p, mir-126, mir-126*, mir-129, mir-133a, mir-137, |
| | mir-138, mir-140, mir-143, mir-145, mir-146a, mir-146b, mir-148a, mir- |
| | 148b, mir-149, mir-152, mir-153, mir-154, mir-155, mir-15a, mir-16, |
| | mir-17-5p, mir-181a-1, mir-181a-2, mir-181b, mir-181b-1, mir-181b-2, |
| | mir-181c, mir-181d, mir-184, mir-186, mir-193b, mir-195, mir-199a, mir- |
| | 204, mir-212, mir-221, mir-224, mir-26b, mir-27a, mir-27b, mir-29a, mir- |
| | 29b, mir-29c, mir-30a, mir-30b, mir-30c, mir-30d, mir-30d-5p, mir-30e- |
| | 5p, mir-32, mir-335, mir-338-3p, mir-340, mir-342-3p, mir-34a, mir-34b, |
| | mir-361-3p, mir-365, mir-373, mir-375, mir-429, mir-449a, mir-4500, |
| | mir-451, mir-4782-3p, mir-497, mir-503, mir-512-3p, mir-520a-3p, mir- |
| | 526b, mir-625*, mir-96, mir-99a |
| oral cancer | let-7d, mir-218, mir-34a, mir-375, mir-494 |
| oral carcinoma | mir-375 |
| oral squamous cell carcinoma | mir-100, mir-124, mir-1250, mir-125b, mir-126, mir-1271, mir-136, mir- |
| | 138, mir-145, mir-147, mir-148a, mir-181a, mir-206, mir-220a, mir-26a, |
| | mir-26b, mir-29a, mir-32, mir-323-5p, mir-329, mir-338, mir-370, mir- |
| | 410, mir-429, mir-433, mir-499a-5p, mir-503, mir-506, mir-632, mir-646, |
| | mir-668, mir-877, mir-9 |
| osteosarcoma | let-7a, mir-1, mir-100, mir-101, mir-122, mir-124, mir-125b, mir-126, |
| | mir-127-3p, mir-132, mir-133a, mir-141, mir-142-3p, mir-142-5p, mir- |
| | 143, mir-144, mir-145, mir-153, mir-16, mir-183, mir-194, mir-195, mir- |
| | 199a-3p, mir-204, mir-212, mir-217, mir-218, mir-22, mir-23a, mir-24, |
| | mir-26a, mir-26b, mir-29b, mir-32, mir-320, mir-335, mir-33b, mir-340, |
| | mir-34a, mir-34b, mir-34c, mir-375, mir-376c, mir-382, mir-3928, mir- |
| | 424, mir-429, mir-449a, mir-451, mir-454, mir-503, mir-519d, mir-646 |
| ovarian cancer | let-7i, mir-100, mir-124, mir-125b, mir-129-5p, mir-130b, mir-133a, mir- |
| | 137, mir-138, mir-141, mir-145, mir-148a, mir-152, mir-153, mir-155, |
| | mir-199a, mir-200a, mir-200b, mir-200c, mir-212, mir-335, mir-34a, mir- |
| | 34b, mir-34c, mir-409-3p, mir-411, mir-429, mir-432, mir-449a, mir-494, |
| | mir-497, mir-498, mir-519d, mir-655, mir-9, mir-98 |
| ovarian carcinoma | mir-100, mir-101, mir-34b, mir-34c, mir-532-5p |
| pancreatic cancer | mir-101, mir-1181, mir-124, mir-1247, mir-133a, mir-141, mir-145, mir- |
| | 146a, mir-148a, mir-148b, mir-150*, mir-150-5p, mir-152, mir-15a, mir- |
| | 198, mir-203, mir-214, mir-216a, mir-29c, mir-335, mir-34a, mir-34b, |
| | mir-34c, mir-373, mir-375, mir-410, mir-497, mir-615-5p, mir-630, mir- |
| | 96 |
| pancreatic carcinoma | mir-132, mir-375 |
| pancreatic ductal adenocarcinoma | let-7a, let-7a-1, let-7a-2, let-7a-3, let-7b, let-7c, let-7d, let-7e, let-7f-1, let- |
| | 7f-2, let-7g, let-7i, mir-126, mir-135a, mir-143, mir-144, mir-145, mir- |
| | 148a, mir-150, mir-15a, mir-16, mir-200a, mir-200b, mir-200c, mir-217, |
| | mir-218, mir-337, mir-375, mir-494, mir-615-5p, mir-98 |
| papillary thyroid carcinoma | mir-101, mir-130b, mir-138, mir-146a, mir-16, mir-195, mir-199a-3p, |
| | mir-204-5p, mir-219-5p, mir-26a, mir-34b, mir-613 |
| primary cns lymphomas | mir-145, mir-193b, mir-199a, mir-214 |
| primary gallbladder carcinoma | mir-335 |
| primary thyroid lymphoma | mir-26a |
| prostate cancer | let-7a-3p, let-7c, mir-100, mir-101, mir-105, mir-124, mir-128, mir-1296, |
| | mir-130b, mir-133a-1, mir-133a-2, mir-133b, mir-135a, mir-143, mir- |
| | 145, mir-146a, mir-154, mir-15a, mir-187, mir-188-5p, mir-199b, mir- |
| | 200b, mir-203, mir-205, mir-212, mir-218, mir-221, mir-224, mir-23a, |
| | mir-23b, mir-25, mir-26a, mir-26b, mir-29b, mir-302a, mir-30a, mir-30b, |
| | mir-30c-1, mir-30c-2, mir-30d, mir-30e, mir-31, mir-330, mir-331-3p, |
| | mir-34a, mir-34b, mir-34c, mir-374b, mir-449a, mir-4723-5p, mir-497, |
| | mir-628-5p, mir-642a-5p, mir-765, mir-940 |
| prostate carcinoma | mir-107 |
| renal cell carcinoma | let-7a, let-7d, mir-1, mir-106a*, mir-126, mir-1285, mir-129-3p, mir- |
| | 1291, mir-133a, mir-135a, mir-138, mir-141, mir-143, mir-145, mir-182- |
| | 5p, mir-199a-3p, mir-200a, mir-205, mir-218, mir-28-5p, mir-30a, mir- |
| | 30c, mir-30d, mir-34a, mir-378, mir-429, mir-509-3p, mir-509-5p, mir- |
| | 646 |
| renal clear cell carcinoma | let-7b, let-7c, mir-138, mir-141, mir-200c, mir-204, mir-218, mir-335, |
| | mir-377, mir-506 |
| retinoblastoma | mir-101, mir-183, mir-204, mir-34a, mir-365b-3p, mir-486-3p, mir-532- |
| | 5p |
| rhabdomyosarcoma | mir-203 |
| small cell lung cancer | mir-126, mir-138, mir-27a |
| splenic marginal zone lymphoma | mir-223 |
| squamous carcinoma | mir-15a, mir-16, mir-203, mir-205, mir-375 |
| t-cell lymphoma | mir-22 |
| thyroid cancer | mir-144, mir-886-3p |
| tongue cancer | mir-15b, mir-200b |
| uterine leiomyoma | mir-197 |
| uveal melanoma | mir-137, mir-144, mir-145, mir-182, mir-34a, mir-34b, mir-34c, mir-9 |

TABLE 13
Exemplary oncogenic miRs

| Cancer | miRNA |
|---|---|
| colorectal cancer | let-7a, mir-103, mir-106a, mir-10b, mir-1179, mir-1229, mir-1246, mir-125b-2*, mir- |
| | 1269a, mir-130b, mir-133b, mir-135a, mir-135a-1, mir-135a-2, mir-135b, mir-139-3p, |
| | mir-145, mir-150, mir-150*, mir-155, mir-17, mir-181a, mir-182, mir-183, mir-18a, |
| | mir-191, mir-196a, mir-196b, mir-19a, mir-19b, mir-200b, mir-200c, mir-203, mir- |
| | 204-5p, mir-20a, mir-20a-5p, mir-21, mir-210, mir-211, mir-221, mir-223, mir-224, |
| | mir-23a, mir-25, mir-27a, mir-29a, mir-301a, mir-31, mir-32, mir-320b, mir-326, mir- |
| | 424, mir-429, mir-494, mir-497, mir-499-5p, mir-592, mir-630, mir-720, mir-892a, |
| | mir-92, mir-92a, mir-93, mir-95, mir-96 |
| papillary thyroid | let-7e, mir-146b, mir-146b-5p, mir-151-5p, mir-155, mir-181a-1, mir-181a-2, mir- |
| carcinoma | 181b-1, mir-181b-2, mir-181c, mir-181d, mir-182, mir-183, mir-199b-5p, mir-21, mir- |
| | 221, mir-222, mir-339-5p, mir-34a |
| esophageal squamous | mir-100, mir-1179, mir-1290, mir-130b, mir-145, mir-16, mir-17, mir-183, mir-18a, |
| cell carcinoma | mir-19a, mir-19b, mir-208, mir-20a, mir-21, mir-218, mir-223, mir-25, mir-30a-5p, |
| | mir-31, mir-330-3p, mir-373, mir-9, mir-92a, mir-942 |
| gastric cancer | mir-100, mir-103, mir-106a, mir-106b, mir-107, mir-10a, mir-10b, mir-1259, mir- |
| | 125b, mir-126, mir-1274a, mir-1303, mir-130b*, mir-135a-5p, mir-135b, mir-138, |
| | mir-143, mir-146a, mir-147, mir-148a, mir-150, mir-17, mir-17-5p, mir-181a, mir- |
| | 181a-2*, mir-181a-5p, mir-181c, mir-183, mir-185, mir-18a, mir-191, mir-192, mir- |
| | 196a, mir-196a*, mir-196a-5p, mir-196b, mir-199a, mir-199a-3p, mir-199a-5p, mir- |
| | 19a, mir-19b, mir-200b, mir-20a, mir-21, mir-214, mir-215, mir-221, mir-221*, mir- |
| | 222, mir-223, mir-224, mir-23a, mir-23b, mir-25, mir-27a, mir-27b, mir-296-5p, mir- |
| | 301a, mir-302f, mir-337-3p, mir-340*, mir-34a, mir-362-3p, mir-370, mir-374a, mir- |
| | 377, mir-421, mir-425, mir-500, mir-520c-3p, mir-544, mir-575, mir-601, mir-616*, |
| | mir-650, mir-92, mir-98, mir-99a |
| renal cell carcinoma | mir-100, mir-1233, mir-1260b, mir-146a, mir-146b, mir-16, mir-193a-3p, mir-203a, |
| | mir-21, mir-210, mir-27a, mir-362, mir-572, mir-7 |
| esophageal cancer | mir-101, mir-10b, mir-130a, mir-141, mir-143, mir-146b, mir-15a, mir-183, mir-196b, |
| | mir-200a, mir-203, mir-205, mir-21, mir-210, mir-221, mir-27a, mir-28-3p, mir-31, |
| | mir-452, mir-96, mir-99b |
| bladder cancer | mir-103a-3p, mir-10b, mir-135a, mir-137, mir-141, mir-155, mir-17-5p, mir-182, mir- |
| | 182-5p, mir-183, mir-185, mir-19a, mir-203, mir-205, mir-210, mir-221, mir-222, mir- |
| | 223, mir-23a, mir-23b, mir-26b, mir-639, mir-96 |
| endometrial cancer | mir-106a, mir-145, mir-155, mir-182, mir-200b, mir-200c, mir-205, mir-21, mir-222- |
| | 3p, mir-25, mir-93 |
| ovarian cancer | mir-106a, mir-141, mir-148b, mir-181b, mir-182, mir-200a, mir-200c, mir-205, mir- |
| | 20a, mir-21, mir-210, mir-214, mir-221, mir-224-5p, mir-23b, mir-25, mir-26a, mir- |
| | 27a, mir-27b, mir-346, mir-378, mir-424, mir-503, mir-572, mir-9, mir-96 |
| glioma | mir-106b, mir-106b-5p, mir-10b, mir-125b, mir-132, mir-155, mir-17, mir-181a, mir- |
| | 182, mir-183, mir-193b, mir-19a, mir-19b, mir-20a, mir-210, mir-214, mir-221, mir- |
| | 222, mir-224, mir-23a, mir-24, mir-24-3p, mir-25, mir-26a, mir-27a-3p, mir-27b, mir- |
| | 30a-5p, mir-30e, mir-30e*, mir-328, mir-335, mir-33a, mir-372, mir-486, mir-494, |
| | mir-497, mir-566, mir-603, mir-650, mir-675, mir-9, mir-92b, mir-93, mir-96 |
| head and neck | mir-106b, mir-134, mir-16, mir-184, mir-196a, mir-21, mir-25, mir-30a-5p, mir-31, |
| squamous cell | mir-372, mir-93 |
| carcinoma | |
| hepatocellular | mir-106b, mir-10b, mir-122, mir-1228, mir-1269, mir-128a, mir-130a, mir-130b, mir- |
| carcinoma | 146a, mir-153, mir-155, mir-17-5p, mir-181a, mir-181a-1, mir-181a-2, mir-181b, mir- |
| | 181b-1, mir-181b-2, mir-181c, mir-181d, mir-182, mir-183, mir-184, mir-190b, mir- |
| | 191, mir-20a, mir-20b, mir-21, mir-210, mir-214, mir-215, mir-216a, mir-217, mir- |
| | 221, mir-222, mir-223, mir-224, mir-23a, mir-24, mir-25, mir-27a, mir-301a, mir-30d, |
| | mir-31, mir-3127, mir-32, mir-331-3p, mir-362-3p, mir-362-5p, mir-371-5p, mir-372, |
| | mir-373, mir-423, mir-429, mir-452, mir-483-3p, mir-483-5p, mir-485-3p, mir-490- |
| | 3p, mir-494, mir-495, mir-500, mir-501, mir-501-5p, mir-519d, mir-520g, mir-574-3p, |
| | mir-590-5p, mir-630, mir-650, mir-657, mir-664, mir-885-5p, mir-9, mir-92a, mir-96 |
| laryngeal carcinoma | mir-106b, mir-16, mir-21, mir-27a, mir-423-3p |
| medulloblastoma | mir-106b, mir-17, mir-18a, mir-19a, mir-19b, mir-20a, mir-30b, mir-30d, mir-92 |
| pituitary carcinoma | mir-106b, mir-122, mir-17-5p, mir-20a, mir-493 |
| cervical cancer | mir-10a, mir-155, mir-181a, mir-181b, mir-196a, mir-19a, mir-19b, mir-205, mir-20a, |
| | mir-21, mir-215, mir-224, mir-31, mir-494, mir-590-5p, mir-92a, mir-944 |
| pancreatic cancer | mir-10a, mir-10b, mir-132, mir-15a, mir-17-5p, mir-181a, mir-18a, mir-191, mir-196a, |
| | mir-21, mir-212, mir-214, mir-221, mir-222, mir-27a, mir-301a, mir-301a-3p, mir- |
| | 367, mir-424-5p, mir-7, mir-92, mir-99a |
| breast cancer | mir-10b, mir-125a, mir-135a, mir-140, mir-141, mir-142, mir-150, mir-155, mir-17, |
| | mir-17-5p, mir-181a, mir-181b, mir-182, mir-18a, mir-18b, mir-191, mir-196a, mir- |
| | 197, mir-19a, mir-19b, mir-200a, mir-200b, mir-200c, mir-203, mir-205, mir-20a, mir- |
| | 20b, mir-21, mir-217, mir-221, mir-222, mir-224, mir-23a, mir-24, mir-24-2-5p, mir- |
| | 24-3p, mir-27a, mir-29a, mir-29b-1, mir-29b-2, mir-29c, mir-373, mir-378, mir-423, |
| | mir-429, mir-495, mir-503, mir-510, mir-520c, mir-526b, mir-96 |
| glioblastoma | mir-10b, mir-125b, mir-127-3p, mir-148a, mir-18a, mir-196a, mir-196a-1, mir-196a-2, |
| | mir-196b, mir-21, mir-210, mir-210-3p, mir-223, mir-340, mir-576-5p, mir-626, mir- |
| | 92b |
| lung cancer | mir-10b, mir-135b, mir-150, mir-155, mir-17, mir-182, mir-183-3p, mir-18a, mir-197, |
| | mir-19a, mir-19b, mir-205, mir-20a, mir-21, mir-210, mir-24, mir-30d, mir-4423, mir- |
| | 5100, mir-570, mir-663, mir-7, mir-92a |
| nasopharyngeal | mir-10b, mir-144, mir-149, mir-155, mir-18a, mir-21, mir-214, mir-24, mir-421, mir- |
| carcinoma | 663, mir-744, mir-93 |
| non-small cell lung | mir-10b, mir-125a-5p, mir-1280, mir-136, mir-140, mir-141, mir-142-3p, mir-145, |
| cancer | mir-146a, mir-150, mir-18a, mir-196a, mir-19a, mir-200a, mir-200c, mir-205, mir- |
| | 205-3p, mir-205-5p, mir-21, mir-212, mir-22, mir-221, mir-222, mir-24, mir-25, mir- |
| | 29c, mir-31, mir-328, mir-330-3p, mir-339, mir-34a, mir-375, mir-494, mir-675-5p, |
| | mir-9, mir-92b, mir-93, mir-95 |
| oral cancer | mir-10b, mir-196a-1, mir-196a-2, mir-196b, mir-21 |
| pancreatic ductal | mir-10b, mir-186, mir-18a, mir-192, mir-194, mir-196a, mir-198, mir-203, mir-21, |
| adenocarcinoma | mir-212, mir-30b-5p, mir-31, mir-34a, mir-369-5p, mir-376a, mir-541 |
| renal clear cell | mir-122, mir-155, mir-210, mir-630 |
| carcinoma | |
| mantle cell lymphoma | mir-124a, mir-155, mir-17, mir-18a, mir-19a, mir-19b, mir-20a, mir-92a |
| acute myeloid | mir-125b, mir-126-5p, mir-128, mir-155, mir-29a, mir-32, mir-331, mir-370, mir-378 |
| leukemia | |
| follicular cancer | mir-125b |
| neuroblastoma | mir-125b, mir-15a, mir-15b, mir-16-1, mir-16-2, mir-18a, mir-195, mir-19a, mir-23a, |
| | mir-421, mir-92 |
| oral squamous cell | mir-125b, mir-126, mir-146a, mir-146b, mir-155, mir-181b, mir-196a-1, mir-196a-2, |
| carcinoma | mir-196b, mir-21, mir-221, mir-222, mir-24, mir-27b, mir-31, mir-345 |
| prostate cancer | mir-125b, mir-141, mir-153, mir-155, mir-181a-1, mir-181a-2, mir-181b, mir-181b-1, |
| | mir-181b-2, mir-181c, mir-181d, mir-182, mir-182-5p, mir-183, mir-18a, mir-204, |
| | mir-20a, mir-21, mir-221, mir-223-3p, mir-31, mir-429, mir-96 |
| mesenchymal cancer | mir-125b-1-3p, mir-182 |
| malignant melanoma | mir-126, mir-141, mir-15b, mir-17, mir-17-5p, mir-182, mir-18a, mir-193b, mir-200a, |
| | mir-200b, mir-200c, mir-20a, mir-21, mir-210, mir-214, mir-221, mir-222, mir-429, |
| | mir-455-5p, mir-532-5p, mir-638, mir-92a |
| acute lymphoblastic | mir-128 |
| leukemia | |
| osteosarcoma | mir-128, mir-151-3p, mir-17, mir-181a, mir-181b, mir-181c, mir-18a, mir-191, mir- |
| | 195-5p, mir-199a-3p, mir-19a, mir-19b, mir-20a, mir-21, mir-210, mir-214, mir-221, |
| | mir-27a, mir-300, mir-320a, mir-374a-5p, mir-802, mir-9, mir-92a |
| colon cancer | mir-1290, mir-145, mir-155, mir-181a, mir-18a, mir-200c, mir-31, mir-675 |
| liver cancer | mir-1301, mir-155, mir-21, mir-221, mir-27a, mir-525-3p |
| cervical carcinoma | mir-133b, mir-21, mir-25, mir-373 |
| squamous carcinoma | mir-137, mir-155, mir-184, mir-196a, mir-203, mir-21, mir-221, mir-27a, mir-34a |
| chordoma | mir-140-3p, mir-148a |
| clear cell renal cell | mir-142-5p, mir-155, mir-21-5p |
| cancer | |
| malt lymphoma | mir-142-5p, mir-155 |
| anaplastic thyroid | mir-146b, mir-221, mir-222 |
| carcinoma | |
| follicular thyroid | mir-146b, mir-183, mir-197, mir-221, mir-346 |
| carcinoma | |
| primary thyroid | mir-146b |
| lymphoma | |
| ovarian carcinoma | mir-148b, mir-182 |
| adult t-cell leukemia | mir-150 |
| chronic lymphocytic | mir-150, mir-155 |
| leukemia | |
| lung adenocarcinoma | mir-150, mir-155, mir-31 |
| anaplastic large-cell | mir-155 |
| lymphoma | |
| cutaneous t-cell | mir-155 |
| lymphoma | |
| diffuse large B-cell | mir-155, mir-21 |
| lymphoma | |
| gallbladder carcinoma | mir-155, mir-182 |
| rectal cancer | mir-155, mir-200c, mir-21-5p, mir-34a |
| b-cell lymphoma | mir-17, mir-18a, mir-19a, mir-19b, mir-20a, mir-92a |
| breast carcinoma | mir-17, mir-18a, mir-19a, mir-19b, mir-20a, mir-24, mir-92a |
| cholangiocarcinoma | mir-17, mir-18a, mir-19a, mir-19b, mir-20a, mir-21, mir-26a, mir-92a |
| colorectal carcinoma | mir-17, mir-182, mir-191, mir-21, mir-95 |
| nasopharyngeal cancer | mir-17, mir-20a |
| acute promyelocytic | mir-181a, mir-181b, mir-92a |
| leukemia | |
| retinoblastoma | mir-181b, mir-21 |
| colorectal | mir-182 |
| adenocarcinoma | |
| kidney cancer | mir-183, mir-21, mir-210, mir-223 |
| medullary thyroid | mir-183 |
| carcinoma | |
| esophageal | mir-196a, mir-199a-3p, mir-199a-5p, mir-199b-3p, mir-200a, mir-223 |
| adenocarcinoma | |
| gastrointestinal stromal | mir-196a |
| tumor | |
| hypopharyngeal cancer | mir-203 |
| pancreatic | mir-203, mir-301a |
| adenocarcinoma | |
| cervical squamous cell | mir-205 |
| carcinoma | |
| adrenal cortical | mir-21, mir-210, mir-483-3p, mir-483-5p |
| carcinoma | |
| head and neck cancer | mir-21 |
| hypopharyngeal | mir-21 |
| squamous cell | |
| carcinoma | |
| laryngeal squamous | mir-21, mir-9, mir-93 |
| cell carcinoma | |
| multiple myeloma | mir-21 |
| colon carcinoma | mir-221, mir-23a |
| thyroid carcinoma | mir-221, mir-222 |
| meningioma | mir-224, mir-335 |
| gastric | mir-23a, mir-27a, mir-373 |
| adenocarcinoma | |
| laryngeal cancer | mir-23a |
| small cell lung cancer | mir-25 |
| pancreatic carcinoma | mir-301b |
| oral carcinoma | mir-31 |
| astrocytoma | mir-335 |
| epithelial ovarian | mir-372, mir-373 |
| cancer | |
| chronic myelogenous | mir-424, mir-96 |
| leukemia | |
| hepatoblastoma | mir-492 |

TABLE 14
Tumor suppressor miRs that are downregulated in specific cancer types

| Cancer | miRNA |
|---|---|
| Bladder | mir-1; mir-101; mir-1180; mir-1236; mir-124-3p; mir-125b; mir-126; mir- |
| | 1280; mir-133a; mir-133b; mir-141; mir-143; mir-144; mir-145; mir-155; |
| | mir-16; mir-18a; mir-192; mir-195; mir-200a; mir-200b; mir-200c; mir-203; |
| | mir-205; mir-214; mir-218; mir-23b; mir-26a; mir-29c; mir-320c; mir-34a; |
| | mir-370; mir-409-3p; mir-429; mir-451; mir-490-5p; mir-493; mir-576-3p; |
| | mir-99a |
| Brain | let-7g-5p; mir-100; mir-101; mir-106a; mir-124; mir-124a; mir-125a; mir- |
| (Astrocytoma, | 125a-5p; mir-125b; mir-127-3p; mir-128; mir-129; mir-136; mir-137; mir- |
| Glioblastoma, | 139-5p; mir-142-3p; mir-143; mir-145; mir-146b-5p; mir-149; mir-152; |
| Glioma) | mir-153; mir-195; mir-21; mir-212-3p; mir-219-5p; mir-222; mir-29b; mir- |
| | 31; mir-3189-3p; mir-320; mir-320a; mir-326; mir-330; mir-331-3p; mir- |
| | 340; mir-342; mir-34a; mir-376a; mir-449a; mir-483-5p; mir-503; mir-577; |
| | mir-663; mir-7; mir-7-5p; mir-873; let-7a; let-7f; mir-107; mir-122; mir- |
| | 124-5p; mir-139; mir-146a; mir-146b; mir-15b; mir-16; mir-181a; mir- |
| | 181a-1; mir-181a-2; mir-181b; mir-181b-1; mir-181b-2; mir-181c; mir- |
| | 181d; mir-184; mir-185; mir-199a-3p; mir-200a; mir-200b; mir-203; mir- |
| | 204; mir-205; mir-218; mir-23b; mir-26b; mir-27a; mir-29c; mir-328; mir- |
| | 34c-3p; mir-34c-5p; mir-375; mir-383; mir-451; mir-452; mir-495; mir-584; |
| | mir-622; mir-656; mir-98; mir-124-3p; mir-181b-5p; mir-200b; mir-3189-3p |
| Breast | mir-193b; let-7a; let-7a-1; let-7a-2; let-7a-3; let-7b; let-7c; let-7d; let-7e; let- |
| | 7f-1; let-7f-2; let-7g; let-7i; mir-100; mir-107; mir-10a; mir-10b; mir-122; |
| | mir-124; mir-1258; mir-125a-5p; mir-125b; mir-126; mir-127; mir-129; |
| | mir-130a; mir-132; mir-133a; mir-143; mir-145; mir-146a; mir-146b; mir- |
| | 147; mir-148a; mir-149; mir-152; mir-153; mir-15a; mir-16; mir-17-5p; mir- |
| | 181a; mir-1826; mir-183; mir-185; mir-191; mir-193a-3p; mir-195; mir- |
| | 199b-5p; mir-19a-3p; mir-200a; mir-200b; mir-200c; mir-205; mir-206; |
| | mir-211; mir-216b; mir-218; mir-22; mir-26a; mir-26b; mir-300; mir-30a; |
| | mir-31; mir-335; mir-339-5p; mir-33b; mir-34a; mir-34b; mir-34c; mir- |
| | 374a; mir-379; mir-381; mir-383; mir-425; mir-429; mir-450b-3p; mir-494; |
| | mir-495; mir-497; mir-502-5p; mir-517a; mir-574-3p; mir-638; mir-7; mir- |
| | 720; mir-873; mir-874; mir-92a; mir-98; mir-99a; mmu-mir-290-3p; mmu- |
| | mir-290-5p |
| Cervical | mir-143; mir-145; mir-17-5p; mir-203; mir-214; mir-218; mir-335; mir-342- |
| | 3p; mir-372; mir-424; mir-491-5p; mir-497; mir-7; mir-99a; mir-99b; mir- |
| | 100; mir-101; mir-15a; mir-16; mir-34a; mir-886-5p; mir-106a; mir-124; |
| | mir-148a; mir-29a; mir-375 |
| Colon/Colorectal | let-7a-1; let-7a-2; let-7a-3; let-7b; let-7c; let-7d; let-7e; let-7f-1; let-7f-2; let- |
| | 7g; let-7i; mir-100; mir-101; mir-126; mir-142-3p; mir-143; mir-145; mir- |
| | 192; mir-200c; mir-21; mir-214; mir-215; mir-22; mir-25; mir-302a; mir- |
| | 320; mir-320a; mir-34a; mir-34c; mir-365; mir-373; mir-424; mir-429; mir- |
| | 455; mir-484; mir-502; mir-503; mir-93; mir-98; mir-186; mir-30a-5p; mir- |
| | 627; let-7a; mir-1; mir-124; mir-125a; mir-129; mir-1295b-3p; mir-1307; |
| | mir-130b; mir-132; mir-133a; mir-133b; mir-137; mir-138; mir-139; mir- |
| | 139-5p; mir-140-5p; mir-148a; mir-148b; mir-149; mir-150-5p; mir-154; |
| | mir-15a; mir-15b; mir-16; mir-18a; mir-191; mir-193a-5p; mir-194; mir- |
| | 195; mir-196a; mir-198; mir-199a-5p; mir-203; mir-204-5p; mir-206; mir- |
| | 212; mir-218; mir-224; mir-24-3p; mir-26b; mir-27a; mir-28-3p; mir-28-5p; |
| | mir-29b; mir-30a-3p; mir-30b; mir-328; mir-338-3p; mir-342; mir-345; mir- |
| | 34a-5p; mir-361-5p; mir-375; mir-378; mir-378a-3p; mir-378a-5p; mir-409- |
| | 3p; mir-422a; mir-4487; mir-483; mir-497; mir-498; mir-518a-3p; mir-551a; |
| | mir-574-5p; mir-625; mir-638; mir-7; mir-96-5p; mir-202-3p; mir-30a; mir- |
| | 451 |
| Endometrial | mir-101; mir-130a; mir-130b; mir-134; mir-143; mir-145; mir-152; mir-205; |
| | mir-223; mir-301a; mir-301b; mir-30c; mir-34a; mir-34c; mir-424; mir- |
| | 449a; mir-543; mir-34b |
| Hematologic | mir-125b; mir-138; mir-15a; mir-15b; mir-16; mir-16-1; mir-16-1-3p; mir- |
| (Leukemia, | 16-2; mir-181a; mir-181b; mir-195; mir-223; mir-29b; mir-34b; mir-34c; |
| Lymphoma, | mir-424; mir-10a; mir-146a; mir-150; mir-151; mir-155; mir-2278; mir-26a; |
| Myeloma) | mir-30e; mir-31; mir-326; mir-564; mir-27a; let-7b; mir-124a; mir-142-3p; |
| | let-7c; mir-17; mir-20a; mir-29a; mir-30c; mir-720; mir-107; mir-342; mir- |
| | 34a; mir-202; mir-142-5p; mir-29c; mir-145; mir-193b; mir-199a; mir-214; |
| | mir-22; mir-137; mir-197 |
| Kidney | mir-1; mir-145; mir-1826; mir-199a; mir-199a-3p; mir-203; mir-205; mir- |
| | 497; mir-508-3p; mir-509-3p; let-7a; let-7d; mir-106a *; mir-126; mir-1285; |
| | mir-129-3p; mir-1291; mir-133a; mir-135a; mir-138; mir-141; mir-143; mir- |
| | 182-5p; mir-200a; mir-218; mir-28-5p; mir-30a; mir-30c; mir-30d; mir-34a; |
| | mir-378; mir-429; mir-509-5p; mir-646; mir-133b; let-7b; let-7c; mir-200c; |
| | mir-204; mir-335; mir-377; mir-506 |
| Liver | mir-137; mir-138; mir-139; mir-139-5p; mir-140-5p; mir-141; mir-142-3p; |
| (Hepatocellular | mir-143; mir-144; mir-145; mir-146a; mir-148a; mir-148b; mir-150-5p; mir- |
| Carcinoma) | 15b; mir-16; mir-181a-5p; mir-185; mir-188-5p; mir-193b; mir-195; mir- |
| | 195-5p; mir-197; mir-198; mir-199a; mir-199a-5p; mir-199b; mir-199b-5p; |
| | mir-200a; mir-200b; mir-200c; mir-202; mir-203; mir-204-3p; mir-205; mir- |
| | 206; mir-20a; mir-21; mir-21-3p; mir-211; mir-212; mir-214; mir-217; mir- |
| | 218; mir-219-5p; mir-22; mir-223; mir-26a; mir-26b; mir-29a; mir-29b-1; |
| | mir-29b-2; mir-29c; mir-302b; mir-302c; mir-30a; mir-30a-3p; mir-335; |
| | mir-338-3p; mir-33a; mir-34a; mir-34b; mir-365; mir-370; mir-372; mir- |
| | 375; mir-376a; mir-377; mir-422a; mir-424; mir-424-5p; mir-433; mir- |
| | 4458; mir-448; mir-450a; mir-451; mir-485-5p; mir-486-5p; mir-497; mir- |
| | 503; mir-506; mir-519d; mir-520a; mir-520b; mir-520c-3p; mir-582-5p; |
| | mir-590-5p; mir-610; mir-612; mir-625; mir-637; mir-675; mir-7; mir-877; |
| | mir-940; mir-941; mir-98; mir-99a; mir-132; mir-31 |
| Lung | mir-1297; mir-141; mir-145; mir-16; mir-200a; mir-200b; mir-200c; mir- |
| | 29b; mir-381; mir-409-3p; mir-429; mir-451; mir-511; mir-99a; let-7a-1; |
| | let-7a-2; let-7a-3; let-7b; let-7c; let-7d; let-7e; let-7f-1; let-7f-2; let-7g; let- |
| | 7i; mir-1; mir-101; mir-133b; mir-138; mir-142-5p; mir-144; mir-1469; mir- |
| | 146a; mir-153; mir-15a; mir-15b; mir-16-1; mir-16-2; mir-182; mir-192; |
| | mir-193a-3p; mir-194; mir-195; mir-198; mir-203; mir-217; mir-218; mir- |
| | 22; mir-223; mir-26a; mir-26b; mir-29c; mir-33a; mir-34a; mir-34b; mir- |
| | 34c; mir-365; mir-449a; mir-449b; mir-486-5p; mir-545; mir-610; mir-614; |
| | mir-630; mir-660; mir-7515; mir-9500; mir-98; mir-99b; mir-133a; let-7a; |
| | mir-100; mir-106a; mir-107; mir-124; mir-125a-3p; mir-125a-5p; mir-126; |
| | mir-126 *; mir-129; mir-137; mir-140; mir-143; mir-146b; mir-148a; mir- |
| | 148b; mir-149; mir-152; mir-154; mir-155; mir-17-5p; mir-181a-1; mir- |
| | 181a-2; mir-181b; mir-181b-1; mir-181b-2; mir-181c; mir-181d; mir-184; |
| | mir-186; mir-193b; mir-199a; mir-204; mir-212; mir-221; mir-224; mir-27a; |
| | mir-27b; mir-29a; mir-30a; mir-30b; mir-30c; mir-30d; mir-30d-5p; mir- |
| | 30e-5p; mir-32; mir-335; mir-338-3p; mir-340; mir-342-3p; mir-361-3p; |
| | mir-373; mir-375; mir-4500; mir-4782-3p; mir-497; mir-503; mir-512-3p; |
| | mir-520a-3p; mir-526b; mir-625 *; mir-96 |
| Melanoma | let-7b; mir-101; mir-125b; mir-1280; mir-143; mir-146a; mir-146b; mir- |
| | 155; mir-17; mir-184; mir-185; mir-18b; mir-193b; mir-200c; mir-203; mir- |
| | 204; mir-205; mir-206; mir-20a; mir-211; mir-218; mir-26a; mir-31; mir- |
| | 33a; mir-34a; mir-34c; mir-376a; mir-376c; mir-573; mir-7-5p; mir-9; mir- |
| | 98 |
| Oral Cancer | let-7d; mir-218; mir-34a; mir-375; mir-494; mir-100; mir-124; mir-1250; |
| | mir-125b; mir-126; mir-1271; mir-136; mir-138; mir-145; mir-147; mir- |
| | 148a; mir-181a; mir-206; mir-220a; mir-26a; mir-26b; mir-29a; mir-32; mir- |
| | 323-5p; mir-329; mir-338; mir-370; mir-410; mir-429; mir-433; mir-499a- |
| | 5p; mir-503; mir-506; mir-632; mir-646; mir-668; mir-877; mir-9 |
| Ovarian | let-7i; mir-100; mir-124; mir-125b; mir-129-5p; mir-130b; mir-133a; mir- |
| | 137; mir-138; mir-141; mir-145; mir-148a; mir-152; mir-153; mir-155; mir- |
| | 199a; mir-200a; mir-200b; mir-200c; mir-212; mir-335; mir-34a; mir-34b; |
| | mir-34c; mir-409-3p; mir-411; mir-429; mir-432; mir-449a; mir-494; mir- |
| | 497; mir-498; mir-519d; mir-655; mir-9; mir-98; mir-101; mir-532-5p; mir- |
| | 124a; mir-192; mir-193a; mir-7 |
| Pancreatic | mir-101; mir-1181; mir-124; mir-1247; mir-133a; mir-141; mir-145; mir- |
| | 146a; mir-148a; mir-148b; mir-150 *; mir-150-5p; mir-152; mir-15a; mir- |
| | 198; mir-203; mir-214; mir-216a; mir-29c; mir-335; mir-34a; mir-34b; mir- |
| | 34c; mir-373; mir-375; mir-410; mir-497; mir-615-5p; mir-630; mir-96; |
| | mir-132; let-7a; let-7a-1; let-7a-2; let-7a-3; let-7b; let-7c; let-7d; let-7e; let- |
| | 7f-1; let-7f-2; let-7g; let-7i; mir-126; mir-135a; mir-143; mir-144; mir-150; |
| | mir-16; mir-200a; mir-200b; mir-200c; mir-217; mir-218; mir-337; mir-494; |
| | mir-98 |
| Prostate | let-7a-3p; let-7c; mir-100; mir-101; mir-105; mir-124; mir-128; mir-1296; |
| | mir-130b; mir-133a-1; mir-133a-2; mir-133b; mir-135a; mir-143; mir-145; |
| | mir-146a; mir-154; mir-15a; mir-187; mir-188-5p; mir-199b; mir-200b; mir- |
| | 203; mir-205; mir-212; mir-218; mir-221; mir-224; mir-23a; mir-23b; mir- |
| | 25; mir-26a; mir-26b; mir-29b; mir-302a; mir-30a; mir-30b; mir-30c-1; mir- |
| | 30c-2; mir-30d; mir-30e; mir-31; mir-330; mir-331-3p; mir-34a; mir-34b; |
| | mir-34c; mir-374b; mir-449a; mir-4723-5p; mir-497; mir-628-5p; mir-642a- |
| | 5p; mir-765; mir-940 |
| Retinoblastoma | mir-101; mir-183; mir-204; mir-34a; mir-365b-3p; mir-486-3p; mir-532-5p |

1. A recombinant herpesvirus, wherein the viral genome of the recombinant herpesvirus:
(a) comprises one or more transgenes, optionally wherein the open reading frame(s) (ORFs) of the transgene(s) have a G/C content of at least 60%, at least 61%, at least 62%, at least 63%, or at least 64%;
(b) comprises one or more miRNA target sequences;
(c) comprises a polynucleotide encoding a retargeting domain;
(d) comprises a UL30 viral gene encoding a DNA polymerase catalytic subunit (DPCS) and a UL23 viral gene encoding a thymidine kinase (TK), wherein the DPCS comprises a mutation, and wherein the TK comprises a mutation;
(e) encodes a first gB and/or a second gB, wherein the first gB comprises a syncytial mutation, and wherein the second gB comprises no syncytial mutation;
(f) encodes a first gK and/or a second gK, wherein the first gK comprises a syncytial mutation, and wherein the second gK comprises no syncytial mutation;
(g) encodes a first gH and/or a second gH, wherein the first gH comprises a syncytial mutation, and wherein the second gH comprises no syncytial mutation;
(h) encodes a first UL20 and/or a second UL20, wherein the first UL20 comprises a syncytial mutation, and wherein the second UL20 comprises no syncytial mutation;
and/or
(i) encodes a first UL24 and/or a second UL24, wherein the first UL24 comprises a syncytial mutation, and wherein the second UL24 comprises no syncytial mutation.

2. The recombinant herpesvirus of claim 1, wherein the viral genome of the recombinant herpesvirus comprises the one or more transgenes, wherein the one or more transgenes encode one or more payload proteins selected from 15-hydroxyprostaglandin dehydrogenase [NAD(+)] (HPGD), adenosine deaminase 2 (ADA2), hyaluronidase-1 (HYAL1), hemotaxis inhibitory protein (CHP), C-C motif chemokine 21 (CCL21), interleukin-12 (IL-12), a CD47 antagonist, a transforming growth factor beta (TGFβ) antagonist, a programmed death-1 (PD1) antagonist, a triggering receptor expressed on myeloid cells-2 (TREM2) antagonist, a biomolecule comprising chlorotoxin (CTX), or any combinations thereof.

3. The recombinant herpesvirus of claim 2, wherein the one or more payload proteins comprise or consist of IL-12, a PD1 antagonist, and a TREM2 antagonist.

4. The recombinant herpesvirus of claim 3, wherein the one or more payload proteins comprise HPGD.

5. The recombinant herpesvirus of claim 3 or 4, wherein the one or more payload proteins comprise a biomolecule comprising CTX.

6. The recombinant herpesvirus of claim 2, wherein the one or more payload proteins comprise or consist of one of the combinations of payload proteins listed in Tables 4-7.

7. The recombinant herpesvirus of any one of claims 2-6, wherein the one or more payload proteins comprise HPGD.

8. The recombinant herpesvirus of any one of claims 2-7, wherein the one or more payload proteins comprise ADA2.

9. The recombinant herpesvirus of any one of claims 2-8, wherein the one or more payload proteins comprise HYAL1.

10. The recombinant herpesvirus of any one of claims 2-9, wherein the one or more payload proteins comprise CHP.

11. The recombinant herpesvirus of any one of claims 2-10, wherein the one or more payload proteins comprise CCL21.

12. The recombinant herpesvirus of any one of claims 2-11, wherein the one or more payload proteins comprise IL-12.

13. The recombinant herpesvirus of any one of claims 2-12, wherein the one or more payload proteins comprise the CD47 antagonist.

14. The recombinant herpesvirus of any one of claims 2-13, wherein the one or more payload proteins comprise the TGFβ antagonist.

15. The recombinant herpesvirus of any one of claims 2-14, wherein the one or more payload proteins comprise the PD1 antagonist.

16. The recombinant herpesvirus of any one of claims 2-15, wherein the one or more payload proteins comprise the TREM2 antagonist.

17. The recombinant herpesvirus of any one of claims 2-16, wherein the antagonist comprises an antibody or antigen binding fragment thereof.

18. The recombinant herpesvirus of any one of claims 2-17, wherein the one or more payload proteins comprise the biomolecule comprising CTX.

19. The recombinant herpesvirus of claim 5 or 18, wherein the biomolecule comprising CTX further comprises a T-cell engager moiety specifically binding to a protein expressed on the surface of the T-cell.

20. The recombinant herpesvirus of claim 19, wherein the protein expressed on the surface of the T-cell is CD3.

21. The recombinant herpesvirus of claim 20, wherein the T-cell engager moiety comprises an amino acid sequence at least 90%, at least 95%, at least 97%, at least 98%, at least 99%, or 100% identical to SEQ ID NO: 914.

22. The recombinant herpesvirus of any one of claims 18-21, wherein the CTX comprises or consists of an amino acid sequence at least 95% identical to SEQ ID NO: 913.

23. The recombinant herpesvirus of any one of claims 2-22, wherein:
(i) the HPGD comprises an amino acid sequence at least 90%, at least 95%, at least 97%, at least 98%, at least 99%, or 100% identical to SEQ ID NO: 875;
(ii) the ADA2 comprises an amino acid sequence at least 90%, at least 95%, at least 97%, at least 98%, at least 99%, or 100% identical to SEQ ID NO: 877;
(iii) the HYAL1 comprises an amino acid sequence at least 90%, at least 95%, at least 97%, at least 98%, at least 99%, or 100% identical to SEQ ID NO: 878;
(iv) the CHP comprises an amino acid sequence at least 90%, at least 95%, at least 97%, at least 98%, at least 99%, or 100% identical to SEQ ID NO: 880;
(v) the CCL21 comprises an amino acid sequence at least 90%, at least 95%, at least 97%, at least 98%, at least 99%, or 100% identical to SEQ ID NO: 881;
(vi) the IL-12 comprises a subunit alpha comprising an amino acid sequence at least 90%, at least 95%, at least 97%, at least 98%, at least 99%, or 100% identical to SEQ ID NO: 883 and a subunit beta comprising an amino acid sequence at least 90%, at least 95%, at least 97%, at least 98%, at least 99%, or 100% identical to SEQ ID NO: 884;
(vii) the CD47 antagonist comprises VHH CDR1 of SEQ ID NO: 895, VHH CDR2 of SEQ ID NO: 896, VHH CDR3 of SEQ ID NO: 897, and/or an amino acid sequence at least 90%, at least 95%, at least 97%, at least 98%, at least 99%, or 100% identical to SEQ ID NO: 887 or 888;
(viii) the TGFβ antagonist comprises a heavy chain variable domain (VH) comprising CDR1 of SEQ ID NO: 898, CDR2 of SEQ ID NO: 899, and CDR3 of SEQ ID NO: 900, and/or a light chain variable domain (VL) comprising CDR1 of SEQ ID NO: 901, CDR2 of SEQ ID NO: 902, and CDR3 of SEQ ID NO: 903; optionally wherein the TGFβ antagonist comprises an amino acid sequence at least 90%, at least 95%, at least 97%, at least 98%, at least 99%, or 100% identical to SEQ ID NO: 889 or 890;
(ix) the PD1 antagonist comprises VHH CDR1 of SEQ ID NO: 904, VHH CDR2 of SEQ ID NO: 905, VHH CDR3 of SEQ ID NO: 906, and/or an amino acid sequence at least 90%, at least 95%, at least 97%, at least 98%, at least 99%, or 100% identical to SEQ ID NO: 891 or 892; and/or
(x) the biomolecule comprising CTX comprises an amino acid sequence at least 90%, at least 95%, at least 97%, at least 98%, at least 99%, or 100% identical to SEQ ID NO: 915 or 916.

24. The recombinant herpesvirus of any one of claims 1-23, wherein the ORF of at least one of the transgene(s) has the G/C content of at least 60%, at least 61%, at least 62%, at least 63%, or at least 64%.

25. The recombinant herpesvirus of claim 24, wherein the ORFs of all of the transgene(s) have the G/C content of at least 60%, at least 61%, at least 62%, at least 63%, or at least 64%.

26. The recombinant herpesvirus of claim 24, wherein the ORFs of the transgene(s) encoding IL-12, the PD1 antagonist, the TREM2 antagonist, HPGD, and/or the biomolecule comprising CTX have the G/C content of at least 60%, at least 61%, at least 62%, at least 63%, or at least 64%.

27. The recombinant herpesvirus of any one of claims 24-26, wherein the expression of a payload protein encoded by the ORF of the transgene is at least 2-fold, at least 3-fold, at least 5-fold, at least 8-fold, or at least 10-fold higher than the expression of the payload protein encoded by a control ORF having a G/C content of about 52% in a control recombinant herpesvirus; optionally wherein the control ORF is codon optimized based on the codon usage of Homo sapiens.

28. The recombinant herpesvirus of any one of claims 24-27, wherein the ORF(s) of the transgene(s) are codon optimized based on the codon usage of Anaeromyxobacter dehalogenans.

29. The recombinant herpesvirus of any one of claims 24-28, wherein the transgene(s) encode an antibody or antigen binding fragment thereof.

30. The recombinant herpesvirus of claim 29, wherein the antibody or antigen binding fragment thereof comprises a heavy chain variable domain (VH) and a light chain variable domain (VL).

31. The recombinant herpesvirus of claim 29 or 30, wherein the antibody or antigen binding fragment thereof comprises a VHH domain derived from a single domain antibody (sdAb).

32. The recombinant herpesvirus of claim 30 or 31, wherein the antibody or antigen binding fragment thereof comprises an IgG-Fc, optionally wherein the IgG is IgG1.

33. The recombinant herpesvirus of any one of claims 23 and 29-32, wherein the transgene encoding the PD1 antagonist comprises a polynucleotide sequence at least 90%, at least 95%, at least 97%, at least 98%, at least 99%, or 100% identical to SEQ ID NO: 937.

34. The recombinant herpesvirus of any one of claims 23 and 29-33, wherein the transgene encoding the biomolecule comprising CTX comprises a polynucleotide sequence at least 90%, at least 95%, at least 97%, at least 98%, at least 99%, or 100% identical to SEQ ID NO: 940 or 941.

35. The recombinant herpesvirus of any one of claims 24-34, comprising the transgene(s) encoding a cytokine, a chemokine, a receptor, a receptor ligand, an enzyme, and/or a reporter protein.

36. The recombinant herpesvirus of claim 23 or 35, wherein the transgene encoding IL-12 comprise a polynucleotide sequence at least 90%, at least 95%, at least 97%, at least 98%, at least 99%, or 100% identical to SEQ ID NO: 936.

37. The recombinant herpesvirus of any one of claims 23 and 35-36, wherein the transgene encoding HPGD comprises a polynucleotide sequence at least 90%, at least 95%, at least 97%, at least 98%, at least 99%, or 100% identical to SEQ ID NO: 939.

38. The recombinant herpesvirus of any one of claims 1-37, comprising the miRNA target sequences for miR-34b-5p, miR-34b-3p, miR-34c-5p, miR-122-5p, miR-124-3p, miR-128T, miR-129-5p, miR-129-2-3p, miR-132-3p, miR-137-3p, miR-145-5p, or any combination thereof.

39. The recombinant herpesvirus of claim 38, comprising the miRNA target sequences for miR-34b-5p, miR-34c-5p, miR-122-5p, miR-124-3p, miR-128T, miR-129-2-3p, miR-132-3p, miR-137-3p, miR-145-5p, or any combination thereof.

40. The recombinant herpesvirus of claim 38, comprising the miRNA target sequences for miR-34b-5p, miR-34c-5p, miR-122-5p, miR-124-3p, miR-128T, miR-129-2-3p, miR-132-3p, miR-137-3p, and miR-145-5p.

41. The recombinant herpesvirus of any one of claims 38-40, comprising:
(a) one or more miRNA target sequences for miR-122-5p;
(b) one or more miRNA target sequences for miR-145-5p;
(c) one or more miRNA target sequences for miR-124-3p, miR-128T, miR-137-3p, or any combination thereof;
(d) one or more miRNA target sequences for miR-34b-5p, miR-34c-5p, or any combination thereof; and
(e) one or more miRNA target sequences for miR-129-2-3p, miR-132-3p, or any combination thereof.

42. The recombinant herpesvirus of any one of claims 38-41, comprising a first miR-TS cassette inserted into a first viral gene, wherein the first miR-TS cassette comprises one or more miRNA target sequences for each of miR-34c-5p, miR-124-3p, miR-129-2-3p, and miR-132-3p.

43. The recombinant herpesvirus of claim 42, wherein the miRNA target sequences in the first miR-TS cassette are arranged as (34c-5p)-(124-3p)-(132-3p)-(129-2-3p)-(34c-5p)-(124-3p)-(129-2-3p)-(132-3p)-(124-3p)-(129-2-3p)-(132-3p)-(34c-5p).

44. The recombinant herpesvirus of claim 42 or 43, wherein the first miR-TS cassette comprises a nucleic acid sequence at least 90%, at least 95%, at least 96%, at least 97%, at least 98%, at least 99%, or 100% identical to SEQ ID NO: 859.

45. The recombinant herpesvirus of any one of claims 42-44, wherein the first viral gene is ICP8.

46. The recombinant herpesvirus of any one of claims 38-45, comprising a second miR-TS cassette inserted into a second viral gene, wherein the second miR-TS cassette comprises one or more miRNA target sequences for each of miR-122-5p, miR-124-3p, miR-128T, and miR-137-3p.

47. The recombinant herpesvirus of claim 46, wherein the miRNA target sequences in the second miR-TS cassette are arranged as (137-3p)-(128T)-(122-5p)-(124-3p)-(122-5p)-(128T)-(137-3p)-(124-3p)-(128T)-(137-3p)-(124-3p)-(122-5p).

48. The recombinant herpesvirus of claim 46 or 47, wherein the second miR-TS cassette comprises a nucleic acid sequence at least 90%, at least 95%, at least 96%, at least 97%, at least 98%, at least 99%, or 100% identical to SEQ ID NO: 858.

49. The recombinant herpesvirus of any one of claims 46-48, wherein the second viral gene is ICP4.

50. The recombinant herpesvirus of claim 49, comprising the second miR-TS cassette in both ICP4 viral genes of the viral genome.

51. The recombinant herpesvirus of any one of claims 1-50, comprising one or more miRNA target sequences in both ICP4 viral genes of the viral genome; optionally wherein the miRNA target sequences are the same in both said ICP4 viral genes.

52. The recombinant herpesvirus of any one of claims 38-51, comprising a third miR-TS cassette inserted into a third viral gene, wherein the third miR-TS cassette comprises one or more miRNA target sequences for each of miR-34c-5p, miR-124-3p, miR-128T, and miR-137-3p.

53. The recombinant herpesvirus of claim 52, wherein the miRNA target sequences in the third miR-TS cassette are arranged as (124-3p)-(128T)-(34c-5p)-(137-3p)-(128T)-(34c-5p)-(137-3p)-(124-3p)-(128T)-(137-3p)-(124-3p)-(34c-5p).

54. The recombinant herpesvirus of claim 52 or 53, wherein the third miR-TS cassette comprises a nucleic acid sequence at least 90%, at least 95%, at least 96%, at least 97%, at least 98%, at least 99%, or 100% identical to SEQ ID NO: 873.

55. The recombinant herpesvirus of any one of claims 38-51, comprising a third miR-TS cassette inserted into a third viral gene, wherein the third miR-TS cassette comprises one or more miRNA target sequences for each of miR-34b-3p, miR-34c-5p, miR-128T, miR-137-3p.

56. The recombinant herpesvirus of any one of claims 52-55, wherein the third viral gene is ICP27.

57. The recombinant herpesvirus of any one of claims 38-56, comprising a fourth miR-TS cassette inserted into a fourth viral gene, wherein:
(i) the fourth miR-TS cassette comprises one or more miRNA target sequences for each of miR-34b-5p, miR-34c-5p, miR-129-5p, and miR-145-5p;
(ii) the fourth miR-TS cassette comprises one or more miRNA target sequences for each of miR-34b-5p, miR-34c-5p, miR-132-3p, and miR-145-5p; or
(iii) the fourth miR-TS cassette comprises one or more miRNA target sequences for each of miR-34b-3p, miR-34c-5p, miR-132-3p, and miR-145-5p.

58. The recombinant herpesvirus of any one of claims 38-56, comprising a fourth miR-TS cassette inserted into a fourth viral gene, wherein the fourth miR-TS cassette comprises one or more miRNA target sequences for each of miR-34b-5p, miR-34c-5p, miR-132-3p, and miR-145-5p.

59. The recombinant herpesvirus of claim 58, wherein the miRNA target sequences in the fourth miR-TS cassette are arranged as (145-5p)-(34b-5p)-(132-3p)-(34c-5p)-(145-5p)-(34c-5p)-(34b-5p)-(132-3p)-(34b-5p)-(145-5p)-(132-3p)-(34c-5p).

60. The recombinant herpesvirus of claim 58 or 59, wherein the fourth miR-TS cassette comprises a nucleic acid sequence at least 90%, at least 95%, at least 96%, at least 97%, at least 98%, at least 99%, or 100% identical to SEQ ID NO: 874.

61. The recombinant herpesvirus of any one of claims 57-60, wherein the fourth viral gene is UL8.

62. The recombinant herpesvirus of any one of claims 42-61, wherein each of the miR-TS cassettes comprises at least 2, at least 3, or at least 4 copies of each of the miRNA target sequences.

63. The recombinant herpesvirus of any one of claims 42-61, wherein each of the miR-TS cassettes comprises 3 copies of each of the miRNA target sequences.

64. The recombinant herpesvirus of any one of claims 38-63, wherein the replication of the recombinant HSV is reduced in a non-cancerous cell compared to the replication of the recombinant HSV in a cancerous cell; optionally wherein the cancerous cell is a glioblastoma cell.

65. The recombinant herpesvirus of claim 64, wherein the non-cancerous cell is selected from the group consisting of a neuron, an ependymal cell, an oligodendrocyte, an endothelial cell, a hepatocyte, an astrocyte, and any combination thereof.

66. The recombinant herpesvirus of claim 64, wherein the non-cancerous cell is an astrocyte.

67. The recombinant herpesvirus of any one of claims 38-66, wherein:
(a) the one or more miRNA target sequences for miR-34b-5p comprise or consist of SEQ ID NO: 867;
(b) the one or more miRNA target sequences for miR-34b-3p comprise or consist of SEQ ID NO: 868;
(c) the one or more miRNA target sequences for miR-34c-5p comprise or consist of SEQ ID NO: 869;
(d) the one or more miRNA target sequences for miR-122-5p comprise or consist of SEQ ID NO: 804;
(e) the one or more miRNA target sequences for miR-124-3p comprise or consist of SEQ ID NO: 805;
(f) the one or more miRNA target sequences for miR-128T comprise or consist of SEQ ID NO: 870;
(g) the one or more miRNA target sequences for miR-129-5p comprise or consist of SEQ ID NO: 813;
(h) the one or more miRNA target sequences for miR-129-2-3p comprise or consist of SEQ ID NO: 871;
(i) the one or more miRNA target sequences for miR-132-3p comprise or consist of SEQ ID NO: 872;
(j) the one or more miRNA target sequences for miR-137-3p comprise or consist of SEQ ID NO: 819; and/or
(k) the one or more miRNA target sequences for miR-145-5p comprise or consist of SEQ ID NO: 823.

68. The recombinant herpesvirus of any one of claims 1-67, comprising the polynucleotide encoding the retargeting domain, wherein the retargeting domain specifically binds a target protein expressed by a target cell.

69. The recombinant herpesvirus of claim 68, wherein the polynucleotide encoding the retargeting domain is inserted into the open reading frame of a US6 gene encoding a glycoprotein D (gD).

70. The recombinant herpesvirus of claim 69, wherein the polynucleotide encoding the retargeting domain replaces the US6 gene region encoding an amino acid sequence corresponding to amino acids 6-24 of SEQ ID NO: 921.

71. The recombinant herpesvirus of any one of claims 68-70, wherein the target protein expressed by the target cell comprises integrin α5β1, integrin αvβ1, integrin αvβ3, integrin αvβ6, or a combination thereof.

72. The recombinant herpesvirus of any one of claims 68-71, wherein the target protein expressed by the target cell comprises epidermal growth factor receptor (EGFR).

73. The recombinant herpesvirus of any one of claims 68-72, wherein the retargeting domain comprises a knottin peptide capable of specifically binding to the target protein expressed by the target cell.

74. The recombinant herpesvirus of claim 73, wherein the retargeting domain comprises no more than 50, no more than 45, no more than 40, or no more than 35 amino acids.

75. The recombinant herpesvirus of claim 73 or 74, wherein the retargeting domain comprises an amino acid sequence having at least 80%, at least 85%, at least 90%, or 100% identity to SEQ ID NO: 922.

76. The recombinant herpesvirus of any one of claims 68-75, wherein the retargeting domain comprises an immunoglobulin domain capable of specifically binding to the target protein expressed by the target cell.

77. The recombinant herpesvirus of any one of claims 68-76, wherein the retargeting domain comprises a binding domain of, or a binding domain derived from, a variable domain of a heavy chain-only antibody (VHH) or a variable domain of new antigen receptor immunoglobulin (V-NAR).

78. The recombinant herpesvirus of claim 76 or 77, wherein the retargeting domain comprises no more than 150, no more than 140, or no more than 130 amino acids.

79. The recombinant herpesvirus of any one of claims 76-78, wherein the retargeting domain comprises an amino acid sequence having at least 80%, at least 85%, at least 90%, or 100% identity to SEQ ID NO: 923.

80. The recombinant herpesvirus of any one of claims 68-79, wherein the herpesvirus is capable of infecting the target cell expressing the target protein.

81. The recombinant herpesvirus of any one of claims 68-80, wherein the herpesvirus is capable of infecting cells without Nectin-1 expression; optionally the cells are Vero cells.

82. The recombinant herpesvirus of any one of claims 1-81, comprising the UL30 viral gene encoding the DPCS comprising the mutation and the UL23 viral gene encoding the TK comprising the mutation.

83. The recombinant herpesvirus of claim 82, wherein the mutation in the DPCS increases DNA replication fidelity of the herpesvirus by at least 10%, at least 20%, at least 30%, at least 50%, at least 100%, at least 2-fold, at least 3-fold, or at least 5-fold.

84. The recombinant herpesvirus of claim 82 or 83, wherein the mutation in the DPCS is at an amino acid position corresponding to L774 of SEQ ID NO: 917; preferably, the mutation is an amino acid substitution.

85. The recombinant herpesvirus of claim 84, wherein the mutation in the DPCS is the amino acid substitution corresponding to L774F of SEQ ID NO: 917.

86. The recombinant herpesvirus of any one of claims 82-85, wherein the DPCS comprises an amino acid sequence at least 90%, at least 95%, at least 97%, at least 98%, at least 99%, or 100% identical to SEQ ID NO: 917, except for the mutation in the DPCS.

87. The recombinant herpesvirus of any one of claims 82-86, wherein the IC50 of acyclovir is less than 0.5 ug/ml, less than 1.0 ug/ml, less than 1.5 ug/ml, or less than 2.0 ug/ml for the herpesvirus.

88. The recombinant herpesvirus of any one of claims 82-87, wherein the mutation in the TK decreases the IC50 of acyclovir for the herpesvirus by at least 1-fold, at least 2-fold, at least 5-fold, at least 10-fold, at least 20-fold, at least 50-fold, or at least 100-fold.

89. The recombinant herpesvirus of any one of claims 82-88, wherein the mutation in the TK is at one or more amino acid positions corresponding to L159, 1160, F161, A168 and/or L169 of SEQ ID NO: 918; preferably, the mutation is amino acid substitution.

90. The recombinant herpesvirus of claim 89, wherein the mutation in the TK comprises one or more amino acid substitutions of:
(a) L159I or L159L;
(b) I160L or I160F;
(c) F161A, F161V, F161P, or F161L;
(d) A168D, A168Y, A168V or A168F; and/or
(e) L169F, L169Y, L169L, L169I, L169M, L169N, or L169K.

91. The recombinant herpesvirus of any one of claims 82-90, wherein the mutation in the TK comprises amino acid substitutions corresponding to L159I, 1160F, F161L, A168F and L169M of SEQ ID NO: 918.

92. The recombinant herpesvirus of any one of claims 82-90, wherein the mutation in the TK comprises amino acid substitutions corresponding to 1160F, F161A, and A168F of SEQ ID NO: 918.

93. The recombinant herpesvirus of any one of claims 82-90, wherein the mutation in the TK comprises amino acid substitutions corresponding to I160F, F161L, A168F, and L169N of SEQ ID NO: 918.

94. The recombinant herpesvirus of any one of claims 82-93, wherein the TK comprises an amino acid sequence at least 90%, at least 95%, at least 97%, at least 98%, at least 99%, or 100% identical to SEQ ID NO: 918, except the mutation in the TK.

95. The recombinant herpesvirus of any one of claims 1-94, wherein the viral genome of the herpesvirus encodes:
(i) the first gB and/or the second gB, wherein the first gB comprises the syncytial mutation, and wherein the second gB comprises no syncytial mutation;
(ii) the first gK and/or the second gK, wherein the first gK comprises the syncytial mutation, and wherein the second gK comprises no syncytial mutation;
(iii) the first gH and/or the second gH, wherein the first gH comprises the syncytial mutation, and wherein the second gH comprises no syncytial mutation;
(iv) the first UL20 and/or the second UL20, wherein the first UL20 comprises the syncytial mutation, and wherein the second UL20 comprises no syncytial mutation;
and/or
(v) the first UL24 and/or the second UL24, wherein the first UL24 comprises the syncytial mutation, and wherein the second UL24 comprises no syncytial mutation.

96. The recombinant herpesvirus of claim 95, wherein the first gB is encoded by an endogenous gB-encoding gene locus and the second gB is encoded by an exogenous expression cassette.

97. The recombinant herpesvirus of claim 95, wherein the first gB is encoded by an exogenous expression cassette and the second gB is encoded by an endogenous gB-encoding gene locus.

98. The recombinant herpesvirus of any one of claims 95-97, wherein the first gK is encoded by an endogenous gK-encoding gene locus and the second gK is encoded by an exogenous expression cassette.

99. The recombinant herpesvirus of any one of claims 95-97, wherein the first gK is encoded by an exogenous expression cassette and the second gK is encoded by an endogenous gK-encoding gene locus.

100. The recombinant herpesvirus of any one of claims 95-99, wherein the first gH is encoded by an endogenous gH-encoding gene locus and the second gH is encoded by an exogenous expression cassette.

101. The recombinant herpesvirus of any one of claims 95-99, wherein the first gH is encoded by an exogenous expression cassette and the second gH is encoded by an endogenous gH-encoding gene locus.

102. The recombinant herpesvirus of any one of claims 95-101, wherein the first UL20 is encoded by an endogenous UL20 gene locus and the second UL20 is encoded by an exogenous expression cassette.

103. The recombinant herpesvirus of any one of claims 95-101, wherein the first UL20 is encoded by an exogenous expression cassette and the second UL20 is encoded by an endogenous UL20 gene locus.

104. The recombinant herpesvirus of any one of claims 95-103, wherein the first UL24 is encoded by an endogenous UL24 gene locus and the second UL24 is encoded by an exogenous expression cassette.

105. The recombinant herpesvirus of any one of claims 95-103, wherein the first UL24 is encoded by an exogenous expression cassette and the second UL24 is encoded by an endogenous UL24 gene locus.

106. The recombinant herpesvirus of any one of claims 95-105, wherein the viral genome of the herpesvirus encodes the first gB but not the second gB.

107. The recombinant herpesvirus of any one of claims 95-106, wherein the viral genome of the herpesvirus encodes the first gK but not the second gK.

108. The recombinant herpesvirus of any one of claims 95-107, wherein the viral genome of the herpesvirus encodes the first gH but not the second gH.

109. The recombinant herpesvirus of any one of claims 95-108, wherein the viral genome of the herpesvirus encodes the first UL20 but not the second UL20

110. The recombinant herpesvirus of any one of claims 95-109, wherein the viral genome of the herpesvirus encodes the first UL24 but not the second UL24.

111. The recombinant herpesvirus of any one of claims 95-110, wherein the viral genome of the herpesvirus encodes the first gB and the first gK; optionally, wherein the viral genome of the herpesvirus further encodes the first gH and the first UL24.

112. The recombinant herpesvirus of any one of claims 96-111, wherein the exogenous expression cassette is located at UL3-UL4 intergenic region.

113. The recombinant herpesvirus of any one of claims 96-111, wherein the exogenous expression cassette is located at UL50-UL51 intergenic region.

114. The recombinant herpesvirus of any one of claims 95-113, wherein the recombinant herpesvirus displays syncytial phenotype in cancer cells.

115. A cell, comprising a recombinant nucleic acid encoding the recombinant herpesvirus of any one of claims 95-114.

116. A cell, comprising a first nucleic acid encoding a recombinant herpesvirus and a second nucleic acid, wherein:
(i) the viral genome of the herpesvirus encodes a first gB, wherein the first gB comprises a syncytial mutation, the second nucleic acid encodes a second gB, wherein the second gB comprises no syncytial mutation; or, the viral genome of the herpesvirus encodes a second gB, wherein the second gB comprises no syncytial mutation, the second nucleic acid encodes a first gB, wherein the first gB comprises a syncytial mutation;
(ii) the viral genome of the herpesvirus encodes a first gK, wherein the first gK comprises a syncytial mutation, the second nucleic acid encodes a second gK, wherein the second gK comprises no syncytial mutation; or the viral genome of the herpesvirus encodes a second gK, wherein the second gK comprises no syncytial mutation, the second nucleic acid encodes a first gK, wherein the first gK comprises a syncytial mutation;
(iii) the viral genome of the herpesvirus encodes a first gH, wherein the first gH comprises a syncytial mutation, the second nucleic acid encodes a second gH, wherein the second gH comprises no syncytial mutation; or the viral genome of the herpesvirus encodes a second gH, wherein the second gH comprises no syncytial mutation, the second nucleic acid encodes a first gH, wherein the first gH comprises a syncytial mutation;
(iv) the viral genome of the herpesvirus encodes a first UL20, wherein the first UL20 comprises a syncytial mutation, the second nucleic acid encodes a second UL20, wherein the second UL20 comprises no syncytial mutation; or the viral genome of the herpesvirus encodes a second UL20, wherein the second UL20 comprises no syncytial mutation, the second nucleic acid encodes a first UL20, wherein the first UL20 comprises a syncytial mutation; and/or
(v) the viral genome of the herpesvirus encodes a first UL24, wherein the first UL24 comprises a syncytial mutation, the second nucleic acid encodes a second UL24, wherein the second UL24 comprises no syncytial mutation; or the viral genome of the herpesvirus encodes a second UL24, wherein the second UL24 comprises no syncytial mutation, the second nucleic acid encodes a first UL24, wherein the first UL24 comprises a syncytial mutation.

117. The cell of claim 116, wherein the viral genome of the herpesvirus encodes the first gB, wherein the first gB comprises the syncytial mutation, wherein the second nucleic acid encodes the second gB, wherein the second gB comprises no syncytial mutation.

118. The cell of claim 116, wherein the viral genome of the herpesvirus encodes the second gB, wherein the second gB comprises no syncytial mutation, wherein the second nucleic acid encodes the first gB, wherein the first gB comprises the syncytial mutation.

119. The cell of any one of claims 116-118, wherein the viral genome of the herpesvirus encodes the first gK, wherein the first gK comprises the syncytial mutation, wherein the second nucleic acid encodes the second gK, wherein the second gK comprises no syncytial mutation.

120. The cell of any one of claims 116-118, wherein the viral genome of the herpesvirus encodes the second gK, wherein the second gK comprises no syncytial mutation, wherein the second nucleic acid encodes the first gK, wherein the first gK comprises the syncytial mutation.

121. The cell of any one of claims 116-120, wherein the viral genome of the herpesvirus encodes the first gH, wherein the first gH comprises the syncytial mutation, wherein the second nucleic acid encodes the second gH, wherein the second gH comprises no syncytial mutation.

122. The cell of any one of claims 116-120, wherein the viral genome of the herpesvirus encodes the second gH, wherein the second gH comprises no syncytial mutation, wherein the second nucleic acid encodes the first gH, wherein the first gH comprises the syncytial mutation.

123. The cell of any one of claims 116-122, wherein the viral genome of the herpesvirus encodes the first UL20, wherein the first UL20 comprises the syncytial mutation, wherein the second nucleic acid encodes the second UL20, wherein the second UL20 comprises no syncytial mutation.

124. The cell of any one of claims 116-122, wherein the viral genome of the herpesvirus encodes the second UL20, wherein the second UL20 comprises no syncytial mutation, wherein the second nucleic acid encodes the first UL20, wherein the first UL20 comprises the syncytial mutation.

125. The cell of any one of claims 116-124, wherein the viral genome of the herpesvirus encodes the first UL24, wherein the first UL24 comprises the syncytial mutation, wherein the second nucleic acid encodes the second UL24, wherein the second UL24 comprises no syncytial mutation.

126. The cell of any one of claims 116-124, wherein the viral genome of the herpesvirus encodes the second UL24, wherein the second UL24 comprises no syncytial mutation, wherein the second nucleic acid encodes the first UL24, wherein the first UL24 comprises the syncytial mutation.

127. The cell of any one of claims 116-126, wherein the recombinant herpesvirus comprises a single copy of gB-encoding viral gene, a single copy of gK-encoding viral gene, a single copy of gH-encoding viral gene, a single copy of UL20 viral gene, and/or a single copy of UL24 viral gene.

128. The cell of any of claims 116-127, wherein the first nucleic acid and the second nucleic acid are comprised within a single polynucleotide molecule.

129. The cell of any of claims 116-127, wherein the first nucleic acid and the second nucleic acid are comprised within two different polynucleotide molecules.

130. The cell of any of claims 115-129, wherein the cell is a Vero cell.

131. The recombinant herpesvirus of any of claims 95-114 or the cell of any of claims 115-130, wherein the gB syncytial mutation comprises a mutation at one or more amino acid residues corresponding to R796, R800, T813, L817, S854, A855, R858, or A874, an insertion between E816 and L817, a deletion of S869 to C-terminus, a deletion of T877 to C-terminus, or a combination thereof, of SEQ ID NO: 919.

132. The recombinant herpesvirus of any of claims 95-114 or the cell of any of claims 115-130, wherein the gB syncytial mutation comprises one or more mutations corresponding to R796C, R800W, T813I, L817H, L817P, S854F, A855V, R858C, R858H, A874P, an insertion of VN or VNVN between E816 and L817, a deletion of S869 to C-terminus, or a deletion of T877 to C-terminus, of SEQ ID NO: 919.

133. The recombinant herpesvirus of any of claims 95-114 or the cell of any of claims 115-130, wherein the gB syncytial mutation comprises a deletion of T877 to C-terminus according to SEQ ID NO: 919.

134. The recombinant herpesvirus of any of claims 95-114 and 131-133, or the cell of any of claims 115-133, wherein the first and/or the second gB comprise a mutation corresponding to D285N and/or A549T of SEQ ID NO: 919.

135. The recombinant herpesvirus of any of claims 95-114 and 131-134, or the cell of any of claims 115-134, wherein the gK syncytial mutation comprises a mutation at one or more amino acid residues corresponding to P33, A40, L86, D99, A111, L118, T121, C243, L304, 1307, or R310 of SEQ ID NO: 920.

136. The recombinant herpesvirus of any of claims 95-114 and 131-134, or the cell of any of claims 115-134, wherein the gK syncytial mutation comprises one or more mutations corresponding to P33S, A40V, A40T, L86P, D99N, A111V, L118Q, T121I, C243Y, L304P, 1307N, or R310L of SEQ ID NO: 920.

137. The recombinant herpesvirus of any of claims 95-114 and 131-134, or the cell of any of claims 115-134, wherein the gK syncytial mutation comprises 1307N according to SEQ ID NO: 920.

138. The recombinant herpesvirus of any of claims 95-114 and 131-137, or the cell of any of claims 115-137, wherein the gH syncytial mutation comprises a mutation at one or more amino acid residues corresponding to N753 or A778 of SEQ ID NO: 943.

139. The recombinant herpesvirus of any of claims 95-114 and 131-137, or the cell of any of claims 115-137, wherein the gH syncytial mutation comprises one or more mutations corresponding to N753K or A778V of SEQ ID NO: 943.

140. The recombinant herpesvirus of any of claims 95-114 and 131-139, or the cell of any of claims 115-139, wherein the UL20 syncytial mutation comprises a mutation at one or more amino acid residues corresponding to Y49, S50, R51, R209, T212, R213, or C-terminal deletion after N217, of SEQ ID NO: 944.

141. The recombinant herpesvirus of any of claims 95-114 and 131-139, or the cell of any of claims 115-139, wherein the UL20 syncytial mutation comprises one or more mutations corresponding to Y49A, S50A, R51A, R209A, T212A, R213A, or C-terminal deletion after N217, of SEQ ID NO: 944.

142. The recombinant herpesvirus of any of claims 95-114 and 131-141, or the cell of any of claims 115-141, wherein the UL24 syncytial mutation comprises a mutation at one or more amino acid residues corresponding to T64, R63, or V64 of SEQ ID NO: 942.

143. The recombinant herpesvirus of any of claims 95-114 and 131-141, or the cell of any of claims 115-141, wherein the UL24 syncytial mutation comprises one or more mutations corresponding to T64G, R63V, or V64S of SEQ ID NO: 942.

144. The recombinant herpesvirus of any of claims 95-114 and 131-143, or the cell of any of claims 115-143, wherein the open reading frame encoding the first gB is operably linked to a CMV promoter and/or a bGH poly A tail.

145. The recombinant herpesvirus of any of claims 95-114 and 131-143, or the cell of any of claims 115-143, wherein the open reading frame encoding the second gB is operably linked to a CMV promoter and/or a bGH poly A tail.

146. The recombinant herpesvirus of any of claims 95-114 and 131-145, or the cell of any of claims 115-145, wherein the open reading frame encoding the first gK is operably linked to a CMV promoter and/or a bGH poly A tail.

147. The recombinant herpesvirus of any of claims 95-114 and 131-145, or the cell of any of claims 115-145, wherein the open reading frame encoding the second gK is operably linked to a CMV promoter and/or a bGH poly A tail.

148. The recombinant herpesvirus of any of claims 95-114 and 131-147, or the cell of any of claims 115-147, wherein the yield of the recombinant herpesvirus is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than the yield of a control herpesvirus or a control cell that does not encode the second gB, the second gK, the second gH, the second UL20, or the second UL24.

149. The recombinant herpesvirus of any of claims 95-114 and 131-148, or the cell of any of claims 115-148, wherein the gene encoding the first gB comprises a miRNA target sequence (miR-TS) cassette, wherein the miR-TS cassette comprises one or more target sequences of one or more miRNAs.

150. The recombinant herpesvirus of any of claims 95-114 and 131-149, or the cell of any of claims 115-149, wherein the gene encoding the first gK comprises a miRNA target sequence (miR-TS) cassette, wherein the miR-TS cassette comprises one or more target sequences of one or more miRNAs.

151. The recombinant herpesvirus of any of claims 95-114 and 131-150, or the cell of any of claims 115-150, wherein the gene encoding the first gH comprises a miRNA target sequence (miR-TS) cassette, wherein the miR-TS cassette comprises one or more target sequences of one or more miRNAs.

152. The recombinant herpesvirus of any of claims 95-114 and 131-151, or the cell of any of claims 115-151, wherein the gene encoding the first UL20 comprises a miRNA target sequence (miR-TS) cassette, wherein the miR-TS cassette comprises one or more target sequences of one or more miRNAs.

153. The recombinant herpesvirus of any of claims 95-114 and 131-152, or the cell of any of claims 115-152, wherein the gene encoding the first UL24 comprises a miRNA target sequence (miR-TS) cassette, wherein the miR-TS cassette comprises one or more target sequences of one or more miRNAs.

154. The recombinant herpesvirus of any one of claims 149-153, or the cell of any one of claims 149-153, wherein the one or more miRNAs comprise at least one of miR-34c-5p, miR-299-5p, and miR-582-5p.

155. The recombinant herpesvirus of any one of claims 149-153, or the cell of any one of claims 149-153, wherein the one or more miRNAs comprise at least two of miR-34c-5p, miR-299-5p, and miR-582-5p.

156. The recombinant herpesvirus of any one of claims 149-153, or the cell of any one of claims 149-153, wherein the one or more miRNAs comprise miR-34c-5p, miR-299-5p, and miR-582-5p.

157. The recombinant herpesvirus or the cell of any of claims 149-156, wherein the miR-TS cassette comprises at least three copies, or at least four copies of the target sequences of each of the miRNA separated by a 4 bp spacer.

158. The recombinant herpesvirus or the cell of any of claims 149-157, wherein the miR-TS cassette is located at the 3′UTR of the gene.

159. The recombinant herpesvirus or the cell of any of claims 149-158, wherein the target sequence of the miRNA comprises or consists of the reverse complement of the miRNA.

160. The recombinant herpesvirus or the cell of any of claims 149-159, wherein the miR-TS cassette comprises the polynucleotide sequence of SEQ ID NO: 930.

161. The recombinant herpesvirus or the cell of any of claims 149-160, wherein the yield of the recombinant herpesvirus is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than the yield of a control herpesvirus or a control cell that does not comprise the miR-TS cassette.

162. A recombinant herpesvirus produced by culturing the cell of any of claims 115-161 and recovering the recombinant herpesvirus from the cell culture.

163. A recombinant herpesvirus, wherein the viral genome of the herpesvirus encodes a gK comprising a syncytial mutation corresponding to 1307N of SEQ ID NO: 920.

164. The recombinant herpesvirus of any one of claims 1-114 and 131-163, or the cell of any of claims 115-161, wherein the herpesvirus is an alphaherpesvirus.

165. The recombinant herpesvirus or the cell of claim 164, wherein the alphaherpesvirus is a herpes simplex virus.

166. The recombinant herpesvirus or the cell of claim 165, wherein the herpes simplex virus is a herpes simplex virus-1 (HSV-1).

167. The recombinant herpesvirus of any one of claims 1-114 and 131-166, or the cell of any of claims 115-161 and 164-166, wherein the recombinant herpesvirus is oncolytic.

168. The recombinant herpesvirus or the cell of any one of claims 164-167, wherein the recombinant herpesvirus is derived from an encephalitic HSV isolate according to SEQ ID NO: 857; optionally wherein the recombinant herpesvirus comprises a polynucleotide sequence having at least 80%, at least 85%, at least 90%, at least 95%, at least 96%, at least 97%, at least 98%, at least 99%, at least 99.5%, or 100% identity to SEQ ID NO: 857.

169. The recombinant herpesvirus or the cell of any one of claims 164-168, wherein the recombinant herpesvirus is defective for anterograde transport.

170. The recombinant herpesvirus of any one of claims 1-114 and 131-169, comprising a mutation in the UL37 viral gene.

171. The recombinant herpesvirus of claim 170, wherein the UL37 viral gene encodes a UL37 protein comprising a mutation at at least 1, at least 2, at least 3, at least 4, or all 5 amino acid positions corresponding to Q403, E452, Q455, Q511, and R515 of SEQ ID NO: 856.

172. The recombinant herpesvirus of claim 171, where the mutation in the UL37 viral gene comprises Q403A, E452A, Q455A, Q511A, and R515A according to SEQ ID NO: 856.

173. The recombinant herpesvirus of any one of claims 1-114 and 131-172, encoding a gB comprising the mutations corresponding to A549T/D285N of SEQ ID NO: 919.

174. The recombinant herpesvirus of any one of claims 1-114 and 131-173, wherein the recombinant herpesvirus retains the function of ICP6, ICP34.5, and/or ICP47.

175. The recombinant herpesvirus of any one of claims 1-114 and 131-174, wherein the one or more transgenes are inserted in the UL50-UL51 intergenic region.

176. A recombinant virus comprising one or more transgenes encoding one or more payload proteins selected from HPGD, ADA2, HYAL1, CHP, CCL21, IL-12, a CD47 antagonist, a TGFβ antagonist, a PD1 antagonist, a TREM2 antagonist, a biomolecule comprising chlorotoxin (CTX), or any combinations thereof.

177. The recombinant virus of claim 176, wherein the one or more payload proteins comprise or consist of IL-12, a PD1 antagonist, and a TREM2 antagonist.

178. The recombinant virus of claim 177, wherein the one or more payload proteins comprise HPGD.

179. The recombinant virus of claim 177 or 178, wherein the one or more payload proteins comprise a biomolecule comprising CTX.

180. The recombinant virus of claim 176, wherein the one or more payload proteins comprise or consist of one of the combinations of payload proteins listed in Tables 4-7.

181. The recombinant virus of any one of claims 176-180, wherein the one or more payload proteins comprise HPGD.

182. The recombinant virus of any one of claims 176-181, wherein the one or more payload proteins comprise ADA2.

183. The recombinant virus of any one of claims 176-182, wherein the one or more payload proteins comprise HYAL1.

184. The recombinant virus of any one of claims 176-183, wherein the one or more payload proteins comprise CHP.

185. The recombinant virus of any one of claims 176-184, wherein the one or more payload proteins comprise CCL21.

186. The recombinant virus of any one of claims 176-185, wherein the one or more payload proteins comprise IL-12.

187. The recombinant virus of any one of claims 176-186, wherein the one or more payload proteins comprise the CD47 antagonist.

188. The recombinant virus of any one of claims 176-187, wherein the one or more payload proteins comprise the TGFβ antagonist.

189. The recombinant virus of any one of claims 176-188, wherein the one or more payload proteins comprise the PD1 antagonist.

190. The recombinant virus of any one of claims 176-189, wherein the one or more payload proteins comprise the TREM2 antagonist.

191. The recombinant virus of any one of claims 176-190, wherein the antagonist comprises an antibody or antigen binding fragment thereof.

192. The recombinant virus of any one of claims 176-191, wherein the one or more payload proteins comprise the biomolecule comprising CTX.

193. The recombinant virus of claim 179 or 192, wherein the biomolecule comprising CTX further comprises a T-cell engager moiety specifically binding to a protein expressed on the surface of the T-cell.

194. The recombinant virus of claim 193, wherein the protein expressed on the surface of the T-cell is CD3.

195. A recombinant virus comprising:
(a) one or more miRNA target sequences for miR-122-5p;
(b) one or more miRNA target sequences for miR-145-5p;
(c) one or more miRNA target sequences for miR-124-3p, miR-128T, miR-137-3p, or any combination thereof;
(d) one or more miRNA target sequences for miR-34b-5p, miR-34c-5p, or any combination thereof; and
(e) one or more miRNA target sequences for miR-129-2-3p, miR-132-3p, or any combination thereof.

196. The recombinant virus of claim 195, comprising the miRNA target sequences for miR-34b-5p, miR-34c-5p, miR-122-5p, miR-124-3p, miR-128T, miR-129-2-3p, miR-132-3p, miR-137-3p, and miR-145-5p.

197. A recombinant virus, wherein the viral genome of the recombinant virus encodes a protein comprising a syncytial mutation and a counterpart protein without the syncytial mutation.

198. The recombinant virus of claim 197, wherein the protein comprising the syncytial mutation and the counterpart protein without the syncytial mutation share at least 90%, at least 95%, at least 96%, at least 97%, at least 98%, at least 99%, or 100% sequence identity, except the syncytial mutation.

199. The recombinant virus of claim 197 or 198, wherein the protein comprising the syncytial mutation is encoded by an endogenous viral gene and the counterpart protein without the syncytial mutation is encoded by an exogenous expression cassette.

200. The recombinant virus of claim 197 or 198, wherein the protein comprising the syncytial mutation is encoded by an exogenous expression cassette and the counterpart protein without the syncytial mutation is encoded by an endogenous viral gene.

201. The recombinant virus of claim 197 or 198, wherein both the protein comprising the syncytial mutation and the counterpart protein without the syncytial mutation are encoded by one exogenous expression cassette or by different exogenous expression cassettes.

202. A cell, comprising a recombinant nucleic acid encoding the recombinant virus of any one of claims 197-201.

203. A cell, comprising a first nucleic acid encoding a recombinant virus and a second nucleic acid,
wherein the viral genome of the recombinant virus encodes a protein comprising a syncytial mutation,
wherein the second nucleic acid encodes a a counterpart protein without the syncytial mutation.

204. The cell of claim 203, wherein the protein comprising the syncytial mutation and the counterpart protein without the syncytial mutation share at least 90%, at least 95%, at least 96%, at least 97%, at least 98%, at least 99%, or 100% sequence identity, except the syncytial mutation.

205. The recombinant virus of any one of claims 197-201 or the cell of any one of claims 202-204, wherein the yield of the virus is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than the yield of a control virus or a control cell that does not encodes the counterpart protein without the syncytial mutation.

206. The recombinant virus of any one of claims 197-201 and 205, or the cell of any one of claims 202-205, wherein the gene encoding the protein comprising the syncytial mutation comprises a miRNA target sequence (miR-TS) cassette, wherein the miR-TS cassette comprises one or more target sequences of one or more miRNAs.

207. The recombinant virus or the cell of claim 206, wherein the one or more miRNAs comprise at least one, at least two, or all of miRNAs selected from miR-34c-5p, miR-299-5p, and miR-582-5p.

208. The recombinant virus or the cell of claim 206 or 207, wherein the yield of the recombinant virus is at least 1-fold, at least 2-fold, at least 3-fold, at least 5-fold, at least 8-fold, at least 10-fold, at least 20-fold, at least 50-fold, at least 100-fold, at least 200-fold, at least 500-fold, or at least 1000-fold higher than the yield of a control virus or a control cell that does not comprise the miR-TS cassette.

209. The recombinant virus produced by culturing the cell of any of claims 202-208 and recovering the recombinant herpesvirus from the cell culture.

210. The recombinant virus of any one of claims 176-201 and 205-209, or the cell of any of claims 202-208, wherein the recombinant virus is derived from a herpes simplex virus, an adenovirus, a polio virus, a vaccinia virus, a measles virus, a vesicular stomatitis virus, an orthomyxovirus, a parvovirus, a maraba virus, or a coxsackievirus.

211. The recombinant virus of any one of claims 176-201 and 205-210, or the cell of any of claims 202-208, wherein the recombinant virus is oncolytic.

212. A nucleic acid molecule encoding the recombinant herpesvirus of any one of claims 1-114 and 131-175, or the recombinant virus of any one of claims 176-201 and 205-211.

213. The nucleic acid molecule of claim 212, wherein the nucleic acid molecule is DNA.

214. The nucleic acid molecule of claim 212, wherein the nucleic acid molecule is RNA.

215. A viral stock comprising the recombinant herpesvirus of any one of claims 1-114 and 131-175, or the recombinant virus of any one of claims 176-201 and 205-211.

216. A particle comprising the nucleic acid molecule of any one of claims 212-214.

217. The particle of claim 216, wherein the particle is selected from the group consisting of a nanoparticle, an exosome, a liposome, and a lipoplex.

218. The particle of claim 216, wherein the particle is a lipid nanoparticle.

219. The particle of any one of claims 216-218, wherein contacting a eukaryotic cell with the particle results in production of infectious virus particles by the eukaryotic cell.

220. A pharmaceutical composition comprising:
(i) the recombinant herpesvirus of any one of claims 1-114 and 131-175, the recombinant virus of any one of claims 176-201 and 205-211, the nucleic acid molecule of any one of claims 212-214, or the particle of any one of claims 216-219; and
(ii) a pharmaceutically-acceptable carrier.

221. A method of killing a cancerous cell, comprising exposing the cancerous cell to the recombinant herpesvirus of any one of claims 1-114 and 131-175, the recombinant virus of any one of claims 176-201 and 205-211, the particle of any one of claims 216-219, or the pharmaceutical composition of claim 220, under conditions sufficient for the virus or particle to infect and the virus to replicate within said cancerous cell, and wherein replication of the virus within the cancerous cell results in cell death.

222. The method of claim 221, wherein the cell is in vitro or in vivo.

223. The method of claim 221 or 222, wherein the cancerous cell has a reduced expression of a miRNA capable of binding to the one or more miRNA target sequences compared to the expression of the miRNA in a non-cancerous cell.

224. The method of any one of claims 221-223, wherein replication of the virus is increased or maintained in the cancerous cell with a reduced expression of the miR capable of binding to the one or more miRNA target sequences.

225. The method of any one of claims 221-224, wherein the cancerous cell is a cell of lung cancer, breast cancer, ovarian cancer, cervical cancer, prostate cancer, testicular cancer, colorectal cancer, colon cancer, pancreatic cancer, liver cancer, gastric cancer, head and neck cancer, thyroid cancer, malignant glioma, glioblastoma, melanoma, non-melanoma skin cancer, B-cell chronic lymphocytic leukemia, diffuse large B-cell lymphoma (DLBCL), or marginal zone lymphoma (MZL).

226. The method of any one of claims 221-224, wherein the cancerous cell is a glioblastoma cell.

227. A method of treating cancer in a subject in need thereof, comprising administering the recombinant herpesvirus of any one of claims 1-114 and 131-175, the recombinant virus of any one of claims 176-201 and 205-211, the particle of any one of claims 216-219, or the pharmaceutical composition of claim 220 to the subject.

228. The method of claim 227, wherein the virus, the particle, or the composition is administered intravenously, subcutaneously, intratumorally, intramuscularly, or intranasally.

229. The method of claim 227, wherein the virus, the particle, or the composition is administered intratumorally.

230. The method of claim 227, wherein the virus, the particle, or the composition is administered intravenously.

231. The method of any one of claims 227-230, wherein the virus, the particle, or the composition is administered only once.

232. The method of any one of claims 227-231, wherein the cancer is selected from the group consisting of lung cancer, breast cancer, ovarian cancer, cervical cancer, prostate cancer, testicular cancer, colorectal cancer, colon cancer, pancreatic cancer, liver cancer, gastric cancer, head and neck cancer, thyroid cancer, malignant glioma, glioblastoma, melanoma, non-melanoma skin cancer, B-cell chronic lymphocytic leukemia, diffuse large B-cell lymphoma (DLBCL), and marginal zone lymphoma (MZL).

233. The method of any one of claims 227-231, wherein the cancer is glioblastoma.

234. A cell line, comprising the cell of any of claims 115-160 and 202-208.

235. A method of producing a recombinant herpesvirus, comprising culturing the cell of any of claims 115-160 and 202-208, or the cell line of claim 234, and recovering the recombinant herpesvirus from the cell culture.

236. The recombinant herpesvirus of any one of claims 82-114 and 131-175 for use in combination with a small molecule for imaging the infection site of the herpesvirus.

237. A method of imaging the infection site of an herpesvirus in vivo, comprising administering the recombinant herpesvirus of any one of claims 82-114 and 131-175 and a small molecule.

238. The recombinant virus for use of claim 236 or the method of claim 237, wherein the small molecule is radioisotope labeled acyclovir; optionally wherein the radioisotope label comprises fluorine-18 (¹⁸F) label.
